(19) United States
(12) Patent Application Publication
(10) Pub. No.: US 2022/0306715 A1
(43) Pub. Date: Sep. 29, 2022

(54) CHIMERIC PROTEINS IN AUTOIMMUNITY

(71) Applicant: Shattuck Labs, Inc., Austin, TX (US)

(72) Inventors: Taylor SCHREIBER, Austin, TX (US); George FROMM, Austin, TX (US); Suresh DE SILVA, Austin, TX (US); Louis GONZALEZ, Austin, TX (US)

(21) Appl. No.: 17/638,925

(22) PCT Filed: Aug. 28, 2020
(86) PCT No.: PCT/US20/48600

§ 371 Date: Feb. 28, 2022

Related U.S. Application Data
(60) Provisional application No. 62/894,481, filed on Aug. 30, 2019.

(51) Int. Cl. C07K 14/55; C07K 14/705; C07K 14/715; C07K 14/54; C07K 14/71; A61P 37/06; A61K 45/06; A61K 38/17; A61K 38/20; C07K 14/53; A61K 38/19; C07K 14/525

(52) U.S. Cl. CPC C07K 14/55; C07K 14/70521; C07K 14/70532; C07K 14/7155; C07K 14/5434; C07K 14/7151; C07K 14/71; C07K 14/54; C07K 14/70503; C07K 14/70546; A61P 37/06; A61K 45/06; A61K 38/1774; A61K 38/1793; A61K 38/2013; A61K 38/208; A61K 38/20; C07K 14/53; A61K 38/193; A61K 38/191; C07K 14/525; C07K 2319/03; C07K 2317/53; C07K 2317/524; C07K 2317/526; C07K 2319/30; C07K 2317/21; A61K 2039/545

(57) ABSTRACT

The present invention relates, inter alia, to compositions and methods, including chimeric proteins having a first domain comprising an extracellular domain of a first transmembrane protein, a first secreted protein, or a first membrane-anchored extracellular protein and a second domain comprising an extracellular domain of a second transmembrane protein, a second secreted protein, or a second membrane-anchored extracellular protein, in which either or both of the first domain and the second domain decreases self-directed immune system activity when bound to its ligand/receptor. Accordingly, the present invention find use in the treatment of autoimmune diseases.

### PRIORITY

This application claims the benefit of, and priority to, US Application Nos. 62/894,481, filed Aug. 30, 2019, which is hereby incorporated by reference in its entirety.

### TECHNICAL FIELD

The present invention relates to, inter alia, compositions and methods, including chimeric proteins that find use in the treatment of disease, such as in immunotherapies for treating autoimmunity.

### DESCRIPTION OF THE TEXT FILE SUBMITTED ELECTRONICALLY

This application contains a sequence listing. It has been submitted electronically via EFS-Web as an ASCII text file entitled “SHK-018PC_SequenceListing_ST25”. The sequence listing is 243,589 bytes in size, and was prepared on or about Aug. 30, 2020. The sequence listing is hereby incorporated by reference in its entirety.

### BACKGROUND

The most important function of an immune system is to protect a subject against infection by foreign agents. Thus, a healthy immune system must discriminate between the healthy tissue (“self”) and foreign agents (“non-self”). Non-limiting examples of foreign agents can be microorganisms, pollen, and transplanted tissues from another individual. Autoimmune disease occurs when an immune system mounts an attack against healthy tissue since the system does not recognize the healthy tissue as “self”. Unfortunately, once an autoimmune disease has been diagnosed, it becomes or has become a chronic problem. A standard treatment for autoimmune diseases is a generalized suppression of the immune system. Unfortunately, such non-specific therapies may inhibit the immune system's ability to recognize and attack actual foreign agents which places the subject at risk for infections and cancerous malignancies. Accordingly, there is an unmet need for autoimmune therapies that effectively treat autoimmune disease yet minimized risk for infections and malignancy outgrowth.

### SUMMARY

In various aspects, the present invention provides for compositions and methods that are useful for immunotherapies for treating an autoimmune disease. For instance, the present invention, in part, relates to specific chimeric proteins comprising two domains that each or both domains decrease self-directed immune system activity when bound to its ligand/receptor. Importantly, when each or both domains decreases immune system activity by activating an immune inhibitory signal or inhibiting an immune activating signal. Accordingly, the present chimeric proteins, compositions, and methods overcome various deficiencies in bi-specific agents directed to treat autoimmunity.

An aspect of the present invention is a chimeric protein of a general structure of: N terminus-(a)-(b)-(c)-C terminus in which (a) is a first domain comprising a portion of the extracellular domain of a transmembrane protein, a secreted protein, or a membrane-anchored extracellular protein, (c) is a second domain comprising a portion of the extracellular domain of a transmembrane protein, a secreted protein, or a membrane-anchored extracellular protein, and (b) is a linker adjoining the first domain and the second domain. In this aspect, either or both of the first domain and the second domain decreases self-directed immune system activity when bound to its ligand/receptor.

Another aspect of the present invention is a chimeric protein comprising: (a) a first domain comprising a portion of VSIG4 that is capable of binding a VSIG4 ligand/receptor, (b) a second domain comprising a portion of IL2 that is capable of binding an IL2 receptor, and (c) a linker linking the first domain and the second domain and comprising a hinge-CH2-CH3 Fc domain.

Yet another aspect of the present invention is a chimeric protein comprising: (a) a first domain comprising a portion of CTLA4 that is capable of binding a CTLA4 ligand/receptor, (b) a second domain comprising a portion of IL2 that is capable of binding an IL2 receptor, and (c) a linker linking the first domain and the second domain and comprising a hinge-CH2-CH3 Fc domain. In embodiments, the IL2 receptor is a high-affinity IL2 receptor that is expressed by regulatory T cells, e.g., the portion of IL2 comprises one or more mutations relative to a corresponding portion of wild-type IL2 which provides preferential binding to the high-affinity IL2 receptor that is expressed by regulatory T cells.

In an aspect, the present invention provides a chimeric protein comprising: (a) a first domain comprising a portion of CTLA4 that is capable of binding a CTLA4 ligand/receptor, (b) a second domain comprising a portion of PD-L1 that is capable of binding PD-1, and (c) a linker linking the first domain and the second domain and comprising a hinge-CH2-CH3 Fc domain.

In another aspect, the present invention provides a chimeric protein comprising: (a) a first domain comprising a portion of B7H3 that is capable of binding a B7H3 ligand/receptor, (b) a second domain comprising a portion of PD-L1 that is capable of binding PD-1, and (c) a linker linking the first domain and the second domain and comprising a hinge-CH2-CH3 Fc domain.

In yet another aspect, the present invention provides a chimeric protein comprising: (a) a first domain comprising a portion of B7H4 that is capable of binding a B7H4 ligand/receptor, (b) a second domain comprising a portion of PD-L1 that is capable of binding PD-1, and (c) a linker linking the first domain and the second domain and comprising a hinge-CH2-CH3 Fc domain.

An aspect of the present invention is a chimeric protein comprising: (a) a first domain comprising a portion of ICOSL that is capable of binding an ICOSL ligand/receptor, (b) a second domain comprising a portion of PD-L1 that is capable of binding PD-1, and (c) a linker linking the first domain and the second domain and comprising a hinge-CH2-CH3 Fc domain.

Another aspect of the present invention is chimeric protein comprising: (a) a first domain comprising a portion of ILDR2 that is capable of binding an ILDR2 ligand/receptor, (b) a second domain comprising a portion of PD-L1 that is capable of binding PD-1, and (c) a linker linking the first domain and the second domain and comprising a hinge-CH2-CH3 Fc domain.

Yet another aspect of the present invention is chimeric protein comprising: (a) a first domain comprising a portion of BTNL2 that is capable of binding a BTNL2 ligand/receptor, (b) a second domain comprising a portion of PD-L1 that is capable of binding PD-1, and (c) a linker linking the first domain and the second domain and comprising a hinge-CH2-CH3 Fc domain.

In an aspect, the present invention provides a chimeric protein comprising: (a) a first domain comprising a portion of PD-L1 that is capable of binding PD-1, (b) a second domain comprising a portion of BTNL2 that is capable of binding a BTNL2 ligand/receptor, and (c) a linker linking the first domain and the second domain and comprising a hinge-CH2-CH3 Fc domain.

In another aspect, the present invention provides a chimeric protein comprising: (a) a first domain comprising a portion of CSF3 that is capable of binding a CSF3 ligand/receptor, (b) a second domain comprising a portion of TL1A that is capable of binding a TL1A ligand/receptor, and (c) a linker linking the first domain and the second domain and comprising a hinge-CH2-CH3 Fc domain.

In yet another aspect, the present invention provides a chimeric protein comprising: (a) a first domain comprising a portion of CTLA4 that is capable of binding a CTLA4 ligand/receptor, (b) a second domain comprising a portion of TL1A that is capable of binding a TL1A ligand/receptor, and (c) a linker linking the first domain and the second domain and comprising a hinge-CH2-CH3 Fc domain.

Another aspect of the present invention is a chimeric protein comprising: (a) a first domain comprising a portion of CTLA4 that is capable of binding a CTLA4 ligand/receptor, (b) a second domain comprising a portion of SEMA3E that is capable of binding a SEMA3E ligand/receptor, and (c) a linker linking the first domain and the second domain and comprising a hinge-CH2-CH3 Fc domain.

Another aspect of the present invention is a chimeric protein comprising: (a) a first domain comprising a portion of MadCAM that is capable of binding a MadCAM ligand/receptor, (b) a second domain comprising a portion of CCL25 that is capable of binding a CCL25 ligand/receptor, and (c) a linker linking the first domain and the second domain and comprising a hinge-CH2-CH3 Fc domain.

Another aspect of the present invention is a chimeric protein comprising: (a) a first domain comprising a portion of TNFR2 that is capable of binding a TNFR2 ligand/receptor, (b) a second domain comprising a portion of TGFβ that is capable of binding a TGFβ ligand/receptor, and (c) a linker linking the first domain and the second domain and comprising a hinge-CH2-CH3 Fc domain.

Another aspect of the present invention is a chimeric protein comprising: (a) a first domain comprising an extracellular domain of IL-6R that is capable of binding a IL-6R ligand/receptor, (b) a second domain comprising a portion of IL-35 that is capable of binding a IL-35 ligand/receptor, and (c) a linker linking the first domain and the second domain and comprising a hinge-CH2-CH3 Fc domain. In embodiments, the first domain comprises an extracellular domain of IL-6ST and/or IL-6R. In embodiments, the second domain comprises a portion of EBI3 and/or IL-12A. In embodiments, the chimeric protein is heterodimeric.

Another aspect of the present invention is a chimeric protein comprising: (a) a first domain comprising an extracellular domain of integrin α4β7 that is capable of binding an integrin α4β7 ligand/receptor, (b) a second domain comprising a portion of IL-35 that is capable of binding a IL-35 ligand/receptor, and (c) a linker linking the first domain and the second domain and comprising a hinge-CH2-CH3 Fc domain. In embodiments, the first domain comprises an extracellular domain of integrin α4 and/or integrin β7. In embodiments, the second domain comprises a portion of EBI3 and/or IL-12A. In embodiments, the chimeric protein is heterodimeric.

The chimeric protein of any of the above aspects or embodiments may be a recombinant fusion protein.

The chimeric protein of any of the above aspects or embodiments may be used as a medicament in the treatment of an autoimmune disease, e.g., selected from ankylosing spondylitis, diabetes mellitus, Grave's disease, Hashimoto's thyroiditis, hypersensitivity reactions (e.g., allergies, hay fever, asthma, and acute edema cause type I hypersensitivity reactions), inflammatory bowel diseases (e.g., colitis ulcerosa and Crohn's disease), multiple sclerosis, psoriasis, psoriasis, rheumatoid arthritis, sarcoidosis, Sjögren's syndrome, systemic lupus erythematosus, and vasculitis.

The present invention includes the use of the chimeric protein of any of the above aspects or embodiments in the manufacture of a medicament.

An aspect of the present invention is an expression vector comprising a nucleic acid encoding the chimeric protein of any of the above aspects or embodiments.

Another aspect of the present invention is a host cell comprising the expression vector of the preceding aspect.

Yet another aspect of the present invention is a pharmaceutical composition comprising a therapeutically effective amount of the chimeric protein of any of the herein disclosed aspects or embodiments.

An aspect of the present invention is a method of treating an autoimmune disease comprising administering to a subject in need thereof an effective amount of a pharmaceutical composition comprising a therapeutically effective amount of the chimeric protein of any of the herein disclosed aspects or embodiments.

Any aspect or embodiment disclosed herein can be combined with any other aspect or embodiment as disclosed herein.

### BRIEF DESCRIPTION OF THE DRAWINGS

FIG. 1A to FIG. 1C show schematic illustrations of proteins that may be used in chimeric proteins of the present disclosure. FIG. 1A shows a Type I transmembrane protein (left protein) and Type II transmembrane protein (right proteins); these proteins differ in that Type I proteins have their amino terminus (“N-”), which comprises its ligand/receptor binding site, directed extracellularly whereas Type II proteins have their carboxy terminus (“C-”), which comprises its ligand/receptor binding site, directed extracellularly. FIG. 1B shows two membrane-anchored extracellular proteins; the illustrated proteins have a ligand/receptor binding site at its amino terminus (“N-”) and is membrane anchored via its carboxy terminus (left protein) or have a ligand/receptor binding site at its carboxy terminus (“C-”) and is membrane anchored via its amino terminus (right protein); however, membrane-anchored extracellular proteins may be membrane anchored via other locations along the protein's amino acid sequence. FIG. 1C shows two secreted proteins (which lack a transmembrane domain or a membrane anchorage); the left protein has its ligand/receptor binding site at it amino terminus (“N-”) and the right protein has its ligand/receptor binding site at its carboxy terminus (“C-”).

FIG. 2A to FIG. 2D show schematic illustrations of chimeric proteins of the present disclosure. FIG. 2A shows a chimeric protein comprising a first domain with a ligand/receptor binding site at its amino terminus and a second domain with a ligand/receptor binding site at its carboxy terminus. Non-limiting examples of this configuration of chimeric protein include a chimeric protein comprising a portion of a Type I transmembrane protein as its first domain and a portion of a Type II transmembrane protein as its second domain and a chimeric protein comprising a portion of a Type I transmembrane protein as its first domain and a portion of a secreted protein as its second domain. FIG. 2B shows a chimeric protein comprising a first domain with a ligand/receptor binding site at its amino terminus and a second domain with a ligand/receptor binding site at its amino terminus. Non-limiting examples of this configuration of chimeric protein include a chimeric protein comprising a portion of a Type I transmembrane protein as its first domain and a portion of a Type I transmembrane protein as its second domain and a chimeric protein comprising a portion of a Type I transmembrane protein as its first domain and a portion of a secreted protein as its second domain. FIG. 2C shows a chimeric protein comprising a first domain with a ligand/receptor binding site at its carboxy terminus and a second domain with a ligand/receptor binding site at its carboxy terminus. Non-limiting examples of this configuration of chimeric protein include a chimeric protein comprising a portion of a membrane anchored protein as its first domain and a portion of secreted protein as its second domain and a chimeric protein comprising a portion of secreted protein as its first domain and a portion of a Type II transmembrane protein as its second domain. FIG. 2D shows a chimeric protein comprising a first domain with a ligand/receptor binding site at its carboxy terminus and a second domain with a ligand/receptor binding site at its amino terminus. Non-limiting examples of this configuration of chimeric protein include a chimeric protein comprising a portion of secreted protein as its first domain and a portion of a membrane anchored protein as its second domain and a chimeric protein comprising a portion of Type II transmembrane protein as its first domain and a portion of a Type I transmembrane protein as its second domain.

FIG. 3 is a schematic of a CSF3- and TL1A-based chimeric protein of the present disclosure and shows characterization of a murine CSF3-Fc-TL1A chimeric protein by western blot demonstrating the chimeric proteins native state and tendency to form a multimer. Untreated samples (i.e., without reducing agent or deglycosylation agent) of the CSF3-Fc-TL1A chimeric protein, e.g., control, were loaded into lane 2 in all the blots. Samples in lane 3 were treated with the reducing agent, 3-mercaptoethanol. Samples in lane 4 were treated with a deglycosylation agent and the reducing agent. Each individual domain of the chimeric protein was probed using an anti-CSF3, anti-Fc, or anti-TL1A antibody, respectively.

FIG. 4A to FIG. 4D show ELISA assays demonstrating the binding affinity of the CSF3 domain of mCSF3-Fc-TL1A (FIG. 4A and FIG. 4B), the Fc domain of mCSF3-Fc-TL1A (FIG. 4C), and of the TL1A domain of mCSF3-Fc-TL1A (FIG. 4D) for their respective binding partners.

FIG. 5 is a graph demonstrating the in vivo ability of the mCSF3-Fc-TL1A chimeric protein to increase the frequency of regulatory T cells (Treg) relative to blood stem cells.

FIG. 6 is a graph demonstrating the in vivo ability of the mCSF3-Fc-TL1A chimeric protein to increase the frequency of regulatory T cells (Treg) relative to other CD4+ T cells. Here, the treatments administered, from left to right, are: control (PBS), anti-DR3 antibody (100 μg), G-CSF (10 μg manufactured by LSbio), G-CSF (10 μg manufactured by Biolegend), G-CSF (50 μg manufactured by Biolegend), a combination of the anti-DR3 antibody (100 μg) and G-CSF (10 μg manufactured by Biolegend), or the mCSF3-Fc-TL1A chimeric protein (at 100 μg or 300 μg).

FIG. 7A is a schematic of a VISG4- and IL2-based chimeric protein of the present disclosure. FIG. 7B shows characterization of a murine VSIG4-Fc-IL2 chimeric protein by western blot. Untreated samples (i.e., without reducing agent or deglycosylation agent “NR”) of the mVSIG4-Fc-IL2 chimeric protein, samples treated with the reducing agent, β-mercaptoethanol (“R”), and samples treated with a deglycosylation agent and the reducing agent (“DG”) are shown. The blot was probed using an anti-Fc antibody. FIG. 7C shows an ELISA of the mVSIG4-Fc-IL2 chimeric protein captured by its Fc domain.

FIG. 8A is a schematic of a PD-L1- and BTNL2-based chimeric protein of the present disclosure. FIG. 8B shows characterization of a murine PD-L1-Fc-BTNL2 chimeric protein by western blot. FIG. 8C shows an ELISA of the mPD-L1-Fc-BTNL2 chimeric protein captured by its Fc domain.

FIG. 9A is a schematic of a CTLA4- and SEMA3E-based chimeric protein of the present disclosure. FIG. 9B shows characterization of a human CTLA4-Fc-SEMA3E chimeric protein by western blot probed using an anti-Fc antibody. FIG. 9C shows an ELISA of the hCTLA4-Fc-SEMA3E chimeric protein captured by its Fc domain.

FIG. 10A is a schematic of an ILDR2- and PD-L1-based chimeric protein of the present disclosure. FIG. 10B shows characterization of a human ILDR2-Fc-PD-L1 chimeric protein by western blot probed using an anti-Fc antibody. FIG. 10C shows an ELISA of the h ILDR2-Fc-PD-L1 chimeric protein captured by its Fc domain.

FIG. 11A and FIG. 11B show chromatographs for the human IL-6R-Fc-IL-35 chimeric proteins run on size exclusion chromatography (SEC).

FIG. 12 shows the induction of apoptosis as measured by a luciferase assay in DS-1 cells cultured for 24 hours in the presence of increasing molar ratios of the indicated molecules to IL-6. Caspase 3/7 activity was plotted.

FIG. 13A to FIG. 13G show the change in the relative levels of mRNA, as measured by qRT-PCR, of EBI3 (FIG. 13A), IL-12A (FIG. 13B), FOXP3 (FIG. 13C), TOP2A (FIG. 13D), TGF-β (FIG. 13E), IL-10 (FIG. 13F), and IL-6 (FIG. 13G) in purified, naïve splenic CD4 T cells that were cultured in the presence of anti-CD3/anti-CD28 beads and vehicle alone control (Unstim), IL-35, the human IL-6R-Fc-IL-35 chimeric protein, an unrelated control chimeric protein, IL-2, or TGF-β/Retinoic Acid (RA) for 9 days.

FIG. 14A and FIG. 14B show the effect of the CD4 T cells generated as described for FIG. 13A to FIG. 13G on the proliferation of syngeneic CD8 cells when mixed at CD4: CD8 ratio of 2:1 (FIG. 14A) or 0.5:1 (FIG. 14B).

FIG. 15A shows a schematic of an IL-6R- and IL-35-based chimeric protein of the present disclosure. FIG. 15B shows characterization of a murine IL-6R-Fc-IL-35 chimeric protein by western blot demonstrating the chimeric proteins native state and tendency to form a multimer. Untreated samples (i.e., without reducing agent or deglycosylation agent) of the IL-6R-Fc-IL-35 chimeric protein were loaded into lane NR in each blot. Samples in lane R were treated with β-mercaptoethanol, a reducing agent. Samples in lane DG were treated with a deglycosylation agent and the reducing agent. Each individual domain of the chimeric protein was probed using an anti-IL-6ST, anti-IL-6R, anti-Fc, anti-EBI3, or anti-IL-12A antibodies, respectively.

FIG. 16A and FIG. 16B show the results of sandwich ELISA performed on the MSD platform to determine the relative abundance of the heterodimeric IL-6R-Fc-IL-35 chimeric protein in the purified preparations. For FIG. 16A, an anti-IL-6ST antibody was coated on plates. Increasing amounts of the IL-6R-Fc-IL-35 chimeric protein or the TNFR2-Fc-TGFβ chimeric protein were added to the plate for capture by the plate-bound anti-IL-6ST antibody. The binding was detected using an anti-IL-12A antibody. For FIG. 16B, an anti-IL-6R antibody was coated on plates. Increasing amounts of the IL-6R-Fc-IL-35 chimeric protein or the TNFR2-Fc-TGFβ chimeric protein were added to the plate for capture by the plate-bound anti-IL-6R antibody. The binding was detected using an anti-IL-27B antibody.

FIG. 17A shows a schematic of a MadCAM- and CCL25-based chimeric protein of the present disclosure. FIG. 17B shows characterization of a murine MadCAM-Fc-CCL25 chimeric protein by western blot demonstrating the chimeric proteins native state and tendency to form a multimer. Untreated samples (i.e., without reducing agent or deglycosylation agent) of the MadCAM-Fc-CCL25 chimeric protein were loaded into lane NR in each blot. Samples in lane R were treated with 3-mercaptoethanol, a reducing agent. Samples in lane DG were treated with a deglycosylation agent and the reducing agent. Each individual domain of the chimeric protein was probed using an anti-MadCAM, anti-Fc, or anti-CCL25 antibodies, respectively.

FIG. 18A and FIG. 18B show the results of sandwich ELISA performed on the MSD platform to determine the relative abundance of the heterodimeric MadCAM-Fc-CCL25 chimeric protein in the purified preparations. For FIG. 18A, an anti-CCL25 antibody was coated on plates. Increasing amounts of the MadCAM-Fc-CCL25 chimeric protein or the TNFR2-Fc-TGFβ chimeric protein were added to the plate for capture by the plate-bound anti-CCL25 antibody. The binding was detected using an anti-MadCAM antibody. For FIG. 18B, an anti-MadCAM antibody was coated on plates. Increasing amounts of the MadCAM-Fc-CCL25 chimeric protein or the TNFR2-Fc-TGFβ chimeric protein were added to the plate for capture by the plate-bound anti-MadCAM antibody. The binding was detected using an anti-CCL25 antibody.

FIG. 19A shows a schematic of an α4β7- and IL-35-based chimeric protein of the present disclosure. FIG. 19B shows characterization of a murine α4β7-Fc-IL-35 chimeric protein by western blot demonstrating the chimeric proteins native state and tendency to form a multimer. Untreated samples (i.e., without reducing agent or deglycosylation agent) of the α4β7-Fc-IL-35 chimeric protein were loaded into lane NR in each blot. Samples in lane R were treated with 3-mercaptoethanol, a reducing agent. Samples in lane DG were treated with a deglycosylation agent and the reducing agent. Each individual domain of the chimeric protein was probed using an anti-α4, anti-β7, anti-Fc, anti-EBI3 or anti-IL-12A antibodies, respectively.

FIG. 20A and FIG. 20B show the results of sandwich ELISA performed on the MSD platform to determine the relative abundance of the hetrodimeric α4β7-Fc-IL-35 chimeric protein in the purified preparations. For FIG. 20A, an anti-α4 antibody was coated on plates. Increasing amounts of the α4β7-Fc-IL-35 chimeric protein or the TNFR2-Fc-TGFβ chimeric protein were added to the plate for capture by the plate-bound anti-α4 antibody. The binding was detected using an anti-IL27B antibody. For FIG. 20B, an anti-IL-12A antibody was coated on plates. Increasing amounts of the α4β7-Fc-IL-35 chimeric protein or the TNFR2-Fc-TGFβ chimeric protein were added to the plate for capture by the plate-bound anti-IL-12A antibody. The binding was detected using an anti-β7 antibody.

FIG. 21A shows a schematic of a TNFR2- and TGFβ-based chimeric protein of the present disclosure. FIG. 21B shows characterization of a murine TNFR2-Fc-TGFβ chimeric protein by western blot demonstrating the chimeric proteins native state and tendency to form a multimer. Untreated samples (i.e., without reducing agent or deglycosylation agent) of the TNFR2-Fc-TGFβ chimeric protein were loaded into lane NR in each blot. Samples in lane R were treated with β-mercaptoethanol, a reducing agent. Samples in lane DG were treated with a deglycosylation agent and the reducing agent. Each individual domain of the chimeric protein was probed using an anti-TNFR2, anti-Fc, or anti-TGFβ antibodies, respectively.

FIG. 22A and FIG. 22B show the results of sandwich ELISA performed on the MSD platform to determine the relative abundance of the hetrodimeric TNFR2-Fc-TGFβ chimeric protein in the purified preparations. For FIG. 22A, an anti-TGFβ antibody was coated on plates. Increasing amounts of the TNFR2-Fc-TGFβ chimeric protein or the MadCAM-Fc-CCL chimeric protein were added to the plate for capture by the plate-bound anti-TGFβ antibody. The binding was detected using an anti-TNFR2 antibody. For FIG. 22B, an anti-TNFR2 antibody was coated on plates. Increasing amounts of the TNFR2-Fc-TGFβ chimeric protein or the MadCAM-Fc-CCL chimeric protein were added to the plate for capture by the plate-bound anti-TNFR2 antibody. The binding was detected using an anti-TGFβ antibody.

FIG. 23 shows a t-distributed stochastic neighbor embedding (t-SNE) plot illustrating dextran sodium sulfate (DSS) induced colitis in mice. Mice were untreated or received 3% DSS in their drinking water ad libitum for 8 days starting day 0. On Day 9, DSS containing drinking water was replaced with unmodified drinking water. On day 11, all animals were sacrificed, and mesenteric lymph nodes (MLN) were harvested. Cells from MLN were subjected to flow cytometry using a 15-parameter FACS panel to phenotypically characterize the cellular composition of the MLN. The data were analyzed on FlowJo, and was subjected to dimensionality reduction with t-SNE and phenotypic populations mapped with X-Shift.

FIG. 24A to FIG. 24C illustrate the phenotypic differences in cells from MLN of mice induced to have colitis using DSS and treated with the chimeric proteins of the present disclosure. Mice were left untreated or untreated as discussed for FIG. 23. On days 0, 1, and 2, experimental treatment group animals were administered 100 μg of the indicated therapeutic molecule, once daily, intraperitoneally. Control animals were administered 100 μg of murine IgG. Cells from MLN were subjected to flow cytometry using the 15-parameter FACS panel to phenotypically characterize the cellular composition of the MLN. The data were analyzed on FlowJo, and was subjected to dimensionality reduction with t-SNE and phenotypic populations mapped with X-Shift. FIG. 24A shows the t-SNE density plot overlays. FIG. 24B illustrates the differences between the density plot overlays. For example, the treatment with the MadCAM-Fc-CCL25, IL-6R-Fc-IL-35, and TNFR2-Fc-TGFβ chimeric proteins decreased the cells within the area marked with lighter outlined shapes and increased the cells within the area marked with a black outlined shape. FIG. 24C is a table showing the differences in relative abundance of the indicated cell types.

FIG. 25 illustrates that the TNFR2-Fc-TGFβ chimeric protein protects cells from TNF-α mediated apoptosis in L929 cells, which are known to be highly sensitive to TNF-α induced apoptosis. Fixed numbers of L929 cells were incubated in microtiter plates with 10 ng/ml of TNF-α for 24 hours. Increasing molar ratios of the TNFR2-Fc-TGFβ chimeric protein or an irrelevant chimeric protein (OH) that is not known to protect cells from apoptosis were titrated into the plates. After 24 hours, the cells were assessed for cell death using the Caspase 3/7 CytoGlo system on the Promega GloMax Luminometer.

FIG. 26 shows a plot of body weights of mice that induced to have colitis using 2,4,6-trinitrobenzenesulfonic acid (TNBS). 2.5% in ethanol was administered on day 0. The negative control animals were administered colonic instillation of ethanol alone. 100 μg of the TNFR2-Fc-TGFβ or CLTA4-Fc-TL1A chimeric proteins, or vehicle only were administered on days 0, 1, and 2, once daily, intraperitoneally.

FIG. 27A to FIG. 27C illustrate the phenotypic differences in the cells from MLN of control mice, mice induced to have colitis by colonic instillation of TNBS as discussed for FIG. 26, and treated with the TNFR2-Fc-TGFβ chimeric protein as discussed for FIG. 26. FIG. 27A shows the t-SNE density plot of mice induced to have colitis by colonic instillation of TNBS treated with the TNFR2-Fc-TGFβ chimeric protein. FIG. 27B illustrates the differences between the density plot overlays. FIG. 27C is a table showing the differences in relative abundance of the indicated cell types.

FIG. 28A to FIG. 28F illustrate the relative change in the relative levels of mRNA, as measured by qRT-PCR, of TLR5 (FIG. 28A), IL-17A (FIG. 28B), IL-4 (FIG. 28C), IL-1B (FIG. 28D), CCL-2 (FIG. 28E), and IL-6 (FIG. 28F) in the cells from MLN of control mice, mice induced to have colitis by colonic instillation of TNBS as discussed for FIG. 26, and mice induced to have colitis by colonic instillation of TNBS treated with the TNFR2-Fc-TGFβ chimeric protein as discussed for FIG. 26.

FIG. 29A to FIG. 29C illustrate the phenotypic differences in the CD4 T cells were isolated from the spleens of FoxP3 RFP knock-in mice (FIR mice), that were cultured for 5 days with activating anti-CD3/anti-CD28 beads in the presence of a IL4, TGFβ, MadCAM-fc-CCL25, TNFR2-fc-TGFβ, α4β7-fc-IL35, or IL6R-fc-IL35. FIG. 29A shows the t-SNE density plot of showing eight distinct phenotypic populations of CD4 cells. FIG. 29B illustrates the differences between the density plot overlays when the CD4 cell were treated with the indicated protein. FIG. 29C is a table showing the differences in relative abundance of the indicated cell types.

### DETAILED DESCRIPTION

The present invention is based, in part, on the discovery that chimeric proteins can be engineered from a first domain comprising an extracellular domain of a first transmembrane protein, a first secreted protein, or a first membrane-anchored extracellular protein and a second domain comprising an extracellular domain of a second transmembrane protein, a second secreted protein, or a second membrane-anchored extracellular protein. In these chimeric proteins, either or both of the first domain and the second domain decreases self-directed immune system activity when bound to its ligand/receptor. Accordingly, the present invention find use in the treatment of an autoimmune disease, which occurs when a subject's own antigens become targets for an immune response.

The present chimeric proteins provide advantages including, without limitation, ease of use and ease of production. This is because two distinct immunotherapy agents are combined into a single product which may allow for a single manufacturing process instead of two independent manufacturing processes. In addition, administration of a single agent instead of two separate agents allows for easier administration and greater patient compliance. Further, in contrast to, for example, monoclonal antibodies, which are large multimeric proteins containing numerous disulfide bonds and post-translational modifications such as glycosylation, the present chimeric proteins are easier and more cost effective to manufacture.

Importantly, since a chimeric protein of the present invention comprises two ligand/receptor binding domains, it is capable of, via two cellular pathways, decreasing immune system activity by activating an immune inhibitory signal and/or by inhibiting an immune activating signal. This dual-action is more likely to provide any anti-autoimmune effect in a subject. Moreover, since the chimeric proteins and methods using the chimeric proteins operate by multiple distinct pathways, they can be efficacious, at least, in patients who do not respond, respond poorly, or become resistant to treatments that target one of the pathways. Thus, a patient who is a poor responder to treatments acting via one of the two pathways, can receive a therapeutic benefit by targeting multiple pathways.

### Chimeric Proteins

An aspect of the present invention is a chimeric protein of a general structure of: N terminus-(a)-(b)-(c)-C terminus in which (a) is a first domain comprising a portion of the extracellular domain of a transmembrane protein, a secreted protein, or a membrane-anchored extracellular protein, (c) is a second domain comprising a portion of the extracellular domain of a transmembrane protein, a secreted protein, or a membrane-anchored extracellular protein, and (b) is a linker adjoining the first domain and the second domain. In this aspect, either or both of the first domain and the second domain decreases self-directed immune system activity when bound to its ligand/receptor.

In embodiments, the portion of the first domain is capable of binding the native ligand/receptor for the transmembrane protein, the secreted protein, or the membrane-anchored extracellular protein.

In embodiments, the portion of the second domain is capable of binding the native ligand/receptor for the transmembrane protein, the secreted protein, or the membrane-anchored extracellular protein.

In embodiments, the first domain comprises substantially the entire extracellular domain of the transmembrane protein, substantially the entire secreted protein, or substantially the entire membrane-anchored extracellular protein.

In embodiments, the second domain comprises substantially the entire extracellular domain of the transmembrane protein, substantially the entire secreted protein, or substantially the entire membrane-anchored extracellular protein.

In embodiments, the binding of the portion of the first domain to its ligand/receptor decreases immune system activity by activating an immune inhibitory signal or inhibiting an immune activating signal.

In embodiments, the binding of the portion of the second domain to its ligand/receptor decreases immune system activity by activating an immune inhibitory signal or by inhibiting an immune activating signal.

In embodiments, the portion of the first domain comprises a transmembrane protein, a secreted protein, or a membrane-anchored extracellular protein selected from B7H3, B7H4, BTNL2, CTLA4, CSF3, ICOSL, ILDR2, PD-L1, TNFR2, and VSIG4.

In embodiments, the portion of the second domain comprises a transmembrane protein, a secreted protein, or a membrane-anchored extracellular protein selected from BTNL2, IL2, PD-L1, SEMA3E, IL-35, CCL25, TGFβ, and TL1A.

In embodiments, the first domain comprises a portion of VSIG4 and the second domain comprises a portion of IL2.

In embodiments, the first domain comprises a portion of CTLA4 and the second domain comprises a portion of IL2, e.g., the portion of IL2 comprises one or more mutations relative to a corresponding portion of wild-type IL2 wherein the one or more mutations provide preferential binding to a high-affinity IL2 receptor that is expressed by regulatory T cells.

In embodiments, the first domain comprises a portion of CTLA4 and the second domain comprises a portion of PD-L1.

In embodiments, the first domain comprises a portion of B7H3 and the second domain comprises a portion of PD-L1.

In embodiments, the first domain comprises a portion of B7H4 and the second domain comprises a portion of PD-L1.

In embodiments, the first domain comprises a portion of ICOSL and the second domain comprises a portion of PD-L1.

In embodiments, the first domain comprises a portion of ILDR2 and the second domain comprises a portion of PD-L1.

In embodiments, the first domain comprises a portion BTNL2 of and the second domain comprises a portion of PD-L1 or the first domain comprises a portion of PD-L1 and the second domain comprises a portion of BTNL2.

In embodiments, the first domain comprises a portion of CSF3 and the second domain comprises a portion of TL1A.

In embodiments, the first domain comprises a portion of CTLA4 and the second domain comprises a portion of TL1A.

In embodiments, the first domain comprises a portion of CTLA4 and the second domain comprises a portion of SEMA3E.

In embodiments, the first domain comprises a portion of TNFR2 and the second domain comprises an extracellular domain of a transmembrane protein selected from 4-1BBL, APRIL, BAFF, BTNL2, CD28, CD30L, CD40L, CD70, C-type lectin domain (CLEC) family members, FasL, GITRL, LIGHT, LTa, LTa1b2, NKG2A, NKG2C, NKG2D, OX40L, RANKL, TL1A, TNFa, and TRAIL; in embodiments, the second domain comprises an extracellular domain of GITRL or TL1A. In embodiments, the CLEC family member is selected from AlCL/CLEC-2B, ASGR1/ASGPR1, ASGR2, C1q R1/CD93, CD161, CD161/NK1.1, CD23/Fc epsilon RII, CD302/CLEC13A, CD72, CD94, Chondrolectin, CLEC-1, CLEC10A/CD301, CLEC12B, CLEC14A, CLEC16A, CLEC17A, CLEC18A, CLEC18B, CLEC18C, CLEC-2/CLEC1B, CLEC-2A, CLEC3A, CLEC3B/Tetranectin, CLEC4B2/mDCAR1, CLEC4D/CLECSF8, CLEC4E, CLEC4F/CLECSF13, CLEC9a, CLECL1/DCAL-1, CL-K1/COLEC11, CL-L1/COLEC10, CL-P1/COLEC12, DCAR/CLEC4B, DCIR/CLEC4A, DCIR4/CLEC4A1, DC-SIGN/CD209, DC-SIGN+FDC-SIGNR, DC-SIGNR/CD299, DC-SIGNR/CD299, DEC-205/CD205, Dectin-1/CLEC7A, Dectin-2/CLEC6A, DLEC/CLEC4C/BDCA-2, Ficolin-1, Ficolin-2, Ficolin-3, Klre-1, KLRG2, Langerin/CD207, Layilin, LOX-1/OLR1, LSECtin/CLEC4G, MBL, MBL-1, MBL-2, MDL-1/CLEC5A, MGL1/2 (CD301a/b), MGL1/CD301a, MGL2/CD301b, MGL2/CD301b, MICL/CLEC12A, MMR/CD206, Mrc2, NKG2A/CD159a, NKG2A/NKG2B Isoform 2, NKG2C/CD159c, NKG2D/CD314, NKG2E, NKG2H, NKp80/KLRF1, OCIL/CLEC2d, OCILRP2/CLEC2i, PLA2R1, QBRICK/FREM1, Reg1, Reg1A, Reg1B, Reg2, Reg3A, Reg3B, Reg3D, Reg3G, Reg4, SCGF/CLEC11a, SFTPA1, SIGNR1/CD209b, SIGNR3/CD209d, SIGNR4/CD209e, SIGNR7/CD209g, and SP-D.

In embodiments, the first domain comprises a portion of CTLA4 and the second domain comprises an extracellular domain of a transmembrane protein selected from 4-1BBL, APRIL, BAFF, BTNL2, CD28, CD30L, CD40L, CD70, C-type lectin domain (CLEC) family members, FasL, GITRL, LIGHT, LTa, LTa1b2, NKG2A, NKG2C, NKG2D, OX40L, RANKL, TL1A, TNFa, and TRAIL; in embodiments, the second domain comprises an extracellular domain of GITRL or TL1A. In embodiments, the CLEC family member is selected from AlCL/CLEC-2B, ASGR1/ASGPR1, ASGR2, C1q R1/CD93, CD161, CD161/NK1.1, CD23/Fc epsilon RII, CD302/CLEC13A, CD72, CD94, Chondrolectin, CLEC-1, CLEC10A/CD301, CLEC12B, CLEC14A, CLEC16A, CLEC17A, CLEC18A, CLEC18B, CLEC18C, CLEC-2/CLEC1B, CLEC-2A, CLEC3A, CLEC3B/Tetranectin, CLEC4B2/mDCAR1, CLEC4D/CLECSF8, CLEC4E, CLEC4F/CLECSF13, CLEC9a, CLECL1/DCAL-1, CL-K1/COLEC11, CL-L1/COLEC10, CL-P1/COLEC12, DCAR/CLEC4B, DCIR/CLEC4A, DCIR4/CLEC4A1, DC-SIGN/CD209, DC-SIGN+FDC-SIGNR, DC-SIGNR/CD299, DC-SIGNR/CD299, DEC-205/CD205, Dectin-1/CLEC7A, Dectin-2/CLEC6A, DLEC/CLEC4C/BDCA-2, Ficolin-1, Ficolin-2, Ficolin-3, Klre-1, KLRG2, Langerin/CD207, Layilin, LOX-1/OLR1, LSECtin/CLEC4G, MBL, MBL-1, MBL-2, MDL-1/CLEC5A, MGL1/2 (CD301a/b), MGL1/CD301a, MGL2/CD301b, MGL2/CD301b, MICL/CLEC12A, MMR/CD206, Mrc2, NKG2A/CD159a, NKG2A/NKG2B Isoform 2, NKG2C/CD159c, NKG2D/CD314, NKG2E, NKG2H, NKp80/KLRF1, OCIL/CLEC2d, OCILRP2/CLEC2i, PLA2R1, QBRICK/FREM1, Reg1, Reg1A, Reg1B, Reg2, Reg3A, Reg3B, Reg3D, Reg3G, Reg4, SCGF/CLEC11a, SFTPA1, SIGNR1/CD209b, SIGNR3/CD209d, SIGNR4/CD209e, SIGNR7/CD209g, and SP-D.

In embodiments, the binding of either or both of the first domain and the second domains to its ligand/receptor occurs with slow off rates (Koff), which provides a long interaction of a receptor and its ligand. In embodiments, the long interaction provides a prolonged decrease in immune system activity which comprises sustained activation of an immune inhibitory signal and/or a sustained inhibition of an immune activating signal. In embodiments, the sustained activation of the immune inhibitory signal and/or the sustained inhibition of the immune activating signal reduces the activity or proliferation of an immune cell, e.g., a B cell or a T cell. In embodiments, the sustained activation of the immune inhibitory signal and/or the sustained inhibition of the immune activating signal decreases synthesis and/or decreases release of a pro-inflammatory cytokine. In embodiments, the sustained activation of the immune inhibitory signal and/or the sustained inhibition of the immune activating signal increases synthesis and/or increases release of an anti-inflammatory cytokine. In embodiments, the sustained activation of the immune inhibitory signal and/or the sustained inhibition of the immune activating signal decreases antibody production and/or decreases secretion of antibodies by a B cell, e.g., an antibody that recognizes a self-antigen. In embodiments, the sustained activation of the immune inhibitory signal and/or the sustained inhibition of the immune activating signal decreases the activity of and/or decreases the number of T cytotoxic cells, e.g., which recognize a self-antigen and kill cells presenting or expressing the self-antigen. In embodiments, the sustained activation of the immune inhibitory signal and/or the sustained inhibition of the immune activating signal increases the activity and/or increases the number of T regulatory cells.

In embodiments, the linker is a polypeptide selected from a flexible amino acid sequence, an IgG hinge region, and an antibody sequence.

In embodiments, the linker comprises at least one cysteine residue capable of forming a disulfide bond and/or comprises a hinge-CH2-CH3 Fc domain, e.g., a hinge-CH2-CH3 Fc domain is derived from IgG (e.g., IgG1, IgG2, IgG3, and IgG4), IgA (e.g., IgA1 and IgA2), IgD, or IgE. In embodiments, the IgG is IgG4, e.g., a human IgG4. In embodiments, the linker comprises an amino acid sequence that is at least 95% identical to the amino acid sequence of SEQ ID NO: 1, SEQ ID NO: 2, or SEQ ID NO: 3.

Another aspect of the present invention is a chimeric protein comprising: (a) a first domain comprising a portion of VSIG4 that is capable of binding a VSIG4 ligand/receptor, (b) a second domain comprising a portion of IL2 that is capable of binding an IL2 receptor, and (c) a linker linking the first domain and the second domain and comprising a hinge-CH2-CH3 Fc domain. In embodiments, this chimeric protein is referred to as VSIG4-Fc-IL2.

Yet another aspect of the present invention is a chimeric protein comprising: (a) a first domain comprising a portion of CTLA4 that is capable of binding a CTLA4 ligand/receptor, (b) a second domain comprising a portion of IL2 that is capable of binding an IL2 receptor, and (c) a linker linking the first domain and the second domain and comprising a hinge-CH2-CH3 Fc domain. In embodiments, the IL2 receptor is a high-affinity IL2 receptor that is expressed by regulatory T cells, e.g., the portion of IL2 comprises one or more mutations relative to a corresponding portion of wild-type IL2 which provides preferential binding to the high-affinity IL2 receptor that is expressed by regulatory T cells. In embodiments, this chimeric protein is referred to as CTLA4-Fc-IL2.

In an aspect, the present invention provides a chimeric protein comprising: (a) a first domain comprising a portion of CTLA4 that is capable of binding a CTLA4 ligand/receptor, (b) a second domain comprising a portion of PD-L1 that is capable of binding PD-1, and (c) a linker linking the first domain and the second domain and comprising a hinge-CH2-CH3 Fc domain. In embodiments, this chimeric protein is referred to as CTLA4-Fc-PD-L1.

In another aspect, the present invention provides a chimeric protein comprising: (a) a first domain comprising a portion of B7H3 that is capable of binding a B7H3 ligand/receptor, (b) a second domain comprising a portion of PD-L1 that is capable of binding PD-1, and (c) a linker linking the first domain and the second domain and comprising a hinge-CH2-CH3 Fc domain. In embodiments, this chimeric protein is referred to as B7H3-Fc-PD-L1.

In yet another aspect, the present invention provides a chimeric protein comprising: (a) a first domain comprising a portion of B7H4 that is capable of binding a B7H4 ligand/receptor, (b) a second domain comprising a portion of PD-L1 that is capable of binding PD-1, and (c) a linker linking the first domain and the second domain and comprising a hinge-CH2-CH3 Fc domain. In embodiments, this chimeric protein is referred to as B7H4-Fc-PD-L1.

An aspect of the present invention is a chimeric protein comprising: (a) a first domain comprising a portion of ICOSL that is capable of binding an ICOSL ligand/receptor, (b) a second domain comprising a portion of PD-L1 that is capable of binding PD-1, and (c) a linker linking the first domain and the second domain and comprising a hinge-CH2-CH3 Fc domain. In embodiments, this chimeric protein is referred to as ICOSL-Fc-PD-L1.

Another aspect of the present invention is chimeric protein comprising: (a) a first domain comprising a portion of ILDR2 that is capable of binding an ILDR2 ligand/receptor, (b) a second domain comprising a portion of PD-L1 that is capable of binding PD-1, and (c) a linker linking the first domain and the second domain and comprising a hinge-CH2-CH3 Fc domain. In embodiments, this chimeric protein is referred to as ILDR2-Fc-PD-L1.

Yet another aspect of the present invention is chimeric protein comprising: (a) a first domain comprising a portion of BTNL2 that is capable of binding a BTNL2 ligand/receptor, (b) a second domain comprising a portion of PD-L1 that is capable of binding PD-1, and (c) a linker linking the first domain and the second domain and comprising a hinge-CH2-CH3 Fc domain. In embodiments, this chimeric protein is referred to as BTNL2-Fc-PD-L1.

In an aspect, the present invention provides a chimeric protein comprising: (a) a first domain comprising a portion of PD-L1 that is capable of binding PD-1, (b) a second domain comprising a portion of BTNL2 that is capable of binding a BTNL2 ligand/receptor, and (c) a linker linking the first domain and the second domain and comprising a hinge-CH2-CH3 Fc domain. In embodiments, this chimeric protein is referred to as PD-L1-Fc-BTNL2.

In another aspect, the present invention provides a chimeric protein comprising: (a) a first domain comprising a portion of CSF3 that is capable of binding a CSF3 ligand/receptor, (b) a second domain comprising a portion of TL1A that is capable of binding a TL1A ligand/receptor, and (c) a linker linking the first domain and the second domain and comprising a hinge-CH2-CH3 Fc domain. In embodiments, this chimeric protein is referred to as CSF3-Fc-TL1A.

In yet another aspect, the present invention provides a chimeric protein comprising: (a) a first domain comprising a portion of CTLA4 that is capable of binding a CTLA4 ligand/receptor, (b) a second domain comprising a portion of TL1A that is capable of binding a TL1A ligand/receptor, and (c) a linker linking the first domain and the second domain and comprising a hinge-CH2-CH3 Fc domain. In embodiments, this chimeric protein is referred to as CTLA4-Fc-TL1A.

Another aspect of the present invention is a chimeric protein comprising: (a) a first domain comprising a portion of CTLA4 that is capable of binding a CTLA4 ligand/receptor, (b) a second domain comprising a portion of SEMA3E that is capable of binding a SEMA3E ligand/receptor, and (c) a linker linking the first domain and the second domain and comprising a hinge-CH2-CH3 Fc domain. In embodiments, this chimeric protein is referred to as CTLA4-Fc-SEMA3E.

In embodiments, the hinge-CH2-CH3 Fc domain comprises at least one cysteine residue capable of forming a disulfide bond. In embodiments, the hinge-CH2-CH3 Fc domain is derived from IgG (e.g., IgG1, IgG2, IgG3, and IgG4), IgA (e.g., IgA1 and IgA2), IgD, or IgE. In embodiments, the IgG is IgG4, e.g., a human IgG4. In embodiments, the linker comprises an amino acid sequence that is at least 95% identical to the amino acid sequence of SEQ ID NO: 1, SEQ ID NO: 2, or SEQ ID NO: 3.

In a chimeric protein of the present invention, the chimeric protein is a recombinant fusion protein, e.g., a single polypeptide having the extracellular domains disclosed herein. For example, in embodiments, the chimeric protein is translated as a single unit in a prokaryotic cell, a eukaryotic cell, or a cell-free expression system.

In embodiments, the present chimeric protein is producible in a mammalian host cell as a secretable and fully functional single polypeptide chain.

In embodiments, chimeric protein refers to a recombinant protein of multiple polypeptides, e.g., multiple extracellular domains disclosed herein, that are combined (via covalent or no-covalent bonding) to yield a single unit, e.g., in vitro (e.g., with one or more synthetic linkers disclosed herein).

In embodiments, the chimeric protein is chemically synthesized as one polypeptide or each domain may be chemically synthesized separately and then combined. In embodiments, a portion of the chimeric protein is translated and a portion is chemically synthesized.

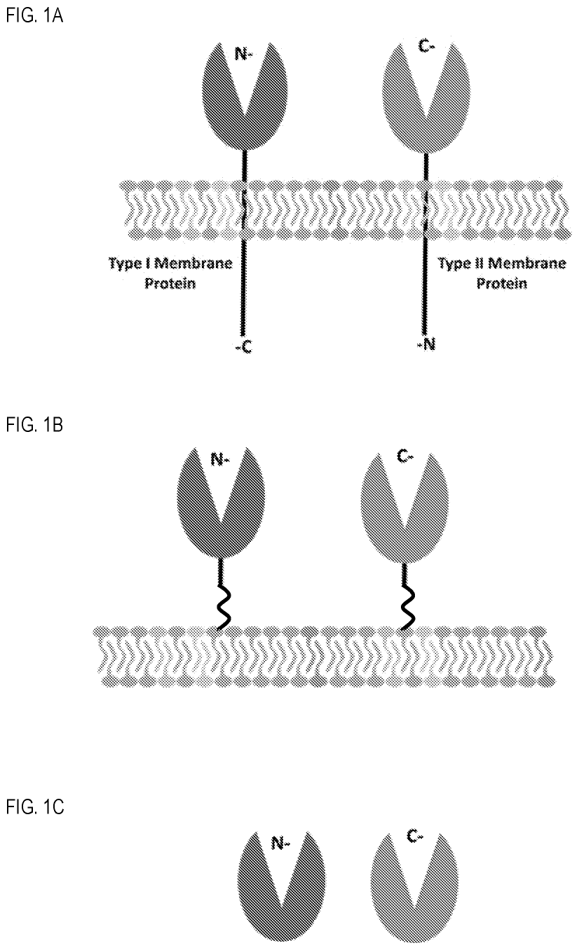

Transmembrane proteins typically consist of an extracellular domain, one or a series of transmembrane domains, and an intracellular domain. Without wishing to be bound by theory, the extracellular domain of a transmembrane protein is responsible for interacting with a soluble receptor or ligand or membrane-bound receptor or ligand (i.e., a membrane of an adjacent cell). Without wishing to be bound by theory, the trans-membrane domain(s) is responsible for localizing the transmembrane protein to the plasma membrane. Without wishing to be bound by theory, the intracellular domain of a transmembrane protein is responsible for coordinating interactions with cellular signaling molecules to coordinate intracellular responses with the extracellular environment (or visa-versa). Illustrations of transmembrane proteins are shown in FIG. 1A.

In contrast to transmembrane proteins, membrane-anchored extracellular proteins lack a transmembrane domain that spans, at least part, of a cell's lipid bilayer. Instead, these proteins are associated with the extracellular face of a cell's membrane. The association may be a result of hydrophobic interactions between the bilayer and exposed nonpolar residues at the surface of a protein, by specific non-covalent binding interactions with regulatory lipids, or through their attachment to covalently bound lipid anchors (including the lipids glycosylphosphatidylinositol (GPI) and cholesterol). Alternately, membrane-anchored extracellular proteins may indirectly be associated with the cell's lipid bilayer via another protein that is directly associated with the membrane, including transmembrane proteins. Illustrations of membrane-anchored extracellular proteins are shown in FIG. 1B.

A secreted protein can be defined as a protein which is actively transported out of the cell. Medically important secreted proteins include cytokines, coagulation factors, enzymes, growth factors, hormones, and other signaling molecules.

Often secreted proteins have an amino terminal comprising a signal sequence consisting of 6 to 12 amino acids with hydrophobic side chains. The signal sequence, at least, permits packaging of secreted proteins into vesicles which, when fused with the cell's membrane, the secreted protein leaves the cell. Illustrations of secreted proteins are shown in FIG. 1C.

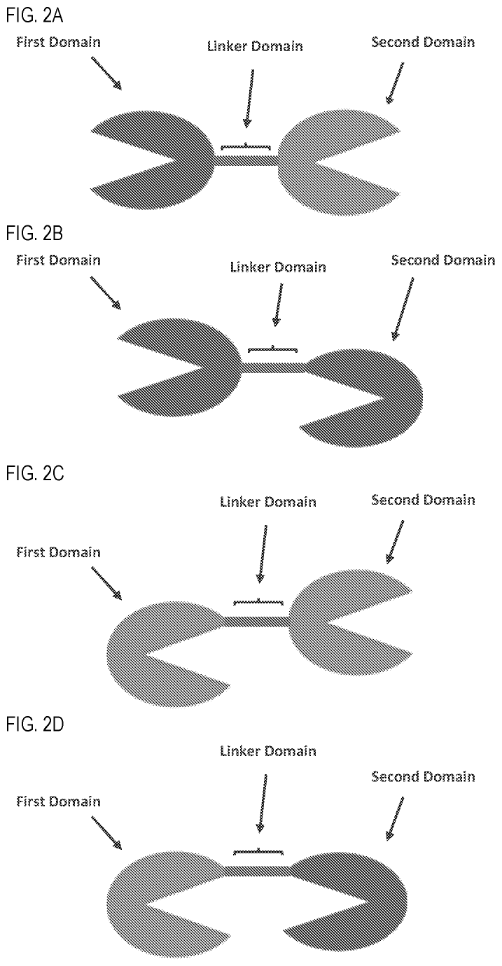

FIG. 2A to FIG. 2D show schematic illustrations of chimeric proteins of the present invention. FIG. 2A shows a chimeric protein comprising a first domain with a ligand/receptor binding site at its amino terminus and a second domain with a ligand/receptor binding site at its carboxy terminus. Non-limiting examples of chimeric proteins of the present invention which may have this configuration include CSF3-Fc-TL1A, CTLA4-Fc-IL2, CTLA4-Fc-SEMA3E, CTLA4-Fc-TL1A, PD-L1-Fc-BTNL2, and VSIG4-Fc-IL2. FIG. 2B shows a chimeric protein comprising a first domain with a ligand/receptor binding site at its amino terminus and a second domain with a ligand/receptor binding site at its amino terminus. Non-limiting examples of chimeric proteins of the present invention which may have this configuration include B7H3-Fc-PD-L1, B7H4-Fc-PD-L1, CTLA4-Fc-IL2, CTLA4-Fc-PD-L1, CTLA4-Fc-SEMA3E, ICOSL-Fc-PD-L1, ILDR2-Fc-PD-L1, and VSIG4-Fc-IL2. FIG. 2C shows a chimeric protein comprising a first domain with a ligand/receptor binding site at its carboxy terminus and a second domain with a ligand/receptor binding site at its carboxy terminus. Non-limiting examples of chimeric proteins of the present invention which may have this configuration include CSF3-Fc-TL1A. FIG. 2D shows a chimeric protein comprising a first domain with a ligand/receptor binding site at its carboxy terminus and a second domain with a ligand/receptor binding site at its amino terminus. Non-limiting examples of chimeric proteins of the present invention which may have this configuration include BTNL2-Fc-PD-L1.

Chimeric proteins of the present invention have a first domain which is sterically capable of binding its ligand/receptor and/or a second domain which is sterically capable of binding its ligand/receptor. This means that there is sufficient overall flexibility in the chimeric protein and/or physical distance between a first domain (or portion thereof) and the rest of the chimeric protein such that the ligand/receptor binding domain of the first domain is not sterically hindered from binding its ligand/receptor and/or there is sufficient physical distance between a second domain (or portion thereof) and the rest of the chimeric protein such that the ligand/receptor binding domain of the second domain is not sterically hindered from binding its ligand/receptor. This flexibility and/or physical distance (which is herein referred to as “slack”) may be normally present in the first and/or second domain(s), normally present in the linker, and/or normally present in the chimeric protein (as a whole). Alternately, or additionally, the chimeric protein may be modified by including one or more additional amino acid sequences (e.g., the joining linkers described below) or synthetic linkers (e.g., a polyethylene glycol (PEG) linker) which provide additional slack needed to avoid steric hindrance. Further description of linkers useful in the present invention, and especially the linkers of SEQ ID NO: 1 to SEQ ID NO: 3, are included in the next section of this disclosure entitled “Linkers”.

In embodiments, the chimeric protein is capable of contemporaneously binding the CSF3 receptor and the TL1A receptor. In embodiments, the CSF3 receptor is granulocyte colony-stimulating factor receptor (G-CSF-R) also known as CD114 (Cluster of Differentiation 114) and the TL1A receptor is TNFRSF25/DR3 or TNFRSF21/DR6/DcR3. CSF3 is a cytokine that controls the production, differentiation, and function of two related white cell populations of the blood, the granulocytes and the monocytes-macrophages and is capable of suppressing many autoimmune diseases like Crohn's disease, Type-1 diabetes, Myasthenia gravis and experimental autoimmune thyroiditis. TL1A binding to its receptor promotes, at least, expansion of activated and regulatory T cells, which can decrease an autoimmune response. Accordingly, a chimeric protein comprising a portion of CSF3 which includes its receptor-binding domain and the extracellular domain of TL1A is capable of contemporaneously stimulating granulocytosis and mobilizing stem cells from the bone marrow (via CSF3) and inhibiting an immune activating signal (via TL1A). In embodiments, this chimeric protein is referred to herein as CSF3-Fc-TL1A.

In embodiments, the chimeric proteins of the present invention comprise variants of a portion of CSF3 which includes its receptor-binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the portion of CSF3, e.g., human CSF3, which comprises its receptor-binding domain.

In embodiments, the portion of CSF3 comprising its receptor-binding domain has the following amino acid sequence:

(SEQ ID NO: 57)
ATPLGPASSLPQSFLLKCLEQVRKIQGDGAALQEKLVSECATYKLCHPEE
LVLLGHSLGIPWAPLSSCPSQALQLAGCLSQLHSGLFLYQGLLQALEGIS
PELGPTLDTLQLDVADFATTIWQQMEELGMAPALQPTQGAMPAFASAFQR
RAGGVLVASHLQSFLEVSYRVLRHLAQP.

In embodiments, a chimeric protein comprises a variant of the portion of CSF3 comprising its receptor-binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 57.

In embodiments, the first domain of a chimeric protein comprises an amino acid sequence that is at least 95% identical to the amino acid sequence of SEQ ID NO: 57.

One of ordinary skill may select variants of the known amino acid sequence of CSF3 by consulting the literature, e.g., Aritomi et al., “Atomic structure of the GCSF-receptor complex showing a new cytokine-receptor recognition scheme.” Nature 401:713-717 (1999); Tamada et al., “Homodimeric cross-over structure of the human granulocyte colony-stimulating factor (GCSF) receptor signaling complex.” Proc. Natl. Acad. Sci. U.S.A. 103:3135-3140 (2006); Zink et al., “Secondary structure of human granulocyte colony-stimulating factor derived from NMR spectroscopy.” FEBS Lett. 314 (3), 435-439 (1992); and Battacharya et al., “GM-CSF: An Immune Modulatory Cytokine that can Suppress Autoimmunity.” Cytokine. 2015 October; 75(2): 261-271, each of which is incorporated by reference in its entirety.

In embodiments, the chimeric proteins of the present invention comprise variants of the extracellular domain, which includes the receptor-binding domain, of TL1A. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of TL1A, e.g., human TL1A.

In embodiments, the extracellular domain of TL1A has the following amino acid sequence:

(SEQ ID NO: 58)
SQLRAQGEACVQFQALKGQEFAPSHQQVYAPLRADGDKPRAHLTVVRQTP
TQHFKNQFPALHWEHELGLAFTKNRMNYTNKFLLIPESGDYFIYSQVTFR
GMTSECSEIRQAGRPNKPDSITVVITKVIDSYPEPTQLLMGTKSVCEVGS
NWFQPIYLGAMFSLQEGDKLMVNVSDISLVDYTKEDKTFFGAFLL.

In embodiments, a chimeric protein comprises a variant of the extracellular domain of TL1A. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 58.

In embodiments, the second domain of a chimeric protein comprises an amino acid sequence that is at least 95% identical to the amino acid sequence of SEQ ID NO: 58.

One of ordinary skill may select variants of the known amino acid sequence of TL1A by consulting the literature, e.g., Yue et al., “TL1, a novel tumor necrosis factor-like cytokine, induces apoptosis in endothelial cells. Involvement of activation of stress protein kinases (stress-activated protein kinase and p38 mitogen-activated protein kinase) and caspase-3-like protease.” J. Biol. Chem. 274 (3), 1479-1486 (1999); Richard et al., “Reduced monocyte and macrophage TNFSF15/TL1A expression is associated with susceptibility to inflammatory bowel disease.” PLoS Genet. 14 (9), e1007458 (2018); Migone et al., “TL1A is a TNF-like ligand for DR3 and TR6/DcR3 and functions as a T cell costimulator.” Immunity 16:479-492(2002); Jin et al., “X-ray crystal structure of TNF ligand family member TL1A at 2.1A.” Biochem. Biophys. Res. Commun. 364:1-6(2007); Zhan et al., “Decoy strategies: the structure of TL1A:DcR3 complex.” Structure 19:162-171(2011); Khan et al., “TL1A-Ig induces transplantation tolerance”, J Immunol May 1, 2013, 190 (1 Supplement) 113.2; Khan et al., “Cloning, Expression, and Functional Characterization of TL1A-Ig” J Immunol Feb. 15, 2013, 190 (4) 1540-1550; and Schreiber et al. “Therapeutic Treg expansion in mice by TNFRSF25 prevents allergic lung inflammation” Clin Invest. 2010; 120(10):3629-3640, each of which is incorporated by reference in its entirety.

In embodiments, a chimeric protein of the present invention comprises: (1) a first domain comprising the amino acid sequence of SEQ ID NO: 57, (b) a second domain comprises the amino acid sequence of SEQ ID NO: 58, and (c) a linker comprises an amino acid sequence that is at least 95% identical to SEQ ID NO: 1, SEQ ID NO: 2, or SEQ ID NO: 3.

In embodiments, a CSF3-Fc-TL1A chimeric protein of the present invention has the following amino acid sequence:

(SEQ ID NO: 59)
ATPLGPASSLPQSFLLKCLEQVRKIQGDGAALQEKLVSECATYKLCHPEE
LVLLGHSLGIPWAPLSSCPSQALQLAGCLSQLHSGLFLYQGLLQALEGIS
PELGPTLDTLQLDVADFATTIWQQMEELGMAPALQPTQGAMPAFASAFQR
RAGGVLVASHLQSFLEVSYRVLRHLAQPSKYGPPCPPCPAPEFLGGPSVF
LFPPKPKDQLMISRTPEVICVVVDVSQEDPEVQFNWYVDGVEVHNAKTKP
REEQFNSTYRWSVLTVLHQDWLSGKEYKCKVSSKGLPSSIEKTISNATGQ
PREPQVYTLPPSQEEMTKNQVSLTCLVKGFYPSDIAVEWESNGQPENNYK
TTPPVLDSDGSFFLYSRLTVDKSRWQEGNVFSCSVLHEALHNHYTQKSLS
LSLGKIEGRMDSQLRAQGEACVQFQALKGQEFAPSHQQVYAPLRADGDKP
RAHLTWRQTPTQHFKNQFPALHWEHELGLAFTKNRMNYTNKFLLIPESGD
YFIYSQVTFRGMTSECSEIRQAGRPNKPDSITWITKVIDSYPEPTQLLMG
TKSVCEVGSNWFQPIYLGAMFSLQEGDKLMVNVSDISLVDYTKEDKTFFG
AFLL.

In embodiments, a chimeric protein comprises a variant of a CSF3-Fc-TL1A chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 59.

In embodiments, the chimeric protein is capable of contemporaneously binding the CTLA4 ligand and the TL1A receptor. In embodiments, the CTLA4 ligand is CD80 or CD86 and the TL1A receptor is TNFRSF25/DR3 or TNFRSF21/DR6/DcR3. CTLA4 acts as an “off” switch when bound to its ligand on the surface of antigen-presenting cells (APCs). CTLA4 is a protein receptor that functions as an immune checkpoint and downregulates immune responses. When TL1A binds to its receptor promotes, at least, expansion of activated and regulatory T cells. Accordingly, a chimeric protein comprising the extracellular domains of CTLA4 and TL1A is capable of contemporaneously competitively inhibiting an immune activating signal (via CTLA4) and activating an immune receptor TNFRSF25 (via TL1A), which stimulates regulatory T cell proliferation. In embodiments, this chimeric protein is referred to herein as CTLA4-Fc-TL1A.

In embodiments, the chimeric proteins of the present invention comprise variants of the extracellular domain, which includes the ligand/receptor binding domain, of CTLA4. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of CTLA4, e.g., human CTLA4.

In embodiments, the extracellular domain of CTLA4 has the following amino acid sequence:

(SEQ ID NO: 60)
KAMHVAQPAVVLASSRGIASFVCEYASPGKATEVRVTVLRQADSQVTEVC
AATYMMGNELTFLDDSICTGTSSGNQVNLTIQGLRAMDTGLYICKVELMY
PPPYYLGIGNGTQIYVIDPEPCPDSD.

In embodiments, a chimeric protein comprises a variant of the extracellular domain of CTLA4. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 60.

In embodiments, the first domain of a chimeric protein comprises an amino acid sequence that is at least 95% identical to the amino acid sequence of SEQ ID NO: 60.

One of ordinary skill may select variants of the known amino acid sequence of CTLA4 by consulting the literature, e.g., Linsley et al., “CTLA-4 is a second receptor for the B cell activation antigen B7.” J. Exp. Med. 174 (3), 561-569 (1991); Dariavach et al., “Human Ig superfamily CTLA-4 gene: chromosomal localization and identity of protein sequence between murine and human CTLA-4 cytoplasmic domains”. Eur. J. Immunol. 18 (12), 1901-1905 (1988); Teft et al., “A molecular perspective of CTLA-4 function.” Annu. Rev. Immunol. 24:65-97 (2006); Ramagopal et al., “Structural basis for cancer immunotherapy by the first-in-class checkpoint inhibitor ipilimumab.” Proc. Natl. Acad. Sci. U.S.A. 114:E4223-E4232 (2017); Schwartz et al., “Structural basis for co-stimulation by the human CTLA-4/B7-2 complex.” Nature 410:604-608 (2001); Stamper et al., “Crystal structure of the B7-1/CTLA-4 complex that inhibits human immune responses.” Nature 410:608-611 (2001); and Yu et al., “Rigid-body ligand recognition drives cytotoxic T-lymphocyte antigen 4 (CTLA-4) receptor triggering.” J. Biol. Chem. 286:6685-6696 (2011), each of which is incorporated by reference in its entirety.

In embodiments, the chimeric proteins of the present invention comprise variants of the extracellular domain, which includes the receptor-binding domain, of TL1A. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of TL1A, e.g., human TL1A.

In embodiments, the extracellular domain of TL1A has the following amino acid sequence:

(SEQ ID NO: 58)
SQLRAQGEACVQFQALKGQEFAPSHQQVYAPLRADGDKPRAHLTVVRQTP
TQHFKNQFPALHWEHELGLAFTKNRMNYTNKFLLIPESGDYFIYSQVTFR
GMTSECSEIRQAGRPNKPDSITVVITKVIDSYPEPTQLLMGTKSVCEVGS
NWFQPIYLGAMFSLQEGDKLMVNVSDISLVDYTKEDKTFFGAFLL.

In embodiments, a chimeric protein comprises a variant of the extracellular domain of TL1A. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 58.

In embodiments, the second domain of a chimeric protein comprises an amino acid sequence that is at least 95% identical to the amino acid sequence of SEQ ID NO: 58.

One of ordinary skill may select variants of the known amino acid sequence of TL1A by consulting the literature, e.g., Yue et al., “TL1, a novel tumor necrosis factor-like cytokine, induces apoptosis in endothelial cells. Involvement of activation of stress protein kinases (stress-activated protein kinase and p38 mitogen-activated protein kinase) and caspase-3-like protease.” J. Biol. Chem. 274 (3), 1479-1486 (1999); Richard et al., “Reduced monocyte and macrophage TNFSF15/TL1A expression is associated with susceptibility to inflammatory bowel disease.” PLoS Genet. 14 (9), e1007458 (2018); Migone et al., “TL1A is a TNF-like ligand for DR3 and TR6/DcR3 and functions as a T cell costimulator.” Immunity 16:479-492(2002); Jin et al., “X-ray crystal structure of TNF ligand family member TL1A at 2.1A.” Biochem. Biophys. Res. Commun. 364:1-6(2007); Zhan et al., “Decoy strategies: the structure of TL1A:DcR3 complex.” Structure 19:162-171(2011); Khan et al., “TL1A-Ig induces transplantation tolerance”, J Immunol May 1, 2013, 190 (1 Supplement) 113.2; Khan et al., “Cloning, Expression, and Functional Characterization of TL1A-Ig” J Immunol Feb. 15, 2013, 190 (4) 1540-1550; and Schreiber et al. “Therapeutic Treg expansion in mice by TNFRSF25 prevents allergic lung inflammation” Clin Invest. 2010; 120(10):3629-3640, each of which is incorporated by reference in its entirety.

In embodiments, a chimeric protein of the present invention comprises: (1) a first domain comprising the amino acid sequence of SEQ ID NO: 60, (b) a second domain comprises the amino acid sequence of SEQ ID NO: 58, and (c) a linker comprises an amino acid sequence that is at least 95% identical to SEQ ID NO: 1, SEQ ID NO: 2, or SEQ ID NO: 3.

In embodiments, a CTLA4-Fc-TL1A chimeric protein of the present invention has the following amino acid sequence:

(SEQ ID NO: 61)
KAMHVAQPAVVLASSRGIASFVCEYASPGKATEVRVTVLRQADSQVTEVC
AATYMMGNELTFLDDSICTGTSSGNQVNLTIQGLRAMDTGLYICKVELMY
PPPYYLGIGNGTQIYVIDPEPCPDSDSKYGPPCPPCPAPEFLGGPSVFLF
PPKPKDQLMISRTPEVTCWVDVSQEDPEVQFNWYVDGVEVHNAKTKPREE
QFNSTYRVVSVLTVLHQDWLSGKEYKCKVSSKGLPSSIEKTISNATGQPR
EPQVYTLPPSQEEMTKNQVSLTCLVKGFYPSDIAVEWESNGQPENNYKTT
PPVLDSDGSFFLYSRLTVDKSRWQEGNVFSCSVLHEALHNHYTQKSLSLS
LGKIEGRMDSQLRAQGEACVQFQALKGQEFAPSHQQVYAPLRADGDKPRA
HLTVVRQTPTQHFKNQFPALHWEHELGLAFTKNRMNYTNKFLLIPESGDY
FIYSQVTFRGMTSECSEIRQAGRPNKPDSITVVITKVTDSYPEPTQLLMG
TKSVCEVGSNWFQPIYLGAMFSLQEGDKLMVNVSDISLVDYTKEDKTFFG
AFLL.

In embodiments, a chimeric protein comprises a variant of a CTLA4-Fc-TL1A chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 61.

In embodiments, the chimeric protein is capable of contemporaneously binding the CTLA4 ligand and the PD-L1 receptor. In embodiments, the CTLA4 ligand is CD80 or CD86 and the PD-L1 receptor is PD-1. CTLA4 acts as an “off” switch when bound to its ligand on the surface of antigen-presenting cells (APCs). CTLA4 is a protein receptor that functions as an immune checkpoint and downregulates immune responses. PD-L1 plays a critical role in induction and maintenance of immune tolerance to self, in part, by acting as a ligand for the inhibitory receptor PD-1; it modulates the activation threshold of T-cells and limits T-cell effector response, including cytotoxic T lymphocytes (CTLs) effector function Accordingly, a chimeric protein comprising the extracellular domains of CTLA4 and PD-L1 is capable of contemporaneously competitively inhibiting an immune activating signal (via CTLA4) and activating an immune inhibitory signal (via PD-L1). In embodiments, this chimeric protein is referred to herein as CTLA4-Fc-PD-L1.

In embodiments, the chimeric proteins of the present invention comprise variants of the extracellular domain, which includes the ligand/receptor binding domain, of CTLA4. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of CTLA4, e.g., human CTLA4.

In embodiments, the extracellular domain of CTLA4 has the following amino acid sequence:

(SEQ ID NO: 60)
KAMHVAQPAVVLASSRGIASFVCEYASPGKATEVRVTVLRQADSQVTEVC
AATYMMGNELTFLDDSICTGTSSGNQVNLTIQGLRAMDTGLYICKVELMY
PPPYYLGIGNGTQIYVIDPEPCPDSD.

In embodiments, a chimeric protein comprises a variant of the extracellular domain of CTLA4. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 60.

In embodiments, the first domain of a chimeric protein comprises an amino acid sequence that is at least 95% identical to the amino acid sequence of SEQ ID NO: 60.

One of ordinary skill may select variants of the known amino acid sequence of CTLA4 by consulting the literature, e.g., Linsley et al., “CTLA-4 is a second receptor for the B cell activation antigen B7.” J. Exp. Med. 174 (3), 561-569 (1991); Dariavach et al., “Human Ig superfamily CTLA-4 gene: chromosomal localization and identity of protein sequence between murine and human CTLA-4 cytoplasmic domains”. Eur. J. Immunol. 18 (12), 1901-1905 (1988); Teft et al., “A molecular perspective of CTLA-4 function.” Annu. Rev. Immunol. 24:65-97 (2006); Ramagopal et al., “Structural basis for cancer immunotherapy by the first-in-class checkpoint inhibitor ipilimumab.” Proc. Natl. Acad. Sci. U.S.A. 114:E4223-E4232 (2017); Schwartz et al., “Structural basis for co-stimulation by the human CTLA-4/B7-2 complex.” Nature 410:604-608 (2001); Stamper et al., “Crystal structure of the B7-1/CTLA-4 complex that inhibits human immune responses.” Nature 410:608-611 (2001); and Yu et al., “Rigid-body ligand recognition drives cytotoxic T-lymphocyte antigen 4 (CTLA-4) receptor triggering.” J. Biol. Chem. 286:6685-6696 (2011), each of which is incorporated by reference in its entirety.

In embodiments, the chimeric proteins of the present invention comprise variants of the extracellular domain, which includes the receptor-binding domain, of PD-L1. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of PD-L1, e.g., human PD-L1.

In embodiments, the extracellular domain of PD-L1 has the following amino acid sequence:

(SEQ ID NO: 62)
FTVTVPKDLYVVEYGSNMTIECKFPVEKQLDLAALIVYWEMEDKNIIQFV
HGEEDLKVQHSSYRQRARLLKDQLSLGNAALQITDVKLQDAGVYRCMISY
GGADYKRITVKVNAPYNKINQRILVVDPVTSEHELTCQAEGYPKAEVIWT
SSDHQVLSGKTTTTNSKREEKLFNVTSTLRINTTTNEIFYCTFRRLDPEE
NHTAELVIPELPLAHPPNER.

In embodiments, a chimeric protein comprises a variant of the extracellular domain of PD-L1. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 62.

In embodiments, the second domain of a chimeric protein comprises an amino acid sequence that is at least 95% identical to the amino acid sequence of SEQ ID NO: 62.

One of ordinary skill may select variants of the known amino acid sequence of PD-L1 by consulting the literature, e.g., Freeman et al., “Engagement of the PD-1 immunoinhibitory receptor by a novel B7-family member leads to negative regulation of lymphocyte activation.” J. Exp. Med. 192:1027-1034 (2000); Burr, et al, “CMTM6 maintains the expression of PD-L1 and regulates anti-tumour immunity.” Nature 549 (7670), 101-105 (2017); Lin et al., “The PD-1/PD-L1 complex resembles the antigen-binding Fv domains of antibodies and T cell receptors.” Proc. Natl. Acad. Sci. U.S.A. 105 (8), 3011-3016 (2008); and Zak et al., “Structure of the Complex of Human Programmed Death 1, PD-1, and Its Ligand PD-L1.” Structure 23 (12), 2341-2348 (2015), each of which is incorporated by reference in its entirety.

In embodiments, a chimeric protein of the present invention comprises: (1) a first domain comprising the amino acid sequence of SEQ ID NO: 60, (b) a second domain comprises the amino acid sequence of SEQ ID NO: 62, and (c) a linker comprises an amino acid sequence that is at least 95% identical to SEQ ID NO: 1, SEQ ID NO: 2, or SEQ ID NO: 3.

In embodiments, a CTLA4-Fc-PD-L1 chimeric protein of the present invention has the following amino acid sequence:

(SEQ ID NO: 63)
KAMHVAQPAVVLASSRGIASFVCEYASPGKATEVRVTVLRQADSQVTEVC
AATYMMGNELTFLDDSICTGTSSGNQVNLTIQGLRAMDTGLYICKVELMY
PPPYYLGIGNGTQIYVIDPEPCPDSDSKYGPPCPPCPAPEFLGGPSVFLF
PPKPKDQLMISRTPEVICVVVDVSQEDPEVQFNWYVDGVEVHNAKTKPRE
EQFNSTYRVVSVLTVLHQDWLSGKEYKCKVSSKGLPSSIEKTISNATGQP
REPQVYTLPPSQEEMTKNQVSLTCLVKGFYPSDIAVEWESNGQPENNYKT
TPPVLDSDGSFFLYSRLTVDKSRWQEGNVFSCSVLHEALHNHYTQKSLSL
SLGKIEGRMDFTVTVPKDLYVVEYGSNMTIECKFPVEKQLDLAALIVYWE
MEDKNIIQFVHGEEDLKVQHSSYRQRARLLKDQLSLGNAALQITDVKLQD
AGVYRCMISYGGADYKRITVKVNAPYNKINQRILVVDPVTSEHELTCQAE
GYPKAEVIWTSSDHQVLSGKTTTTNSKREEKLFNVTSTLRINTTTNEIFY
CTFRRLDPEENHTAELVIPELPLAHPPNER.

In embodiments, a chimeric protein comprises a variant of a CTLA4-Fc-PD-L1 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 63.

In embodiments, the chimeric protein is capable of contemporaneously binding the B7H3 receptor and the PD-L1 receptor. In embodiments, the PD-L1 receptor is PD-1; however, the B7H3 receptor has not been characterized. B7H3 (CD276) is an important immune checkpoint member of the B7 and CD28 families, many of whom interact with known checkpoint markers including CTLA4, PD-1, and CD28. B7-H3 plays an important role in the inhibition of T-cell function. PD-L1 plays a critical role in induction and maintenance of immune tolerance to self, in part, by acting as a ligand for the inhibitory receptor PD-1; it modulates the activation threshold of T-cells and limits T-cell effector response, including cytotoxic T lymphocytes (CTLs) effector function Accordingly, a chimeric protein comprising the extracellular domains of B7H3 and PD-L1 is capable of contemporaneously activating an immune inhibitory signal (via B7H3 receptors) and activating an immune inhibitory signal (via PD-L1). In embodiments, this chimeric protein is referred to herein as B7H3-Fc-PD-L1.

In embodiments, the chimeric proteins of the present invention comprise variants of the extracellular domain, which includes the receptor-binding domain, of B7H3. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of B7H3, e.g., human B7H3.

In embodiments, the extracellular domain of B7H3 has the following amino acid sequence:

(SEQ ID NO: 64)
LEVQVPEDPVVALVGTDATLCCSFSPEPGFSLAQLNLIWQLTDTKQLVHS
FAEGQDQGSAYANRTALFPDLLAQGNASLRLQRVRVADEGSFTCFVSIRD
FGSAAVSLQVAAPYSKPSMTLEPNKDLRPGDTVTITCSSYQGYPEAEVFW
QDGQGVPLIGNVITSQMANEQGLFDVHSILRWLGANGTYSCLVRNPVLQQ
DAHSSVTITPQRSPTGAVEVQVPEDPVVALVGTDATLRCSFSPEPGFSLA
QLNLIWQLTDTKQLVHSFTEGRDQGSAYANRTALFPDLLAQGNASLRLQR
VRVADEGSFTCFVSIRDFGSAAVSLQVAAPYSKPSMTLEPNKDLRPGDTV
TITCSSYRGYPEAEVFWQDGQGVPLIGNVITSQMANEQGLFDVHSVLRWL
GANGTYSCLVRNPVLQQDAHGSVTITGQPMTFPPEA.

In embodiments, a chimeric protein comprises a variant of the extracellular domain of B7H3. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 64.

In embodiments, the first domain of a chimeric protein comprises an amino acid sequence that is at least 95% identical to the amino acid sequence of SEQ ID NO: 64.

One of ordinary skill may select variants of the known amino acid sequence of B7H3 by consulting the literature, e.g., Chapoval et al., “B7-H3: a costimulatory molecule for T cell activation and IFN-gamma production.” Nat. Immunol. 2:269-274 (2001); Steinberger et al., “Molecular characterization of human 4Ig-B7-H3, a member of the B7 family with four Ig-like domains.” J. Immunol. 172:2352-2359 (2004); Wang et al., “B7-H3 promotes acute and chronic allograft rejection.” Eur. J. Immunol. 35:428-438 (2005); Castellanos et al., “B7-H3 role in the immune landscape of cancer” Am J Clin Exp Immunol. 2017; 6(4): 66-75; and Vigdorovich et al., “Structure and T cell inhibition properties of B7 family member, B7-H3.” Structure. 2013 May 7; 21(5):707-17, each of which is incorporated by reference in its entirety.

In embodiments, the chimeric proteins of the present invention comprise variants of the extracellular domain, which includes the receptor-binding domain, of PD-L1. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of PD-L1, e.g., human PD-L1.

In embodiments, the extracellular domain of PD-L1 has the following amino acid sequence:

(SEQ ID NO: 62)
FTITAPKDLYVVEYGSNVTMECRFPVERELDLLALVVYWEKEDEQVIQFV
AGEEDLKPQHSNFRGRASLPKDQLLKGNAALQITDVKLQDAGVYCCIISY
GGADYKRITLKVNAPYRKINQRISVDPATSEHELICQAEGYPEAEVIWTN
SDHQPVSGKRSVTTSRTEGMLLNVTSSLRVNATANDVFYCTFWRSQPGQN
HTAELIIPELPATHPPQNRTH.

In embodiments, a chimeric protein comprises a variant of the extracellular domain of PD-L1. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 62.

In embodiments, the second domain of a chimeric protein comprises an amino acid sequence that is at least 95% identical to the amino acid sequence of SEQ ID NO: 62.

One of ordinary skill may select variants of the known amino acid sequence of PD-L1 by consulting the literature, e.g., Freeman et al., “Engagement of the PD-1 immunoinhibitory receptor by a novel B7-family member leads to negative regulation of lymphocyte activation.” J. Exp. Med. 192:1027-1034 (2000); Burr, et al, “CMTM6 maintains the expression of PD-L1 and regulates anti-tumour immunity.” Nature 549 (7670), 101-105 (2017); Lin et al., “The PD-1/PD-L1 complex resembles the antigen-binding Fv domains of antibodies and T cell receptors.” Proc. Natl. Acad. Sci. U.S.A. 105 (8), 3011-3016 (2008); and Zak et al., “Structure of the Complex of Human Programmed Death 1, PD-1, and Its Ligand PD-L1.” Structure 23 (12), 2341-2348 (2015), each of which is incorporated by reference in its entirety.

In embodiments, a chimeric protein of the present invention comprises: (1) a first domain comprising the amino acid sequence of SEQ ID NO: 64, (b) a second domain comprises the amino acid sequence of SEQ ID NO: 62, and (c) a linker comprises an amino acid sequence that is at least 95% identical to SEQ ID NO: 1, SEQ ID NO: 2, or SEQ ID NO: 3.

In embodiments, a B7H3-Fc-PD-L1 chimeric protein of the present invention has the following amino acid sequence:

(SEQ ID NO: 65)
LEVQVPEDPVVALVGTDATLCCSFSPEPGFSLAQLNLIWQLTDTKQLVHS
FAEGQDQGSAYANRTALFPDLLAQGNASLRLQRVRVADEGSFTCFVSIRD
FGSAAVSLQVAAPYSKPSMTLEPNKDLRPGDTVTITCSSYQGYPEAEVFW
QDGQGVPLTGNVTTSQMANEQGLFDVHSILRVVLGANGTYSCLVRNPVLQ
QDAHSSVTITPQRSPTGAVEVQVPEDPWALVGTDATLRCSFSPEPGFSLA
QLNLIWQLTDTKQLVHSFTEGRDQGSAYANRTALFPDLLAQGNASLRLQR
VRVADEGSFTCFVSIRDFGSAAVSLQVAAPYSKPSMTLEPNKDLRPGDTV
TITCSSYRGYPEAEVFWQDGQGVPLTGNVTTSQMANEQGLFDVHSVLRVV
LGANGTYSCLVRNPVLQQDAHGSVTITGQPMTFPPEASKYGPPCPPCPAP
EFLGGPSVFLFPPKPKDQLMISRTPEVTCVVVDVSQEDPEVQFNWYVDGV
EVHNAKTKPREEQFNSTYRVVSVLTVLHQDWLSGKEYKCKVSSKGLPSSI
EKTISNATGQPREPQVYTLPPSQEEMTKNQVSLTCLVKGFYPSDIAVEWE
SNGQPENNYKTTPPVLDSDGSFFLYSRLTVDKSRWQEGNVFSCSVLHEAL
HNHYTQKSLSLSLGKIEGRMDFTVTVPKDLYVVEYGSNMTIECKFPVEKQ
LDLAALIVYWEMEDKNIIQFVHGEEDLKVQHSSYRQRARLLKDQLSLGNA
ALQITDVKLQDAGVYRCMISYGGADYKRITVKVNAPYNKINQRILVVDPV
TSEHELTCQAEGYPKAEVIWTSSDHQVLSGKTTTTNSKREEKLFNVTSTL
RINTTTNEIFYCTFRRLDPEENHTAELVIPELPLAHPPNER.

In embodiments, a chimeric protein comprises a variant of a B7H3-Fc-PD-L1 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 65.

In embodiments, the chimeric protein is capable of contemporaneously binding the B7H4 receptor and the PD-L1 receptor. In embodiments, the B7H4 ligand is CD28 and MIM 186760 and the PD-L1 receptor is PD-1. B7H4 is an immune checkpoint molecule that negatively regulates T-cell-mediated immune response by inhibiting T-cell activation, proliferation, cytokine production and development of cytotoxicity. PD-L1 plays a critical role in induction and maintenance of immune tolerance to self, in part, by acting as a ligand for the inhibitory receptor PD-1; it modulates the activation threshold of T-cells and limits T-cell effector response, including cytotoxic T lymphocytes (CTLs) effector function Accordingly, a chimeric protein comprising the extracellular domains of B7H4 and PD-L1 is capable of contemporaneously activating an immune inhibitory signal (via B7H4 receptors) and activating an immune inhibitory signal (via PD-L1). In embodiments, this chimeric protein is referred to herein as B7H4-Fc-PD-L1.

In embodiments, the chimeric proteins of the present invention comprise variants of the extracellular domain, which includes the ligand-binding domain, of B7H4. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of B7H4, e.g., human B7H4.

In embodiments, the extracellular domain of B7H4 has the following amino acid sequence:

(SEQ ID NO: 66)
LIIGFGISGRHSITVTTVASAGNIGEDGILSCTFEPDIKLSDIVIQWLKE
GVLGLVHEFKEGKDELSEQDEMFRGRTAVFADQVIVGNASLRLKNVQLTD
AGTYKCYIITSKGKGNANLEYKTGAFSMPEVNVDYNASSETLRCEAPRWF
PQPTVVWASQVDQGANFSEVSNTSFELNSENVTMKVVSVLYNVTINNTYS
CMIENDIAKATGDIKVTESEIKRRSH.

In embodiments, a chimeric protein comprises a variant of the extracellular domain of B7H4. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 66.

In embodiments, the first domain of a chimeric protein comprises an amino acid sequence that is at least 95% identical to the amino acid sequence of SEQ ID NO: 66.

One of ordinary skill may select variants of the known amino acid sequence of B7H4 by consulting the literature, e.g., Salceda et al., “The immunomodulatory protein B7-H4 is overexpressed in breast and ovarian cancers and promotes epithelial cell transformation.” Exp. Cell Res. 306:128-141 (2005); Kryczek et al., “B7-H4 expression identifies a novel suppressive macrophage population in human ovarian carcinoma.” J. Exp. Med. 203:871-881 (2006); and Vigdorovich et al., “Structure and cancer immunotherapy of the B7 family member B7x”, Cell Rep (2014) 9 p. 1089-98; each of which is incorporated by reference in its entirety.

In embodiments, the chimeric proteins of the present invention comprise variants of the extracellular domain, which includes the receptor-binding domain, of PD-L1. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of PD-L1, e.g., human PD-L1.

In embodiments, the extracellular domain of PD-L1 has the following amino acid sequence:

(SEQ ID NO: 62)
FTITAPKDLYVVEYGSNVTMECRFPVERELDLLALVVYWEKEDEQVIQFV
AGEEDLKPQHSNFRGRASLPKDQLLKGNAALQITDVKLQDAGVYCCIISY
GGADYKRITLKVNAPYRKINQRISVDPATSEHELICQAEGYPEAEVIWTN
SDHQPVSGKRSVTTSRTEGMLLNVTSSLRVNATANDVFYCTFWRSQPGQN
HTAELIIPELPATHPPQNRTH.

In embodiments, a chimeric protein comprises a variant of the extracellular domain of PD-L1. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 62.

In embodiments, the second domain of a chimeric protein comprises an amino acid sequence that is at least 95% identical to the amino acid sequence of SEQ ID NO: 62.

One of ordinary skill may select variants of the known amino acid sequence of PD-L1 by consulting the literature, e.g., Freeman et al., “Engagement of the PD-1 immunoinhibitory receptor by a novel B7-family member leads to negative regulation of lymphocyte activation.” J. Exp. Med. 192:1027-1034 (2000); Burr, et al, “CMTM6 maintains the expression of PD-L1 and regulates anti-tumour immunity.” Nature 549 (7670), 101-105 (2017); Lin et al., “The PD-1/PD-L1 complex resembles the antigen-binding Fv domains of antibodies and T cell receptors.” Proc. Natl. Acad. Sci. U.S.A. 105 (8), 3011-3016 (2008); and Zak et al., “Structure of the Complex of Human Programmed Death 1, PD-1, and Its Ligand PD-L1.” Structure 23 (12), 2341-2348 (2015), each of which is incorporated by reference in its entirety.

In embodiments, a chimeric protein of the present invention comprises: (1) a first domain comprising the amino acid sequence of SEQ ID NO: 66, (b) a second domain comprises the amino acid sequence of SEQ ID NO: 62, and (c) a linker comprises an amino acid sequence that is at least 95% identical to SEQ ID NO: 1, SEQ ID NO: 2, or SEQ ID NO: 3.

In embodiments, a B7H4-Fc-PD-L1 chimeric protein of the present invention has the following amino acid sequence:

(SEQ ID NO: 67)
LIIGFGISGRHSITVTTVASAGNIGEDGILSCTFEPDIKLSDIVIQWLKE
GVLGLVHEFKEGKDELSEQDEMFRGRTAVFADQVIVGNASLRLKNVQLTD
AGTYKCYIITSKGKGNANLEYKTGAFSMPEVNVDYNASSETLRCEAPRWF
PQPTVVWASQVDQGANFSEVSNTSFELNSENVTMKVVSVLYNVTINNTYS
CMIENDIAKATGDIKVTESEIKRRSHSKYGPPCPPCPAPEFLGGPSVFLF
PPKPKDQLMISRTPEVICVVVDVSQEDPEVQFNWYVDGVEVHNAKTKPRE
EQFNSTYRVVSVLTVLHQDWLSGKEYKCKVSSKGLPSSIEKTISNATGQP
REPQVYTLPPSQEEMTKNQVSLTCLVKGFYPSDIAVEWESNGQPENNYKT
TPPVLDSDGSFFLYSRLTVDKSRWQEGNVFSCSVLHEALHNHYTQKSLSL
SLGKIEGRMDFTVTVPKDLYVVEYGSNMTIECKFPVEKQLDLAALIVYWE
MEDKNIIQFVHGEEDLKVQHSSYRQRARLLKDQLSLGNAALQITDVKLQD
AGVYRCMISYGGADYKRITVKVNAPYNKINQRILVVDPVTSEHELTCQAE
GYPKAEVIWTSSDHQVLSGKTTTTNSKREEKLFNVTSTLRINTTTNEIFY
CTFRRLDPEENHTAELVIPELPLAHPPNER.

In embodiments, a chimeric protein comprises a variant of a B7H4-Fc-PD-L1 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 67.

In embodiments, the chimeric protein is capable of contemporaneously binding the ILDR2 receptor and the PD-L1 receptor. In embodiments, the PD-L1 receptor is PD-1; however, the ILDR2 ligand/receptor is presently unknown. ILDR2 is B7-like protein with robust T cell inhibitory activity. PD-L1 plays a critical role in induction and maintenance of immune tolerance to self, in part, by acting as a ligand for the inhibitory receptor PD-1; it modulates the activation threshold of T-cells and limits T-cell effector response, including cytotoxic T lymphocytes (CTLs) effector function Accordingly, a chimeric protein comprising the extracellular domains of ILDR2 and PD-L1 is capable of contemporaneously activating an immune inhibitory signal (via ILDR2) and activating an immune inhibitory signal (via PD-L1). In embodiments, this chimeric protein is referred to herein as ILDR2-Fc-PD-L1.

In embodiments, the chimeric proteins of the present invention comprise variants of the extracellular domain, which includes the ligand/receptor-binding domain, of ILDR2. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of ILDR2, e.g., human ILDR2.

In embodiments, the extracellular domain of ILDR2 has the following amino acid sequence:

(SEQ ID NO: 70)
LQVTVPDKKKVAMLFQPTVLRCHFSTSSHQPAVVQWKFKSYCQDRMGESL
GMSSTRAQSLSKRNLEWDPYLDCLDSRRTVRVVASKQGSTVTLGDFYRGR
EITIVHDADLQIGKLMWGDSGLYYCIITTPDDLEGKNEDSVELLVLGRTG
LLADLLPSFAVEIMPE.

In embodiments, a chimeric protein comprises a variant of the extracellular domain of ILDR2. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 70.

In embodiments, the first domain of a chimeric protein comprises an amino acid sequence that is at least 95% identical to the amino acid sequence of SEQ ID NO: 70.

One of ordinary skill may select variants of the known amino acid sequence of ILDR2 by consulting the literature, e.g., Hecht et al., “ILDR2 Is a Novel B7-like Protein That Negatively Regulates T Cell Responses”. J Immunol. 2018 Mar. 15; 200(6):2025-2037; Podojil et al., “ILDR2-Fc Is a Novel Regulator of Immune Homeostasis and Inducer of Antigen-Specific Immune Tolerance.” J Immunol. 2018 Mar. 15; 200(6):2013-2024; and Watanabe et al., “ILDR2: An Endoplasmic Reticulum Resident Molecule Mediating Hepatic Lipid Homeostasis.” PLoS One. 2013; 8(6): e67234, each of which is incorporated by reference in its entirety.

In embodiments, the chimeric proteins of the present invention comprise variants of the extracellular domain, which includes the receptor-binding domain, of PD-L1. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of PD-L1, e.g., human PD-L1.

In embodiments, the extracellular domain of PD-L1 has the following amino acid sequence:

(SEQ ID NO: 62)
FTITAPKDLYVVEYGSNVTMECRFPVERELDLLALVVYWEKEDEQVIQFV
AGEEDLKPQHSNFRGRASLPKDQLLKGNAALQITDVKLQDAGVYCCIISY
GGADYKRITLKVNAPYRKINQRISVDPATSEHELICQAEGYPEAEVIWTN
SDHQPVSGKRSVTTSRTEGMLLNVTSSLRVNATANDVFYCTFWRSQPGQN
HTAELIIPELPATHPPQNRTH.

In embodiments, a chimeric protein comprises a variant of the extracellular domain of PD-L1. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 62.

In embodiments, the second domain of a chimeric protein comprises an amino acid sequence that is at least 95% identical to the amino acid sequence of SEQ ID NO: 62.

One of ordinary skill may select variants of the known amino acid sequence of PD-L1 by consulting the literature, e.g., Freeman et al., “Engagement of the PD-1 immunoinhibitory receptor by a novel B7-family member leads to negative regulation of lymphocyte activation.” J. Exp. Med. 192:1027-1034 (2000); Burr, et al, “CMTM6 maintains the expression of PD-L1 and regulates anti-tumour immunity.” Nature 549 (7670), 101-105 (2017); Lin et al., “The PD-1/PD-L1 complex resembles the antigen-binding Fv domains of antibodies and T cell receptors.” Proc. Natl. Acad. Sci. U.S.A. 105 (8), 3011-3016 (2008); and Zak et al., “Structure of the Complex of Human Programmed Death 1, PD-1, and Its Ligand PD-L1.” Structure 23 (12), 2341-2348 (2015), each of which is incorporated by reference in its entirety.

In embodiments, a chimeric protein of the present invention comprises: (1) a first domain comprising the amino acid sequence of SEQ ID NO: 70, (b) a second domain comprises the amino acid sequence of SEQ ID NO: 62, and (c) a linker comprises an amino acid sequence that is at least 95% identical to SEQ ID NO: 1, SEQ ID NO: 2, or SEQ ID NO: 3.

In embodiments, an ILDR2-Fc-PD-L1 chimeric protein of the present invention has the following amino acid sequence:

(SEQ ID NO: 71)
LQVTVPDKKKVAMLFQPTVLRCHFSTSSHQPAVVQWKFKSYCQDRMGESL
GMSSTRAQSLSKRNLEWDPYLDCLDSRRTVRVVASKQGSTVTLGDFYRGR
EITIVHDADLQIGKLMWGDSGLYYCIITTPDDLEGKNEDSVELLVLGRTG
LLADLLPSFAVEIMPEVPRDCGCKPCICTVPEVSSVFIFPPKPKDVLTIT
LTPKVTCVVVDISKDDPEVQFSWFVDDVEVHTAQTQPREEQFNSTFRSVS
ELPIMHQDWLNGKEFKCRVNSAAFPAPIEKTISKTKGRPKAPQVYTIPPP
KEQMAKDKVSLTCMITDFFPEDITVEWQWNGQPAENYKNTQPIMDTDGSY
FVYSKLNVQKSNWEAGNTFTCSVLHEGLHNHHTEKSLSHSPGIIEGRMDF
TITAPKDLYVVEYGSNVTMECRFPVERELDLLALVVYWEKEDEQVIQFVA
GEEDLKPQHSNFRGRASLPKDQLLKGNAALQITDVKLQDAGVYCCIISYG
GADYKRITLKVNAPYRKINQRISVDPATSEHELICQAEGYPEAEVIWTNS
DHQPVSGKRSVTTSRTEGMLLNVTSSLRVNATANDVFYCTFWRSQPGQNH
TAELIIPELPATHPPQNRTH.

In embodiments, a chimeric protein comprises a variant of an ILDR2-Fc-PD-L1 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 71.

In embodiments, the chimeric protein is capable of contemporaneously binding the BTNL2 receptor and the PD-L1 receptor. In embodiments, the PD-L1 receptor is PD-1; however, the BTNL2 ligand/receptor is presently unknown. BTNL2 may be involved in immune surveillance, serving as a negative T-cell regulator by decreasing T-cell proliferation and cytokine release. PD-L1 plays a critical role in induction and maintenance of immune tolerance to self, in part, by acting as a ligand for the inhibitory receptor PD-1; it modulates the activation threshold of T-cells and limits T-cell effector response, including cytotoxic T lymphocytes (CTLs) effector function Accordingly, a chimeric protein comprising the extracellular domains of BTNL2 and PD-L1 is capable of contemporaneously activating an immune inhibitory signal (via BTNL2) and activating an immune inhibitory signal (via PD-L1). In embodiments, this chimeric protein is referred to herein as BTNL2-Fc-PD-L1.

In embodiments, the chimeric proteins of the present invention comprise variants of the extracellular domain, which includes the ligand/receptor-binding domain, of BTNL2. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of BTNL2, e.g., human BTNL2.

In embodiments, the extracellular domain of BTNL2 has the following amino acid sequence:

(SEQ ID NO: 72)
KQSEDFRVIGPAHPILAGVGEDALLTCQLLPKRTTMHVEVRWYRSEPSTP
VFVHRDGVEVTEMQMEEYRGWVEWIENGIAKGNVALKIHNIQPSDNGQYW
CHFQDGNYCGETSLLLKVAGLGSAPSIHMEGPGESGVQLVCTARGWFPEP
QVYWEDIRGEKLLAVSEHRIQDKDGLFYAEATLVVRNASAESVSCLVHNP
VLTEEKGSVISLPEKLQTELASLKVNGPSQPILVRVGEDIQLTCYLSPKA
NAQSMEVRWDRSHRYPAVHVYMDGDHVAGEQMAEYRGRTVLVSDAIDEGR
LTLQILSARPSDDGQYRCLFEKDDVYQEASLDLKVVSLGSSPLITVEGQE
DGEMQPMCSSDGWFPQPHVPWRDMEGKTIPSSSQALTQGSHGLFHVQTLL
RVTNISAVDVTCSISIPFLGEEKIATFSLSGW.

In embodiments, a chimeric protein comprises a variant of the extracellular domain of BTNL2. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 72.

In embodiments, the first domain of a chimeric protein comprises an amino acid sequence that is at least 95% identical to the amino acid sequence of SEQ ID NO: 72.

One of ordinary skill may select variants of the known amino acid sequence of BTNL2 by consulting the literature, e.g., Valentonyte et al., “Sarcoidosis is associated with a truncating splice site mutation in BTNL2.” Nat. Genet. 37:357-364 (2005); Nguyen et al., “BTNL2, a butyrophilin-like molecule that functions to inhibit T cell activation.” J Immunol. 2006 Jun. 15; 176(12):7354-60; Arnett et al., “BTNL2, a butyrophilin/B7-like molecule, is a negative costimulatory molecule modulated in intestinal inflammation.” J. Immunol. 178 (3), 1523-1533 (2007); Rhodes et al., “Regulation of Immunity by Butyrophilins.” Annu Rev Immunol. 2016 May 20; 34:151-72; and Cui et al., “In vivo administration of recombinant BTNL2-Fc fusion protein ameliorates graft-versus-host disease in mice.” Cell Immunol. 2019 January; 335:22-29, each of which is incorporated by reference in its entirety.

In embodiments, the chimeric proteins of the present invention comprise variants of the extracellular domain, which includes the receptor-binding domain, of PD-L1. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of PD-L1, e.g., human PD-L1.

In embodiments, the extracellular domain of PD-L1 has the following amino acid sequence:

(SEQ ID NO: 62)
FTITAPKDLYVVEYGSNVTMECRFPVERELDLLALVVYWEKEDEQVIQFV
AGEEDLKPQHSNFRGRASLPKDQLLKGNAALQITDVKLQDAGVYCCIISY
GGADYKRITLKVNAPYRKINQRISVDPATSEHELICQAEGYPEAEVIWTN
SDHQPVSGKRSVTTSRTEGMLLNVTSSLRVNATANDVFYCTFWRSQPGQN
HTAELIIPELPATHPPQNRTH.

In embodiments, a chimeric protein comprises a variant of the extracellular domain of PD-L1. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 62.

In embodiments, the second domain of a chimeric protein comprises an amino acid sequence that is at least 95% identical to the amino acid sequence of SEQ ID NO: 62.

One of ordinary skill may select variants of the known amino acid sequence of PD-L1 by consulting the literature, e.g., Freeman et al., “Engagement of the PD-1 immunoinhibitory receptor by a novel B7-family member leads to negative regulation of lymphocyte activation.” J. Exp. Med. 192:1027-1034 (2000); Burr, et al, “CMTM6 maintains the expression of PD-L1 and regulates anti-tumour immunity.” Nature 549 (7670), 101-105 (2017); Lin et al., “The PD-1/PD-L1 complex resembles the antigen-binding Fv domains of antibodies and T cell receptors.” Proc. Natl. Acad. Sci. U.S.A. 105 (8), 3011-3016 (2008); and Zak et al., “Structure of the Complex of Human Programmed Death 1, PD-1, and Its Ligand PD-L1.” Structure 23 (12), 2341-2348 (2015), each of which is incorporated by reference in its entirety.

In embodiments, a chimeric protein of the present invention comprises: (1) a first domain comprising the amino acid sequence of SEQ ID NO: 72, (b) a second domain comprises the amino acid sequence of SEQ ID NO: 62, and (c) a linker comprises an amino acid sequence that is at least 95% identical to SEQ ID NO: 1, SEQ ID NO: 2, or SEQ ID NO: 3.

In embodiments, a BTNL2-Fc-PD-L1 chimeric protein of the present invention has the following amino acid sequence:

(SEQ ID NO: 73)
KQSEDFRVIGPAHPILAGVGEDALLTCQLLPKRTTMHVEVRWYRSEPSTP
VFVHRDGVEVTEMQMEEYRGWVEWIENGIAKGNVALKIHNIQPSDNGQYW
CHFQDGNYCGETSLLLKVAGLGSAPSIHMEGPGESGVQLVCTARGWFPEP
QVYWEDIRGEKLLAVSEHRIQDKDGLFYAEATLVVRNASAESVSCLVHNP
VLTEEKGSVISLPEKLQTELASLKVNGPSQPILVRVGEDIQLTCYLSPKA
NAQSMEVRWDRSHRYPAVHVYMDGDHVAGEQMAEYRGRTVLVSDAIDEGR
LTLQILSARPSDDGQYRCLFEKDDVYQEASLDLKVVSLGSSPLITVEGQE
DGEMQPMCSSDGWFPQPHVPWRDMEGKTIPSSSQALTQGSHGLFHVQTLL
RVTNISAVDVTCSISIPFLGEEKIATFSLSGWSKYGPPCPPCPAPEFLGG
PSVFLFPPKPKDQLMISRTPEVTCVVVDVSQEDPEVQFNWYVDGVEVHNA
KTKPREEQFNSTYRVVSVLTVLHQDWLSGKEYKCKVSSKGLPSSIEKTIS
NATGQPREPQVYTLPPSQEEMTKNQVSLTCLVKGFYPSDIAVEWESNGQP
ENNYKTTPPVLDSDGSFFLYSRLTVDKSRWQEGNVFSCSVLHEALHNHYT
QKSLSLSLGKIEGRMDFTVTVPKDLYVVEYGSNMTIECKFPVEKQLDLAA
LIVYWEMEDKNIIQFVHGEEDLKVQHSSYRQRARLLKDQLSLGNAALQIT
DVKLQDAGVYRCMISYGGADYKRITVKVNAPYNKINQRILVVDPVTSEHE
LTCQAEGYPKAEVIWTSSDHQVLSGKTTTTNSKREEKLFNVTSTLRINTT
TNEIFYCTFRRLDPEENHTAELVIPELPLAHPPNER.

In embodiments, a chimeric protein comprises a variant of a BTNL2-Fc-PD-L1 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 73.

In embodiments, the chimeric protein is capable of contemporaneously binding the BTNL2 receptor and the PD-L1 receptor. In embodiments, the PD-L1 receptor is PD-1; however, the BTNL2 ligand/receptor is presently unknown. BTNL2 may be involved in immune surveillance, serving as a negative T-cell regulator by decreasing T-cell proliferation and cytokine release. PD-L1 plays a critical role in induction and maintenance of immune tolerance to self, in part, by acting as a ligand for the inhibitory receptor PD-1; it modulates the activation threshold of T-cells and limits T-cell effector response, including cytotoxic T lymphocytes (CTLs) effector function Accordingly, a chimeric protein comprising the extracellular domains of BTNL2 and PD-L1 is capable of contemporaneously activating an immune inhibitory signal or competitively inhibiting an immune activating signal (via BTNL2) and activating an immune inhibitory signal (via PD-L1). In embodiments, this chimeric protein is referred to herein as PD-L1-Fc-BTNL2.

In embodiments, the chimeric proteins of the present invention comprise variants of the extracellular domain, which includes the receptor-binding domain, of PD-L1. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of PD-L1, e.g., human PD-L1.

In embodiments, the extracellular domain of PD-L1 has the following amino acid sequence:

(SEQ ID NO: 62)
FTITAPKDLYVVEYGSNVTMECRFPVERELDLLALVVYWEKEDEQVIQFV
AGEEDLKPQHSNFRGRASLPKDQLLKGNAALQITDVKLQDAGVYCCIISY
GGADYKRITLKVNAPYRKINQRISVDPATSEHELICQAEGYPEAEVIWTN
SDHQPVSGKRSVTTSRTEGMLLNVTSSLRVNATANDVFYCTFWRSQPGQN
HTAELIIPELPATHPPQNRTH.

In embodiments, a chimeric protein comprises a variant of the extracellular domain of PD-L1. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 62.

In embodiments, the second domain of a chimeric protein comprises an amino acid sequence that is at least 95% identical to the amino acid sequence of SEQ ID NO: 62.

One of ordinary skill may select variants of the known amino acid sequence of PD-L1 by consulting the literature, e.g., Freeman et al., “Engagement of the PD-1 immunoinhibitory receptor by a novel B7-family member leads to negative regulation of lymphocyte activation.” J. Exp. Med. 192:1027-1034 (2000); Burr, et al, “CMTM6 maintains the expression of PD-L1 and regulates anti-tumour immunity.” Nature 549 (7670), 101-105 (2017); Lin et al., “The PD-1/PD-L1 complex resembles the antigen-binding Fv domains of antibodies and T cell receptors.” Proc. Natl. Acad. Sci. U.S.A. 105 (8), 3011-3016 (2008); and Zak et al., “Structure of the Complex of Human Programmed Death 1, PD-1, and Its Ligand PD-L1.” Structure 23 (12), 2341-2348 (2015), each of which is incorporated by reference in its entirety.

In embodiments, the chimeric proteins of the present invention comprise variants of the extracellular domain, which includes the ligand/receptor-binding domain, of BTNL2. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of BTNL2, e.g., human BTNL2.

In embodiments, the extracellular domain of BTNL2 has the following amino acid sequence:

(SEQ ID NO: 72)
KQSEDFRVIGPAHPILAGVGEDALLTCQLLPKRTTMHVEVRWYRSEPSTP
VFVHRDGVEVTEMQMEEYRGWVEWIENGIAKGNVALKIHNIQPSDNGQYW
CHFQDGNYCGETSLLLKVAGLGSAPSIHMEGPGESGVQLVCTARGWFPEP
QVYWEDIRGEKLLAVSEHRIQDKDGLFYAEATLVVRNASAESVSCLVHNP
VLTEEKGSVISLPEKLQTELASLKVNGPSQPILVRVGEDIQLTCYLSPKA
NAQSMEVRWDRSHRYPAVHVYMDGDHVAGEQMAEYRGRTVLVSDAIDEGR
LTLQILSARPSDDGQYRCLFEKDDVYQEASLDLKVVSLGSSPLITVEGQE
DGEMQPMCSSDGWFPQPHVPWRDMEGKTIPSSSQALTQGSHGLFHVQTLL
RVTNISAVDVTCSISIPFLGEEKIATFSLSGW.

In embodiments, a chimeric protein comprises a variant of the extracellular domain of BTNL2. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 72.

In embodiments, the first domain of a chimeric protein comprises an amino acid sequence that is at least 95% identical to the amino acid sequence of SEQ ID NO: 72.

One of ordinary skill may select variants of the known amino acid sequence of BTNL2 by consulting the literature, e.g., Valentonyte et al., “Sarcoidosis is associated with a truncating splice site mutation in BTNL2.” Nat. Genet. 37:357-364 (2005); Nguyen et al., “BTNL2, a butyrophilin-like molecule that functions to inhibit T cell activation.” J Immunol. 2006 Jun. 15; 176(12):7354-60; Arnett et al., “BTNL2, a butyrophilin/B7-like molecule, is a negative costimulatory molecule modulated in intestinal inflammation.” J. Immunol. 178 (3), 1523-1533 (2007); Rhodes et al., “Regulation of Immunity by Butyrophilins.” Annu Rev Immunol. 2016 May 20; 34:151-72; and Cui et al., “In vivo administration of recombinant BTNL2-Fc fusion protein ameliorates graft-versus-host disease in mice.” Cell Immunol. 2019 January; 335:22-29, each of which is incorporated by reference in its entirety.

In embodiments, a chimeric protein of the present invention comprises: (1) a first domain comprising the amino acid sequence of SEQ ID NO: 72, (b) a second domain comprises the amino acid sequence of SEQ ID NO: 62, and (c) a linker comprises an amino acid sequence that is at least 95% identical to SEQ ID NO: 1, SEQ ID NO: 2, or SEQ ID NO: 3.

In embodiments, a PD-L1-Fc-BTNL2 chimeric protein of the present invention has the following amino acid sequence:

(SEQ ID NO: 74)
FTVTVPKDLYVVEYGSNMTIECKFPVEKQLDLAALIVYWEMEDKNIIQFV
HGEEDLKVQHSSYRQRARLLKDQLSLGNAALQITDVKLQDAGVYRCMISY
GGADYKRITVKVNAPYNKINQRILVVDPVTSEHELTCQAEGYPKAEVIWT
SSDHQVLSGKTTTTNSKREEKLFNVTSTLRINTTTNEIFYCTFRRLDPEE
NHTAELVIPELPLAHPPNERSKYGPPCPPCPAPEFLGGPSVFLFPPKPKD
QLMISRTPEVTCVVVDVSQEDPEVQFNWYVDGVEVHNAKTKPREEQFNST
YRVVSVLTVLHQDWLSGKEYKCKVSSKGLPSSIEKTISNATGQPREPQVY
TLPPSQEEMTKNQVSLTCLVKGFYPSDIAVEWESNGQPENNYKTTPPVLD
SDGSFFLYSRLTVDKSRWQEGNVFSCSVLHEALHNHYTQKSLSLSLGKIE
GRMDKQSEDFRVIGPAHPILAGVGEDALLTCQLLPKRTTMHVEVRWYRSE
PSTPVFVHRDGVEVTEMQMEEYRGWVEWIENGIAKGNVALKIHNIQPSDN
GQYWCHFQDGNYCGETSLLLKVAGLGSAPSIHMEGPGESGVQLVCTARGW
FPEPQVYWEDIRGEKLLAVSEHRIQDKDGLFYAEATLWRNASAESVSCLV
HNPVLTEEKGSVISLPEKLQTELASLKVNGPSQPILVRVGEDIQLTCYLS
PKANAQSMEVRWDRSHRYPAVHVYMDGDHVAGEQMAEYRGRTVLVSDAID
EGRLTLQILSARPSDDGQYRCLFEKDDVYQEASLDLKVVSLGSSPLITVE
GQEDGEMQPMCSSDGWFPQPHVPWRDMEGKTIPSSSQALTQGSHGLFHVQ
TLLRVTNISAVDVTCSISIPFLGEEKIATFSLSGW.

In embodiments, a chimeric protein comprises a variant of a PD-L1-Fc-BTNL2 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 74.

In embodiments, the chimeric protein is capable of contemporaneously binding the CTLA4 ligand and the IL2 receptor. In embodiments, the CTLA4 ligand is CD80 or CD86 and IL2 binds to the IL2 receptor, which has three forms, generated by different combinations of three different proteins, often referred to as “chains”: IL2Rα, IL2Rβ, and IL2Rγ. CTLA4 acts as an “off” switch when bound to its ligand on the surface of antigen-presenting cells (APCs). CTLA4 is a protein receptor that functions as an immune checkpoint and downregulates immune responses. Certain “High Affinity” IL2 expands and activates Tregs which help prevent autoimmunity and control inflammation. Accordingly, a chimeric protein comprising the portion of IL2 capable binding its receptor and the extracellular domain of CTLA4 is capable of contemporaneously competitively inhibiting an immune activating signal (via CTLA4) and stimulating regulatory T cells (via IL2). In embodiments, this chimeric protein is referred to herein as CTLA4-Fc-IL2.

In embodiments, the chimeric proteins of the present invention comprise variants of the extracellular domain, which includes the ligand/receptor binding domain, of CTLA4. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of CTLA4, e.g., human CTLA4.

In embodiments, the extracellular domain of CTLA4 has the following amino acid sequence:

(SEQ ID NO: 60)
KAMHVAQPAVVLASSRGIASFVCEYASPGKATEVRVTVLRQADSQVTEVC
AATYMMGNELTFLDDSICTGTSSGNQVNLTIQGLRAMDTGLYICKVELMY
PPPYYLGIGNGTQIYVIDPEPCPDSD.

In embodiments, a chimeric protein comprises a variant of the extracellular domain of CTLA4. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 60.

In embodiments, the first domain of a chimeric protein comprises an amino acid sequence that is at least 95% identical to the amino acid sequence of SEQ ID NO: 60.

One of ordinary skill may select variants of the known amino acid sequence of CTLA4 by consulting the literature, e.g., Linsley et al., “CTLA-4 is a second receptor for the B cell activation antigen B7.” J. Exp. Med. 174 (3), 561-569 (1991); Dariavach et al., “Human Ig superfamily CTLA-4 gene: chromosomal localization and identity of protein sequence between murine and human CTLA-4 cytoplasmic domains”. Eur. J. Immunol. 18 (12), 1901-1905 (1988); Teft et al., “A molecular perspective of CTLA-4 function.” Annu. Rev. Immunol. 24:65-97 (2006); Ramagopal et al., “Structural basis for cancer immunotherapy by the first-in-class checkpoint inhibitor ipilimumab.” Proc. Natl. Acad. Sci. U.S.A. 114:E4223-E4232 (2017); Schwartz et al., “Structural basis for co-stimulation by the human CTLA-4/B7-2 complex.” Nature 410:604-608 (2001); Stamper et al., “Crystal structure of the B7-1/CTLA-4 complex that inhibits human immune responses.” Nature 410:608-611 (2001); and Yu et al., “Rigid-body ligand recognition drives cytotoxic T-lymphocyte antigen 4 (CTLA-4) receptor triggering.” J. Biol. Chem. 286:6685-6696 (2011), each of which is incorporated by reference in its entirety.

In embodiments, the chimeric proteins of the present invention comprises variants of IL2 comprising its receptor-binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the portion IL2, e.g., human IL2, comprising its receptor-binding domain.

In embodiments, the portion of IL2 comprising its receptor-binding domain, relevant to the present invention, has one the following amino acid sequences:

(SEQ ID NO: 75)
APTSSSTKKTQLQLEHLLLDLQMILNGINNYKNPKLTRMLTFKFYMPKKA
TELKHLQCLEEELKPLEEVLNLAQSKNFHLRPRDLISRINVIVLELKGSE
TTFMCEYACETATIVEFLNRWITFSQSIISTLT;
(SEQ ID NO: 76)
APTSSSTKKTQLQLEHLLLHLQMILNGINNYKNPKLTRMLTFKFYMPKKA
TELKHLQCLEEELKPLEEVLNLAQSKNFHLRPRDLISRINVIVLELKGSE
TTFMCEYADETATIVEFLNRWITFSLSIISTLT;
(SEQ ID NO: 77)
APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRWITFCQSIISTLT;
and
(SEQ ID NO: 78)
APTSSSTKKTQLQLEHLLLDLQMILNGINNYKNPKLTRMLTFKFYMPKKA
TELKHLQCLEEELKPLEEVLNLAQSKNFHFDPRDVVSNINVFVLELKGSE
TTFMCEYADETATIVEFLNRWITFCQSIISTLT

The IL2 variants of SEQ ID NO: 75 and SEQ ID NO: 76 are “high affinity” IL2s, which is preferentially expressed by regulatory T cells.

In embodiments, a chimeric protein comprises a variant of a portion of IL2 comprising its receptor-binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with one of SEQ ID NO: 75 to SEQ ID NO: 78.

In embodiments, the second domain of a chimeric protein comprises an amino acid sequence that is at least 95% identical to the amino acid sequence of one of SEQ ID NO: 75 to SEQ ID NO: 78.

One of ordinary skill may select variants of the known amino acid sequence of IL2 by consulting the literature, e.g., Taniguchi et al., “Structure and expression of a cloned cDNA for human interleukin-2.” Nature 302 (5906), 305-310 (1983); Brandhuber et al., “Three-dimensional structure of interleukin-2” Science 238 (4834), 1707-1709 (1987); Bazan “Unraveling the structure of IL-2” Science 257 (5068), 410-413 (1992); Mot et al., “Secondary structure of human interleukin 2 from 3D heteronuclear NMR experiments.” Biochemistry 31 (33), 7741-7744 (1992); Wang et al., “Structure of the quaternary complex of interleukin-2 with its alpha, beta, and gammac receptors.” Science 310 (5751), 1159-1163 (2005); and Stauber et al., “Crystal structure of the IL-2 signaling complex: paradigm for a heterotrimeric cytokine receptor.” Proc. Natl. Acad. Sci. U.S.A. 103 (8), 2788-2793 (2006), each of which is incorporated by reference in its entirety.

In embodiments, a chimeric protein of the present invention comprises: (1) a first domain comprising the amino acid sequence of SEQ ID NO: 60, (b) a second domain comprises the amino acid sequence of one of SEQ ID NO: 75 to SEQ ID NO: 78, and (c) a linker comprises an amino acid sequence that is at least 95% identical to SEQ ID NO: 1, SEQ ID NO: 2, or SEQ ID NO: 3.

In embodiments, a CTLA4-Fc-IL2 chimeric protein of the present invention has the one of following amino acid sequences:

(SEQ ID NO: 79)
KAMHVAQPAVVLASSRGIASFVCEYASPGKATEVR
VTVLRQADSQVTEVCAATYMMGNELTFLDDSICTG
TSSGNQVNLTIQGLRAMDTGLYICKVELMYPPPYY
LGIGNGTQIYVIDPEPCPDSDSKYGPPCPPCPAPE
FLGGPSVFLFPPKPKDQLMISRTPEVTCVVVDVSQ
EDPEVQFNWYVDGVEVHNAKTKPREEQFNSTYRVV
SVLTVLHQDWLSGKEYKCKVSSKGLPSSIEKTISN
ATGQPREPQVYTLPPSQEEMTKNQVSLTCLVKGFY
PSDIAVEWESNGQPENNYKTTPPVLDSDGSFFLYS
RLTVDKSRWQEGNVFSCSVLHEALHNHYTQKSLSL
SLGKIEGRMDAPTSSSTKKTQLQLEHLLLDLQMIL
NGINNYKNPKLTRMLTFKFYMPKKATELKHLQCLE
EELKPLEEVLNLAQSKNFHLRPRDLISRINVIVLE
LKGSETTFMCEYACETATIVEFLNRWITFSQSIIS
TLT;
(SEQ ID NO: 80)
KAMHVAQPAVVLASSRGIASFVCEYASPGKATEVR
VTVLRQADSQVTEVCAATYMMGNELTFLDDSICTG
TSSGNQVNLTIQGLRAMDTGLYICKVELMYPPPYY
LGIGNGTQIYVIDPEPCPDSDSKYGPPCPPCPAPE
FLGGPSVFLFPPKPKDQLMISRTPEVTCVVVDVSQ
EDPEVQFNWYVDGVEVHNAKTKPREEQFNSTYRVV
SVLTVLHQDWLSGKEYKCKVSSKGLPSSIEKTISN
ATGQPREPQVYTLPPSQEEMTKNQVSLTCLVKGFY
PSDIAVEWESNGQPENNYKTTPPVLDSDGSFFLYS
RLTVDKSRWQEGNVFSCSVLHEALHNHYTQKSLSL
SLGKIEGRMDAPTSSSTKKTQLQLEHLLLHLQMIL
NGINNYKNPKLTRMLTFKFYMPKKATELKHLQCLE
EELKPLEEVLNLAQSKNFHLRPRDLISRINVIVLE
LKGSETTFMCEYADETATIVEFLNRWITFSLSIIS
TLT;
(SEQ ID NO: 81)
KAMHVAQPAVVLASSRGIASFVCEYASPGKATEVR
VTVLRQADSQVTEVCAATYMMGNELTFLDDSICTG
TSSGNQVNLTIQGLRAMDTGLYICKVELMYPPPYY
LGIGNGTQIYVIDPEPCPDSDSKYGPPCPPCPAPE
FLGGPSVFLFPPKPKDQLMISRTPEVTCVVVDVSQ
EDPEVQFNWYVDGVEVHNAKTKPREEQFNSTYRVV
SVLTVLHQDWLSGKEYKCKVSSKGLPSSIEKTISN
ATGQPREPQVYTLPPSQEEMTKNQVSLTCLVKGFY
PSDIAVEWESNGQPENNYKTTPPVLDSDGSFFLYS
RLTVDKSRWQEGNVFSCSVLHEALHNHYTQKSLSL
SLGKIEGRMDAPTSSSTKKTQLQLEHLLLDLQMIL
NGINNYKNPKLTRMLTFKFYMPKKATELKHLQCLE
EELKPLEEVLNLAQSKNFHLRPRDLISNINVIVLE
LKGSETTFMCEYADETATIVEFLNRWITFCQSIIS
TLT;
or
(SEQ ID NO: 82)
KAMHVAQPAVVLASSRGIASFVCEYASPGKATEVR
VTVLRQADSQVTEVCAATYMMGNELTFLDDSICTG
TSSGNQVNLTIQGLRAMDTGLYICKVELMYPPPYY
LGIGNGTQIYVIDPEPCPDSDSKYGPPCPPCPAPE
FLGGPSVFLFPPKPKDQLMISRTPEVTCVVVDVSQ
EDPEVQFNWYVDGVEVHNAKTKPREEQFNSTYRVV
SVLTVLHQDWLSGKEYKCKVSSKGLPSSIEKTISN
ATGQPREPQVYTLPPSQEEMTKNQVSLTCLVKGFY
PSDIAVEWESNGQPENNYKTTPPVLDSDGSFFLYS
RLTVDKSRWQEGNVFSCSVLHEALHNHYTQKSLSL
SLGKIEGRMDAPTSSSTKKTQLQLEHLLLDLQMIL
NGINNYKNPKLTRMLTFKFYMPKKATELKHLQCLE
EELKPLEEVLNLAQSKNFHFDPRDVVSNINVFVLE
LKGSETTFMCEYADETATIVEFLNRWITFCQSIIS
TLT

In embodiments, a chimeric protein comprises a variant of a CTLA4-Fc-IL2 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with one of SEQ ID NO: 79 to SEQ ID NO: 82.

In embodiments, the chimeric protein is capable of contemporaneously binding the CTLA4 ligand and the SEMA3E receptor. In embodiments, the CTLA4 ligand is CD80 or CD86 and the SEMA3E receptor is PlexinD1. CTLA4 acts as an “off” switch when bound to its ligand on the surface of antigen-presenting cells (APCs). CTLA4 is a protein receptor that functions as an immune checkpoint and downregulates immune responses. SEMA3E may act as a secreted chemorepellent in neutrophil migration and recent in vitro and in vivo experimental evidence demonstrates a key regulator role of SEMA3E on airway inflammation, hyperresponsiveness and remodeling in allergic asthma. Accordingly, a chimeric protein comprising the extracellular domains of CTLA4 and a portion of SEMA3E which includes its receptor-binding domain is capable of contemporaneously competitively inhibiting an immune activating signal (via CTLA4) and activating an immune inhibitory signal (via SEMA3E). In embodiments, this chimeric protein is referred to herein as CTLA4-Fc-SEMA3E.

In embodiments, the chimeric proteins of the present invention comprise variants of the extracellular domain, which includes the ligand/receptor binding domain, of CTLA4. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of CTLA4, e.g., human CTLA4.

In embodiments, the extracellular domain of CTLA4 has the following amino acid sequence:

(SEQ ID NO: 60)
KAMHVAQPAVVLASSRGIASFVCEYASPGKATEVR
VTVLRQADSQVTEVCAATYMMGNELTFLDDSICTG
TSSGNQVNLTIQGLRAMDTGLYICKVELMYPPPYY
LGIGNGTQIYVIDPEPCPDSD.

In embodiments, a chimeric protein comprises a variant of the extracellular domain of CTLA4. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 60.

In embodiments, the first domain of a chimeric protein comprises an amino acid sequence that is at least 95% identical to the amino acid sequence of SEQ ID NO: 60.

One of ordinary skill may select variants of the known amino acid sequence of CTLA4 by consulting the literature, e.g., Linsley et al., “CTLA-4 is a second receptor for the B cell activation antigen B7.” J. Exp. Med. 174 (3), 561-569 (1991); Dariavach et al., “Human Ig superfamily CTLA-4 gene: chromosomal localization and identity of protein sequence between murine and human CTLA-4 cytoplasmic domains”. Eur. J. Immunol. 18 (12), 1901-1905 (1988); Teft et al., “A molecular perspective of CTLA-4 function.” Annu. Rev. Immunol. 24:65-97 (2006); Ramagopal et al., “Structural basis for cancer immunotherapy by the first-in-class checkpoint inhibitor ipilimumab.” Proc. Natl. Acad. Sci. U.S.A. 114:E4223-E4232 (2017); Schwartz et al., “Structural basis for co-stimulation by the human CTLA-4/B7-2 complex.” Nature 410:604-608 (2001); Stamper et al., “Crystal structure of the B7-1/CTLA-4 complex that inhibits human immune responses.” Nature 410:608-611 (2001); and Yu et al., “Rigid-body ligand recognition drives cytotoxic T-lymphocyte antigen 4 (CTLA-4) receptor triggering.” J. Biol. Chem. 286:6685-6696 (2011), each of which is incorporated by reference in its entirety.

In embodiments, the chimeric proteins of the present invention comprise variants of the portion of SEMA3E which includes the receptor-binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the portion of SEMA3E, e.g., human SEMA3E, which comprises its receptor-binding domain.

In embodiments, the portion of SEMA3E which comprises the receptor-binding domain has the following amino acid sequence:

(SEQ ID NO: 83)
ADTTHPRLRLSHKELLNLNRTSIFHSPFGFLDLHTM
LLDEYQERLFVGGRDLVYSLSLERISDGYKEIHWP
STALKMEECIMKGKDAGECANYVRVLHHYNRTHLL
TCGTGAFDPVCAFIRVGYHLEDPLFHLESPRSERG
RGRCPFDPSSSFISTLIGSELFAGLYSDYWSRDAA
IFRSMGRLAHIRTEHDDERLLKEPKFVGSYMIPDN
EDRDDNKVYFFFTEKALEAENNAHAIYTRVGRLCV
NDVGGQRILVNKWSTFLKARLVCSVPGMNGIDTYF
DELEDVFLLPTRDHKNPVIFGLFNTTSNIFRGHAI
CVYHMSSIRAAFNGPYAHKEGPEYHWSVYEGKVPY
PRPGSCASKVNGGRYGTTKDYPDDAIRFARSHPLM
YQAIKPAHKKPILVKTDGKYNLKQIAVDRVEAEDG
QYDVLFIGTDNGIVLKVITIYNQEMESMEEVILEE
LQIFKDPVPIISMEISSKRQQLYIGSASAVAQVRF
HHCDMYGSACADCCLARDPYCAWDGISCSRYYPTG
THAKRRFRRQDVRHGNAAQQCFGQQFVGDALDKTE
EHLAYGIENNSTLLECTPRSLQAKVIWFVQKGRET
RKEEVKTDDRVVKMDLGLLFLRLHKSDAGTYFCQT
VEHSFVHTVRKITLEWEEEKVEDMFNKDDEEDRHH
RMPCPAQSSISQGAKPWYKEFLQLIGYSNFQRVEE
YCEKVWCTDRKRKKLKMSPSKWKYANPQEKKLRSK
PEHYRLPRHTLDS.

In embodiments, a chimeric protein comprises a variant of the portion of SEMA3E which includes the receptor-binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 83.

In embodiments, the second domain of a chimeric protein comprises an amino acid sequence that is at least 95% identical to the amino acid sequence of SEQ ID NO: 83.

One of ordinary skill may select variants of the known amino acid sequence of SEMA3E by consulting the literature, e.g., Lalani et al., “SEMA3E mutation in a patient with CHARGE syndrome.” J. Med. Genet. 41 (7), e94 (2004); Ota et al., “Complete sequencing and characterization of 21,243 full-length human cDNAs.” Nat. Genet. 36:40-45(2004); Movassagh et al., “The regulatory role of semaphorin 3E in allergic asthma” Int. J. Biochem. Cell Biol. 106, 68-73 (2019); Movassagh et al., “Chemorepellent Semaphorin 3E Negatively Regulates Neutrophil Migration In Vitro and In Vivo.” J. Immunol. 198 (3), 1023-1033 (2017); Siebold and Jones “Structural insights into semaphorins and their receptors” Semin. Cell Dev. Biol., 24 (2013), pp. 139-145, each of which is incorporated by reference in its entirety.

In embodiments, a chimeric protein of the present invention comprises: (1) a first domain comprising the amino acid sequence of SEQ ID NO: 60, (b) a second domain comprises the amino acid sequence of SEQ ID NO: 83, and (c) a linker comprises an amino acid sequence that is at least 95% identical to SEQ ID NO: 1, SEQ ID NO: 2, or SEQ ID NO: 3.

In embodiments, a CTLA4-Fc-SEMA3E chimeric protein of the present invention has the following amino acid sequence:

(SEQ ID NO: 84)
KAMHVAQPAVVLASSRGIASFVCEYASPGKATEVR
VTVLRQADSQVTEVCAATYMMGNELTFLDDSICTG
TSSGNQVNLTIQGLRAMDTGLYICKVELMYPPPYY
LGIGNGTQIYVIDPEPCPDSDSKYGPPCPPCPAPE
FLGGPSVFLFPPKPKDQLMISRTPEVTCVVVDVSQ
EDPEVQFNWYVDGVEVHNAKTKPREEQFNSTYRVV
SVLTVLHQDWLSGKEYKCKVSSKGLPSSIEKTISN
ATGQPREPQVYTLPPSQEEMTKNQVSLTCLVKGFY
PSDIAVEWESNGQPENNYKTTPPVLDSDGSFFLYS
RLTVDKSRWQEGNVFSCSVLHEALHNHYTQKSLSL
SLGKIEGRMDADTTHPRLRLSHKELLNLNRTSIFH
SPFGFLDLHTMLLDEYQERLFVGGRDLVYSLSLER
ISDGYKEIHWPSTALKMEECIMKGKDAGECANYVR
VLHHYNRTHLLTCGTGAFDPVCAFIRVGYHLEDPL
FHLESPRSERGRGRCPFDPSSSFISTLIGSELFAG
LYSDYWSRDAAIFRSMGRLAHIRTEHDDERLLKEP
KFVGSYMIPDNEDRDDNKVYFFFTEKALEAENNAH
AIYTRVGRLCVNDVGGQRILVNKWSTFLKARLVCS
VPGMNGIDTYFDELEDVFLLPTRDHKNPVIFGLFN
TTSNIFRGHAICVYHMSSIRAAFNGPYAHKEGPEY
HWSVYEGKVPYPRPGSCASKVNGGRYGTTKDYPDD
AIRFARSHPLMYQAIKPAHKKPILVKTDGKYNLKQ
IAVDRVEAEDGQYDVLFIGTDNGIVLKVITIYNQE
MESMEEVILEELQIFKDPVPIISMEISSKRQQLYI
GSASAVAQVRFHHCDMYGSACADCCLARDPYCAWD
GISCSRYYPTGTHAKRRFRRQDVRHGNAAQQCFGQ
QFVGDALDKTEEHLAYGIENNSTLLECTPRSLQAK
VIWFVQKGRETRKEEVKTDDRVVKMDLGLLFLRLH
KSDAGTYFCQTVEHSFVHTVRKITLEVVEEEKVED
MFNKDDEEDRHHRMPCPAQSSISQGAKPWYKEFLQ
LIGYSNFQRVEEYCEKVWCTDRKRKKLKMSPSKWK
YANPQEKKLRSKPEHYRLPRHTLDS.

In embodiments, a chimeric protein comprises a variant of a CTLA4-Fc-SEMA3E chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 84.

In embodiments, the chimeric protein is capable of contemporaneously binding the VSIG4 ligand and the IL2 receptor. In embodiments, the VSIG4 ligand is C3b or an unidentified T-cell ligand or receptor and IL2 binds to the IL2 receptor, which has three forms, generated by different combinations of three different proteins, often referred to as “chains”: a (alpha) (also called IL2Rα, CD25, or Tac antigen), β (beta) (also called IL2Rβ, or CD122), and γ (gamma) (also called IL2Rγ, γc, common gamma chain, or CD132). VSIG4 is a phagocytic receptor, strong negative regulator of T-cell proliferation and IL2 production; it is a potent inhibitor of the alternative complement pathway convertases. Low-dose IL2 has been shown to expand and activate Tregs which helps prevent autoimmunity and control inflammation. Accordingly, a chimeric protein comprising the extracellular domains of VSIG4 and IL2 is capable of contemporaneously activating an immune inhibitory signal (via VSIG4) and activating regulatory T cells (via IL2). In embodiments, this chimeric protein is referred to herein as VSIG4-Fc-IL2.

In embodiments, the chimeric proteins of the present invention comprise variants of the extracellular domain, which includes the receptor-binding domain, of VSIG4. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of VSIG4, e.g., human VSIG4.

In embodiments, the extracellular domain of VSIG4 has the following amino acid sequence:

(SEQ ID NO: 85)
RPILEVPESVTGPWKGDVNLPCTYDPLQGYTQVLVK
WLVQRGSDPVTIFLRDSSGDHIQQAKYQGRLHVSHK
VPGDVSLQLSTLEMDDRSHYTCEVTWQTPDGNQVVR
DKITELRVQKLSVSKPTVTTGSGYGFTVPQGMRISL
QCQARGSPPISYIWYKQQTNNQEPIKVATLSTLLFK
PAVIADSGSYFCTAKGQVGSEQHSDIVKFVVKDSSK
LLKTKTEAPTTMTYPLKATSTVKQSWDWTTDMDGYL
GETSAGPGKSLP.

In embodiments, a chimeric protein comprises a variant of the extracellular domain of VSIG4. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 85.

In embodiments, the first domain of a chimeric protein comprises an amino acid sequence that is at least 95% identical to the amino acid sequence of SEQ ID NO: 85.

One of ordinary skill may select variants of the known amino acid sequence of VSIG4 by consulting the literature, e.g., Vogt et al., “VSIG4, a B7 family-related protein, is a negative regulator of T cell activation.” J. Clin. Invest. 116:2817-2826 (2006); Wiesmann et al., “Structure of C3b in complex with CRIg gives insights into regulation of complement activation.” Nature 444:217-220 (2006); and Zhang and Henzel “Signal peptide prediction based on analysis of experimentally verified cleavage sites.” Protein Sci. 13 (10), 2819-2824 (2004), each of which is incorporated by reference in its entirety.

In embodiments, the chimeric proteins of the present invention comprise variants of the receptor-binding domain, of IL2. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the portion of IL2, e.g., human IL2, which comprises its receptor-binding domain.

In embodiments, the portion of IL2 comprising its receptor-binding domain, relevant to the present invention, has one the following amino acid sequences:

(SEQ ID NO: 75)
APTSSSTKKTQLQLEHLLLDLQMILNGINNYKNPKLT
RMLTFKFYMPKKATELKHLQCLEEELKPLEEVLNLAQ
SKNFHLRPRDLISRINVIVLELKGSETTFMCEYACET
ATIVEFLNRWITFSQSIISTLT;
(SEQ ID NO: 76)
APTSSSTKKTQLQLEHLLLHLQMILNGINNYKNPKLT
RMLTFKFYMPKKATELKHLQCLEEELKPLEEVLNLAQ
SKNFHLRPRDLISRINVIVLELKGSETTFMCEYADET
ATIVEFLNRWITFSLSIISTLT;
(SEQ ID NO: 77)
APTSSSTKKTQLQLEHLLLDLQMILNGINNYKNPKLT
RMLTFKFYMPKKATELKHLQCLEEELKPLEEVLNLAQ
SKNFHLRPRDLISNINVIVLELKGSETTFMCEYADET
ATIVEFLNRWITFCQSIISTLT;
and
(SEQ ID NO: 78)
APTSSSTKKTQLQLEHLLLDLQMILNGINNYKNPKLT
RMLTFKFYMPKKATELKHLQCLEEELKPLEEVLNLAQ
SKNFHFDPRDWSNINVFVLELKGSETTFMCEYADETA
TIVEFLNRWITFCQSIISTLT

The IL2 variants of SEQ ID NO: 75 and SEQ ID NO: 76 are “high affinity” IL2s, which is preferentially expressed by regulatory T cells.

In embodiments, a chimeric protein comprises a variant of the receptor-binding domain of IL2. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with one of SEQ ID NO: 75 to SEQ ID NO: 78.

In embodiments, the second domain of a chimeric protein comprises an amino acid sequence that is at least 95% identical to the amino acid sequence of one of SEQ ID NO: 75 to SEQ ID NO: 78.

One of ordinary skill may select variants of the known amino acid sequence of IL2 by consulting the literature, e.g., Taniguchi et al., “Structure and expression of a cloned cDNA for human interleukin-2.” Nature 302 (5906), 305-310 (1983); Brandhuber et al., “Three-dimensional structure of interleukin-2” Science 238 (4834), 1707-1709 (1987); Bazan “Unraveling the structure of IL-2” Science 257 (5068), 410-413 (1992); Mot et al., “Secondary structure of human interleukin 2 from 3D heteronuclear NMR experiments.” Biochemistry 31 (33), 7741-7744 (1992); Wang et al., “Structure of the quaternary complex of interleukin-2 with its alpha, beta, and gammac receptors.” Science 310 (5751), 1159-1163 (2005); and Stauber et al., “Crystal structure of the IL-2 signaling complex: paradigm for a heterotrimeric cytokine receptor.” Proc. Natl. Acad. Sci. U.S.A. 103 (8), 2788-2793 (2006), each of which is incorporated by reference in its entirety.

In embodiments, a chimeric protein of the present invention comprises: (1) a first domain comprising the amino acid sequence of SEQ ID NO: 85, (b) a second domain comprises the amino acid sequence of one of SEQ ID NO: 75 to SEQ ID NO: 78, and (c) a linker comprises an amino acid sequence that is at least 95% identical to SEQ ID NO: 1, SEQ ID NO: 2, or SEQ ID NO: 3.

In embodiments, a VSIG4-Fc-IL2 chimeric protein of the present invention has the one of following amino acid sequences:

(SEQ ID NO: 86)
RPILEVPESVTGPWKGDVNLPCTYDPLQGYTQVLV
KWLVQRGSDPVTIFLRDSSGDHIQQAKYQGRLHVS
HKVPGDVSLQLSTLEMDDRSHYTCEVTWQTPDGNQ
VVRDKITELRVQKLSVSKPTVTTGSGYGFTVPQGM
RISLQCQARGSPPISYIWYKQQTNNQEPIKVATLS
TLLFKPAVIADSGSYFCTAKGQVGSEQHSDIVKFV
VKDSSKLLKTKTEAPTTMTYPLKATSTVKQSWDWT
TDMDGYLGETSAGPGKSLPSKYGPPCPPCPAPEFL
GGPSVFLFPPKPKDQLMISRTPEVTCVVVDVSQED
PEVQFNWYVDGVEVHNAKTKPREEQFNSTYRVVSV
LTVLHQDWLSGKEYKCKVSSKGLPSSIEKTISNAT
GQPREPQVYTLPPSQEEMTKNQVSLTCLVKGFYPS
DIAVEWESNGQPENNYKTTPPVLDSDGSFFLYSRL
TVDKSRWQEGNVFSCSVLHEALHNHYTQKSLSLSL
GKIEGRMDAPTSSSTKKTQLQLEHLLLDLQMILNG
INNYKNPKLTRMLTFKFYMPKKATELKHLQCLEEE
LKPLEEVLNLAQSKNFHLRPRDUSRINVIVLELKG
SETTFMCEYACETATIVEFLNRWITFSQSIISTLT;
(SEQ ID NO: 87)
RPILEVPESVTGPWKGDVNLPCTYDPLQGYTQVLV
KWLVQRGSDPVTIFLRDSSGDHIQQAKYQGRLHVS
HKVPGDVSLQLSTLEMDDRSHYTCEVTWQTPDGNQ
VVRDKITELRVQKLSVSKPTVTTGSGYGFTVPQGM
RISLQCQARGSPPISYIWYKQQTNNQEPIKVATLS
TLLFKPAVIADSGSYFCTAKGQVGSEQHSDIVKFV
VKDSSKLLKTKTEAPTTMTYPLKATSTVKQSWDWT
TDMDGYLGETSAGPGKSLPSKYGPPCPPCPAPEFL
GGPSVFLFPPKPKDQLMISRTPEVTCVVVDVSQED
PEVQFNWYVDGVEVHNAKTKPREEQFNSTYRVVSV
LTVLHQDWLSGKEYKCKVSSKGLPSSIEKTISNAT
GQPREPQVYTLPPSQEEMTKNQVSLTCLVKGFYPS
DIAVEWESNGQPENNYKTTPPVLDSDGSFFLYSRL
TVDKSRWQEGNVFSCSVLHEALHNHYTQKSLSLSL
GKIEGRMDAPTSSSTKKTQLQLEHLLLHLQMILNG
INNYKNPKLTRMLTFKFYMPKKATELKHLQCLEEE
LKPLEEVLNLAQSKNFHLRPRDUSRINVIVLELKG
SETTFMCEYADETATIVEFLNRWITFSLSIISTLT;
(SEQ ID NO: 88)
RPILEVPESVTGPWKGDVNLPCTYDPLQGYTQVLV
KWLVQRGSDPVTIFLRDSSGDHIQQAKYQGRLHVS
HKVPGDVSLQLSTLEMDDRSHYTCEVTWQTPDGNQ
VVRDKITELRVQKLSVSKPTVTTGSGYGFTVPQGM
RISLQCQARGSPPISYIWYKQQTNNQEPIKVATLS
TLLFKPAVIADSGSYFCTAKGQVGSEQHSDIVKFV
VKDSSKLLKTKTEAPTTMTYPLKATSTVKQSWDWT
TDMDGYLGETSAGPGKSLPSKYGPPCPPCPAPEFL
GGPSVFLFPPKPKDQLMISRTPEVTCVVVDVSQED
PEVQFNWYVDGVEVHNAKTKPREEQFNSTYRVVSV
LTVLHQDWLSGKEYKCKVSSKGLPSSIEKTISNAT
GQPREPQVYTLPPSQEEMTKNQVSLTCLVKGFYPS
DIAVEWESNGQPENNYKTTPPVLDSDGSFFLYSRL
fVDKSRWQEGNVFSCSVLHEALHNHYTQKSLSLSLG
KIEGRMDAPTSSSTKKTQLQLEHLLLDLQMILNGI
NNYKNPKLTRMLTFKFYMPKKATELKHLQCLEEEL
KPLEEVLNLAQSKNFHLRPRDLISNINVIVLELKG
SETTFMCEYADETATIVEFLNRWITFCQSIISTLT;
or
(SEQ ID NO: 89)
RPILEVPESVTGPWKGDVNLPCTYDPLQGYTQVLV
KWLVQRGSDPVTIFLRDSSGDHIQQAKYQGRLHVS
HKVPGDVSLQLSTLEMDDRSHYTCEVTWQTPDGNQ
VVRDKITELRVQKLSVSKPTVTTGSGYGFTVPQGM
RISLQCQARGSPPISYIWYKQQTNNQEPIKVATLS
TLLFKPAVIADSGSYFCTAKGQVGSEQHSDIVKFV
VKDSSKLLKTKTEAPTTMTYPLKATSTVKQSWD
WTTDMD
GYLGETSAGPGKSLPSKYGPPCPPCPAPEFLGGPS
VFLFPPKPKDQLMISRTPEVTCVVVDVSQEDPEVQ
FNWYVDGVEVHNAKTKPREEQFNSTYRVVSVLTVL
HQDWLSGKEYKCKVSSKGLPSSIEKTISNATGQPR
EPQVYTLPPSQEEMTKNQVSLTCLVKGFYPSDIAV
EWESNGQPENNYKTTPPVLDSDGSFFLYSRLTVDK
SRWQEGNVFSCSVLHEALHNHYTQKSLSLSLGKIE
GRMDAPTSSSTKKTQLQLEHLLLDLQMILNGINNY
KNPKLTRMLTFKFYMPKKATELKHLQCLEEELKPL
EEVLNLAQSKNFHFDPRDWSNINVFVLELKGSETT
FMCEYADETATIVEFLNRWITFCQSIISTLT

In embodiments, a chimeric protein comprises a variant of a VSIG4-Fc-IL2 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with one of SEQ ID NO: 86 to SEQ ID NO: 89.

In embodiments, the chimeric protein is capable of contemporaneously binding the CTLA4 ligand and a ligand/receptor of a Type II transmembrane protein selected from BTNL2, C-type lectin domain (CLEC) family members, GITRL, TL1A, IL-10, TGF-beta, In embodiments, the CLEC family member is selected from AlCL/CLEC-2B, ASGR1/ASGPR1, ASGR2, C1q R1/CD93, CD161, CD161/NK1.1, CD23/Fc epsilon RII, CD302/CLEC13A, CD72, CD94, Chondrolectin, CLEC-1, CLEC10A/CD301, CLEC12B, CLEC14A, CLEC16A, CLEC17A, CLEC18A, CLEC18B, CLEC18C, CLEC-2/CLEC1B, CLEC-2A, CLEC3A, CLEC3B/Tetranectin, CLEC4B2/mDCAR1, CLEC4D/CLECSF8, CLEC4E, CLEC4F/CLECSF13, CLEC9a, CLECL1/DCAL-1, CL-K1/COLEC11, CL-L1/COLEC10, CL-P1/COLEC12, DCAR/CLEC4B, DCIR/CLEC4A, DCIR4/CLEC4A1, DC-SIGN/CD209, DC-SIGN+FDC-SIGNR, DC-SIGNR/CD299, DC-SIGNR/CD299, DEC-205/CD205, Dectin-1/CLEC7A, Dectin-2/CLEC6A, DLEC/CLEC4C/BDCA-2, Ficolin-1, Ficolin-2, Ficolin-3, Klre-1, KLRG2, Langerin/CD207, Layilin, LOX-1/OLR1, LSECtin/CLEC4G, MBL, MBL-1, MBL-2, MDL-1/CLEC5A, MGL1/2 (CD301a/b), MGL1/CD301a, MGL2/CD301b, MGL2/CD301b, MICL/CLEC12A, MMR/CD206, Mrc2, NKG2A/CD159a, NKG2A/NKG2B Isoform 2, NKG2C/CD159c, NKG2D/CD314, NKG2E, NKG2H, NKp80/KLRF1, OCIL/CLEC2d, OCILRP2/CLEC2i, PLA2R1, QBRICK/FREM1, Reg1, Reg1A, Reg1B, Reg2, Reg3A, Reg3B, Reg3D, Reg3G, Reg4, SCGF/CLEC11a, SFTPA1, SIGNR1/CD209b, SIGNR3/CD209d, SIGNR4/CD209e, SIGNR7/CD209g, and SP-D. In embodiments, this chimeric protein is referred to herein as CTLA4-Fc-Type II.

In embodiments, the chimeric proteins of the present invention comprise variants of the extracellular domain, which includes the ligand-binding domain, of CTLA4. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of CTLA4, e.g., human CTLA4.

In embodiments, the extracellular domain of CTLA4 has the following amino acid sequence:

(SEQ ID NO: 60)
KAMHVAQPAVVLASSRGIASFVCEYASPGKATEVR
VTVLRQADSQVTEVCAATYMMGNELTFLDDSICTG
TSSGNQVNLTIQGLRAMDTGLYICKVELMYPPPYY
LGIGNGTQIYVIDPEPCPDSD.

In embodiments, a chimeric protein comprises a variant of the extracellular domain of CTLA4. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 60.

In embodiments, the first domain of a chimeric protein comprises an amino acid sequence that is at least 95% identical to the amino acid sequence of SEQ ID NO: 60.

One of ordinary skill may select variants of the known amino acid sequence of CTLA4 by consulting the literature, e.g., Linsley et al., “CTLA-4 is a second receptor for the B cell activation antigen B7.” J. Exp. Med. 174 (3), 561-569 (1991); Dariavach et al., “Human Ig superfamily CTLA-4 gene: chromosomal localization and identity of protein sequence between murine and human CTLA-4 cytoplasmic domains”. Eur. J. Immunol. 18 (12), 1901-1905 (1988); Teft et al., “A molecular perspective of CTLA-4 function.” Annu. Rev. Immunol. 24:65-97 (2006); Ramagopal et al., “Structural basis for cancer immunotherapy by the first-in-class checkpoint inhibitor ipilimumab.” Proc. Natl. Acad. Sci. U.S.A. 114:E4223-E4232 (2017); Schwartz et al., “Structural basis for co-stimulation by the human CTLA-4/B7-2 complex.” Nature 410:604-608 (2001); Stamper et al., “Crystal structure of the B7-1/CTLA-4 complex that inhibits human immune responses.” Nature 410:608-611 (2001); and Yu et al., “Rigid-body ligand recognition drives cytotoxic T-lymphocyte antigen 4 (CTLA-4) receptor triggering.” J. Biol. Chem. 286:6685-6696 (2011), each of which is incorporated by reference in its entirety.

In embodiments, the chimeric proteins of the present invention comprise variants of the extracellular domain of a herein-described Type II transmembrane protein, i.e., selected from 4-1BBL, APRIL, BAFF, BTNL2, CD28, CD30L, CD40L, CD70, C-type lectin domain (CLEC) family members, FasL, GITRL, LIGHT, LTa, LTa1b2, NKG2A, NKG2C, NKG2D, OX40L, RANKL, TL1A, TNFa, and TRAIL. In embodiments, the CLEC family member is selected from AlCL/CLEC-2B, ASGR1/ASGPR1, ASGR2, C1q R1/CD93, CD161, CD161/NK1.1, CD23/Fc epsilon RII, CD302/CLEC13A, CD72, CD94, Chondrolectin, CLEC-1, CLEC10A/CD301, CLEC12B, CLEC14A, CLEC16A, CLEC17A, CLEC18A, CLEC18B, CLEC18C, CLEC-2/CLEC1B, CLEC-2A, CLEC3A, CLEC3B/Tetranectin, CLEC4B2/mDCAR1, CLEC4D/CLECSF8, CLEC4E, CLEC4F/CLECSF13, CLEC9a, CLECL1/DCAL-1, CL-K1/COLEC11, CL-L1/COLEC10, CL-P1/COLEC12, DCAR/CLEC4B, DCIR/CLEC4A, DCIR4/CLEC4A1, DC-SIGN/CD209, DC-SIGN+FDC-SIGNR, DC-SIGNR/CD299, DC-SIGNR/CD299, DEC-205/CD205, Dectin-1/CLEC7A, Dectin-2/CLEC6A, DLEC/CLEC4C/BDCA-2, Ficolin-1, Ficolin-2, Ficolin-3, Klre-1, KLRG2, Langerin/CD207, Layilin, LOX-1/OLR1, LSECtin/CLEC4G, MBL, MBL-1, MBL-2, MDL-1/CLEC5A, MGL1/2 (CD301a/b), MGL1/CD301a, MGL2/CD301b, MGL2/CD301b, MICL/CLEC12A, MMR/CD206, Mrc2, NKG2A/CD159a, NKG2A/NKG2B Isoform 2, NKG2C/CD159c, NKG2D/CD314, NKG2E, NKG2H, NKp80/KLRF1, OCIL/CLEC2d, OCILRP2/CLEC2i, PLA2R1, QBRICK/FREM1, Reg1, Reg1A, Reg1B, Reg2, Reg3A, Reg3B, Reg3D, Reg3G, Reg4, SCGF/CLEC11a, SFTPA1, SIGNR1/CD209b, SIGNR3/CD209d, SIGNR4/CD209e, SIGNR7/CD209g, and SP-D. The amino acid sequence of the herein-described Type II transmembrane protein are publically available, see, e.g., at the World Wide Web (www) uniprot.org and at the World Wide Web (www) ncbi.nlm.nih.gov/protein and in one or more of WO2018/157162; WO2018/157165; WO2018/157164; WO2018/157163; and WO2017/059168, the contents relevant to this embodiment are incorporated herein by reference in its entirety. Moreover, many of the herein-described Type II transmembrane proteins have been structurally characterized, e.g., by predictive algorithms and/or x-ray crystallography; again see (www) uniprot.org; the contents relevant to this embodiment are incorporated herein by reference in its entirety.

Based on the published amino acid sequences and structural characterizations, a skilled artisan could readily determine sequence variants of the herein-described Type II transmembrane protein which retain (or enhance) the native ligand/receptor binding affinity or the Type II transmembrane protein. Examples of such variants may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of a herein-described Type II transmembrane protein, e.g., the human Type II transmembrane protein.

In embodiments, a chimeric protein of the present invention comprises: (1) a first domain comprising the amino acid sequence of SEQ ID NO: 60 or a variant thereof, as described above, (b) a second domain comprises the amino acid sequence of a portion of the extracellular domain of a herein-described Type II transmembrane protein, or a variant thereof, as described above, and (c) a linker comprises an amino acid sequence that is at least 95% identical to SEQ ID NO: 1, SEQ ID NO: 2, or SEQ ID NO: 3.

In embodiments, the chimeric protein is capable of contemporaneously binding the TNFR2 ligand and a ligand/receptor of a Type II transmembrane protein selected from BTNL2C-type lectin domain (CLEC) family members, GITRL TL1A, IL-10, TGF-beta. In embodiments, the CLEC family member is selected from AlCL/CLEC-2B, ASGR1/ASGPR1, ASGR2, C1q R1/CD93, CD161, CD161/NK1.1, CD23/Fc epsilon RII, CD302/CLEC13A, CD72, CD94, Chondrolectin, CLEC-1, CLEC10A/CD301, CLEC12B, CLEC14A, CLEC16A, CLEC17A, CLEC18A, CLEC18B, CLEC18C, CLEC-2/CLEC1B, CLEC-2A, CLEC3A, CLEC3B/Tetranectin, CLEC4B2/mDCAR1, CLEC4D/CLECSF8, CLEC4E, CLEC4F/CLECSF13, CLEC9a, CLECL1/DCAL-1, CL-K1/COLEC11, CL-L1/COLEC10, CL-P1/COLEC12, DCAR/CLEC4B, DCIR/CLEC4A, DCIR4/CLEC4A1, DC-SIGN/CD209, DC-SIGN+FDC-SIGNR, DC-SIGNR/CD299, DC-SIGNR/CD299, DEC-205/CD205, Dectin-1/CLEC7A, Dectin-2/CLEC6A, DLEC/CLEC4C/BDCA-2, Ficolin-1, Ficolin-2, Ficolin-3, Klre-1, KLRG2, Langerin/CD207, Layilin, LOX-1/OLR1, LSECtin/CLEC4G, MBL, MBL-1, MBL-2, MDL-1/CLEC5A, MGL1/2 (CD301a/b), MGL1/CD301a, MGL2/CD301b, MGL2/CD301b, MICL/CLEC12A, MMR/CD206, Mrc2, NKG2A/CD159a, NKG2A/NKG2B Isoform 2, NKG2C/CD159c, NKG2D/CD314, NKG2E, NKG2H, NKp80/KLRF1, OCIL/CLEC2d, OCILRP2/CLEC2i, PLA2R1, QBRICK/FREM1, Reg1, Reg1A, Reg1B, Reg2, Reg3A, Reg3B, Reg3D, Reg3G, Reg4, SCGF/CLEC11a, SFTPA1, SIGNR1/CD209b, SIGNR3/CD209d, SIGNR4/CD209e, SIGNR7/CD209g, and SP-D. In embodiments, this chimeric protein is referred to herein as TNFR2-Fc-Type II.

In embodiments, TNFR2 is the receptor that binds tumor necrosis factor-alpha (TNFα), which is a cytokine produced by lymphocytes and macrophages, that mediates the immune response by attracting additional white blood cells to sites of inflammation and through additional molecular mechanisms that initiate and amplify inflammation. TNFR2's binding to TNFα, helps decrease excess inflammation cause by, as examples, autoimmune diseases such as ankylosing spondylitis, juvenile rheumatoid arthritis, psoriasis, psoriatic arthritis, rheumatoid arthritis, and, potentially, in a variety of other disorders mediated by excess TNFα.

In embodiments, the chimeric proteins of the present invention comprise variants of the extracellular domain, which includes the ligand-binding domain, of TNFR2. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence with the known amino acid sequence of the extracellular domain of TNFR2, e.g., human TNFR2.

In embodiments, the extracellular domain of TNFR2 has the following amino acid sequence:

(SEQ ID NO: 90)
LPAQVAFTPYAPEPGSTCRLREYYDQTAQMCCSKC
SPGQHAKVFCTKTSDTVCDSCEDSTYTQLWNWVPE
CLSCGSRCSSDQVETQACTREQNRICTCRPGWYCA
LSKQEGCRLCAPLRKCRPGFGVARPGTETSDVVCK
PCAPGTFSNTTSSTDICRPHQICNVVAIPGNASMD
AVCTSTSPTRSMAPGAVHLPQPVSTRSQHTQPTPE
PSTAPSTSFLLPMGPSPPAEGSTGD.

In embodiments, a chimeric protein comprises a variant of the extracellular domain of TNFR2. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 90.

In embodiments, the first domain of a chimeric protein comprises an amino acid sequence that is at least 95% identical to the amino acid sequence of SEQ ID NO: 90.

One of ordinary skill may select variants of the known amino acid sequence of TNFR2 by consulting the literature, e.g., Kohno et al., “A second tumor necrosis factor receptor gene product can shed a naturally occurring tumor necrosis factor inhibitor.” Proc. Natl. Acad. Sci. U.S.A. 87 (21), 8331-8335 (1990); Smith et al., “A receptor for tumor necrosis factor defines an unusual family of cellular and viral proteins.” Science 248 (4958), 1019-1023 (1990); Loetscher et al., “Purification and partial amino acid sequence analysis of two distinct tumor necrosis factor receptors from HL60 cells.” J. Biol. Chem. 265 (33), 20131-20138 (1990); Dembic, et al., “Two human TNF receptors have similar extracellular, but distinct intracellular, domain sequences.” Cytokine 2 (4), 231-237 (1990); Pennica et al., “Biochemical properties of the 75-kDa tumor necrosis factor receptor. Characterization of ligand binding, internalization, and receptor phosphorylation.” J. Biol. Chem. 267 (29), 21172-21178 (1992); and Park et al., “Structural basis for self-association and receptor recognition of human TRAF2.” Nature 398 (6727), 533-538 (1999), each of which is incorporated by reference in its entirety.

In embodiments, the chimeric proteins of the present invention comprise variants of the extracellular domain of a herein-described Type II transmembrane protein, i.e., selected from 4-1BBL, APRIL, BAFF, BTNL2, CD28, CD30L, CD40L, CD70, C-type lectin domain (CLEC) family members, FasL, GITRL, LIGHT, LTa, LTa1b2, NKG2A, NKG2C, NKG2D, OX40L, RANKL, TL1A, TNFa, and TRAIL. In embodiments, the CLEC family member is selected from AlCL/CLEC-2B, ASGR1/ASGPR1, ASGR2, C1q R1/CD93, CD161, CD161/NK1.1, CD23/Fc epsilon RII, CD302/CLEC13A, CD72, CD94, Chondrolectin, CLEC-1, CLEC10A/CD301, CLEC12B, CLEC14A, CLEC16A, CLEC17A, CLEC18A, CLEC18B, CLEC18C, CLEC-2/CLEC1B, CLEC-2A, CLEC3A, CLEC3B/Tetranectin, CLEC4B2/mDCAR1, CLEC4D/CLECSF8, CLEC4E, CLEC4F/CLECSF13, CLEC9a, CLECL1/DCAL-1, CL-K1/COLEC11, CL-L1/COLEC10, CL-P1/COLEC12, DCAR/CLEC4B, DCIR/CLEC4A, DCIR4/CLEC4A1, DC-SIGN/CD209, DC-SIGN+FDC-SIGNR, DC-SIGNR/CD299, DC-SIGNR/CD299, DEC-205/CD205, Dectin-1/CLEC7A, Dectin-2/CLEC6A, DLEC/CLEC4C/BDCA-2, Ficolin-1, Ficolin-2, Ficolin-3, Klre-1, KLRG2, Langerin/CD207, Layilin, LOX-1/OLR1, LSECtin/CLEC4G, MBL, MBL-1, MBL-2, MDL-1/CLEC5A, MGL1/2 (CD301a/b), MGL1/CD301a, MGL2/CD301b, MGL2/CD301b, MICL/CLEC12A, MMR/CD206, Mrc2, NKG2A/CD159a, NKG2A/NKG2B Isoform 2, NKG2C/CD159c, NKG2D/CD314, NKG2E, NKG2H, NKp80/KLRF1, OCIL/CLEC2d, OCILRP2/CLEC2i, PLA2R1, QBRICK/FREM1, Reg1, Reg1A, Reg1B, Reg2, Reg3A, Reg3B, Reg3D, Reg3G, Reg4, SCGF/CLEC11a, SFTPA1, SIGNR1/CD209b, SIGNR3/CD209d, SIGNR4/CD209e, SIGNR7/CD209g, and SP-D. The amino acid sequence of the herein-described Type II transmembrane protein are publically available, see, e.g., at the World Wide Web (www) uniprot.org and at the World Wide Web (www) ncbi.nlm.nih.gov/protein and in one or more of WO2018/157162; WO2018/157165; WO2018/157164; WO2018/157163; and WO2017/059168, the contents relevant to this embodiment are incorporated herein by reference in its entirety. Moreover, many of the herein-described Type II transmembrane proteins have been structurally characterized, e.g., by predictive algorithms and/or x-ray crystallography; again see (www) uniprot.org; the contents relevant to this embodiment are incorporated herein by reference in its entirety.

Based on the published amino acid sequences and structural characterizations, a skilled artisan could readily determine sequence variants of the herein-described Type II transmembrane protein which retain (or enhance) the native ligand/receptor binding affinity or the Type II transmembrane protein. Examples of such variants may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of a herein-described Type II transmembrane protein, e.g., the human Type II transmembrane protein.

In embodiments, a chimeric protein of the present invention comprises: (1) a first domain comprising the amino acid sequence of SEQ ID NO: 90 or a variant thereof, as described above, (b) a second domain comprises the amino acid sequence of a portion of the extracellular domain of a herein-described Type II transmembrane protein, or a variant thereof, as described above, and (c) a linker comprises an amino acid sequence that is at least 95% identical to SEQ ID NO: 1, SEQ ID NO: 2, or SEQ ID NO: 3.

In embodiments, the chimeric protein is capable of contemporaneously binding the MadCAM receptor/ligand and the CCL25 receptor/ligand. In embodiments, the MadCAM receptor is leukocyte beta7 integrin LPAM-1 (alpha4/beta7), L-selectin, and VLA-4 (alpha4/beta1) on myeloid cells to direct leukocytes into mucosal and inflamed tissues. MadCAM is a member of the immunoglobulin superfamily and is similar to ICAM-1 and VCAM-1. CCL25, also known as TECK (Thymus-Expressed Chemokine), is a small cytokine of the CC chemokine family. It is chemotactic for thymocytes, macrophages, and dendritic cells. CCL25 elicits its effects by binding to the chemokine receptor CCR9. Accordingly, a chimeric protein comprises an extracellular domain of MadCAM, which includes its receptor-binding domain and a portion of CCL25, which is capable of contemporaneously. In embodiments, this chimeric protein is referred to herein as MadCAM-Fc-CCL25.

In embodiments, the chimeric proteins of the present invention comprise variants of a portion of an extracellular domain of MadCAM which includes its receptor-binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the portion of an extracellular domain of MadCAM, e.g., human MadCAM, which comprises its receptor-binding domain.

In embodiments, the portion of an extracellular domain of MadCAM comprising its receptor-binding domain has the following amino acid sequence:

(SEQ ID NO: 91)
QSLQVKPLQVEPPEPVVAVALGASRQLTCRLACADR
GASVQWRGLDTSLGAVQSDTGRSVLIVRNASLSAA
GTRVCVGSCGGRTFQHTVQLLVYAFPDQLTVSPAA
LVPGDPEVACTAHKVTPVDPNALSFSLLVGGQELE
GAQALGPEVQEEEEEPQGDEDVLFRVTERWRLPPL
GTPVPPALYCQATMRLPGLELSHRQAIPVLHSPTS
PEPPDTTSPESPDTTSPESPDTTSQEPPDTTSPEP
PDKTSPEPAPQQGSTHTPRSPGSTRTRRPEISQAG
PTQGEVIPTGSSKPAGDQ.

In embodiments, a chimeric protein comprises a variant of the portion of an extracellular domain of MadCAM comprising its receptor-binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 91.

In embodiments, the first domain of a chimeric protein comprises an amino acid sequence that is at least 95% identical to the amino acid sequence of SEQ ID NO: 91.

One of ordinary skill may select variants of the known amino acid sequence of an extracellular domain of MadCAM by consulting the literature, e.g., Tan et al., “The structure of immunoglobulin superfamily domains 1 and 2 of MAdCAM-1 reveals novel features important for integrin recognition.” Structure 6: 793-801 (1998); Dando et al., “A Reassessment of the Madcam-1 Structure and its Role in Integrin Recognition.” Acta Crystallogr D Biol Crystallogr 58: 233 (2002), each of which is incorporated by reference in its entirety.

In embodiments, the chimeric proteins of the present invention comprise variants of a portion of CCL25, which includes the receptor-binding domain, of CCL25. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of CCL25, e.g., human CCL25.

In embodiments, the extracellular domain of CCL25 has the following amino acid sequence:

(SEQ ID NO: 92)
MNLWLLACLVAGFLGAWAPAVHTQGVFEDCCLAYH
YPIGWAVLRRAWTYRIQEVSGSCNLPAAIFYLPKR
HRKVCGNPKSREVQRAMKLLDARNKVFAKLHHNTQ
TFQAGPHAVKKLSSGNSKLSSSKFSNPISSSKRNV
SLLISANSGL.

In embodiments, a chimeric protein comprises a variant of CCL25. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 92.

In embodiments, the second domain of a chimeric protein comprises an amino acid sequence that is at least 95% identical to the amino acid sequence of SEQ ID NO: 92.

One of ordinary skill may select variants of the known amino acid sequence of CCL25 by consulting the protein structure homology-model of CCL25 which is available at SWISS-MODEL repository. Bienert et al., “The SWISS-MODEL Repository—new features and functionality.” Nucleic Acids Research, 45(D1): D313-D319 (2017), which is incorporated by reference in its entirety.

In embodiments, a chimeric protein of the present invention comprises: (1) a first domain comprising the amino acid sequence of SEQ ID NO: 91, (b) a second domain comprises the amino acid sequence of SEQ ID NO: 92, and (c) a linker comprises an amino acid sequence that is at least 95% identical to SEQ ID NO: 1, SEQ ID NO: 2, or SEQ ID NO: 3.

In embodiments, a MadCAM-Fc-CCL25 chimeric protein of the present invention has the following amino acid sequence (the extracellular domain of MadCAM is indicated by underline, and the portion of CCL25 is shown in a boldface font):

(SEQ ID NO: 93)
MEWSWVFLFFLSVITGVHSQSLQVKPLQVEPPEPVV
AVALGASRQLTCRLACADRGASVQVRGLDTSLG
AVQSDTGRSVLIVRNASLSAAGTRVCVGSCGGRTF
QHTVQLLVYAFPDQLTVSPAALVPGDPEVACTAHK
VTPVDPNALSFSLLVGGQELEGAQALGPEVQEEEE
EPQGDEDVLFRVTERWRLPPLGTPVPPALYCQATM
RLPGLELSHRQAIPVLHSPTSPEPPDTTSPESPDT
TSPESPDTTSQEPPDTTSPEPPDKTSPEPAPQQGST
HTPRSPGSTRTRRPEISQAGPTQGEVIPTGSSKPAG
DQSKYGPPCPPCPAPEFLGGPSVFLFPPKPKDQLMI
SRTPEVICVVVDVSQEDPEVQFNWYVDGVEVHNAK
TKPREEQFNSTYRVVSVLTVLHQDWLSGKEYKCK
VSSKGLPSSIEKTISKAKGQPREPQVYTLPPSQEE
MTKNQVSLTCLVKGFYPSDIAVEWESNGQPENNYKT
TPPVLDSDGSFFLYSRLTVDKSRWQEGNVFSCSVLH
EALHNHYTQKSLSLSLGKIEGRMDQGVFEDCCLAY
HYPIGWAVLRRAWTYRIQEVSGSCNLPAAIFYLPK
RHRKVCGNPKSREVQRAMKLLDARNKVFAKLHHN
TQTFQAGPHAVKKLSSGNSKLSSSKFSNPISSSKR
NVSLLISANSGL.

In embodiments, a chimeric protein comprises a variant of a MadCAM-Fc-CCL25 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 93.

In embodiments, the chimeric protein is capable of contemporaneously binding the IL-6R receptor/ligand and the IL-35 receptor/ligand. In embodiments, the IL-6R ligand is IL-6. IL-6 plays a pathological role in chronic inflammation and autoimmunity. In some embodiments, the IL-6R part of the chimeric protein comprises IL-6RA (which is also known as IL-6R subunit alpha) and IL-6ST (which is also known as IL-6R subunit beta). IL-6R binds IL-6 with low affinity, but does not transduce a signal. Binding of IL-6R and IL-6ST to IL-6 leads to signal activation. Accordingly, in some embodiments, the IL-6R portion of the chimeric protein comprises an extracellular domain of IL-6RA and/or an extracellular domain of IL-6ST. IL-35 is a Treg-restricted inhibitory cytokine and is capable of exhibiting its suppressive activities in a range of autoimmune diseases. Human IL-35 comprises two subunits: EBI3, which is also known as Interleukin-27 subunit beta, and IL-12A, which is also known as Interleukin-12 subunit alpha. Accordingly, in some embodiments, the IL-35 portion of the chimeric protein comprises a portion of EBI3 and/or a portion of IL-12A. Accordingly, the chimeric protein comprises an extracellular domain of IL-6R which includes its receptor-binding domain and a portion of IL-35, which is capable of contemporaneously inhibiting IL-6 and stimulating IL-35. In embodiments, this chimeric protein is referred to herein as IL-6R-Fc-IL-35.

In embodiments, the chimeric proteins of the present invention comprise variants of an extracellular domain of IL-6RA which includes its receptor-binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of IL-6RA, e.g., human IL-6RA, which comprises its receptor-binding domain.

In embodiments, the extracellular domain of IL-6RA comprising its receptor-binding domain has the following amino acid sequence (the gene is IL6R, the protein is also known as IL6RA or CD126):

(SEQ ID NO: 94)
APRRCPAQEVARGVLTSLPGDSVILTCPGVEPEDN
ATVHWVLRKPAAGSHPSRWAGMGRRLLLRSVQLHD
SGNYSCYRAGRPAGTVHLLVDVPPEEPQLSCFRKS
PLSNVVCEWGPRSTPSLITKAVLLVRKFQNSPAEDF
QEPCQYSQESQKFSCQLAVPEGDSSFYIVSMCVAS
SVGSKFSKTQTFQGCGILQPDPPANITVTAVARNPR
WLSVTWQDPHSWNSSFYRLRFELRYRAERSKTFTT
WMVKDLQHHCVIHDAWSGLRHVVQLRAQEEFGQG
EWSEWSPEAMGTPWTESRSPPAENEVSTPMQALTT
NKDDDNILFRDSANATSLPVQDSSSVPLP.

In embodiments, a chimeric protein comprises a variant of the extracellular domain of IL-6RA comprising its receptor-binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 94.

In embodiments, the first domain of a chimeric protein comprises an amino acid sequence that is at least 95% identical to the amino acid sequence of SEQ ID NO: 94.

One of ordinary skill may select variants of the known amino acid sequence of IL-6RA by consulting the literature, e.g., Varghese et al., Structure of the extracellular domains of the human interleukin-6 receptor alpha-chain.” Proc Natl Acad Sci USA 99: 15959-15964 (2002); Boulanger et al., “Hexameric Structure and Assembly of the Interleukin-6/IL-6 alpha-Receptor/gp130 Complex.” Science 300: 2101-2104 (2003); Hecht et al., “The solution structure of the membrane-proximal cytokine receptor domain of the human interleukin-6 receptor.” Biol Chem 387: 1255-1259 (2006), each of which is incorporated by reference in its entirety.

In embodiments, the IL-6RA part of the chimeric protein further comprises IL-6ST, which is also known as IL-6RA subunit beta.

In embodiments, the chimeric proteins of the present invention comprise variants of an extracellular domain of IL-6ST which includes its receptor-binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of IL-6ST, e.g., human IL-6ST, which comprises its receptor-binding domain.

In embodiments, the extracellular domain of IL-6ST comprising its receptor-binding domain has the following amino acid sequence (the gene is IL6ST, the protein is also known as IL6RB, gp130 or CD130):

(SEQ ID NO: 95)
ELLDPCGYISPESPVVQLHSNFTAVCVLKEKCMDY
FHVNANYIVWKTNHFTIPKEQYTIINRTASSVTFT
DIASLNIQLTCNILTFGQLEQNVYGITIISGLPPE
KPKNLSCIVNEGKKMRCEWDGGRETHLETNFTLKS
EWATHKFADCKAKRDTPTSCTVDYSTVYFVNIEVW
VEAENALGKVTSDHINFDPVYKVKPNPPHNLSVIN
SEELSSILKLTWTNPSIKSVIILKYNIQYRTKDAS
TWSQIPPEDTASTRSSFTVQDLKPFTEYVFRIRCM
KEDGKGYWSDWSEEASGITYEDRPSKAPSFWYKID
PSHTQGYRTVQLVWKTLPPFEANGKILDYEVTLTR
WKSHLQNYTVNATKLTVNLTNDRYLATLTVRNLVG
KSDAAVLTIPACDFQATHPVMDLKAFPKDNMLWVE
WTTPRESVKKYILEWCVLSDKAPCITDWQQEDGTV
HRTYLRGNLAESKCYLITVTPVYADGPGSPESIKA
YLKQAPPSKGPTVRTKKVGKNEAVLEWDQLPVDVQ
NGFIRNYTIFYRTIIGNETAVNVDSSHTEYTLSSL
TSDTLYMVRMAAYTDEGGKDGPEFTFTTPKFAQGE
IE.

In embodiments, a chimeric protein comprises a variant of the extracellular domain of IL-6ST comprising its receptor-binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 95.

In embodiments, the first domain of a chimeric protein comprises an amino acid sequence that is at least 95% identical to the amino acid sequence of SEQ ID NO: 95.

One of ordinary skill may select variants of the known amino acid sequence of IL-6ST by consulting the literature, e.g., Kernebeck et al., “The signal transducer gp130: solution structure of the carboxy-terminal domain of the cytokine receptor homology region.” Protein Sci 8: 5-12 (1999); Boulanger et al., “Hexameric Structure and Assembly of the Interleukin-6/IL-6 alpha-Receptor/gp130 Complex.” Science 300: 2101-2104 (2003); Hecht et al., “The solution structure of the membrane-proximal cytokine receptor domain of the human interleukin-6 receptor.” Biol Chem 387: 1255-1259 (2006); Bravo et al., “Crystal structure of a cytokine-binding region of gp130.” EMBO J 17: 1665-1674 (1998); Chow et al., “Structure of an extracellular gp130 cytokine receptor signaling complex.” Science 291: 2150-2150 (2001); Xu et al., “Crystal structure of the entire ectodomain of gp130: insights into the molecular assembly of the tall cytokine receptor complexes.” J Biol Chem 285: 21214-21218 (2010), each of which is incorporated by reference in its entirety.

In embodiments, the chimeric proteins of the present invention comprise variants of the extracellular domain, which includes the receptor-binding domain, of IL-35. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of IL-35, e.g., human IL-35.

Human IL-35 comprises two subunits: EBI3, which is also known as Interleukin-27 subunit beta, and IL-12A, which is also known as Interleukin-12 subunit alpha. Accordingly, in some embodiments, the IL-35 portion of the chimeric protein comprises a portion of EBI3 and/or a portion of IL-12A.

In embodiments, the portion of EBI3 has the following amino acid sequence (also known as IL27B):

(SEQ ID NO: 96)
MTPQLLLALVLWASCPPCSGRKGPPAALTLPRVQC
RASRYPIAVDCSWTLPPAPNSTSPVSFIATYRLGM
AARGHSWPCLQQTPTSTSCTITDVQLFSMAPYVLNV
TAVHPWGSSSSFVPFITEHIIKPDPPEGVRLSPLAE
RQLQVQWEPPGSWPFPEIFSLKYWIRYKRQGAARFH
RVGPIEATSFILRAVRPRARYYVQVAAQDLTDYGEL
SDWSLPATATMSLGK.

In embodiments, a chimeric protein comprises a variant of the portion of EBI3. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 96.

In embodiments, the second domain of a chimeric protein comprises an amino acid sequence that is at least 95% identical to the amino acid sequence of SEQ ID NO: 96.

One of ordinary skill may select variants of the known amino acid sequence of EBI3 by consulting the protein structure homology-model of CCL25 which is available at SWISS-MODEL repository. Bienert et al., “The SWISS-MODEL Repository—new features and functionality.” Nucleic Acids Research, 45(D1): D313-D319 (2017), which is incorporated by reference in its entirety.

In embodiments, the portion of IL-12A has the following amino acid sequence:

(SEQ ID NO: 97)
MCPARSLLLVATLVLLDHLSLARNLPVATPDPGMFPCLHHSQNLLRAVSN
MLQKARQTLEFYPCTSEEIDHEDITKDKTSTVEACLPLELTKNESCLNSR
ETSFITNGSCLASRKTSFMMALCLSSIYEDLKMYQVEFKTMNAKLLMDPK
RQIFLDQNMLAVIDELMQALNFNSETVPQKSSLEEPDFYKTKIKLCILLH
AFRIRAVTIDRVMSYLNAS.

In embodiments, a chimeric protein comprises a variant of the portion of IL-12A. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 97.

In embodiments, the second domain of a chimeric protein comprises an amino acid sequence that is at least 95% identical to the amino acid sequence of SEQ ID NO: 97.

One of ordinary skill may select variants of the known amino acid sequence of IL12A by consulting the literature, e.g., Yoon et al., “Charged residues dominate a unique interlocking topography in the heterodimeric cytokine interleukin-12.” EMBO J 19: 3530-3541 (2000); Luo et al., “Structural basis for the dual recognition of IL-12 and IL-23 by ustekinumab.” J Mol Biol 402: 797-812 (2010), each of which is incorporated by reference in its entirety.

In embodiments, a chimeric protein of the present invention comprises: (1) a first domain comprising the amino acid sequence of SEQ ID NO: 94, (b) a second domain comprises the amino acid sequence of SEQ ID NO: 96, and (c) a linker comprises an amino acid sequence that is at least 96% identical to SEQ ID NO: 1, SEQ ID NO: 2, or SEQ ID NO: 3.

In embodiments, a chimeric protein of the present invention comprises: (1) a first domain comprising the amino acid sequence of SEQ ID NO: 95, (b) a second domain comprises the amino acid sequence of SEQ ID NO: 97, and (c) a linker comprises an amino acid sequence that is at least 96% identical to SEQ ID NO: 1, SEQ ID NO: 2, or SEQ ID NO: 3.

In embodiments, a chimeric protein of the present invention comprises: (1) a first domain comprising the amino acid sequence of SEQ ID NO: 95, (b) a second domain comprises the amino acid sequence of SEQ ID NO: 96, and (c) a linker comprises an amino acid sequence that is at least 96% identical to SEQ ID NO: 1, SEQ ID NO: 2, or SEQ ID NO: 3.

In embodiments, a chimeric protein of the present invention comprises: (1) a first domain comprising the amino acid sequence of SEQ ID NO: 94, (b) a second domain comprises the amino acid sequence of SEQ ID NO: 97, and (c) a linker comprises an amino acid sequence that is at least 96% identical to SEQ ID NO: 1, SEQ ID NO: 2, or SEQ ID NO: 3.

In embodiments, the IL-6R-Fc-IL-35 comprises a heterodimer of an Alpha Chain and a Beta Chain, wherein the Alpha Chain comprises: (1) a first domain comprising the amino acid sequence of SEQ ID NO: 94, (b) a second domain comprises the amino acid sequence of SEQ ID NO: 96, and (c) a linker comprises an amino acid sequence that is at least 96% identical to SEQ ID NO: 1, SEQ ID NO: 2, or SEQ ID NO: 3; and the Beta Chain comprises (1) a first domain comprising the amino acid sequence of SEQ ID NO: 95, (b) a second domain comprises the amino acid sequence of SEQ ID NO: 97, and (c) a linker comprises an amino acid sequence that is at least 96% identical to SEQ ID NO: 1, SEQ ID NO: 2, or SEQ ID NO: 3.

In embodiments, the IL-6R-Fc-IL-35 comprises a heterodimer of an Alpha Chain and a Beta Chain, wherein the Alpha Chain comprises: 1) a first domain comprising the amino acid sequence of SEQ ID NO: 95, (b) a second domain comprises the amino acid sequence of SEQ ID NO: 96, and (c) a linker comprises an amino acid sequence that is at least 96% identical to SEQ ID NO: 1, SEQ ID NO: 2, or SEQ ID NO: 3; and the Beta Chain comprises (1) a first domain comprising the amino acid sequence of SEQ ID NO: 94, (b) a second domain comprises the amino acid sequence of SEQ ID NO: 97, and (c) a linker comprises an amino acid sequence that is at least 96% identical to SEQ ID NO: 1, SEQ ID NO: 2, or SEQ ID NO: 3.

In embodiments, an IL-6R-Fc-IL-35 chimeric protein of the present invention has two chains: an Alpha Chain and a Beta Chain. In embodiments, an Alpha Chain (gp130-Fc-IL-12A or IL-6ST-Fc-IL-12A) of the IL-6R-Fc-IL-35 chimeric protein of the present invention has the following sequence (the extracellular domain of IL-6ST (gp130) is indicated by underline, and the portion of IL-12A is shown in a boldface font):

(SEQ ID NO: 98)
MEWSWVFLFFLSVITGVHSELLDPCGYISPESPWQLHSNFTAVCVLKEKC
MDYFHVNANYIVWKINHFTIPKEQYTIINRTASSVTFTDIASLNIQLTCN
ILTFGQLEQNVYGITIISGLPPEKPKNLSCIVNEGKKMRCEWDGGRETHL
ETNFTLKSEWATHKFADCKAKRDTPTSCTVDYSTVYFVNIEVWVEAENAL
GKVTSDHINFDPVYKVKPNPPHNLSVINSEELSSILKLTWTNPSIKSVII
LKYNIQYRTKDASTWSQIPPEDTASTRSSFTVQDLKPFTEYVFRIRCMKE
DGKGYWSDWSEEASGITYEDRPSKAPSFWYKIDPSHTQGYRTVQLVWKTL
PPFEANGKILDYEVTLTRWKSHLQNYTVNATKLTVNLTNDRYLATLTVRN
LVGKSDAAVLTIPACDFQATHPVMDLKAFPKDNMLWVEWTTPRESVKKYI
LEWCVLSDKAPCITDWQQEDGTVHRTYLRGNLAESKCYLITVTPVYADGP
GSPESIKAYLKQAPPSKGPTVRTKKVGKNEAVLEWDQLPVDVQNGFIRNY
TIFYRTIIGNETAVNVDSSHTEYTLSSLTSDTLYMVRMAAYTDEGGKDGP
EFTFTTPKFAQGEIEGSGSRKGGKRGSKYGPPCPPCPAPEFLGGPSVFLF
PPKPKDQLMISRTPEVICWVDVSQEDPEVQFNWYVDGVEVHNAKTKPREE
QFNSTYRWSVLTVLHQDWLSGKEYKCKVSSKGLPSSIEKTISNATGQPRE
PQVYTLPPSQEEMTKNQVSLTCLVKGFYPSDIAVEWESNGQPENNYKTTP
PVLDSDGSFFLYSRLTVDKSRWQEGNVFSCSVLHEALHNHYTQKSLSLSL
GKDEGGEDGSGSRNLPVATPDPGMFPCLHHSQNLLRAVSNMLQKARQTLE
FYPCTSEEIDHEDITKDKTSTVEACLPLELTKNESCLNSRETSFITNGSC
LASRKTSFMMALCLSSIYEDLKMYQVEFKTMNAKLLMDPKRQIFLDQNML
AVIDELMQALNFNSETVPQKSSLEEPDFYKTKIKLCILLHAFRIRAVTID
RVMSYLNAS.

In embodiments, a Beta Chain (IL-6RA-Fc-IL27B or IL-6RA-Fc-EBI3) of the IL-6R-Fc-IL-35 chimeric protein of the present invention has the following sequence (the extracellular domain of IL-6RA is indicated by underline, and the portion of IL27B (EBI3) is shown in a boldface font):

(SEQ ID NO: 99)
MEWSWVFLFFLSVTTGVHSLAPRRCPAQEVARGVLTSLPGDSVTLTCPGV
EPEDNATVHWVLRKPAAGSHPSRWAGMGRRLLLRSVQLHDSGNYSCYRAG
RPAGTVHLLVDVPPEEPQLSCFRKSPLSNWCEWGPRSTPSLTTKAVLLVR
KFQNSPAEDFQEPCQYSQESQKFSCQLAVPEGDSSFYIVSMCVASSVGSK
FSKTQTFQGCGILQPDPPANITVTAVARNPRWLSVTWQDPHSWNSSFYRL
RFELRYRAERSKTFTTWMVKDLQHHCVIHDAWSGLRHWQLRAQEEFGQGE
WSEWSPEAMGTPWTESRSPPAENEVSTPMQALTINKDDDNILFRDSANAT
SLPVQDSSSVPLPGSGSDEGGEDGSKYGPPCPPCPAPEFLGGPSVFLFPP
KPKDQLMISRTPEVICVVVDVSQEDPEVQFNWYVDGVEVHNAKTKPREEQ
FNSTYRWSVLTVLHQDWLSGKEYKCKVSSKGLPSSIEKTISNATGQPREP
QVYTLPPSQEEMTKNQVSLTCLVKGFYPSDIAVEWESNGQPENNYKTTPP
VLDSDGSFFLYSRLTVDKSRWQEGNVFSCSVLHEALHNHYTQKSLSLSLG
KRKGGKRGSGSRKGPPAALTLPRVQCRASRYPIAVDCSWTLPPAPNSTSP
VSFIATYRLGMAARGHSWPCLQQTPTSTSCTITDVQLFSMAPYVLNVTAV
HPWGSSSSFVPFITEHIIKPDPPEGVRLSPLAERQLQVQWEPPGSWPFPE
IFSLKYWIRYKRQGAARFHRVGPIEATSFILRAVRPRARYYVQVAAQDLT
DYGELSDWSLPATATMSLGK.

In embodiments, a chimeric protein comprises a variant of a IL-6R-Fc-IL-35 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 98 and/or SEQ ID NO: 99.

In embodiments, the chimeric protein is capable of contemporaneously binding the TNFR2 receptor/ligand and the TGFβ receptor/ligand. In embodiments, the TNFR2 ligand is TNFα. Transforming growth factor beta (TGFβ) is an inducer of the regulatory T cells, and thus, has a crucial role in maintaining immune homeostasis. Accordingly, in embodiments, the chimeric protein comprising an extracellular domain of TNFR2 which includes its receptor-binding domain and the portion of TGFβ. In embodiments, this chimeric protein is referred to herein as TNFR2-Fc-TGFβ.

In embodiments, the chimeric proteins of the present invention comprise variants of the extracellular domain, which includes the receptor-binding domain, of TNFR2. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of TNFR2, e.g., human TNFR2.

In embodiments, the extracellular domain of TNFR2 has the following amino acid sequence:

(SEQ ID NO: 100)
LPAQVAFTPYAPEPGSTCRLREYYDQTAQMCCSKCSPGQHAKVFCTKTSD
TVCDSCEDSTYTQLWNWVPECLSCGSRCSSDQVETQACTREQNRICTCRP
GWYCALSKQEGCRLCAPLRKCRPGFGVARPGTETSDVVCKPCAPGIFSNI
TSSTDICRPHQICNVVAIPGNASMDAVCTSTSPTRSMAPGAVHLPQPVST
RSQHTQPTPEPSTAPSTSFLLPMGPSPPAEGSTGD.

In embodiments, a chimeric protein comprises a variant of the extracellular domain of TNFR2. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 100.

In embodiments, the second domain of a chimeric protein comprises an amino acid sequence that is at least 95% identical to the amino acid sequence of SEQ ID NO: 100.

One of ordinary skill may select variants of the known amino acid sequence of TNFR2 by consulting the literature, e.g., Park et al., “Structural basis for self-association and receptor recognition of human TRAF2.” Nature 398: 533-538 (1999); Mukai et al. “Solution of the Structure of the TNF-TNFR2 Complex” Sci Signal 3: ra83-ra83 (2010), each of which is incorporated by reference in its entirety.

In embodiments, the chimeric proteins of the present invention comprise variants of a portion of TGFβ which includes its receptor-binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the portion of TGFβ, e.g., human TGFβ, which comprises its receptor-binding domain.

In embodiments, the portion of TGFβ comprising its receptor-binding domain has the following amino acid sequence:

(SEQ ID NO: 101)
MPPSGLRLLLLLLPLLWLLVLTPGRPAAGLSTCKTIDMELVKRKRIEAIR
GQILSKLRLASPPSQGEVPPGPLPEAVLALYNSTRDRVAGESAEPEPEPE
ADYYAKEVTRVLMVETHNEIYDKFKQSTHSIYMFFNTSELREAVPEPVLL
SRAELRLLRLKLKVEQHVELYQKYSNNSWRYLSNRLLAPSDSPEWLSFDV
TGWRQWLSRGGEIEGFRLSAHCSCDSRDNTLQVDINGFTTGRRGDLATIH
GMNRPFLLLMATPLERAQHLQSSRHRRALDTNYCFSSTEKNCCVRQLYID
FRKDLGWKWIHEPKGYHANFCLGPCPYIWSLDTQYSKVLALYNQHNPGAS
AAPCCVPQALEPLPIVYYVGRKPKVEQLSNMIVRSCKCS.

In embodiments, a chimeric protein comprises a variant of the portion of TGFβ comprising its receptor-binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 101.

In embodiments, the first domain of a chimeric protein comprises an amino acid sequence that is at least 95% identical to the amino acid sequence of SEQ ID NO: 101.

One of ordinary skill may select variants of the known amino acid sequence of TGFβ by consulting the literature, e.g., Hinck et al., “Transforming growth factor beta 1: three-dimensional structure in solution and comparison with the X-ray structure of transforming growth factor beta 2.” Biochemistry 35: 8517-8534 (1996); Hinck et al., “Transforming growth factor beta 1: three-dimensional structure in solution and comparison with the X-ray structure of transforming growth factor beta 2.” Biochemistry 35: 8517-8534 (1996); Radaev et al., “Ternary complex of transforming growth factor-beta1 reveals isoform-specific ligand recognition and receptor recruitment in the superfamily.” J Biol Chem 285: 14806-14814 (2010); Zhao et al., “Prodomain-growth factor swapping in the structure of pro-TGF-beta 1.” J Biol Chem 293: 1579-1589 (2018), each of which is incorporated by reference in its entirety.

In embodiments, a chimeric protein of the present invention comprises: (1) a first domain comprising the amino acid sequence of SEQ ID NO: 100, (b) a second domain comprises the amino acid sequence of SEQ ID NO: 101, and (c) a linker comprises an amino acid sequence that is at least 95% identical to SEQ ID NO: 1, SEQ ID NO: 2, or SEQ ID NO: 3.

In embodiments, a TNFR2-Fc-TGFβ chimeric protein of the present invention has the following amino acid sequence (the extracellular domain of TNFR2 is indicated by underline, and the portion of TGFβ is shown in a boldface font):

(SEQ ID NO: 102)
MEFGLSWVFLVAIIKGVQCLPAQVAFTPYAPEPGSTCRLREYYDQTAQMC
CSKCSPGQHAKVFCTKTSDTVCDSCEDSTYTQLWNWVPECLSCGSRCSSD
QVETQACTREQNRICTCRPGWYCALSKQEGCRLCAPLRKCRPGFGVARPG
TETSDVVCKPCAPGIFSNITSSTDICRPHQICNVVAIPGNASMDAVCTST
SPTRSMAPGAVHLPQPVSTRSQHTQPTPEPSTAPSTSFLLPMGPSPPAEG
STGDSKYGPPCPPCPAPEFLGGPSVFLFPPKPKDQLMISRTPEVICVVVD
VSQEDPEVQFNWYVDGVEVHNAKTKPREEQFNSTYRWSVLTVLHQDWLSG
KEYKCKVSSKGLPSSIEKTISNATGQPREPQVYTLPPSQEEMTKNQVSLT
CLVKGFYPSDIAVEWESNGQPENNYKTTPPVLDSDGSFFLYSRLTVDKSR
WQEGNVFSCSVLHEALHNHYTQKSLSLSLGKIEGRMDALDTNYCFSSTEK
NCCVRQLYIDFRKDLGWKWIHEPKGYHANFCLGPCPYIWSLDTQYSKVLA
LYNQHNPGASAAPCCVPQALEPLPIVYYVGRKPKVEQLSNMIVRSCKCS.

In embodiments, a chimeric protein comprises a variant of a TNFR2-Fc-TGFβ chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 102.

In embodiments, the chimeric protein is capable of contemporaneously binding the integrin α4β7 receptor/ligand and the IL-35 receptor/ligand. In embodiments, the α4β7 receptor is mucosal vascular addressing cell adhesion molecule-1 (MadCAM-1) or vascular cell adhesion molecule-1 (VCAM-1). Integrin α4β7 comprises two subunits α4 and β7. Accordingly, in some embodiments, the IL-35 portion of the chimeric protein comprises an extracellular domain of α4 and/or an extracellular domain of β7. IL-35 is a Treg-restricted inhibitory cytokine and is capable of exhibiting its suppressive activities in a range of autoimmune diseases. Human IL-35 comprises two subunits: EBI3, which is also known as Interleukin-27 subunit beta, and IL-12A, which is also known as Interleukin-12 subunit alpha. Accordingly, in some embodiments, the IL-35 portion of the chimeric protein comprises a portion of EBI3 and/or a portion of IL-12A. Accordingly, the chimeric protein comprising an extracellular domain of α4β7 which includes its receptor-binding domain and a portion of IL-35. In embodiments, this chimeric protein is referred to herein as α4β7-Fc-IL-35.

In embodiments, the chimeric proteins of the present invention comprise variants of a portion of integrin α4, which includes its receptor-binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the portion of integrin α4, e.g., human integrin α4, which comprises its receptor-binding domain.

In embodiments, the portion of integrin α4 comprising its receptor-binding domain has the following amino acid sequence:

(SEQ ID NO: 103)
NVDTESALLYQGPHNTLFGYSVVLHSHGANRWLLVGAPTANWLANASVIN
PGAIYRCRIGKNPGQICEQLQLGSPNGEPCGKTCLEERDNQWLGVTLSRQ
PGENGSIVTCGHRWKNIFYIKNENKLPTGGCYGVPPDLRTELSKRIAPCY
QDYVKKFGENFASCQAGISSFYTKDLIVMGAPGSSYWTGSLFVYNITTNK
YKAFLDKQNQVKFGSYLGYSVGAGHFRSQHTTEVVGGAPQHEQIGKAYIF
SIDEKELNILHEMKGKKLGSYFGASVCAVDLNADGFSDLLVGAPMQSTIR
EEGRVFVYINSGSGAVMNAMETNLVGSDKYAARFGESIVNLGDIDNDGFE
DVAIGAPQEDDLQGAIYIYNGRADGISSTFSQRIEGLQISKSLSMFGQSI
SGQIDADNNGYVDVAVGAFRSDSAVLLRTRPVVIVDASLSHPESVNRTKF
DCVENGWPSVCIDLTLCFSYKGKEVPGYIVLFYNMSLDVNRKAESPPRFY
FSSNGTSDVITGSIQVSSREANCRTHQAFMRKDVRDILTPIQIEAAYHLG
PHVISKRSTEEFPPLQPILQQKKEKDIMKKTINFARFCAHENCSADLQVS
AKIGFLKPHENKTYLAVGSMKTLMLNVSLFNAGDDAYETTLHVKLPVGLY
FIKILELEEKQINCEVTDNSGWQLDCSIGYIYVDHLSRIDISFLLDVSSL
SRAEEDLSITVHATCENEEEMDNLKHSRVTVAIPLKYEVKLTVHGFVNPT
SFVYGSNDENEPETCMVEKMNLTFHVINTGNSMAPNVSVEIMVPNSFSPQ
TDKLFNILDVQTTTGECHFENYQRVCALEQQKSAMQTLKGIVRFLSKTDK
RLLYCIKADPHCLNFLCNFGKMESGKEASVHIQLEGRPSILEMDETSALK
FEIRATGFPEPNPRVIELNKDENVAHVLLEGLHHQRPKRYFT.

In embodiments, a chimeric protein comprises a variant of the portion of integrin α4 comprising its receptor-binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 103.

In embodiments, the first domain of a chimeric protein comprises an amino acid sequence that is at least 95% identical to the amino acid sequence of SEQ ID NO: 103.

One of ordinary skill may select variants of the known amino acid sequence of integrin α4 by consulting the literature, e.g., Yu et al., “Structural specializations of α4b7, an Integrin that Mediates Rolling Adhesion” J Cell Biol 196: 131-146 (2012), which is incorporated by reference in its entirety.

In embodiments, the chimeric proteins of the present invention comprise variants of a portion of integrin β7, which includes its receptor-binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the portion of integrin β7, e.g., human integrin β7, which comprises its receptor-binding domain.

In embodiments, the portion of integrin β7 comprising its receptor-binding domain has the following amino acid sequence:

(SEQ ID NO: 104)
ELDAKIPSTGDATEWRNPHLSMLGSCQPAPSCQKCILSHPSCAWCKQLNF
TASGEAEARRCARREELLARGCPLEELEEPRGQQEVLQDQPLSQGARGEG
ATQLAPQRVRVTLRPGEPQQLQVRFLRAEGYPVDLYYLMDLSYSMKDDLE
RVRQLGHALLVRLQEVTHSVRIGFGSFVDKTVLPFVSTVPSKLRHPCPTR
LERCQSPFSFHHVLSLTGDAQAFEREVGRQSVSGNLDSPEGGFDAILQAA
LCQEQIGWRNVSRLLVFTSDDTFHTAGDGKLGGIFMPSDGHCHLDSNGLY
SRSTEFDYPSVGQVAQALSAANIQPIFAVTSAALPVYQELSKLIPKSAVG
ELSEDSSNWQLIMDAYNSLSSTVTLEHSSLPPGVHISYESQCEGPEKREG
KAEDRGQCNHVRINQTVTFWVSLQATHCLPEPHLLRLRALGFSEELIVEL
HTLCDCNCSDTQPQAPHCSDGQGHLQCGVCSCAPGRLGRLCECSVAELSS
PDLESGCRAPNGTGPLCSGKGHCQCGRCSCSGQSSGHLCECDDASCERHE
GILCGGFGRCQCGVCHCHANRTGRACECSGDMDSCISPEGGLCSGHGRCK
CNRCQCLDGYYGALCDQCPGCKTPCERHRDCAECGAFRTGPLATNCSTAC
AHTNVTLALAPILDDGWCKERTLDNQLFFFLVEDDARGTVVLRVRPQEKG
ADH.

In embodiments, a chimeric protein comprises a variant of the portion of integrin β7 comprising its receptor-binding domain. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 104.

In embodiments, the first domain of a chimeric protein comprises an amino acid sequence that is at least 95% identical to the amino acid sequence of SEQ ID NO: 104.

One of ordinary skill may select variants of the known amino acid sequence of integrin β7 by consulting the literature, e.g., Yu et al., “Structural specializations of α4b7, an Integrin that Mediates Rolling Adhesion” J Cell Biol 196: 131-146 (2012), which is incorporated by reference in its entirety.

In embodiments, the chimeric proteins of the present invention comprise variants of the extracellular domain, which includes the receptor-binding domain, of IL-35. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the known amino acid sequence of the extracellular domain of IL-35, e.g., human IL-35.

Human IL-35 comprises two subunits: EBI3, which is also known as Interleukin-27 subunit beta, and IL-12A, which is also known as Interleukin-12 subunit alpha. Accordingly, in some embodiments, the IL-35 portion of the chimeric protein comprises a portion of EBI3 and/or a portion of IL-12A.

In embodiments, the portion of EBI3 has the following amino acid sequence (also known as IL27B):

(SEQ ID NO: 96)
MTPQLLLALVLWASCPPCSGRKGPPAALTLPRVQCRASRYPIAVDCSWTL
PPAPNSTSPVSFIATYRLGMAARGHSWPCLQQTPTSTSCTITDVQLFSMA
PYVLNVTAVHPWGSSSSFVPFITEHIIKPDPPEGVRLSPLAERQLQVQWE
PPGSWPFPEIFSLKYWIRYKRQGAARFHRVGPIEATSFILRAVRPRARYY
VQVAAQDLTDYGELSDWSLPATATMSLGK.

In embodiments, a chimeric protein comprises a variant of the portion of EBI3. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 96.

In embodiments, the second domain of a chimeric protein comprises an amino acid sequence that is at least 95% identical to the amino acid sequence of SEQ ID NO: 96.

One of ordinary skill may select variants of the known amino acid sequence of EBI3 by consulting the protein structure homology-model of CCL25 which is available at SWISS-MODEL repository. Bienert et al., “The SWISS-MODEL Repository—new features and functionality.” Nucleic Acids Research, 45(D1): D313-D319 (2017), which is incorporated by reference in its entirety.

In embodiments, the portion of IL-12A has the following amino acid sequence:

(SEQ ID NO: 97)
MCPARSLLLVATLVLLDHLSLARNLPVATPDPGMFPCLHHSQNLLRAVSN
MLQKARQTLEFYPCTSEEIDHEDITKDKTSTVEACLPLELTKNESCLNSR
ETSFITNGSCLASRKTSFMMALCLSSIYEDLKMYQVEFKTMNAKLLMDPK
RQIFLDQNMLAVIDELMQALNFNSETVPQKSSLEEPDFYKTKIKLCILLH
AFRIRAVTIDRVMSYLNAS.

In embodiments, a chimeric protein comprises a variant of the portion of IL-12A. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 97.

In embodiments, the second domain of a chimeric protein comprises an amino acid sequence that is at least 95% identical to the amino acid sequence of SEQ ID NO: 97.

One of ordinary skill may select variants of the known amino acid sequence of IL12A by consulting the literature, e.g., Yoon et al., “Charged residues dominate a unique interlocking topography in the heterodimeric cytokine interleukin-12.” EMBO J 19: 3530-3541 (2000); Luo et al., “Structural basis for the dual recognition of IL-12 and IL-23 by ustekinumab.” J Mol Biol 402: 797-812 (2010), each of which is incorporated by reference in its entirety.

In embodiments, a chimeric protein of the present invention comprises: (1) a first domain comprising the amino acid sequence of SEQ ID NO: 103, (b) a second domain comprises the amino acid sequence of SEQ ID NO: 96, and (c) a linker comprises an amino acid sequence that is at least 96% identical to SEQ ID NO: 1, SEQ ID NO: 2, or SEQ ID NO: 3.

In embodiments, a chimeric protein of the present invention comprises: (1) a first domain comprising the amino acid sequence of SEQ ID NO: 104, (b) a second domain comprises the amino acid sequence of SEQ ID NO: 97, and (c) a linker comprises an amino acid sequence that is at least 96% identical to SEQ ID NO: 1, SEQ ID NO: 2, or SEQ ID NO: 3.

In embodiments, a chimeric protein of the present invention comprises: (1) a first domain comprising the amino acid sequence of SEQ ID NO: 104, (b) a second domain comprises the amino acid sequence of SEQ ID NO: 96, and (c) a linker comprises an amino acid sequence that is at least 96% identical to SEQ ID NO: 1, SEQ ID NO: 2, or SEQ ID NO: 3.

In embodiments, a chimeric protein of the present invention comprises: (1) a first domain comprising the amino acid sequence of SEQ ID NO: 103, (b) a second domain comprises the amino acid sequence of SEQ ID NO: 97, and (c) a linker comprises an amino acid sequence that is at least 96% identical to SEQ ID NO: 1, SEQ ID NO: 2, or SEQ ID NO: 3.

In embodiments, the α4β7-Fc-IL-35 comprises a heterodimer of an Alpha Chain and a Beta Chain, wherein the Alpha Chain comprises: (1) a first domain comprising the amino acid sequence of SEQ ID NO: 103, (b) a second domain comprises the amino acid sequence of SEQ ID NO: 96, and (c) a linker comprises an amino acid sequence that is at least 96% identical to SEQ ID NO: 1, SEQ ID NO: 2, or SEQ ID NO: 3; and the Beta Chain comprises (1) a first domain comprising the amino acid sequence of SEQ ID NO: 104, (b) a second domain comprises the amino acid sequence of SEQ ID NO: 97, and (c) a linker comprises an amino acid sequence that is at least 96% identical to SEQ ID NO: 1, SEQ ID NO: 2, or SEQ ID NO: 3.

In embodiments, the α4β7-Fc-IL-35 comprises a heterodimer of an Alpha Chain and a Beta Chain, wherein the Alpha Chain comprises: 1) a first domain comprising the amino acid sequence of SEQ ID NO: 104, (b) a second domain comprises the amino acid sequence of SEQ ID NO: 96, and (c) a linker comprises an amino acid sequence that is at least 96% identical to SEQ ID NO: 1, SEQ ID NO: 2, or SEQ ID NO: 3; and the Beta Chain comprises (1) a first domain comprising the amino acid sequence of SEQ ID NO: 103, (b) a second domain comprises the amino acid sequence of SEQ ID NO: 97, and (c) a linker comprises an amino acid sequence that is at least 96% identical to SEQ ID NO: 1, SEQ ID NO: 2, or SEQ ID NO: 3.

In embodiments, an α4β7-Fc-IL-35 chimeric protein of the present invention has two chains: an Alpha Chain and a Beta Chain. In embodiments, an Alpha Chain (ITG4A-Fc-IL-12A or integrin α4-Fc-IL-12A) of the α4β7-Fc-IL-35 chimeric protein of the present invention has the following sequence (the extracellular domain of ITG4A (integrin α4) is indicated by underline, and the portion of IL-12A is shown in a boldface font):

(SEQ ID NO: 105)
MEFGLSWVFLVAIIKGVQCYNVDTESALLYQGPHNTLFGYSWLHSHGANR
WLLVGAPTANWLANASVINPGAIYRCRIGKNPGQTCEQLQLGSPNGEPCG
KTCLEERDNQWLGVTLSRQPGENGSIVTCGHRWKNIFYIKNENKLPTGGC
YGVPPDLRTELSKRIAPCYQDYVKKFGENFASCQAGISSFYTKDLIVMGA
PGSSYWTGSLFVYNITTNKYKAFLDKQNQVKFGSYLGYSVGAGHFRSQHT
TEWGGAPQHEQIGKAYIFSIDEKELNILHEMKGKKLGSYFGASVCAVDLN
ADGFSDLLVGAPMQSTIREEGRVFVYINSGSGAVMNAMETNLVGSDKYAA
RFGESIVNLGDIDNDGFEDVAIGAPQEDDLQGAIYIYNGRADGISSTFSQ
RIEGLQISKSLSMFGQSISGQIDADNNGYVDVAVGAFRSDSAVLLRTRGS
GSRKGGKRGSKYGPPCPPCPAPEFLGGPSVFLFPPKPKDQLMISRTPEVI
CWVDVSQEDPEVQFNWYVDGVEVHNAKTKPREEQFNSTYRVVSVLTVLHQ
DWLSGKEYKCKVSSKGLPSSIEKTISKAKGQPREPQVYTLPPSQEEMTKN
QVSLTCLVKGFYPSDIAVEWESNGQPENNYKTTPPVLDSDGSFFLYSRLT
VDKSRWQEGNVFSCSVLHEALHNHYTQKSLSLSLGKDEGGEDGSGSRNLP
VATPDPGMFPCLHHSQNLLRAVSNMLQKARQTLEFYPCTSEEIDHEDITK
DKTSTVEACLPLELTKNESCLNSRETSFIINGSCLASRKTSFMMALCLSS
IYEDLKMYQVEFKTMNAKLLMDPKRQIFLDQNMLAVIDELMQALNFNSET
VPQKSSLEEPDFYKTKIKLCILLHAFRIRAVTIDRVMSYLNAS.

In embodiments, a Beta Chain (ITGB7-Fc-IL27B or integrin β7-Fc-EBI3) of the α4β7-Fc-IL-35 chimeric protein of the present invention has the following sequence (the extracellular domain of ITG4A (integrin α4) is indicated by underline, and the portion of IL27B (EBI3) is shown in a boldface font):

(SEQ ID NO: 106)
MEFGLSWVFLVAIIKGVQCMVALPMVLVLLLVLSRGESELDAKIPSTGDA
TEWRNPHLSMLGSCQPAPSCQKCILSHPSCAWCKQLNFTASGEAEARRCA
RREELLARGCPLEELEEPRGQQEVLQDQPLSQGARGEGATQLAPQRVRVT
LRPGEPQQLQVRFLRAEGYPVDLYYLMDLSYSMKDALERVRQLGHALLVR
LQEVTHSVRIGFGSFVDKTVLPFVSTVPSKLRHPCPTRLERCQSPFSFHH
VLSLTGDAQAFEREVGRQSVSGNLDSPEGGFDAILQAALCQEQIGWRNVS
RLLVFTSDDTFHTAGDGKLGGIFMPSDGHCHLDSNGLYSRSTEFDYPSVG
QVAQALSAANIQPIFAVISAALPVYQELSKLIPKSAVGELSEDSSNWQLI
MDAYNSLSSTVTLEHSSLPPGVHISYESQCEGPEKREGKAEDRGQCNHVR
INQTVTFWVSLQATHCLPEPHLLRLRALGFSEELIVELHTLCGSGSDEGG
EDGSKYGPPCPPCPAPEFLGGPSVFLFPPKPKDQLMISRTPEVICWVDVS
QEDPEVQFNWYVDGVEVHNAKTKPREEQFNSTYRWSVLTVLHQDWLSGKE
YKCKVSSKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVLHEALHNHYTQKSLSLSLGKRKGGKRGSGSRKGPPAALTLP
RVQCRASRYPIAVDCSWTLPPAPNSTSPVSFIATYRLGMAARGHSWPCLQ
QTPTSTSCTITDVQLFSMAPYVLNVTAVHPWGSSSSFVPFITEHIIKPDP
PEGVRLSPLAERQLQVQWEPPGSWPFPEIFSLKYWIRYKRQGAARFHRVG
PIEATSFILRAVRPRARYYVQVAAQDLTDYGELSDWSLPATATMSLGK.

In embodiments, a chimeric protein comprises a variant of an α4β7-Fc-IL-35 chimeric protein. As examples, the variant may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with SEQ ID NO: 105 and/or SEQ ID NO: 106.

In any herein-disclosed aspect and embodiment, the chimeric protein may comprise an amino acid sequence having one or more amino acid mutations relative to any of the protein sequences disclosed herein. In embodiments, the one or more amino acid mutations may be independently selected from substitutions, insertions, deletions, and truncations.

In embodiments, the amino acid mutations are amino acid substitutions, and may include conservative and/or non-conservative substitutions. “Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe. As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt α-helices. As used herein, “non-conservative substitutions” are defined as exchanges of an amino acid by another amino acid listed in a different group of the six standard amino acid groups (1) to (6) shown above.

In embodiments, the substitutions may also include non-classical amino acids (e.g., selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

Mutations may also be made to the nucleotide sequences of the chimeric proteins by reference to the genetic code, including taking into account codon degeneracy.

In embodiments, a chimeric protein is capable of binding murine ligand(s)/receptor(s).

In embodiments, a chimeric protein is capable of binding human ligand(s)/receptor(s).

In embodiments, each extracellular domain (or variant thereof) of the chimeric protein binds to its cognate receptor or ligand with a K_{D} of about 1 nM to about 5 nM, for example, about 1 nM, about 1.5 nM, about 2 nM, about 2.5 nM, about 3 nM, about 3.5 nM, about 4 nM, about 4.5 nM, or about 5 nM. In embodiments, the chimeric protein binds to a cognate receptor or ligand with a K_{D} of about 5 nM to about 15 nM, for example, about 5 nM, about 5.5 nM, about 6 nM, about 6.5 nM, about 7 nM, about 7.5 nM, about 8 nM, about 8.5 nM, about 9 nM, about 9.5 nM, about 10 nM, about 10.5 nM, about 11 nM, about 11.5 nM, about 12 nM, about 12.5 nM, about 13 nM, about 13.5 nM, about 14 nM, about 14.5 nM, or about 15 nM.

In embodiments, each extracellular domain (or variant thereof) of the chimeric protein binds to its cognate receptor or ligand with a K_{D} of less than about 1 μM, about 900 nM, about 800 nM, about 700 nM, about 600 nM, about 500 nM, about 400 nM, about 300 nM, about 200 nM, about 150 nM, about 130 nM, about 100 nM, about 90 nM, about 80 nM, about 70 nM, about 60 nM, about 55 nM, about 50 nM, about 45 nM, about 40 nM, about 35 nM, about 30 nM, about 25 nM, about 20 nM, about 15 nM, about 10 nM, or about 5 nM, or about 1 nM (as measured, for example, by surface plasmon resonance or biolayer interferometry). In embodiments, the chimeric protein binds to human CSF1 with a K_{D} of less than about 1 nM, about 900 μM, about 800 μM, about 700 μM, about 600 μM, about 500 μM, about 400 μM, about 300 μM, about 200 μM, about 100 μM, about 90 μM, about 80 μM, about 70 μM, about 60 μM about 55 μM about 50 μM about 45 μM, about 40 μM, about 35 μM, about 30 μM, about 25 μM, about 20 μM, about 15 μM, or about 10 μM, or about 1 μM (as measured, for example, by surface plasmon resonance or biolayer interferometry).

As used herein, a variant of an extracellular domain is capable of binding the receptor/ligand of a native extracellular domain. For example, a variant may include one or more mutations in an extracellular domain which do not affect its binding affinity to its receptor/ligand; alternately, the one or more mutations in an extracellular domain may improve binding affinity for the receptor/ligand; or the one or more mutations in an extracellular domain may reduce binding affinity for the receptor/ligand, yet not eliminate binding altogether. In embodiments, the one or more mutations are located outside the binding pocket where the extracellular domain interacts with its receptor/ligand. In embodiments, the one or more mutations are located inside the binding pocket where the extracellular domain interacts with its receptor/ligand, as long as the mutations do not eliminate binding altogether. Based on the skilled artisan's knowledge and the knowledge in the art regarding receptor-ligand binding, s/he would know which mutations would permit binding and which would eliminate binding.

In embodiments, the chimeric protein exhibits enhanced stability, high-avidity binding characteristics, prolonged off-rate for target binding and protein half-life relative to single-domain fusion protein or antibody controls.

A chimeric protein of the present invention may comprise more than two extracellular domains. For example, the chimeric protein may comprise three, four, five, six, seven, eight, nine, ten, or more extracellular domains. A second extracellular domain may be separated from a third extracellular domain via a linker, as disclosed herein. Alternately, a second extracellular domain may be directly linked (e.g., via a peptide bond) to a third extracellular domain. In embodiments, a chimeric protein includes extracellular domains that are directly linked and extracellular domains that are indirectly linked via a linker, as disclosed herein.

### Linkers

In embodiments, the chimeric protein comprises a linker.

In embodiments, the linker comprising at least one cysteine residue capable of forming a disulfide bond. The at least one cysteine residue is capable of forming a disulfide bond between a pair (or more) of chimeric proteins. Without wishing to be bound by theory, such disulfide bond forming is responsible for maintaining a useful multimeric state of chimeric proteins. This allows for efficient production of the chimeric proteins; it allows for desired activity in vitro and in vivo.

In a chimeric protein of the present invention, the linker is a polypeptide selected from a flexible amino acid sequence, an IgG hinge region, or an antibody sequence.

In embodiments, the linker is derived from naturally-occurring multi-domain proteins or is an empirical linker as described, for example, in Chichili et al, (2013), Protein Sci. 22(2):153-167, Chen et al, (2013), Adv Drug Deliv Rev. 65(10):1357-1369, the entire contents of which are hereby incorporated by reference. In embodiments, the linker may be designed using linker designing databases and computer programs such as those described in Chen et al, (2013), Adv Drug Deliv Rev. 65(10):1357-1369 and Crasto et. al., (2000), Protein Eng. 13(5):309-312, the entire contents of which are hereby incorporated by reference.

In embodiments, the linker comprises a polypeptide. In embodiments, the polypeptide is less than about 500 amino acids long, about 450 amino acids long, about 400 amino acids long, about 350 amino acids long, about 300 amino acids long, about 250 amino acids long, about 200 amino acids long, about 150 amino acids long, or about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long. In embodiments, the linker is not a single amino acid linker, e.g., without limitation, the linker is greater than one amino acid long. In embodiments, the linker has a length of greater than 1-6 amino acids, e.g., without limitation, the linker is greater than seven amino acids long. In embodiments, the linker comprises more than a single glycine residue.

In embodiments, the linker is flexible.

In embodiments, the linker is rigid.

In embodiments, the linker is substantially comprised of glycine and serine residues (e.g., about 30%, or about 40%, or about 50%, or about 60%, or about 70%, or about 80%, or about 90%, or about 95%, or about 97%, or about 98%, or about 99%, or about 100% glycines and serines).

In embodiments, the linker comprises a hinge region of an antibody (e.g., of IgG, IgA, IgD, and IgE, inclusive of subclasses (e.g., IgG1, IgG2, IgG3, and IgG4, and IgA1, and IgA2)). The hinge region, found in IgG, IgA, IgD, and IgE class antibodies, acts as a flexible spacer, allowing the Fab portion to move freely in space. In contrast to the constant regions, the hinge domains are structurally diverse, varying in both sequence and length among immunoglobulin classes and subclasses. For example, the length and flexibility of the hinge region varies among the IgG subclasses. The hinge region of IgG1 encompasses amino acids 216-231 and, because it is freely flexible, the Fab fragments can rotate about their axes of symmetry and move within a sphere centered at the first of two inter-heavy chain disulfide bridges. IgG2 has a shorter hinge than IgG1, with 12 amino acid residues and four disulfide bridges. The hinge region of IgG2 lacks a glycine residue, is relatively short, and contains a rigid poly-proline double helix, stabilized by extra inter-heavy chain disulfide bridges. These properties restrict the flexibility of the IgG2 molecule. IgG3 differs from the other subclasses by its unique extended hinge region (about four times as long as the IgG1 hinge), containing 62 amino acids (including 21 prolines and 11 cysteines), forming an inflexible poly-proline double helix. In IgG3, the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2. The flexibility of the hinge regions reportedly decreases in the order IgG3>IgG1>IgG4>IgG2. In embodiments, the linker may be derived from human IgG4 and contain one or more mutations to enhance dimerization (including S228P) or FcRn binding.

According to crystallographic studies, the immunoglobulin hinge region can be further subdivided functionally into three regions: the upper hinge region, the core region, and the lower hinge region. See Shin et al., 1992 Immunological Reviews 130:87. The upper hinge region includes amino acids from the carboxyl end of CH1 to the first residue in the hinge that restricts motion, generally the first cysteine residue that forms an interchain disulfide bond between the two heavy chains. The length of the upper hinge region correlates with the segmental flexibility of the antibody. The core hinge region contains the inter-heavy chain disulfide bridges, and the lower hinge region joins the amino terminal end of the C_{H2} domain and includes residues in C_{H2}. Id. The core hinge region of wild-type human IgG1 contains the sequence CPPC (SEQ ID NO: 24) which, when dimerized by disulfide bond formation, results in a cyclic octapeptide believed to act as a pivot, thus conferring flexibility. In embodiments, the present linker comprises, one, or two, or three of the upper hinge region, the core region, and the lower hinge region of any antibody (e.g., of IgG, IgA, IgD, and IgE, inclusive of subclasses (e.g., IgG1, IgG2, IgG3, and IgG4, and IgA1 and IgA2)). The hinge region may also contain one or more glycosylation sites, which include a number of structurally distinct types of sites for carbohydrate attachment. For example, IgA1 contains five glycosylation sites within a 17-amino-acid segment of the hinge region, conferring resistance of the hinge region polypeptide to intestinal proteases, considered an advantageous property for a secretory immunoglobulin. In embodiments, the linker of the present invention comprises one or more glycosylation sites.

In embodiments, the linker comprises an Fc domain of an antibody (e.g., of IgG, IgA, IgD, and IgE, inclusive of subclasses (e.g., IgG1, IgG2, IgG3, and IgG4, and IgA1 and IgA2)).

In a chimeric protein of the present invention, the linker comprises a hinge-CH2-CH3 Fc domain derived from IgG4. In embodiments, the linker comprises a hinge-CH2-CH3 Fc domain derived from a human IgG4. In embodiments, the linker comprises an amino acid sequence that is at least 95% identical to the amino acid sequence of any one of SEQ ID NO: 1 to SEQ ID NO: 3, e.g., at least 95% identical to the amino acid sequence of SEQ ID NO: 2. In embodiments, the linker comprises one or more joining linkers, such joining linkers independently selected from SEQ ID NO: 4 to SEQ ID NO: 50 (or a variant thereof). In embodiments, the linker comprises two or more joining linkers each joining linker independently selected from SEQ ID NO: 4 to SEQ ID NO: 50 (or a variant thereof); wherein one joining linker is N terminal to the hinge-CH2-CH3 Fc domain and another joining linker is C terminal to the hinge-CH2-CH3 Fc domain.

In embodiments, the linker comprises a hinge-CH2-CH3 Fc domain derived from a human IgG1 antibody. In embodiments, the Fc domain exhibits increased affinity for and enhanced binding to the neonatal Fc receptor (FcRn). In embodiments, the Fc domain includes one or more mutations that increases the affinity and enhances binding to FcRn. Without wishing to be bound by theory, it is believed that increased affinity and enhanced binding to FcRn increases the in vivo half-life of the present chimeric proteins.

In embodiments, the Fc domain in a linker contains one or more amino acid substitutions at amino acid residue 250, 252, 254, 256, 308, 309, 311, 416, 428, 433 or 434 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference), or equivalents thereof. In embodiments, the amino acid substitution at amino acid residue 250 is a substitution with glutamine. In embodiments, the amino acid substitution at amino acid residue 252 is a substitution with tyrosine, phenylalanine, tryptophan or threonine. In embodiments, the amino acid substitution at amino acid residue 254 is a substitution with threonine. In embodiments, the amino acid substitution at amino acid residue 256 is a substitution with serine, arginine, glutamine, glutamic acid, aspartic acid, or threonine. In embodiments, the amino acid substitution at amino acid residue 308 is a substitution with threonine. In embodiments, the amino acid substitution at amino acid residue 309 is a substitution with proline. In embodiments, the amino acid substitution at amino acid residue 311 is a substitution with serine. In embodiments, the amino acid substitution at amino acid residue 385 is a substitution with arginine, aspartic acid, serine, threonine, histidine, lysine, alanine or glycine. In embodiments, the amino acid substitution at amino acid residue 386 is a substitution with threonine, proline, aspartic acid, serine, lysine, arginine, isoleucine, or methionine. In embodiments, the amino acid substitution at amino acid residue 387 is a substitution with arginine, proline, histidine, serine, threonine, or alanine. In embodiments, the amino acid substitution at amino acid residue 389 is a substitution with proline, serine or asparagine. In embodiments, the amino acid substitution at amino acid residue 416 is a substitution with serine. In embodiments, the amino acid substitution at amino acid residue 428 is a substitution with leucine. In embodiments, the amino acid substitution at amino acid residue 433 is a substitution with arginine, serine, isoleucine, proline, or glutamine. In embodiments, the amino acid substitution at amino acid residue 434 is a substitution with histidine, phenylalanine, or tyrosine.

In embodiments, the Fc domain linker (e.g., comprising an IgG constant region) comprises one or more mutations such as substitutions at amino acid residue 252, 254, 256, 433, 434, or 436 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference). In embodiments, the IgG constant region includes a triple M252Y/S254T/T256E mutation or YTE mutation. In embodiments, the IgG constant region includes a triple H433K/N434F/Y436H mutation or KFH mutation. In embodiments, the IgG constant region includes an YTE and KFH mutation in combination.

In embodiments, the linker comprises an IgG constant region that contains one or more mutations at amino acid residues 250, 253, 307, 310, 380, 428, 433, 434, and 435 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference). Illustrative mutations include T250Q, M428L, T307A, E380A, 1253A, H310A, M428L, H433K, N434A, N434F, N4345, and H435A. In embodiments, the IgG constant region comprises a M428L/N434S mutation or LS mutation. In embodiments, the IgG constant region comprises a T250Q/M428L mutation or QL mutation. In embodiments, the IgG constant region comprises an N434A mutation. In embodiments, the IgG constant region comprises a T307A/E380A/N434A mutation or AAA mutation. In embodiments, the IgG constant region comprises an 1253A/H310A/H435A mutation or IHH mutation. In embodiments, the IgG constant region comprises a H433K/N434F mutation. In embodiments, the IgG constant region comprises a M252Y/S254T/T256E and a H433K/N434F mutation in combination.

Additional exemplary mutations in the IgG constant region are described, for example, in Robbie, et al, Antimicrobial Agents and Chemotherapy (2013), 57(12):6147-6153, Dall'Acqua et al, JBC (2006), 281(33):23514-24, Dall'Acqua et al., Journal of Immunology (2002), 169:5171-80, Ko et al. Nature (2014) 514:642-645, Grevys et al. Journal of Immunology. (2015), 194(11):5497-508, and U.S. Pat. No. 7,083,784, the entire contents of which are hereby incorporated by reference.

An illustrative Fc stabilizing mutant is S228P. Illustrative Fc half-life extending mutants are T250Q, M428L, V308T, L309P, and Q311S and the present linkers may comprise 1, or 2, or 3, or 4, or 5 of these mutants.

In embodiments, the chimeric protein binds to FcRn with high affinity. In embodiments, the chimeric protein may bind to FcRn with a K_{D} of about 1 nM to about 80 nM. For example, the chimeric protein may bind to FcRn with a K_{D} of about 1 nM, about 2 nM, about 3 nM, about 4 nM, about 5 nM, about 6 nM, about 7 nM, about 8 nM, about 9 nM, about 10 nM, about 15 nM, about 20 nM, about 25 nM, about 30 nM, about 35 nM, about 40 nM, about 45 nM, about 50 nM, about 55 nM, about 60 nM, about 65 nM, about 70 nM, about 71 nM, about 72 nM, about 73 nM, about 74 nM, about 75 nM, about 76 nM, about 77 nM, about 78 nM, about 79 nM, or about 80 nM. In embodiments, the chimeric protein may bind to FcRn with a K_{D} of about 9 nM. In embodiments, the chimeric protein does not substantially bind to other Fc receptors (i.e. other than FcRn) with effector function.

In embodiments, the Fc domain in a linker has the amino acid sequence of SEQ ID NO: 1 (see Table 1, below), or at least 90%, or 93%, or 95%, or 97%, or 98%, or 99% identity thereto. In embodiments, mutations are made to SEQ ID NO: 1 to increase stability and/or half-life. For instance, in embodiments, the Fc domain in a linker comprises the amino acid sequence of SEQ ID NO: 2 (see Table 1, below), or at least 90%, or 93%, or 95%, or 97%, or 98%, or 99% identity thereto. For instance, in embodiments, the Fc domain in a linker comprises the amino acid sequence of SEQ ID NO: 3 (see Table 1, below), or at least 90%, or 93%, or 95%, or 97%, or 98%, or 99% identity thereto.

Further, one or more joining linkers may be employed to connect an Fc domain in a linker (e.g., one of SEQ ID NO: 1, SEQ ID NO: 2, SEQ ID NO: 3 or at least 90%, or 93%, or 95%, or 97%, or 98%, or 99% identity thereto) and the extracellular domains. For example, any one of SEQ ID NO: 4, SEQ ID NO: 5, SEQ ID NO: 6, SEQ ID NO: 7, SEQ ID NO: 8, SEQ ID NO: 9, or variants thereof may connect an extracellular domain as disclosed herein and an Fc domain in a linker as disclosed herein. Optionally, any one of SEQ ID NO: 4 to SEQ ID NO: 50, or variants thereof are located between an extracellular domain as disclosed herein and an Fc domain as disclosed herein.

In embodiments, the present chimeric proteins may comprise variants of the joining linkers disclosed in Table 1, below. For instance, a linker may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the amino acid sequence of any one of SEQ ID NO: 4 to SEQ ID NO: 50.

In embodiments, the first and second joining linkers may be different or they may be the same.

Without wishing to be bound by theory, including a linker comprising at least a part of an Fc domain in a chimeric protein, helps avoid formation of insoluble and, likely, non-functional protein concatenated oligomers and/or aggregates. This is in part due to the presence of cysteines in the Fc domain which are capable of forming disulfide bonds between chimeric proteins.

In embodiments, a chimeric protein may comprise one or more joining linkers, as disclosed herein, and lack a Fc domain linker, as disclosed herein.

In embodiments, the first and/or second joining linkers are independently selected from the amino acid sequences of SEQ ID NO: 4 to SEQ ID NO: 50 and are provided in Table 1 below:

TABLE 1
Illustrative linkers (Fc domain linkers and joining linkers)

| SEQ | |
|---|---|
| ID | |
| NO. | Sequence |
| 1 | APEFLGGPSVFLFPPKPKDILMISRTPEVICWVDVSQEDPEVQFNWYVDGVEVHNAKTKPRE |
| | EQFNSTYRWSVLTVLHQDWLSGKEYKCKVSSKGLPSSIEKTISNATGQPREPQVYTLPPSQE |
| | EMTKNQVSLTCLVKGFYPSDIAVEWESNGQPENNYKTTPPVLDSDGSFFLYSRLTVDKSSWQ |
| | EGNVFSCSVMHEALHNHYTQKSLSLSLGK |
| 2 | APEFLGGPSVFLFPPKPKDQLMISRTPEVICWVDVSQEDPEVQFNWYVDGVEVHNAKTKPREE |
| | QFNSTYRWSVLTTPHSDWLSGKEYKCKVSSKGLPSSIEKTISNATGQPREPQVYTLPPSQEEM |
| | TKNQVSLTCLVKGFYPSDIAVEWESNGQPENNYKTTPPVLDSDGSFFLYSRLTVDKSSWQEGN |
| | VFSCSVLHEALHNHYTQKSLSLSLGK |
| 3 | APEFLGGPSVFLFPPKPKDQLMISRTPEVICWVDVSQEDPEVQFNWYVDGVEVHNAKTKPREE |
| | QFNSTYRWSVLTVLHQDWLSGKEYKCKVSSKGLPSSIEKTISNATGQPREPQVYTLPPSQEEM |
| | TKNQVSLTCLVKGFYPSDIAVEWESNGQPENNYKTTPPVLDSDGSFFLYSRLTVDKSRWQEGN |
| | VFSCSVLHEALHNHYTQKSLSLSLGK |
| 4 | SKYGPPCPSCP |
| 5 | SKYGPPCPPCP |
| 6 | SKYGPP |
| 7 | IEGRMD |
| 8 | GGGVPRDCG |
| 9 | IEGRMDGGGGAGGGG |
| 10 | GGGSGGGS |
| 11 | GGGSGGGGSGGG |
| 12 | EGKSSGSGSESKST |
| 13 | GGSG |
| 14 | GGSGGGSGGGSG |
| 15 | EAAAKEAAAKEAAAK |
| 16 | EAAAREAAAREAAAREAAAR |
| 17 | GGGGSGGGGSGGGGSAS |
| 18 | GGGGAGGGG |
| 19 | GS or GGS or LE |
| 20 | GSGSGS |
| 21 | GSGSGSGSGS |
| 22 | GGGGSAS |
| 23 | APAPAPAPAPAPAPAPAPAP |
| 24 | CPPC |
| 25 | GGGGS |
| 26 | GGGGSGGGGS |
| 27 | GGGGSGGGGSGGGGS |
| 28 | GGGGSGGGGSGGGGSGGGGS |
| 29 | GGGGSGGGGSGGGGSGGGGSGGGGS |
| 30 | GGGGSGGGGSGGGGSGGGGSGGGGSGGGGS |
| 31 | GGGGSGGGGSGGGGSGGGGSGGGGSGGGGSGGGGS |
| 32 | GGGGSGGGGSGGGGSGGGGSGGGGSGGGGSGGGGSGGGGS |
| 33 | GGSGGSGGGGSGGGGS |
| 34 | GGGGGGGG |
| 35 | GGGGGG |
| 36 | EAAAK |
| 37 | EAAAKEAAAK |
| 38 | EAAAKEAAAKEAAAK |
| 39 | AEAAAKEAAAKA |
| 40 | AEAAAKEAAAKEAAAKA |
| 41 | AEAAAKEAAAKEAAAKEAAAKA |
| 42 | AEAAAKEAAAKEAAAKEAAAKEAAAKA |
| 43 | AEAAAKEAAAKEAAAKEAAAKALEAEAAAKEAAAKEAAAKEAAAKA |
| 44 | PAPAP |
| 45 | KESGSVSSEQLAQFRSLD |
| 46 | GSAGSAAGSGEF |
| 47 | GGGSE |
| 48 | GSESG |
| 49 | GSEGS |
| 50 | GEGGSGEGSSGEGSSSEGGGSEGGGSEGGGSEGGS |

In embodiments, the joining linker substantially comprises glycine and serine residues (e.g., about 30%, or about 40%, or about 50%, or about 60%, or about 70%, or about 80%, or about 90%, or about 95%, or about 97%, or about 98%, or about 99%, or about 100% glycines and serines). For example, in embodiments, the joining linker is (Gly₄Ser)ₙ, where n is from about 1 to about 8, e.g., 1, 2, 3, 4, 5, 6, 7, or 8 (SEQ ID NO: 25 to SEQ ID NO: 32, respectively). In embodiments, the joining linker sequence is GGSGGSGGGGSGGGGS (SEQ ID NO: 33). Additional illustrative joining linkers include, but are not limited to, linkers having the sequence LE, (EAAAK)ₙ (n=1-3) (SEQ ID NO: 36 to SEQ ID NO: 38), A(EAAAK)ₙA (n=2-5) (SEQ ID NO: 39 to SEQ ID NO: 42), A(EAAAK)₄ALEA(EAAAK)₄A (SEQ ID NO: 43), PAPAP (SEQ ID NO: 44), KESGSVSSEQLAQFRSLD (SEQ ID NO: 45), GSAGSAAGSGEF (SEQ ID NO: 46), and (XP)ₙ, with X designating any amino acid, e.g., Ala, Lys, or Glu. In embodiments, the joining linker is GGS. In embodiments, a joining linker has the sequence (Gly)ₙ where n is any number from 1 to 100, for example: (Gly)₈ (SEQ ID NO: 34) and (Gly)₆ (SEQ ID NO: 35).

In embodiments, the joining linker is one or more of GGGSE (SEQ ID NO: 47), GSESG (SEQ ID NO: 48), GSEGS (SEQ ID NO: 49), GEGGSGEGSSGEGSSSEGGGSEGGGSEGGGSEGGS (SEQ ID NO: 50), and a joining linker of randomly placed G, S, and E every 4 amino acid intervals.

In embodiments, where a chimeric protein comprises a first domain, one joining linker preceding an Fc domain, a second joining linker following the Fc domain, and a second domain, the chimeric protein may comprise the following structure:
First Domain-Joining Linker 1-Fc Domain-Joining Linker 2-Second Domain

The combination of a first joining linker, an Fc Domain linker, and a second joining linker is referend to herein as a “modular linker”. In embodiments, a chimeric protein comprises a modular linker as shown in Table 2:

TABLE 2
Illustrative modular linkers

| | | | Modular Linker = |
|---|---|---|---|
| Joining | | Joining | Joining Linker 1 + |
| Linker 1 | Fc | Linker 2 | Fc + Joining Linker 2 |
| SKYGPPCPSCP | APEFLGGPSVFLFPPKPKDTLMIS | IEGRMD | SKYGPPCPSCPAPEFLGGPSVFL |
| (SEQ ID NO: 4) | RTPEVICWVDVSQEDPEVQFN | (SEQ ID NO: 7) | FPPKPKDILMISRTPEVICVVVDV |
| | WYVDGVEVHNAKTKPREEQFNS | | SQEDPEVQFNWYVDGVEVHNAK |
| | TYRWSVLTVLHQDWLSGKEYKC | | TKPREEQFNSTYRWSVLTVLHQ |
| | KVSSKGLPSSIEKTISNATGQPRE | | DWLSGKEYKCKVSSKGLPSSIEK |
| | PQVYTLPPSQEEMTKNQVSLTCL | | TISNATGQPREPQVYTLPPSQEE |
| | VKGFYPSDIAVEWESNGQPENNY | | MTKNQVSLTCLVKGFYPSDIAVE |
| | KTTPPVLDSDGSFFLYSRLTVDKS | | WESNGQPENNYKTTPPVLDSDG |
| | SWQEGNVFSCSVMHEALHNHYT | | SFFLYSRLTVDKSSWQEGNVFSC |
| | QKSLSLSLGK (SEQ ID NO: 1) | | SVMHEALHNHYTQKSLSLSLGKIE |
| | | | GRMD (SEQ ID NO: 51) |
| SKYGPPCPSCP | APEFLGGPSVFLFPPKPKDQLMIS | IEGRMD | SKYGPPCPSCPAPEFLGGPSVFL |
| (SEQ ID NO: 4) | RTPEVICWVDVSQEDPEVQFN | (SEQ ID NO: 7) | FPPKPKDQLMISRTPEVICVVVD |
| | WYVDGVEVHNAKTKPREEQFNS | | VSQEDPEVQFNWYVDGVEVHNA |
| | TYRWSVLTTPHSDWLSGKEYKC | | KTKPREEQFNSTYRWSVLTTPH |
| | KVSSKGLPSSIEKTISNATGQPRE | | SDWLSGKEYKCKVSSKGLPSSIE |
| | PQVYTLPPSQEEMTKNQVSLTCL | | KTISNATGQPREPQVYTLPPSQE |
| | VKGFYPSDIAVEWESNGQPENNY | | EMTKNQVSLTCLVKGFYPSDIAV |
| | KTTPPVLDSDGSFFLYSRLTVDKS | | EWESNGQPENNYKTTPPVLDSD |
| | SWQEGNVFSCSVLHEALHNHYT | | GSFFLYSRLTVDKSSWQEGNVFS |
| | QKSLSLSLGK (SEQ ID NO: 2) | | CSVLHEALHNHYTQKSLSLSLGKI |
| | | | EGRMD (SEQ ID NO: 52) |
| SKYGPPCPSCP | APEFLGGPSVFLFPPKPKDQLMIS | IEGRMD | SKYGPPCPSCPAPEFLGGPSVFL |
| (SEQ ID NO: 4) | RTPEVICWVDVSQEDPEVQFN | (SEQ ID NO: 7) | FPPKPKDQLMISRTPEVICVVVD |
| | WYVDGVEVHNAKTKPREEQFNS | | VSQEDPEVQFNWYVDGVEVHNA |
| | TYRWSVLTVLHQDWLSGKEYKC | | KTKPREEQFNSTYRWSVLTVLH |
| | KVSSKGLPSSIEKTISNATGQPRE | | QDWLSGKEYKCKVSSKGLPSSIE |
| | PQVYTLPPSQEEMTKNQVSLTCL | | KTISNATGQPREPQVYTLPPSQE |
| | VKGFYPSDIAVEWESNGQPENNY | | EMTKNQVSLTCLVKGFYPSDIAV |
| | KTTPPVLDSDGSFFLYSRLTVDKS | | EWESNGQPENNYKTTPPVLDSD |
| | RWQEGNVFSCSVLHEALHNHYT | | GSFFLYSRLTVDKSRWQEGNVFS |
| | QKSLSLSLGK (SEQ ID NO: 3) | | CSVLHEALHNHYTQKSLSLSLGKI |
| | | | EGRMD (SEQ ID NO: 53) |
| SKYGPPCPPCP | APEFLGGPSVFLFPPKPKDTLMIS | IEGRMD | SKYGPPCPPCPAPEFLGGPSVFL |
| (SEQ ID NO: 5) | RTPEVICWVDVSQEDPEVQFN | (SEQ ID NO: 7) | FPPKPKDILMISRTPEVICVVVDV |
| | WYVDGVEVHNAKTKPREEQFNS | | SQEDPEVQFNWYVDGVEVHNAK |
| | TYRWSVLTVLHQDWLSGKEYKC | | TKPREEQFNSTYRWSVLTVLHQ |
| | KVSSKGLPSSIEKTISNATGQPRE | | DWLSGKEYKCKVSSKGLPSSIEK |
| | PQVYTLPPSQEEMTKNQVSLTCL | | TISNATGQPREPQVYTLPPSQEE |
| | VKGFYPSDIAVEWESNGQPENNY | | MTKNQVSLTCLVKGFYPSDIAVE |
| | KTTPPVLDSDGSFFLYSRLTVDKS | | WESNGQPENNYKTTPPVLDSDG |
| | SWQEGNVFSCSVMHEALHNHYT | | SFFLYSRLTVDKSSWQEGNVFSC |
| | QKSLSLSLGK (SEQ ID NO: 1) | | SVMHEALHNHYTQKSLSLSLGKIE |
| | | | GRMD (SEQ ID NO: 54) |
| SKYGPPCPPCP | APEFLGGPSVFLFPPKPKDQLMIS | IEGRMD | SKYGPPCPPCPAPEFLGGPSVFL |
| (SEQ ID NO: 7) | RTPEVICWVDVSQEDPEVQFN | (SEQ ID NO: 5) | FPPKPKDQLMISRTPEVICVVVD |
| | WYVDGVEVHNAKTKPREEQFNS | | VSQEDPEVQFNWYVDGVEVHNA |
| | TYRWSVLTTPHSDWLSGKEYKC | | KTKPREEQFNSTYRWSVLTTPH |
| | KVSSKGLPSSIEKTISNATGQPRE | | SDWLSGKEYKCKVSSKGLPSSIE |
| | PQVYTLPPSQEEMTKNQVSLTCL | | KTISNATGQPREPQVYTLPPSQE |
| | VKGFYPSDIAVEWESNGQPENNY | | EMTKNQVSLTCLVKGFYPSDIAV |
| | KTTPPVLDSDGSFFLYSRLTVDKS | | EWESNGQPENNYKTTPPVLDSD |
| | SWQEGNVFSCSVLHEALHNHYT | | GSFFLYSRLTVDKSSWQEGNVFS |
| | QKSLSLSLGK (SEQ ID NO: 2) | | CSVLHEALHNHYTQKSLSLSLGKI |
| | | | EGRMD (SEQ ID NO: 55) |
| SKYGPPCPPCP | APEFLGGPSVFLFPPKPKDQLMIS | IEGRMD | SKYGPPCPPCPAPEFLGGPSVFL |
| (SEQ ID NO: 5) | RTPEVICWVDVSQEDPEVQFN | (SEQ ID NO: 7) | FPPKPKDQLMISRTPEVICVVVD |
| | WYVDGVEVHNAKTKPREEQFNS | | VSQEDPEVQFNWYVDGVEVHNA |
| | TYRWSVLTVLHQDWLSGKEYKC | | KTKPREEQFNSTYRWSVLTVLH |
| | KVSSKGLPSSIEKTISNATGQPRE | | QDWLSGKEYKCKVSSKGLPSSIE |
| | PQVYTLPPSQEEMTKNQVSLTCL | | KTISNATGQPREPQVYTLPPSQE |
| | VKGFYPSDIAVEWESNGQPENNY | | EMTKNQVSLTCLVKGFYPSDIAV |
| | KTTPPVLDSDGSFFLYSRLTVDKS | | EWESNGQPENNYKTTPPVLDSD |
| | RWQEGNVFSCSVLHEALHNHYT | | GSFFLYSRLTVDKSRWQEGNVFS |
| | QKSLSLSLGK (SEQ ID NO: 3) | | CSVLHEALHNHYTQKSLSLSLGKI |
| | | | EGRMD (SEQ ID NO: 56) |

In embodiments, the present chimeric proteins may comprise variants of the modular linkers disclosed in Table 2, above. For instance, a linker may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the amino acid sequence of any one of SEQ ID NO: 51 to SEQ ID NO: 56.

In embodiments, the linker may be flexible, including without limitation highly flexible. In embodiments, the linker may be rigid, including without limitation a rigid alpha helix. Characteristics of illustrative joining linkers is shown below in Table 3:

TABLE 3
Characteristics of illustrative joining linkers

| Joining Linker Sequence | Characteristics |
|---|---|
| SKYGPPCPPCP (SEQ ID NO: 5) | IgG4 Hinge Region |
| IEGRMD (SEQ ID NO: 7) | Linker |
| GGGVPRDCG (SEQ ID NO: 8) | Flexible |
| GGGSGGGS (SEQ ID NO: 10) | Flexible |
| GGGSGGGGSGGG (SEQ ID NO: 11) | Flexible |
| EGKSSGSGSESKST (SEQ ID NO: 12) | Flexible + |
| | soluble |
| GGSG (SEQ ID NO: 13) | Flexible |
| GGSGGGSGGGSG (SEQ ID NO: 14) | Flexible |
| EAAAKEAAAKEAAAK (SEQ ID NO: 15) | Rigid Alpha Helix |
| EAAAREAAAREAAAREAAAR | Rigid Alpha Helix |
| (SEQ ID NO: 16) | |
| GGGGSGGGGSGGGGSAS | Flexible |
| (SEQ ID NO: 17) | |
| GGGGAGGGG (SEQ ID NO: 18) | Flexible |
| GS (SEQ ID NO: 19) | Highly flexible |
| GSGSGS (SEQ ID NO: 20) | Highly flexible |
| GSGSGSGSGS (SEQ ID NO: 21) | Highly flexible |
| GGGGSAS (SEQ ID NO: 22) | Flexible |
| APAPAPAPAPAPAPAPAPAP | Rigid |
| (SEQ ID NO: 23) | |

In embodiments, the linker may be functional. For example, without limitation, the linker may function to improve the folding and/or stability, improve the expression, improve the pharmacokinetics, and/or improve the bioactivity of the present chimeric protein. In another example, the linker may function to target the chimeric protein to a particular cell type or location.

In embodiments, a chimeric protein comprises only one joining linkers.

In embodiments, a chimeric protein lacks joining linkers.

In embodiments, the linker is a synthetic linker such as polyethylene glycol (PEG).

In embodiments, a chimeric protein has a first domain which is sterically capable of binding its ligand/receptor and/or the second domain which is sterically capable of binding its ligand/receptor. Thus, there is enough overall flexibility in the chimeric protein and/or physical distance between an extracellular domain (or portion thereof) and the rest of the chimeric protein such that the ligand/receptor binding domain of the extracellular domain is not sterically hindered from binding its ligand/receptor. This flexibility and/or physical distance (which is referred to as “slack”) may be normally present in the extracellular domain(s), normally present in the linker, and/or normally present in the chimeric protein (as a whole). Alternately, or additionally, an amino acid sequence (for example) may be added to one or more extracellular domains and/or to the linker to provide the slack needed to avoid steric hindrance. Any amino acid sequence that provides slack may be added. In embodiments, the added amino acid sequence comprises the sequence (Gly)ₙ where n is any number from 1 to 100. Additional examples of addable amino acid sequence include the joining linkers described in Table 1 and Table 3. In embodiments, a polyethylene glycol (PEG) linker may be added between an extracellular domain and a linker to provide the slack needed to avoid steric hindrance. Such PEG linkers are well known in the art.

### Pharmaceutical Composition

Aspects of the present invention include a pharmaceutical composition comprising a therapeutically effective amount of the chimeric protein of any of the herein disclosed aspects or embodiments.

In embodiments, the chimeric protein in the pharmaceutical composition has a general structure of: N terminus-(a)-(b)-(c)-C terminus in which (a) is a first domain comprising a portion of the extracellular domain of a transmembrane protein, a secreted protein, or a membrane-anchored extracellular protein, (c) is a second domain comprising a portion of the extracellular domain of a transmembrane protein, a secreted protein, or a membrane-anchored extracellular protein, and (b) is a linker adjoining the first domain and the second domain. In this aspect, either or both of the first domain and the second domain decreases self-directed immune system activity when bound to its ligand/receptor.

In embodiments, the portion of the first domain is capable of binding the native ligand/receptor for the transmembrane protein, the secreted protein, or the membrane-anchored extracellular protein.

In embodiments, the portion of the second domain is capable of binding the native ligand/receptor for the transmembrane protein, the secreted protein, or the membrane-anchored extracellular protein.

In embodiments, the first domain comprises substantially the entire extracellular domain of the transmembrane protein, substantially the entire secreted protein, or substantially the entire membrane-anchored extracellular protein.

In embodiments, the second domain comprises substantially the entire extracellular domain of the transmembrane protein, substantially the entire secreted protein, or substantially the entire membrane-anchored extracellular protein.

In embodiments, the binding of the portion of the first domain to its ligand/receptor decreases immune system activity by activating an immune inhibitory signal or inhibiting an immune activating signal.

In embodiments, the binding of the portion of the second domain to its ligand/receptor decreases immune system activity by activating an immune inhibitory signal or by inhibiting an immune activating signal.

In embodiments, the portion of the first domain comprises a transmembrane protein, a secreted protein, or a membrane-anchored extracellular protein selected from B7H3, B7H4, BTNL2, CTLA4, CSF3, ICOSL, ILDR2, PD-L1, TNFR2, IL-6R, MadCAM, integrin α4β7, and VSIG4.

In embodiments, the portion of the second domain comprises a transmembrane protein, a secreted protein, or a membrane-anchored extracellular protein selected from BTNL2, IL2, PD-L1, SEMA3E, IL-35, CCL25, TGFβ, and TL1A.

In embodiments, the first domain comprises a portion of VSIG4 and the second domain comprises a portion of IL2.

In embodiments, the first domain comprises a portion of CTLA4 and the second domain comprises a portion of IL2, e.g., the portion of IL2 comprises one or more mutations relative to a corresponding portion of wild-type IL2 wherein the one or more mutations provide preferential binding to a high-affinity IL2 receptor that is expressed by regulatory T cells.

In embodiments, the first domain comprises a portion of CTLA4 and the second domain comprises a portion of PD-L1.

In embodiments, the first domain comprises a portion of B7H3 and the second domain comprises a portion of PD-L1.

In embodiments, the first domain comprises a portion of B7H4 and the second domain comprises a portion of PD-L1.

In embodiments, the first domain comprises a portion of ICOSL and the second domain comprises a portion of PD-L1.

In embodiments, the first domain comprises a portion of ILDR2 and the second domain comprises a portion of PD-L1.

In embodiments, the first domain comprises a portion BTNL2 of and the second domain comprises a portion of PD-L1 or the first domain comprises a portion of PD-L1 and the second domain comprises a portion of BTNL2.

In embodiments, the first domain comprises a portion of CSF3 and the second domain comprises a portion of TL1A.

In embodiments, the first domain comprises a portion of CTLA4 and the second domain comprises a portion of TL1A.

In embodiments, the first domain comprises a portion of CTLA4 and the second domain comprises a portion of SEMA3E.

In embodiments, the first domain comprises a portion of TNFR2 and the second domain comprises an extracellular domain of a transmembrane protein selected from GITRL and TL1A.

In embodiments, the first domain comprises a portion of CTLA4 and the second domain comprises an extracellular domain of a transmembrane protein selected from GITRL and TL1A.

In embodiments, the first domain comprises an extracellular domain of IL-6R and the second domain comprises a portion of IL-35. In embodiments, the first domain comprises an extracellular domain of IL-6ST and/or IL-6R and/or the second domain comprises a portion of EBI3 and/or IL-12A. In embodiments, the chimeric protein is a heterodimer.

In embodiments, the first domain comprises an extracellular domain of MadCAM and the second domain comprises a portion of CCL25.

In embodiments, the first domain comprises an extracellular domain of TNFR2 and the second domain comprises a portion of TGFβ.

In embodiments, the first domain comprises an extracellular domain of integrin α4β7 and the second domain comprises a portion of IL-35. In embodiments, the first domain comprises an extracellular domain of integrin α4 and/or integrin β7, and/or the second domain comprises a portion of EBI3 and/or IL-12A. In embodiments, the chimeric protein is a heterodimer.

In embodiments, the chimeric protein is capable of contemporaneously binding a TNFR2 ligand and a ligand/receptor of a Type II transmembrane protein selected from BTNL2C-type lectin domain (CLEC) family members, GITRL TL1A, IL-10, or TGF-beta.

In embodiments, the first domain comprises a portion of TNFR2 and the second domain comprises an extracellular domain of a transmembrane protein selected from GITRL and TL1A.

In embodiments, the first domain comprises a portion of CTLA4 and the second domain comprises an extracellular domain of a transmembrane protein selected from GITRL and TL1A.

In embodiments, the first domain comprises an extracellular domain of IL-6R and the second domain comprises a portion of IL-35. In embodiments, the first domain comprises an extracellular domain of IL-6ST and/or IL-6R and/or the second domain comprises a portion of EBI3 and/or IL-12A. In embodiments, the chimeric protein is a heterodimer.

In embodiments, the first domain comprises an extracellular domain of MadCAM and the second domain comprises a portion of CCL25.

In embodiments, the first domain comprises an extracellular domain of TNFR2 and the second domain comprises a portion of TGFβ.

In embodiments, the first domain comprises an extracellular domain of integrin α4β7 and the second domain comprises a portion of IL-35. In embodiments, the first domain comprises an extracellular domain of integrin α4 and/or integrin β7, and/or the second domain comprises a portion of EBI3 and/or IL-12A. In embodiments, the chimeric protein is a heterodimer.

In embodiments, the chimeric protein is capable of contemporaneously binding a TNFR2 ligand and a ligand/receptor of a Type II transmembrane protein selected from BTNL2C-type lectin domain (CLEC) family members, GITRL TL1A, IL-10, or TGF-beta.

In embodiments, the first domain comprises a portion of TNFR2 and the second domain comprises an extracellular domain of a transmembrane protein selected from GITRL and TL1A.

In embodiments, the first domain comprises a portion of CTLA4 and the second domain comprises an extracellular domain of a transmembrane protein selected from GITRL and TL1A.

In embodiments, the first domain comprises an extracellular domain of IL-6R and the second domain comprises a portion of IL-35. In embodiments, the first domain comprises an extracellular domain of IL-6ST and/or IL-6R and/or the second domain comprises a portion of EBI3 and/or IL-12A. In embodiments, the chimeric protein is a heterodimer.

In embodiments, the first domain comprises an extracellular domain of MadCAM and the second domain comprises a portion of CCL25.

In embodiments, the first domain comprises an extracellular domain of TNFR2 and the second domain comprises a portion of TGFβ.

In embodiments, the first domain comprises an extracellular domain of integrin α4β7 and the second domain comprises a portion of IL-35. In embodiments, the first domain comprises an extracellular domain of integrin α4 and/or integrin β7, and/or the second domain comprises a portion of EBI3 and/or IL-12A. In embodiments, the chimeric protein is a heterodimer.

In embodiments, the chimeric protein is capable of contemporaneously binding a TNFR2 ligand and a ligand/receptor of a Type II transmembrane protein selected from BTNL2C-type lectin domain (CLEC) family members, GITRL TL1A, IL-10, or TGF-beta.

In embodiments, the first domain comprises a portion of TNFR2 and the second domain comprises an extracellular domain of a transmembrane protein selected from 4-1BBL, APRIL, BAFF, BTNL2, CD28, CD30L, CD40L, CD70, C-type lectin domain (CLEC) family members, FasL, GITRL, LIGHT, LTa, LTa1b2, NKG2A, NKG2C, NKG2D, OX40L, RANKL, TL1A, TNFa, and TRAIL; in embodiments, the second domain comprises an extracellular domain of GITRL or TL1A. In embodiments, the CLEC family member is selected from AlCL/CLEC-2B, ASGR1/ASGPR1, ASGR2, C1q R1/CD93, CD161, CD161/NK1.1, CD23/Fc epsilon RII, CD302/CLEC13A, CD72, CD94, Chondrolectin, CLEC-1, CLEC10A/CD301, CLEC12B, CLEC14A, CLEC16A, CLEC17A, CLEC18A, CLEC18B, CLEC18C, CLEC-2/CLEC1B, CLEC-2A, CLEC3A, CLEC3B/Tetranectin, CLEC4B2/mDCAR1, CLEC4D/CLECSF8, CLEC4E, CLEC4F/CLECSF13, CLEC9a, CLECL1/DCAL-1, CL-K1/COLEC11, CL-L1/COLEC10, CL-P1/COLEC12, DCAR/CLEC4B, DCIR/CLEC4A, DCIR4/CLEC4A1, DC-SIGN/CD209, DC-SIGN+FDC-SIGNR, DC-SIGNR/CD299, DC-SIGNR/CD299, DEC-205/CD205, Dectin-1/CLEC7A, Dectin-2/CLEC6A, DLEC/CLEC4C/BDCA-2, Ficolin-1, Ficolin-2, Ficolin-3, Klre-1, KLRG2, Langerin/CD207, Layilin, LOX-1/OLR1, LSECtin/CLEC4G, MBL, MBL-1, MBL-2, MDL-1/CLEC5A, MGL1/2 (CD301a/b), MGL1/CD301a, MGL2/CD301b, MGL2/CD301b, MICL/CLEC12A, MMR/CD206, Mrc2, NKG2A/CD159a, NKG2A/NKG2B Isoform 2, NKG2C/CD159c, NKG2D/CD314, NKG2E, NKG2H, NKp80/KLRF1, OCIL/CLEC2d, OCILRP2/CLEC2i, PLA2R1, QBRICK/FREM1, Reg1, Reg1A, Reg1B, Reg2, Reg3A, Reg3B, Reg3D, Reg3G, Reg4, SCGF/CLEC11a, SFTPA1, SIGNR1/CD209b, SIGNR3/CD209d, SIGNR4/CD209e, SIGNR7/CD209g, and SP-D.

In embodiments, the first domain comprises a portion of CTLA4 and the second domain comprises an extracellular domain of a transmembrane protein selected from 4-1BBL, APRIL, BAFF, BTNL2, CD28, CD30L, CD40L, CD70, C-type lectin domain (CLEC) family members, FasL, GITRL, LIGHT, LTa, LTa1b2, NKG2A, NKG2C, NKG2D, OX40L, RANKL, TL1A, TNFa, and TRAIL; in embodiments, the second domain comprises an extracellular domain of GITRL or TL1A. In embodiments, the CLEC family member is selected from AlCL/CLEC-2B, ASGR1/ASGPR1, ASGR2, C1q R1/CD93, CD161, CD161/NK1.1, CD23/Fc epsilon RII, CD302/CLEC13A, CD72, CD94, Chondrolectin, CLEC-1, CLEC10A/CD301, CLEC12B, CLEC14A, CLEC16A, CLEC17A, CLEC18A, CLEC18B, CLEC18C, CLEC-2/CLEC1B, CLEC-2A, CLEC3A, CLEC3B/Tetranectin, CLEC4B2/mDCAR1, CLEC4D/CLECSF8, CLEC4E, CLEC4F/CLECSF13, CLEC9a, CLECL1/DCAL-1, CL-K1/COLEC11, CL-L1/COLEC10, CL-P1/COLEC12, DCAR/CLEC4B, DCIR/CLEC4A, DCIR4/CLEC4A1, DC-SIGN/CD209, DC-SIGN+FDC-SIGNR, DC-SIGNR/CD299, DC-SIGNR/CD299, DEC-205/CD205, Dectin-1/CLEC7A, Dectin-2/CLEC6A, DLEC/CLEC4C/BDCA-2, Ficolin-1, Ficolin-2, Ficolin-3, Klre-1, KLRG2, Langerin/CD207, Layilin, LOX-1/OLR1, LSECtin/CLEC4G, MBL, MBL-1, MBL-2, MDL-1/CLEC5A, MGL1/2 (CD301a/b), MGL1/CD301a, MGL2/CD301b, MGL2/CD301b, MICL/CLEC12A, MMR/CD206, Mrc2, NKG2A/CD159a, NKG2A/NKG2B Isoform 2, NKG2C/CD159c, NKG2D/CD314, NKG2E, NKG2H, NKp80/KLRF1, OCIL/CLEC2d, OCILRP2/CLEC2i, PLA2R1, QBRICK/FREM1, Reg1, Reg1A, Reg1B, Reg2, Reg3A, Reg3B, Reg3D, Reg3G, Reg4, SCGF/CLEC11a, SFTPA1, SIGNR1/CD209b, SIGNR3/CD209d, SIGNR4/CD209e, SIGNR7/CD209g, and SP-D.

In embodiments, the binding of either or both of the first domain and the second domains to its ligand/receptor occurs with slow off rates (Koff), which provides a long interaction of a receptor and its ligand. In embodiments, the long interaction provides a prolonged decrease in immune system activity which comprises sustained activation of an immune inhibitory signal and/or a sustained inhibition of an immune activating signal. In embodiments, the sustained activation of the immune inhibitory signal and/or the sustained inhibition of the immune activating signal reduces the activity or proliferation of an immune cell, e.g., a B cell or a T cell. In embodiments, the sustained activation of the immune inhibitory signal and/or the sustained inhibition of the immune activating signal decreases synthesis and/or decreases release of a pro-inflammatory cytokine. In embodiments, the sustained activation of the immune inhibitory signal and/or the sustained inhibition of the immune activating signal increases synthesis and/or increases release of an anti-inflammatory cytokine. In embodiments, the sustained activation of the immune inhibitory signal and/or the sustained inhibition of the immune activating signal decreases antibody production and/or decreases secretion of antibodies by a B cell, e.g., an antibody that recognizes a self-antigen. In embodiments, the sustained activation of the immune inhibitory signal and/or the sustained inhibition of the immune activating signal decreases the activity of and/or decreases the number of T cytotoxic cells, e.g., which recognize a self-antigen and kill cells presenting or expressing the self-antigen. In embodiments, the sustained activation of the immune inhibitory signal and/or the sustained inhibition of the immune activating signal increases the activity and/or increases the number of T regulatory cells.

In embodiments, the linker is a polypeptide selected from a flexible amino acid sequence, an IgG hinge region, and an antibody sequence.

In embodiments, the linker comprises at least one cysteine residue capable of forming a disulfide bond and/or comprises a hinge-CH2-CH3 Fc domain, e.g., a hinge-CH2-CH3 Fc domain is derived from IgG (e.g., IgG1, IgG2, IgG3, and IgG4), IgA (e.g., IgA1 and IgA2), IgD, or IgE. In embodiments, the IgG is IgG4, e.g., a human IgG4. In embodiments, the IgG is IgG1, e.g., a human IgG1. In embodiments, the linker comprises an amino acid sequence that is at least 95% identical to the amino acid sequence of SEQ ID NO: 1, SEQ ID NO: 2, or SEQ ID NO: 3.

In embodiments, the chimeric protein in the pharmaceutical composition comprises: (a) a first domain comprising a portion of VSIG4 that is capable of binding a VSIG4 ligand/receptor, (b) a second domain comprising a portion of IL2 that is capable of binding an IL2 receptor, and (c) a linker linking the first domain and the second domain and comprising a hinge-CH2-CH3 Fc domain.

In embodiments, the chimeric protein in the pharmaceutical composition comprises: (a) a first domain comprising a portion of CTLA4 that is capable of binding a CTLA4 ligand/receptor, (b) a second domain comprising a portion of IL2 that is capable of binding an IL2 receptor, and (c) a linker linking the first domain and the second domain and comprising a hinge-CH2-CH3 Fc domain. In embodiments, the IL2 receptor is a high-affinity IL2 receptor that is expressed by regulatory T cells, e.g., the portion of IL2 comprises one or more mutations relative to a corresponding portion of wild-type IL2 which provides preferential binding to the high-affinity IL2 receptor that is expressed by regulatory T cells.

In embodiments, the chimeric protein in the pharmaceutical composition comprises: (a) a first domain comprising a portion of CTLA4 that is capable of binding a CTLA4 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein in the pharmaceutical composition comprises: (a) a first domain comprising a portion of B7H3 that is capable of binding a B7H3 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein in the pharmaceutical composition comprises: (a) a first domain comprising a portion of B7H4 that is capable of binding a B7H4 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein in the pharmaceutical composition comprises: (a) a first domain comprising a portion of ICOSL that is capable of binding an ICOSL ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein in the pharmaceutical composition comprises: (a) a first domain comprising a portion of ILDR2 that is capable of binding an ILDR2 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein in the pharmaceutical composition comprises: (a) a first domain comprising a portion of BTNL2 that is capable of binding a BTNL2 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein in the pharmaceutical composition comprises: (a) a first domain comprising a portion of PD-L1 that is capable of binding PD-1, (b) a second domain comprising a portion of BTNL2 that is capable of binding a BTNL2 ligand/receptor, and (c) a linker linking the first domain and the second domain and comprising a hinge-CH2-CH3 Fc domain.

In embodiments, the chimeric protein in the pharmaceutical composition comprises: (a) a first domain comprising a portion of CSF3 that is capable of binding a CSF3 ligand/receptor, (b) a second domain comprising a portion of TL1A that is capable of binding a TL1A ligand/receptor, and (c) a linker linking the first domain and the second domain and comprising a hinge-CH2-CH3 Fc domain.

In embodiments, the chimeric protein in the pharmaceutical composition comprises: (a) a first domain comprising a portion of CTLA4 that is capable of binding a CTLA4 ligand/receptor, (b) a second domain comprising a portion of TL1A that is capable of binding a TL1A ligand/receptor, and (c) a linker linking the first domain and the second domain and comprising a hinge-CH2-CH3 Fc domain.

In embodiments, the chimeric protein in the pharmaceutical composition comprises: (a) a first domain comprising a portion of CTLA4 that is capable of binding a CTLA4 ligand/receptor, (b) a second domain comprising a portion of SEMA3E that is capable of binding a SEMA3E ligand/receptor, and (c) a linker linking the first domain and the second domain and comprising a hinge-CH2-CH3 Fc domain.

In embodiments, the chimeric protein in the pharmaceutical composition comprises: (a) a first domain comprising a portion of MadCAM that is capable of binding a MadCAM ligand/receptor, (b) a second domain comprising a portion of CCL25 that is capable of binding a CCL25 ligand/receptor, and (c) a linker linking the first domain and the second domain and comprising a hinge-CH2-CH3 Fc domain.

In embodiments, the chimeric protein in the pharmaceutical composition comprises: (a) a first domain comprising a portion of TNFR2 that is capable of binding a TNFR2 ligand/receptor, (b) a second domain comprising a portion of TGFβ that is capable of binding a TGFβ ligand/receptor, and (c) a linker linking the first domain and the second domain and comprising a hinge-CH2-CH3 Fc domain.

In embodiments, the chimeric protein in the pharmaceutical composition comprises: (a) a first domain comprising an extracellular domain of IL-6R that is capable of binding a IL-6R ligand/receptor, (b) a second domain comprising a portion of IL-35 that is capable of binding a IL-35 ligand/receptor, and (c) a linker linking the first domain and the second domain and comprising a hinge-CH2-CH3 Fc domain. In embodiments, the first domain comprises an extracellular domain of IL-6ST and/or IL-6R. In embodiments, the second domain comprises a portion of EBI3 and/or IL-12A. In embodiments, the chimeric protein is heterodimeric.

In embodiments, the chimeric protein in the pharmaceutical composition comprises: (a) a first domain comprising an extracellular domain of integrin α4β7 that is capable of binding an integrin α4β7 ligand/receptor, (b) a second domain comprising a portion of IL-35 that is capable of binding a IL-35 ligand/receptor, and (c) a linker linking the first domain and the second domain and comprising a hinge-CH2-CH3 Fc domain. In embodiments, the first domain comprises an extracellular domain of integrin α4 and/or integrin β7. In embodiments, the second domain comprises a portion of EBI3 and/or IL-12A. In embodiments, the chimeric protein is heterodimeric.

In embodiments, the hinge-CH2-CH3 Fc domain comprises at least one cysteine residue capable of forming a disulfide bond. In embodiments, the hinge-CH2-CH3 Fc domain is derived from IgG (e.g., IgG1, IgG2, IgG3, and IgG4), IgA (e.g., IgA1 and IgA2), IgD, or IgE. In embodiments, the IgG is IgG4, e.g., a human IgG4. In embodiments, the IgG is IgG1, e.g., a human IgG1. In embodiments, the linker comprises an amino acid sequence that is at least 95% identical to the amino acid sequence of SEQ ID NO: 1, SEQ ID NO: 2, or SEQ ID NO: 3.

In embodiments, the chimeric protein in the pharmaceutical composition may be a recombinant fusion protein.

In embodiments, the pharmaceutical composition further comprises an immunosuppressive agent. In embodiments, the immunosuppressive agent is selected from the group consisting of an antibody (e.g., basiliximab, daclizumab, and muromonab), an anti-immunophilin (e.g., cyclosporine, tacrolimus, and sirolimus), an antimetabolite (e.g., azathioprine and methotrexate), a cytostatic (such as alkylating agents), a cytotoxic antibiotic, an interferon, a mycophenolate, an opioid, a small biological agent (e.g., fingolimod and myriocin), and a TNF binding protein.

In embodiments, the pharmaceutical composition further comprises an anti-inflammatory drug, e.g., a non-steroidal anti-inflammatory or a corticosteroid. In embodiments, the non-steroidal anti-inflammatory is selected from the group consisting of acetyl salicylic acid (aspirin), benzyl-2,5-diacetoxybenzoic acid, celecoxib, diclofenac, etodolac, etofenamate, fulindac, glycol salicylate, ibuprofen, indomethacin, ketoprofen, methyl salicylate, nabumetone, naproxen, oxaprozin, phenylbutazone, piroxicam, salicylic acid, salicylmides, and Vimovo® (a combination of naproxen and esomeprazole magnesium). In embodiments, the corticosteroid is selected from the group consisting of alpha-methyl dexamethasone, amcinafel, amcinafide, beclomethasone dipropionate, beclomethasone dipropionate, betamethasone and the balance of its esters, betamethasone benzoate, betamethasone dipropionate, betamethasone valerate, beta-methyl betamethasone, bethamethasone, chloroprednisone, clescinolone, clobetasol valerate, clocortelone, cortisone, cortodoxone, desonide, desoxymethasone, dexamethasone, dichlorisone, diflorasone diacetate, diflucortolone valerate, difluorosone diacetate, difluprednate, fluadrenolone, flucetonide, fluclorolone acetonide, flucloronide, flucortine butylester, fludrocortisone, flumethasone pivalate, flunisolide, fluocinonide, fluocortolone, fluoromethalone, fluosinolone acetonide, fluperolone, fluprednidene (fluprednylidene) acetate, fluprednisolone, fluradrenolone acetonide, flurandrenolone, halcinonide, hydrocortisone, hydrocortisone acetate, hydrocortisone butyrate, hydroxyltriamcinolone, medrysone, meprednisone, methylprednisolone, paramethasone, prednisolone, prednisone, triamcinolone, and triamcinolone acetonide.

In embodiments, the pharmaceutical composition further comprises both an immunosuppressive agent and an anti-inflammatory drug.

In embodiments, the chimeric proteins (and/or an anti-inflammatory drug and/or an immunosuppressive agent) disclosed herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

In embodiments, the compositions disclosed herein are in the form of a pharmaceutically acceptable salt.

Further, any chimeric protein (and/or an anti-inflammatory drug and/or an immunosuppressive agent) disclosed herein can be administered to a subject as a component of pharmaceutical composition, that comprises a pharmaceutically acceptable carrier or vehicle. Such pharmaceutical compositions can optionally comprise a suitable amount of a pharmaceutically acceptable excipient so as to provide the form for proper administration. Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In embodiments, the pharmaceutically acceptable excipients are sterile when administered to a subject. Water is a useful excipient when any agent disclosed herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent disclosed herein, if desired, can also comprise minor amounts of wetting or emulsifying agents, or pH buffering agents.

In embodiments, the chimeric proteins disclosed herein are resuspended in a saline buffer (including, without limitation TBS, PBS, and the like).

In embodiments, the chimeric proteins may by conjugated and/or fused with another agent to extend half-life or otherwise improve pharmacodynamic and pharmacokinetic properties. In embodiments, the chimeric proteins may be fused or conjugated with one or more of PEG, XTEN (e.g., as rPEG), polysialic acid (POLYXEN), albumin (e.g., human serum albumin or HAS), elastin-like protein (ELP), PAS, HAP, GLK, CTP, transferrin, and the like. In embodiments, each of the individual chimeric proteins is fused to one or more of the agents described in BioDrugs (2015) 29:215-239, the entire contents of which are hereby incorporated by reference.

The present invention includes the disclosed chimeric protein (and/or an anti-inflammatory drug and/or an immunosuppressive agent) in various formulations of pharmaceutical composition. Any chimeric protein (and/or an anti-inflammatory drug and/or an immunosuppressive agent) disclosed herein can take the form of solutions, suspensions, emulsion, drops, tablets, pills, pellets, capsules, capsules containing liquids, powders, sustained-release formulations, suppositories, emulsions, aerosols, sprays, suspensions, or any other form suitable for use. DNA or RNA constructs encoding the protein sequences may also be used. In embodiments, the composition is in the form of a capsule (see, e.g., U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

Where necessary, the pharmaceutical compositions comprising the chimeric protein (and/or an anti-inflammatory drug and/or an immunosuppressive agent) can also include a solubilizing agent. Also, the agents can be delivered with a suitable vehicle or delivery device as known in the art. Combination therapies outlined herein can be co-delivered in a single delivery vehicle or delivery device. Pharmaceutical compositions for administration can optionally include a local anesthetic such as, for example, lignocaine to lessen pain at the site of the injection.

The pharmaceutical compositions comprising the chimeric protein (and/or an anti-inflammatory drug and/or an immunosuppressive agent) of the present invention may conveniently be presented in unit dosage forms and may be prepared by any of the methods well known in the art of pharmacy. Such methods generally include the step of bringing therapeutic agents into association with a carrier, which constitutes one or more accessory ingredients. Typically, the pharmaceutical compositions are prepared by uniformly and intimately bringing therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art)

In embodiments, any chimeric protein (and/or an anti-inflammatory drug and/or an immunosuppressive agent) disclosed herein is formulated in accordance with routine procedures as a pharmaceutical composition adapted for a mode of administration disclosed herein.

### Methods of Treatment

An aspect of the present invention is a method of treating an autoimmune disease comprising administering to a subject in need thereof an effective amount of a pharmaceutical composition comprising a therapeutically effective amount of the chimeric protein of any of the herein disclosed aspects or embodiments.

In embodiments, the chimeric protein used in the method of treating an autoimmune disease has a general structure of: N terminus-(a)-(b)-(c)-C terminus in which (a) is a first domain comprising a portion of the extracellular domain of a transmembrane protein, a secreted protein, or a membrane-anchored extracellular protein, (c) is a second domain comprising a portion of the extracellular domain of a transmembrane protein, a secreted protein, or a membrane-anchored extracellular protein, and (b) is a linker adjoining the first domain and the second domain. In this aspect, either or both of the first domain and the second domain decreases self-directed immune system activity when bound to its ligand/receptor. In embodiments, the method pushes the T helper axis towards a less inflammatory state and/or decreases inflammation.

In embodiments, the portion of the first domain is capable of binding the native ligand/receptor for the transmembrane protein, the secreted protein, or the membrane-anchored extracellular protein.

In embodiments, the portion of the second domain is capable of binding the native ligand/receptor for the transmembrane protein, the secreted protein, or the membrane-anchored extracellular protein.

In embodiments, the first domain comprises substantially the entire extracellular domain of the transmembrane protein, substantially the entire secreted protein, or substantially the entire membrane-anchored extracellular protein.

In embodiments, the second domain comprises substantially the entire extracellular domain of the transmembrane protein, substantially the entire secreted protein, or substantially the entire membrane-anchored extracellular protein.

In embodiments, the binding of the portion of the first domain to its ligand/receptor decreases immune system activity by activating an immune inhibitory signal or inhibiting an immune activating signal.

In embodiments, the binding of the portion of the second domain to its ligand/receptor decreases immune system activity by activating an immune inhibitory signal or by inhibiting an immune activating signal.

In embodiments, the portion of the first domain comprises a transmembrane protein, a secreted protein, or a membrane-anchored extracellular protein selected from B7H3, B7H4, BTNL2, CTLA4, CSF3, ICOSL, ILDR2, PD-L1, TNFR2, IL-6R, MadCAM, integrin α4β7, and VSIG4.

In embodiments, the portion of the second domain comprises a transmembrane protein, a secreted protein, or a membrane-anchored extracellular protein selected from BTNL2, IL2, PD-L1, SEMA3E, IL-35, CCL25, TGFβ, and TL1A.

In embodiments, the first domain comprises a portion of VSIG4 and the second domain comprises a portion of IL2.

In embodiments, the first domain comprises a portion of CTLA4 and the second domain comprises a portion of IL2, e.g., the portion of IL2 comprises one or more mutations relative to a corresponding portion of wild-type IL2 wherein the one or more mutations provide preferential binding to a high-affinity IL2 receptor that is expressed by regulatory T cells.

In embodiments, the first domain comprises a portion of CTLA4 and the second domain comprises a portion of PD-L1.

In embodiments, the first domain comprises a portion of B7H3 and the second domain comprises a portion of PD-L1.

In embodiments, the first domain comprises a portion of B7H4 and the second domain comprises a portion of PD-L1.

In embodiments, the first domain comprises a portion of ICOSL and the second domain comprises a portion of PD-L1.

In embodiments, the first domain comprises a portion of ILDR2 and the second domain comprises a portion of PD-L1.

In embodiments, the first domain comprises a portion BTNL2 of and the second domain comprises a portion of PD-L1 or the first domain comprises a portion of PD-L1 and the second domain comprises a portion of BTNL2.

In embodiments, the first domain comprises a portion of CSF3 and the second domain comprises a portion of TL1A.

In embodiments, the first domain comprises a portion of CTLA4 and the second domain comprises a portion of TL1A.

In embodiments, the first domain comprises a portion of CTLA4 and the second domain comprises a portion of SEMA3E.

In embodiments, the first domain comprises a portion of TNFR2 and the second domain comprises an extracellular domain of a transmembrane protein selected from GITRL and TL1A.

In embodiments, the first domain comprises a portion of CTLA4 and the second domain comprises an extracellular domain of a transmembrane protein selected from GITRL and TL1A.

In embodiments, the first domain comprises an extracellular domain of IL-6R and the second domain comprises a portion of IL-35. In embodiments, the first domain comprises an extracellular domain of IL-6ST and/or IL-6R and/or the second domain comprises a portion of EBI3 and/or IL-12A. In embodiments, the chimeric protein is a heterodimer.

In embodiments, the first domain comprises an extracellular domain of MadCAM and the second domain comprises a portion of CCL25.

In embodiments, the first domain comprises an extracellular domain of TNFR2 and the second domain comprises a portion of TGFβ.

In embodiments, the first domain comprises an extracellular domain of integrin α4β7 and the second domain comprises a portion of IL-35. In embodiments, the first domain comprises an extracellular domain of integrin α4 and/or integrin β7, and/or the second domain comprises a portion of EBI3 and/or IL-12A. In embodiments, the chimeric protein is a heterodimer.

In embodiments, the chimeric protein is capable of contemporaneously binding a TNFR2 ligand and a ligand/receptor of a Type II transmembrane protein selected from BTNL2C-type lectin domain (CLEC) family members, GITRL TL1A, IL-10, or TGF-beta.

In embodiments, the first domain comprises a portion of TNFR2 and the second domain comprises an extracellular domain of a transmembrane protein selected from 4-1BBL, APRIL, BAFF, BTNL2, CD28, CD30L, CD40L, CD70, C-type lectin domain (CLEC) family members, FasL, GITRL, LIGHT, LTa, LTa1b2, NKG2A, NKG2C, NKG2D, OX40L, RANKL, TL1A, TNFa, and TRAIL; in embodiments, the second domain comprises an extracellular domain of GITRL or TL1A. In embodiments, the CLEC family member is selected from AlCL/CLEC-2B, ASGR1/ASGPR1, ASGR2, C1q R1/CD93, CD161, CD161/NK1.1, CD23/Fc epsilon RII, CD302/CLEC13A, CD72, CD94, Chondrolectin, CLEC-1, CLEC10A/CD301, CLEC12B, CLEC14A, CLEC16A, CLEC17A, CLEC18A, CLEC18B, CLEC18C, CLEC-2/CLEC1B, CLEC-2A, CLEC3A, CLEC3B/Tetranectin, CLEC4B2/mDCAR1, CLEC4D/CLECSF8, CLEC4E, CLEC4F/CLECSF13, CLEC9a, CLECL1/DCAL-1, CL-K1/COLEC11, CL-L1/COLEC10, CL-P1/COLEC12, DCAR/CLEC4B, DCIR/CLEC4A, DCIR4/CLEC4A1, DC-SIGN/CD209, DC-SIGN+FDC-SIGNR, DC-SIGNR/CD299, DC-SIGNR/CD299, DEC-205/CD205, Dectin-1/CLEC7A, Dectin-2/CLEC6A, DLEC/CLEC4C/BDCA-2, Ficolin-1, Ficolin-2, Ficolin-3, Klre-1, KLRG2, Langerin/CD207, Layilin, LOX-1/OLR1, LSECtin/CLEC4G, MBL, MBL-1, MBL-2, MDL-1/CLEC5A, MGL1/2 (CD301a/b), MGL1/CD301a, MGL2/CD301b, MGL2/CD301b, MICL/CLEC12A, MMR/CD206, Mrc2, NKG2A/CD159a, NKG2A/NKG2B Isoform 2, NKG2C/CD159c, NKG2D/CD314, NKG2E, NKG2H, NKp80/KLRF1, OCIL/CLEC2d, OCILRP2/CLEC2i, PLA2R1, QBRICK/FREM1, Reg1, Reg1A, Reg1B, Reg2, Reg3A, Reg3B, Reg3D, Reg3G, Reg4, SCGF/CLEC11a, SFTPA1, SIGNR1/CD209b, SIGNR3/CD209d, SIGNR4/CD209e, SIGNR7/CD209g, and SP-D.

In embodiments, the first domain comprises a portion of CTLA4 and the second domain comprises an extracellular domain of a transmembrane protein selected from 4-1BBL, APRIL, BAFF, BTNL2, CD28, CD30L, CD40L, CD70, C-type lectin domain (CLEC) family members, FasL, GITRL, LIGHT, LTa, LTa1b2, NKG2A, NKG2C, NKG2D, OX40L, RANKL, TL1A, TNFa, and TRAIL; in embodiments, the second domain comprises an extracellular domain of GITRL or TL1A. In embodiments, the CLEC family member is selected from AlCL/CLEC-2B, ASGR1/ASGPR1, ASGR2, C1q R1/CD93, CD161, CD161/NK1.1, CD23/Fc epsilon RII, CD302/CLEC13A, CD72, CD94, Chondrolectin, CLEC-1, CLEC10A/CD301, CLEC12B, CLEC14A, CLEC16A, CLEC17A, CLEC18A, CLEC18B, CLEC18C, CLEC-2/CLEC1B, CLEC-2A, CLEC3A, CLEC3B/Tetranectin, CLEC4B2/mDCAR1, CLEC4D/CLECSF8, CLEC4E, CLEC4F/CLECSF13, CLEC9a, CLECL1/DCAL-1, CL-K1/COLEC11, CL-L1/COLEC10, CL-P1/COLEC12, DCAR/CLEC4B, DCIR/CLEC4A, DCIR4/CLEC4A1, DC-SIGN/CD209, DC-SIGN+FDC-SIGNR, DC-SIGNR/CD299, DC-SIGNR/CD299, DEC-205/CD205, Dectin-1/CLEC7A, Dectin-2/CLEC6A, DLEC/CLEC4C/BDCA-2, Ficolin-1, Ficolin-2, Ficolin-3, Klre-1, KLRG2, Langerin/CD207, Layilin, LOX-1/OLR1, LSECtin/CLEC4G, MBL, MBL-1, MBL-2, MDL-1/CLEC5A, MGL1/2 (CD301a/b), MGL1/CD301a, MGL2/CD301b, MGL2/CD301b, MICL/CLEC12A, MMR/CD206, Mrc2, NKG2A/CD159a, NKG2A/NKG2B Isoform 2, NKG2C/CD159c, NKG2D/CD314, NKG2E, NKG2H, NKp80/KLRF1, OCIL/CLEC2d, OCILRP2/CLEC2i, PLA2R1, QBRICK/FREM1, Reg1, Reg1A, Reg1B, Reg2, Reg3A, Reg3B, Reg3D, Reg3G, Reg4, SCGF/CLEC11a, SFTPA1, SIGNR1/CD209b, SIGNR3/CD209d, SIGNR4/CD209e, SIGNR7/CD209g, and SP-D.

In embodiments, the binding of either or both of the first domain and the second domains to its ligand/receptor occurs with slow off rates (Koff), which provides a long interaction of a receptor and its ligand. In embodiments, the long interaction provides a prolonged decrease in immune system activity which comprises sustained activation of an immune inhibitory signal and/or a sustained inhibition of an immune activating signal. In embodiments, the sustained activation of the immune inhibitory signal and/or the sustained inhibition of the immune activating signal reduces the activity or proliferation of an immune cell, e.g., a B cell or a T cell. In embodiments, the sustained activation of the immune inhibitory signal and/or the sustained inhibition of the immune activating signal decreases synthesis and/or decreases release of a pro-inflammatory cytokine. In embodiments, the sustained activation of the immune inhibitory signal and/or the sustained inhibition of the immune activating signal increases synthesis and/or increases release of an anti-inflammatory cytokine. In embodiments, the sustained activation of the immune inhibitory signal and/or the sustained inhibition of the immune activating signal decreases antibody production and/or decreases secretion of antibodies by a B cell, e.g., an antibody that recognizes a self-antigen. In embodiments, the sustained activation of the immune inhibitory signal and/or the sustained inhibition of the immune activating signal decreases the activity of and/or decreases the number of T cytotoxic cells, e.g., which recognize a self-antigen and kill cells presenting or expressing the self-antigen. In embodiments, the sustained activation of the immune inhibitory signal and/or the sustained inhibition of the immune activating signal increases the activity and/or increases the number of T regulatory cells.

In embodiments, the linker is a polypeptide selected from a flexible amino acid sequence, an IgG hinge region, and an antibody sequence.

In embodiments, the linker comprises at least one cysteine residue capable of forming a disulfide bond and/or comprises a hinge-CH2-CH3 Fc domain, e.g., a hinge-CH2-CH3 Fc domain is derived from IgG (e.g., IgG1, IgG2, IgG3, and IgG4), IgA (e.g., IgA1 and IgA2), IgD, or IgE. In embodiments, the IgG is IgG4, e.g., a human IgG4. In embodiments, the IgG is IgG1, e.g., a human IgG1. In embodiments, the linker comprises an amino acid sequence that is at least 95% identical to the amino acid sequence of SEQ ID NO: 1, SEQ ID NO: 2, or SEQ ID NO: 3.

In embodiments, the chimeric protein used in the method of treating an autoimmune disease comprises: (a) a first domain comprising a portion of VSIG4 that is capable of binding a VSIG4 ligand/receptor, (b) a second domain comprising a portion of IL2 that is capable of binding an IL2 receptor, and (c) a linker linking the first domain and the second domain and comprising a hinge-CH2-CH3 Fc domain.

In embodiments, the chimeric protein used in the method of treating an autoimmune disease comprises: (a) a first domain comprising a portion of CTLA4 that is capable of binding a CTLA4 ligand/receptor, (b) a second domain comprising a portion of IL2 that is capable of binding an IL2 receptor, and (c) a linker linking the first domain and the second domain and comprising a hinge-CH2-CH3 Fc domain. In embodiments, the IL2 receptor is a high-affinity IL2 receptor that is expressed by regulatory T cells, e.g., the portion of IL2 comprises one or more mutations relative to a corresponding portion of wild-type IL2 which provides preferential binding to the high-affinity IL2 receptor that is expressed by regulatory T cells.

In embodiments, the chimeric protein used in the method of treating an autoimmune disease comprises: (a) a first domain comprising a portion of CTLA4 that is capable of binding a CTLA4 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in the method of treating an autoimmune disease comprises: (a) a first domain comprising a portion of B7H3 that is capable of binding a B7H3 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in the method of treating an autoimmune disease comprises: (a) a first domain comprising a portion of B7H4 that is capable of binding a B7H4 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in the method of treating an autoimmune disease comprises: (a) a first domain comprising a portion of ICOSL that is capable of binding an ICOSL ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in the method of treating an autoimmune disease comprises: (a) a first domain comprising a portion of ILDR2 that is capable of binding an ILDR2 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in the method of treating an autoimmune disease comprises: (a) a first domain comprising a portion of BTNL2 that is capable of binding a BTNL2 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in the method of treating an autoimmune disease comprises: (a) a first domain comprising a portion of PD-L1 that is capable of binding PD-1, (b) a second domain comprising a portion of BTNL2 that is capable of binding a BTNL2 ligand/receptor, and (c) a linker linking the first domain and the second domain and comprising a hinge-CH2-CH3 Fc domain.

In embodiments, the chimeric protein used in the method of treating an autoimmune disease comprises: (a) a first domain comprising a portion of CSF3 that is capable of binding a CSF3 ligand/receptor, (b) a second domain comprising a portion of TL1A that is capable of binding a TL1A ligand/receptor, and (c) a linker linking the first domain and the second domain and comprising a hinge-CH2-CH3 Fc domain.

In embodiments, the chimeric protein used in the method of treating an autoimmune disease comprises: (a) a first domain comprising a portion of CTLA4 that is capable of binding a CTLA4 ligand/receptor, (b) a second domain comprising a portion of TL1A that is capable of binding a TL1A ligand/receptor, and (c) a linker linking the first domain and the second domain and comprising a hinge-CH2-CH3 Fc domain.

In embodiments, the chimeric protein used in the method of treating an autoimmune disease comprises: (a) a first domain comprising a portion of CTLA4 that is capable of binding a CTLA4 ligand/receptor, (b) a second domain comprising a portion of SEMA3E that is capable of binding a SEMA3E ligand/receptor, and (c) a linker linking the first domain and the second domain and comprising a hinge-CH2-CH3 Fc domain.

In embodiments, the chimeric protein used in method of treating an autoimmune disease of immune inhibitory cells comprises: (a) a first domain comprising a portion of MadCAM that is capable of binding a MadCAM ligand/receptor, (b) a second domain comprising a portion of CCL25 that is capable of binding a CCL25 ligand/receptor, and (c) a linker linking the first domain and the second domain and comprising a hinge-CH2-CH3 Fc domain.

In embodiments, the chimeric protein used in method of treating an autoimmune disease of immune inhibitory cells comprises: (a) a first domain comprising a portion of TNFR2 that is capable of binding a TNFR2 ligand/receptor, (b) a second domain comprising a portion of TGFβ that is capable of binding a TGFβ ligand/receptor, and (c) a linker linking the first domain and the second domain and comprising a hinge-CH2-CH3 Fc domain.

In embodiments, the chimeric protein used in method of treating an autoimmune disease of immune inhibitory cells comprises: (a) a first domain comprising an extracellular domain of IL-6R that is capable of binding a IL-6R ligand/receptor, (b) a second domain comprising a portion of IL-35 that is capable of binding a IL-35 ligand/receptor, and (c) a linker linking the first domain and the second domain and comprising a hinge-CH2-CH3 Fc domain. In embodiments, the first domain comprises an extracellular domain of IL-6ST and/or IL-6R. In embodiments, the second domain comprises a portion of EBI3 and/or IL-12A. In embodiments, the chimeric protein is heterodimeric.

In embodiments, the chimeric protein used in method of treating an autoimmune disease of immune inhibitory cells comprises: (a) a first domain comprising an extracellular domain of integrin α4β7 that is capable of binding an integrin α4β7 ligand/receptor, (b) a second domain comprising a portion of IL-35 that is capable of binding a IL-35 ligand/receptor, and (c) a linker linking the first domain and the second domain and comprising a hinge-CH2-CH3 Fc domain. In embodiments, the first domain comprises an extracellular domain of integrin α4 and/or integrin β7. In embodiments, the second domain comprises a portion of EBI3 and/or IL-12A. In embodiments, the chimeric protein is heterodimeric.

An aspect of the present invention is a method of increasing the number and/or activity of immune inhibitory cells comprising administering to a subject in need thereof an effective amount of a pharmaceutical composition comprising a therapeutically effective amount of the chimeric protein of any of the herein disclosed aspects or embodiments, wherein the number and/or activity of immune inhibitor cells increases compared to the number and/or activity of immune inhibitory cells prior to the administration of the pharmaceutical composition. An aspect of the present invention is a method of increasing the number and/or activity of immune inhibitory cells comprising administering to a subject in need thereof an effective amount of a pharmaceutical composition comprising a therapeutically effective amount of the chimeric protein of any of the herein disclosed aspects or embodiments, wherein the number and/or activity of immune inhibitor cells increases compared to the number and/or activity of immune inhibitory cells without the administration of the pharmaceutical composition. In embodiments, the immune inhibitory cells are selected from regulatory T cells (Tregs), myeloid-derived suppressor cells (MDSCs), tumor associated neutrophils (TANs), M2 macrophages, tumor associated macrophages (TAMs), or subsets thereof. In embodiments, the immune inhibitory cells are regulatory T cells (Tregs). In embodiments, the immune inhibitory cells are regulatory T cells (Tregs) selected from natural Tregs (nTregs), IL-10-producing type 1 Tregs (Tr1 cells), TGF-β-producing Th3 cells, CD8+ Tregs, NKT regulatory cells and pTreg. In embodiments, the increase in the number and/or activity of immune inhibitory cells is due to the activation and/or increase of inhibitory immune cells or progenitors thereof. In embodiments, the increase in the number and/or activity of immune inhibitory cells is due to the suppression and/or reduction of stimulatory immune cells. In embodiment, the increase in the number and/or activity of immune inhibitory cells is associated with the suppression and/or reduction of stimulatory immune cells. In alternative embodiments, the increase in the number and/or activity of immune inhibitory cells is not associated with the suppression and/or reduction of stimulatory immune cells. In embodiments, the chimeric protein used in the method of increasing the number and/or activity of immune inhibitory cells has a general structure of: N terminus-(a)-(b)-(c)-C terminus in which (a) is a first domain comprising a portion of the extracellular domain of a transmembrane protein, a secreted protein, or a membrane-anchored extracellular protein, (c) is a second domain comprising a portion of the extracellular domain of a transmembrane protein, a secreted protein, or a membrane-anchored extracellular protein, and (b) is a linker adjoining the first domain and the second domain. In this aspect, either or both of the first domain and the second domain decreases self-directed immune system activity when bound to its ligand/receptor. In embodiments, the portion of the first domain comprises a transmembrane protein, a secreted protein, or a membrane-anchored extracellular protein selected from B7H3, B7H4, BTNL2, CTLA4, CSF3, ICOSL, ILDR2, PD-L1, TNFR2, IL-6R, MadCAM, integrin α4β7, and VSIG4. In embodiments, the portion of the second domain comprises a transmembrane protein, a secreted protein, or a membrane-anchored extracellular protein selected from BTNL2, IL2, PD-L1, SEMA3E, IL-35, CCL25, TGFβ, and TL1A. In embodiments, the method pushes the T helper axis towards a less inflammatory state and/or decreases inflammation.

An aspect of the present invention is a method of increasing the number and/or activity of immune inhibitory cells in a subject in need thereof, the method comprising: contacting lymphocytes from the subject with an effective amount of a pharmaceutical composition comprising a therapeutically effective amount of the chimeric protein of any of the herein disclosed aspects or embodiments, wherein the number and/or activity of immune inhibitor cells increases compared to the number and/or activity of immune inhibitory cells prior to the administration of the pharmaceutical composition. An aspect of the present invention is a method of increasing the number and/or activity of immune inhibitory cells in a subject in need thereof, the method comprising: contacting lymphocytes from the subject with an effective amount of a pharmaceutical composition comprising a therapeutically effective amount of the chimeric protein of any of the herein disclosed aspects or embodiments, wherein the number and/or activity of immune inhibitor cells increases compared to the number and/or activity of immune inhibitory cells without the administration of the pharmaceutical composition. In embodiments, the lymphocytes from the subject are contacted with the pharmaceutical composition in vivo. In embodiments, the lymphocytes from the subject are contacted with the pharmaceutical composition ex vivo. In embodiments, the method further comprises extracting peripheral blood mononuclear cells comprising lymphocytes from the subject. In embodiments, the method further comprises culturing and/or expanding the lymphocytes ex vivo.

In embodiments, the method further comprises administering the cultured and/or expanded lymphocytes to the subject. In embodiments, the method pushes the T helper axis towards a less inflammatory state and/or decreases inflammation.

In embodiments, the immune inhibitory cells are selected from regulatory T cells (Tregs), myeloid-derived suppressor cells (MDSCs), tumor associated neutrophils (TANs), M2 macrophages, tumor associated macrophages (TAMs), or subsets thereof. In embodiments, the immune inhibitory cells are regulatory T cells (Tregs). In embodiments, the immune inhibitory cells are regulatory T cells (Tregs) selected from natural Tregs (nTregs), IL-10-producing type 1 Tregs (Tr1 cells), TGF-β-producing Th3 cells, CD8+ Tregs, NKT regulatory cells and pTreg. In embodiments, the increase in the number and/or activity of immune inhibitory cells is due to the activation and/or increase of inhibitory immune cells or progenitors thereof. In embodiments, the increase in the number and/or activity of immune inhibitory cells is due to the suppression and/or reduction of stimulatory immune cells. In embodiment, the increase in the number and/or activity of immune inhibitory cells is associated with the suppression and/or reduction of stimulatory immune cells. In alternative embodiments, the increase in the number and/or activity of immune inhibitory cells is not associated with the suppression and/or reduction of stimulatory immune cells. In embodiments, the chimeric protein used in the method of increasing the number and/or activity of immune inhibitory cells has a general structure of: N terminus-(a)-(b)-(c)-C terminus in which (a) is a first domain comprising a portion of the extracellular domain of a transmembrane protein, a secreted protein, or a membrane-anchored extracellular protein, (c) is a second domain comprising a portion of the extracellular domain of a transmembrane protein, a secreted protein, or a membrane-anchored extracellular protein, and (b) is a linker adjoining the first domain and the second domain. In this aspect, either or both of the first domain and the second domain decreases self-directed immune system activity when bound to its ligand/receptor. In embodiments, the portion of the first domain comprises a transmembrane protein, a secreted protein, or a membrane-anchored extracellular protein selected from B7H3, B7H4, BTNL2, CTLA4, CSF3, ICOSL, ILDR2, PD-L1, TNFR2, IL-6R, MadCAM, integrin α4β7, and VSIG4. In embodiments, the portion of the second domain comprises a transmembrane protein, a secreted protein, or a membrane-anchored extracellular protein selected from BTNL2, IL2, PD-L1, SEMA3E, IL-35, CCL25, TGFβ, and TL1A.

In embodiments, the number and/or activity of immune inhibitory cells is increased by at least 5%, or at least 10%, or at least 20%, or at least 30%, or at least 40%, or at least 50%, or at least 60%, or at least 70%, or at least 80%, or at least 90%, or at least 100%, or at least 150%, or at least 200%, or at least 250%, or at least 300%, or at least 350%, or at least 400%, or at least 450%, or at least 500%. In embodiments, the number and/or activity of CD8+ T cells is decreased by at least 5%, or at least 10%, or at least 20%, or at least 30%, or at least 40%, or at least 50%, or at least 60%, or at least 70%, or at least 80%, or at least 90%.

In embodiments, the chimeric protein used in method of increasing the number and/or activity of immune inhibitory cells comprises: (a) a first domain comprising a portion of VSIG4 that is capable of binding a VSIG4 ligand/receptor, (b) a second domain comprising a portion of IL2 that is capable of binding an IL2 receptor, and (c) a linker linking the first domain and the second domain and comprising a hinge-CH2-CH3 Fc domain.

In embodiments, the chimeric protein used in method of increasing the number and/or activity of immune inhibitory cells comprises: (a) a first domain comprising a portion of CTLA4 that is capable of binding a CTLA4 ligand/receptor, (b) a second domain comprising a portion of IL2 that is capable of binding an IL2 receptor, and (c) a linker linking the first domain and the second domain and comprising a hinge-CH2-CH3 Fc domain. In embodiments, the IL2 receptor is a high-affinity IL2 receptor that is expressed by regulatory T cells, e.g., the portion of IL2 comprises one or more mutations relative to a corresponding portion of wild-type IL2 which provides preferential binding to the high-affinity IL2 receptor that is expressed by regulatory T cells.

In embodiments, the chimeric protein used in method of increasing the number and/or activity of immune inhibitory cells comprises: (a) a first domain comprising a portion of CTLA4 that is capable of binding a CTLA4 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of increasing the number and/or activity of immune inhibitory cells comprises: (a) a first domain comprising a portion of B7H3 that is capable of binding a B7H3 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of increasing the number and/or activity of immune inhibitory cells comprises: (a) a first domain comprising a portion of B7H4 that is capable of binding a B7H4 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of increasing the number and/or activity of immune inhibitory cells comprises: (a) a first domain comprising a portion of ICOSL that is capable of binding an ICOSL ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of increasing the number and/or activity of immune inhibitory cells comprises: (a) a first domain comprising a portion of ILDR2 that is capable of binding an ILDR2 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of increasing the number and/or activity of immune inhibitory cells comprises: (a) a first domain comprising a portion of BTNL2 that is capable of binding a BTNL2 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of increasing the number and/or activity of immune inhibitory cells comprises: (a) a first domain comprising a portion of PD-L1 that is capable of binding PD-1, (b) a second domain comprising a portion of BTNL2 that is capable of binding a BTNL2 ligand/receptor, and (c) a linker linking the first domain and the second domain and comprising a hinge-CH2-CH3 Fc domain.

In embodiments, the chimeric protein used in method of increasing the number and/or activity of immune inhibitory cells comprises: (a) a first domain comprising a portion of CSF3 that is capable of binding a CSF3 ligand/receptor, (b) a second domain comprising a portion of TL1A that is capable of binding a TL1A ligand/receptor, and (c) a linker linking the first domain and the second domain and comprising a hinge-CH2-CH3 Fc domain.

In embodiments, the chimeric protein used in method of increasing the number and/or activity of immune inhibitory cells comprises: (a) a first domain comprising a portion of CTLA4 that is capable of binding a CTLA4 ligand/receptor, (b) a second domain comprising a portion of TL1A that is capable of binding a TL1A ligand/receptor, and (c) a linker linking the first domain and the second domain and comprising a hinge-CH2-CH3 Fc domain.

In embodiments, the chimeric protein used in method of increasing the number and/or activity of immune inhibitory cells comprises: (a) a first domain comprising a portion of CTLA4 that is capable of binding a CTLA4 ligand/receptor, (b) a second domain comprising a portion of SEMA3E that is capable of binding a SEMA3E ligand/receptor, and (c) a linker linking the first domain and the second domain and comprising a hinge-CH2-CH3 Fc domain.

In embodiments, the chimeric protein used in method of increasing the number and/or activity of immune inhibitory cells comprises: (a) a first domain comprising a portion of MadCAM that is capable of binding a MadCAM ligand/receptor, (b) a second domain comprising a portion of CCL25 that is capable of binding a CCL25 ligand/receptor, and (c) a linker linking the first domain and the second domain and comprising a hinge-CH2-CH3 Fc domain.

In embodiments, the chimeric protein used in method of increasing the number and/or activity of immune inhibitory cells comprises: (a) a first domain comprising a portion of TNFR2 that is capable of binding a TNFR2 ligand/receptor, (b) a second domain comprising a portion of TGFβ that is capable of binding a TGFβ ligand/receptor, and (c) a linker linking the first domain and the second domain and comprising a hinge-CH2-CH3 Fc domain.

In embodiments, the chimeric protein used in method of increasing the number and/or activity of immune inhibitory cells comprises: (a) a first domain comprising an extracellular domain of IL-6R that is capable of binding a IL-6R ligand/receptor, (b) a second domain comprising a portion of IL-35 that is capable of binding a IL-35 ligand/receptor, and (c) a linker linking the first domain and the second domain and comprising a hinge-CH2-CH3 Fc domain. In embodiments, the first domain comprises an extracellular domain of IL-6ST and/or IL-6R. In embodiments, the second domain comprises a portion of EBI3 and/or IL-12A. In embodiments, the chimeric protein is heterodimeric.

In embodiments, the chimeric protein used in method of increasing the number and/or activity of immune inhibitory cells comprises: (a) a first domain comprising an extracellular domain of integrin α4β7 that is capable of binding an integrin α4β7 ligand/receptor, (b) a second domain comprising a portion of IL-35 that is capable of binding a IL-35 ligand/receptor, and (c) a linker linking the first domain and the second domain and comprising a hinge-CH2-CH3 Fc domain. In embodiments, the first domain comprises an extracellular domain of integrin α4 and/or integrin β7. In embodiments, the second domain comprises a portion of EBI3 and/or IL-12A. In embodiments, the chimeric protein is heterodimeric. In embodiments, the amount of the immunostimulatory cytokine is decreased by at least 5%, or at least 10%, or at least 20%, or at least 30%, or at least 40%, or at least 50%, or at least 60%, or at least 70%, or at least 80%, or at least 90%.

An aspect of the present invention is a method of reducing the amount a cytokine comprising administering to a subject in need thereof an effective amount of a pharmaceutical composition comprising a therapeutically effective amount of the chimeric protein of any of the herein disclosed aspects or embodiments, wherein the amount a cytokine is decreased compared to the amount a cytokine prior to the administration of the pharmaceutical composition. An aspect of the present invention is a method of reducing the amount a cytokine comprising administering to a subject in need thereof an effective amount of a pharmaceutical composition comprising a therapeutically effective amount of the chimeric protein of any of the herein disclosed aspects or embodiments, wherein the amount a cytokine is decreased compared to the amount a cytokine without the administration of the pharmaceutical composition. In embodiments, the cytokine is selected from an immunostimulatory cytokine. In embodiments, the immunostimulatory cytokine is selected from IL-2, IL-4, IL-6, IL-7, IL-12, IL-17, IL-21, IL-22, IL-35, IFNα, IFNγ, GM-CSF and TNFα. In embodiments, the amount of the immunostimulatory cytokine is decreased by at least 5%, or at least 10%, or at least 20%, or at least 30%, or at least 40%, or at least 50%, or at least 60%, or at least 70%, or at least 80%, or at least 90%. In embodiments, the method pushes the T helper axis towards a less inflammatory state and/or decreases inflammation.

An aspect of the present invention is a method of reducing the amount a cytokine in a subject in need thereof, the method comprising: contacting lymphocytes from the subject with an effective amount of a pharmaceutical composition comprising a therapeutically effective amount of the chimeric protein of any of the herein disclosed aspects or embodiments, wherein the amount a cytokine is decreased compared to the amount a cytokine prior to the administration of the pharmaceutical composition. An aspect of the present invention is a method of reducing the amount a cytokine in a subject in need thereof, the method comprising: contacting lymphocytes from the subject with an effective amount of a pharmaceutical composition comprising a therapeutically effective amount of the chimeric protein of any of the herein disclosed aspects or embodiments, wherein the amount a cytokine is decreased compared to the amount a cytokine without the administration of the pharmaceutical composition. In embodiments, the lymphocytes from the subject are contacted with the pharmaceutical composition in vivo. In embodiments, the lymphocytes from the subject are contacted with the pharmaceutical composition ex vivo. In embodiments, the method further comprises extracting peripheral blood mononuclear cells comprising lymphocytes from the subject. In embodiments, the method further comprises culturing and/or expanding the lymphocytes ex vivo. In embodiments, the method further comprises administering the cultured and/or expanded lymphocytes to the subject. In embodiments, the cytokine is selected from an immunostimulatory cytokine. In embodiments, the immunostimulatory cytokine is selected from IL-2, IL-4, IL-6, IL-7, IL-12, IL-17, IL-21, IL-22, IFNα, IFNγ, GM-CSF and INFα. In embodiments, the cytokine decreases because of binding of the chimeric protein to the cytokine. In embodiments, the method pushes the T helper axis towards a less inflammatory state and/or decreases inflammation.

In embodiments, the chimeric protein used in the method of reducing the amount a cytokine has a general structure of: N terminus-(a)-(b)-(c)-C terminus in which (a) is a first domain comprising a portion of the extracellular domain of a transmembrane protein, a secreted protein, or a membrane-anchored extracellular protein, (c) is a second domain comprising a portion of the extracellular domain of a transmembrane protein, a secreted protein, or a membrane-anchored extracellular protein, and (b) is a linker adjoining the first domain and the second domain. In this aspect, either or both of the first domain and the second domain decreases self-directed immune system activity when bound to its ligand/receptor. In embodiments, the portion of the first domain comprises a transmembrane protein, a secreted protein, or a membrane-anchored extracellular protein selected from B7H3, B7H4, BTNL2, CTLA4, CSF3, ICOSL, ILDR2, PD-L1, TNFR2, IL-6R, MadCAM, integrin α4β7, and VSIG4. In embodiments, the portion of the second domain comprises a transmembrane protein, a secreted protein, or a membrane-anchored extracellular protein selected from BTNL2, IL2, PD-L1, SEMA3E, IL-35, CCL25, TGFβ, and TL1A. In embodiments, the cytokine is decreased because of binding of the chimeric protein to the soluble ligand. In embodiments, the immunostimulatory cytokine is decreased because of binding of the chimeric protein to the soluble ligand.

In embodiments, the chimeric protein used in method of reducing the amount a cytokine comprises: (a) a first domain comprising a portion of VSIG4 that is capable of binding a VSIG4 ligand/receptor, (b) a second domain comprising a portion of IL2 that is capable of binding an IL2 receptor, and (c) a linker linking the first domain and the second domain and comprising a hinge-CH2-CH3 Fc domain.

In embodiments, the chimeric protein used in method of reducing the amount a cytokine comprises: (a) a first domain comprising a portion of CTLA4 that is capable of binding a CTLA4 ligand/receptor, (b) a second domain comprising a portion of IL2 that is capable of binding an IL2 receptor, and (c) a linker linking the first domain and the second domain and comprising a hinge-CH2-CH3 Fc domain. In embodiments, the IL2 receptor is a high-affinity IL2 receptor that is expressed by regulatory T cells, e.g., the portion of IL2 comprises one or more mutations relative to a corresponding portion of wild-type IL2 which provides preferential binding to the high-affinity IL2 receptor that is expressed by regulatory T cells.

In embodiments, the chimeric protein used in method of reducing the amount a cytokine comprises: (a) a first domain comprising a portion of CTLA4 that is capable of binding a CTLA4 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of reducing the amount a cytokine comprises: (a) a first domain comprising a portion of B7H3 that is capable of binding a B7H3 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of reducing the amount a cytokine comprises: (a) a first domain comprising a portion of B7H4 that is capable of binding a B7H4 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of reducing the amount a cytokine comprises: (a) a first domain comprising a portion of ICOSL that is capable of binding an ICOSL ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of reducing the amount a cytokine comprises: (a) a first domain comprising a portion of ILDR2 that is capable of binding an ILDR2 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of reducing the amount a cytokine comprises: (a) a first domain comprising a portion of BTNL2 that is capable of binding a BTNL2 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of reducing the amount a cytokine comprises: (a) a first domain comprising a portion of PD-L1 that is capable of binding PD-1, (b) a second domain comprising a portion of BTNL2 that is capable of binding a BTNL2 ligand/receptor, and (c) a linker linking the first domain and the second domain and comprising a hinge-CH2-CH3 Fc domain.

In embodiments, the chimeric protein used in method of reducing the amount a cytokine comprises: (a) a first domain comprising a portion of CSF3 that is capable of binding a CSF3 ligand/receptor, (b) a second domain comprising a portion of TL1A that is capable of binding a TL1A ligand/receptor, and (c) a linker linking the first domain and the second domain and comprising a hinge-CH2-CH3 Fc domain.

In embodiments, the chimeric protein used in method of reducing the amount a cytokine comprises: (a) a first domain comprising a portion of CTLA4 that is capable of binding a CTLA4 ligand/receptor, (b) a second domain comprising a portion of TL1A that is capable of binding a TL1A ligand/receptor, and (c) a linker linking the first domain and the second domain and comprising a hinge-CH2-CH3 Fc domain.

In embodiments, the chimeric protein used in method of reducing the amount a cytokine comprises: (a) a first domain comprising a portion of CTLA4 that is capable of binding a CTLA4 ligand/receptor, (b) a second domain comprising a portion of SEMA3E that is capable of binding a SEMA3E ligand/receptor, and (c) a linker linking the first domain and the second domain and comprising a hinge-CH2-CH3 Fc domain.

In embodiments, the chimeric protein used in method of reducing the amount a cytokine comprises: (a) a first domain comprising a portion of MadCAM that is capable of binding a MadCAM ligand/receptor, (b) a second domain comprising a portion of CCL25 that is capable of binding a CCL25 ligand/receptor, and (c) a linker linking the first domain and the second domain and comprising a hinge-CH2-CH3 Fc domain.

In embodiments, the chimeric protein used in method of reducing the amount a cytokine comprises: (a) a first domain comprising a portion of TNFR2 that is capable of binding a TNFR2 ligand/receptor, (b) a second domain comprising a portion of TGFβ that is capable of binding a TGFβ ligand/receptor, and (c) a linker linking the first domain and the second domain and comprising a hinge-CH2-CH3 Fc domain.

In embodiments, the chimeric protein used in method of reducing the amount a cytokine comprises: (a) a first domain comprising an extracellular domain of IL-6R that is capable of binding a IL-6R ligand/receptor, (b) a second domain comprising a portion of IL-35 that is capable of binding a IL-35 ligand/receptor, and (c) a linker linking the first domain and the second domain and comprising a hinge-CH2-CH3 Fc domain. In embodiments, the first domain comprises an extracellular domain of IL-6ST and/or IL-6R. In embodiments, the second domain comprises a portion of EBI3 and/or IL-12A. In embodiments, the chimeric protein is heterodimeric.

In embodiments, the chimeric protein used in method of reducing the amount a cytokine comprises: (a) a first domain comprising an extracellular domain of integrin α4β7 that is capable of binding an integrin α4β7 ligand/receptor, (b) a second domain comprising a portion of IL-35 that is capable of binding a IL-35 ligand/receptor, and (c) a linker linking the first domain and the second domain and comprising a hinge-CH2-CH3 Fc domain. In embodiments, the first domain comprises an extracellular domain of integrin α4 and/or integrin β7. In embodiments, the second domain comprises a portion of EBI3 and/or IL-12A. In embodiments, the chimeric protein is heterodimeric.

An aspect of the present invention is a method of suppressing the activity of CD8+ T cells comprising administering to a subject in need thereof an effective amount of a pharmaceutical composition comprising a therapeutically effective amount of the chimeric protein of any of the herein disclosed aspects or embodiments, wherein the amount and/or activity of CD8+ T cells is decreased compared to the amount and/or activity of CD8+ T cells prior to the administration of the pharmaceutical composition. An aspect of the present invention is a method of suppressing the activity of CD8+ T cells comprising administering to a subject in need thereof an effective amount of a pharmaceutical composition comprising a therapeutically effective amount of the chimeric protein of any of the herein disclosed aspects or embodiments, wherein the amount and/or activity of CD8+ T cells is decreased compared to the amount and/or activity of CD8+ T cells without the administration of the pharmaceutical composition. In embodiments, the amount and/or activity of CD8+ T cells is decreased by at least 5%, or at least 10%, or at least 20%, or at least 30%, or at least 40%, or at least 50%, or at least 60%, or at least 70%, or at least 80%, or at least 90%. In embodiments, the method pushes the T helper axis towards a less inflammatory state and/or decreases inflammation.

An aspect of the present invention is a method of suppressing the activity of CD8+ T cells in a subject in need thereof, the method comprising: contacting lymphocytes from the subject with an effective amount of a pharmaceutical composition comprising a therapeutically effective amount of the chimeric protein of any of the herein disclosed aspects or embodiments, wherein the amount and/or activity of CD8+ T cells is decreased compared to the amount and/or activity of CD8+ T cells prior to the administration of the pharmaceutical composition. An aspect of the present invention is a method of suppressing the activity of CD8+ T cells in a subject in need thereof, the method comprising: contacting lymphocytes from the subject with an effective amount of a pharmaceutical composition comprising a therapeutically effective amount of the chimeric protein of any of the herein disclosed aspects or embodiments, wherein the amount and/or activity of CD8+ T cells is decreased compared to the amount and/or activity of CD8+ T cells without the administration of the pharmaceutical composition. In embodiments, the lymphocytes from the subject are contacted with the pharmaceutical composition in vivo. In embodiments, the lymphocytes from the subject are contacted with the pharmaceutical composition ex vivo. In embodiments, the method further comprises extracting peripheral blood mononuclear cells comprising lymphocytes from the subject. In embodiments, the method further comprises culturing and/or expanding the lymphocytes ex vivo. In embodiments, the method further comprises administering the cultured and/or expanded lymphocytes to the subject. In embodiments, the method pushes the T helper axis towards a less inflammatory state and/or decreases inflammation.

In embodiments, the CD8+ T cells are suppressed because of reduction in the amount and/or activity of an immunostimulatory cytokine. In embodiments, the immunostimulatory cytokine is selected from IL-2, IL-4, IL-6, IL-7, IL-12, IL-17, IL-21, IL-22, IFNα, IFNγ, GM-CSF and INFα. In embodiments, the amount of the immunostimulatory cytokine is decreased by at least 5%, or at least 10%, or at least 20%, or at least 30%, or at least 40%, or at least 50%, or at least 60%, or at least 70%, or at least 80%, or at least 90%.

In embodiments, the chimeric protein used in the method of suppressing the activity of CD8+ T cells has a general structure of: N terminus-(a)-(b)-(c)-C terminus in which (a) is a first domain comprising a portion of the extracellular domain of a transmembrane protein, a secreted protein, or a membrane-anchored extracellular protein, (c) is a second domain comprising a portion of the extracellular domain of a transmembrane protein, a secreted protein, or a membrane-anchored extracellular protein, and (b) is a linker adjoining the first domain and the second domain. In this aspect, either or both of the first domain and the second domain decreases self-directed immune system activity when bound to its ligand/receptor. In embodiments, the portion of the first domain comprises a transmembrane protein, a secreted protein, or a membrane-anchored extracellular protein selected from B7H3, B7H4, BTNL2, CTLA4, CSF3, ICOSL, ILDR2, PD-L1, TNFR2, IL-6R, MadCAM, integrin α4β7, and VSIG4. In embodiments, the portion of the second domain comprises a transmembrane protein, a secreted protein, or a membrane-anchored extracellular protein selected from BTNL2, IL2, PD-L1, SEMA3E, IL-35, CCL25, TGFβ, and TL1A. In embodiments, the cytokine is decreased because of binding of the chimeric protein to the soluble ligand. In embodiments, the immunostimulatory cytokine is decreased because of binding of the chimeric protein to the soluble ligand.

In embodiments, the chimeric protein used in method of suppressing the activity of CD8+ T cells comprises: (a) a first domain comprising a portion of VSIG4 that is capable of binding a VSIG4 ligand/receptor, (b) a second domain comprising a portion of IL2 that is capable of binding an IL2 receptor, and (c) a linker linking the first domain and the second domain and comprising a hinge-CH2-CH3 Fc domain.

In embodiments, the chimeric protein used in method of suppressing the activity of CD8+ T cells comprises: (a) a first domain comprising a portion of CTLA4 that is capable of binding a CTLA4 ligand/receptor, (b) a second domain comprising a portion of IL2 that is capable of binding an IL2 receptor, and (c) a linker linking the first domain and the second domain and comprising a hinge-CH2-CH3 Fc domain. In embodiments, the IL2 receptor is a high-affinity IL2 receptor that is expressed by regulatory T cells, e.g., the portion of IL2 comprises one or more mutations relative to a corresponding portion of wild-type IL2 which provides preferential binding to the high-affinity IL2 receptor that is expressed by regulatory T cells.

In embodiments, the chimeric protein used in method of suppressing the activity of CD8+ T cells comprises: (a) a first domain comprising a portion of CTLA4 that is capable of binding a CTLA4 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of suppressing the activity of CD8+ T cells comprises: (a) a first domain comprising a portion of B7H3 that is capable of binding a B7H3 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of suppressing the activity of CD8+ T cells comprises: (a) a first domain comprising a portion of B7H4 that is capable of binding a B7H4 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of suppressing the activity of CD8+ T cells comprises: (a) a first domain comprising a portion of ICOSL that is capable of binding an ICOSL ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of suppressing the activity of CD8+ T cells comprises: (a) a first domain comprising a portion of ILDR2 that is capable of binding an ILDR2 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of suppressing the activity of CD8+ T cells comprises: (a) a first domain comprising a portion of BTNL2 that is capable of binding a BTNL2 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of suppressing the activity of CD8+ T cells comprises: (a) a first domain comprising a portion of PD-L1 that is capable of binding PD-1, (b) a second domain comprising a portion of BTNL2 that is capable of binding a BTNL2 ligand/receptor, and (c) a linker linking the first domain and the second domain and comprising a hinge-CH2-CH3 Fc domain.

In embodiments, the chimeric protein used in method of suppressing the activity of CD8+ T cells comprises: (a) a first domain comprising a portion of CSF3 that is capable of binding a CSF3 ligand/receptor, (b) a second domain comprising a portion of TL1A that is capable of binding a TL1A ligand/receptor, and (c) a linker linking the first domain and the second domain and comprising a hinge-CH2-CH3 Fc domain.

In embodiments, the chimeric protein used in method of suppressing the activity of CD8+ T cells comprises: (a) a first domain comprising a portion of CTLA4 that is capable of binding a CTLA4 ligand/receptor, (b) a second domain comprising a portion of TL1A that is capable of binding a TL1A ligand/receptor, and (c) a linker linking the first domain and the second domain and comprising a hinge-CH2-CH3 Fc domain.

In embodiments, the chimeric protein used in method of suppressing the activity of CD8+ T cells comprises: (a) a first domain comprising a portion of CTLA4 that is capable of binding a CTLA4 ligand/receptor, (b) a second domain comprising a portion of SEMA3E that is capable of binding a SEMA3E ligand/receptor, and (c) a linker linking the first domain and the second domain and comprising a hinge-CH2-CH3 Fc domain.

In embodiments, the chimeric protein used in method of suppressing the activity of CD8+ T cells comprises: (a) a first domain comprising a portion of MadCAM that is capable of binding a MadCAM ligand/receptor, (b) a second domain comprising a portion of CCL25 that is capable of binding a CCL25 ligand/receptor, and (c) a linker linking the first domain and the second domain and comprising a hinge-CH2-CH3 Fc domain.

In embodiments, the chimeric protein used in method of suppressing the activity of CD8+ T cells comprises: (a) a first domain comprising a portion of TNFR2 that is capable of binding a TNFR2 ligand/receptor, (b) a second domain comprising a portion of TGFβ that is capable of binding a TGFβ ligand/receptor, and (c) a linker linking the first domain and the second domain and comprising a hinge-CH2-CH3 Fc domain.

In embodiments, the chimeric protein used in method of suppressing the activity of CD8+ T cells comprises: (a) a first domain comprising an extracellular domain of IL-6R that is capable of binding a IL-6R ligand/receptor, (b) a second domain comprising a portion of IL-35 that is capable of binding a IL-35 ligand/receptor, and (c) a linker linking the first domain and the second domain and comprising a hinge-CH2-CH3 Fc domain. In embodiments, the first domain comprises an extracellular domain of IL-6ST and/or IL-6R. In embodiments, the second domain comprises a portion of EBI3 and/or IL-12A. In embodiments, the chimeric protein is heterodimeric.

In embodiments, the chimeric protein used in method of suppressing the activity of CD8+ T cells comprises: (a) a first domain comprising an extracellular domain of integrin α4β7 that is capable of binding an integrin α4β7 ligand/receptor, (b) a second domain comprising a portion of IL-35 that is capable of binding a IL-35 ligand/receptor, and (c) a linker linking the first domain and the second domain and comprising a hinge-CH2-CH3 Fc domain. In embodiments, the first domain comprises an extracellular domain of integrin α4 and/or integrin β7. In embodiments, the second domain comprises a portion of EBI3 and/or IL-12A. In embodiments, the chimeric protein is heterodimeric.

An aspect of the present invention is a method of stimulating CD4+ cells to suppress the amount and/or activity of CD8+ T cells comprising administering to a subject in need thereof an effective amount of a pharmaceutical composition comprising a therapeutically effective amount of the chimeric protein of any of the herein disclosed aspects or embodiments, wherein the amount and/or activity of CD8+ T cells is decreased compared to the amount and/or activity of CD8+ T cells prior to the administration of the pharmaceutical composition. An aspect of the present invention is a method of stimulating CD4+ cells to suppress the amount and/or activity of CD8+ T cells comprising administering to a subject in need thereof an effective amount of a pharmaceutical composition comprising a therapeutically effective amount of the chimeric protein of any of the herein disclosed aspects or embodiments, wherein the amount and/or activity of CD8+ T cells is decreased compared to the amount and/or activity of CD8+ T cells without the administration of the pharmaceutical composition. In embodiments, the CD4+ cells are regulatory T cells (Tregs) selected from natural Tregs (nTregs), IL-10-producing type 1 Tregs (Tr1 cells), TGF-β-producing Th3 cells, CD8+ Tregs, NKT regulatory cells and pTreg. In embodiments, the amount and/or activity of CD8+ T cells is decreased by at least 5%, or at least 10%, or at least 20%, or at least 30%, or at least 40%, or at least 50%, or at least 60%, or at least 70%, or at least 80%, or at least 90%. In embodiments, the method pushes the T helper axis towards a less inflammatory state and/or decreases inflammation.

An aspect of the present invention is a method of stimulating CD4+ cells to suppress the amount and/or activity of CD8+ T cells in a subject in need thereof, the method comprising: contacting lymphocytes from the subject with an effective amount of a pharmaceutical composition comprising a therapeutically effective amount of the chimeric protein of any of the herein disclosed aspects or embodiments, wherein the amount and/or activity of CD8+ T cells is decreased compared to the amount and/or activity of CD8+ T cells prior to the administration of the pharmaceutical composition. An aspect of the present invention is a method of stimulating CD4+ cells to suppress the amount and/or activity of CD8+ T cells in a subject in need thereof, the method comprising: contacting lymphocytes from the subject with an effective amount of a pharmaceutical composition comprising a therapeutically effective amount of the chimeric protein of any of the herein disclosed aspects or embodiments, wherein the amount and/or activity of CD8+ T cells is decreased compared to the amount and/or activity of CD8+ T cells without the administration of the pharmaceutical composition. In embodiments, the lymphocytes from the subject are contacted with the pharmaceutical composition in vivo. In embodiments, the lymphocytes from the subject are contacted with the pharmaceutical composition ex vivo. In embodiments, the method further comprises extracting peripheral blood mononuclear cells comprising lymphocytes from the subject. In embodiments, the method further comprises culturing and/or expanding the lymphocytes ex vivo. In embodiments, the method further comprises administering the cultured and/or expanded lymphocytes to the subject. In embodiments, the cytokine is selected from an immunostimulatory cytokine. In embodiments, the immunostimulatory cytokine is selected from IL-2, IL-4, IL-6, IL-7, IL-12, IL-17, IL-21, IL-22, IFNα, IFNγ, GM-CSF and INFα. In embodiments, the cytokine decreases because of binding of the chimeric protein to the cytokine. In embodiments, the method pushes the T helper axis towards a less inflammatory state and/or decreases inflammation.

In embodiments, the CD8+ T cells are suppressed because of reduction in the amount and/or activity of an immunostimulatory cytokine. In embodiments, the immunostimulatory cytokine is selected from IL-2, IL-4, IL-6, IL-7, IL-12, IL-17, IL-21, IL-22, IFNα, IFNγ, GM-CSF and INFα. In embodiments, the amount of the immunostimulatory cytokine is decreased by at least 5%, or at least 10%, or at least 20%, or at least 30%, or at least 40%, or at least 50%, or at least 60%, or at least 70%, or at least 80%, or at least 90%.

In embodiments, the chimeric protein used in the method of stimulating CD4+ cells to suppress the amount and/or activity of CD8+ T cells has a general structure of: N terminus-(a)-(b)-(c)-C terminus in which (a) is a first domain comprising a portion of the extracellular domain of a transmembrane protein, a secreted protein, or a membrane-anchored extracellular protein, (c) is a second domain comprising a portion of the extracellular domain of a transmembrane protein, a secreted protein, or a membrane-anchored extracellular protein, and (b) is a linker adjoining the first domain and the second domain. In this aspect, either or both of the first domain and the second domain decreases self-directed immune system activity when bound to its ligand/receptor. In embodiments, the portion of the first domain comprises a transmembrane protein, a secreted protein, or a membrane-anchored extracellular protein selected from B7H3, B7H4, BTNL2, CTLA4, CSF3, ICOSL, ILDR2, PD-L1, TNFR2, IL-6R, MadCAM, integrin α4β7, and VSIG4. In embodiments, the portion of the second domain comprises a transmembrane protein, a secreted protein, or a membrane-anchored extracellular protein selected from BTNL2, IL2, PD-L1, SEMA3E, IL-35, CCL25, TGFβ, and TL1A. In embodiments, the cytokine is decreased because of binding of the chimeric protein to the soluble ligand. In embodiments, the immunostimulatory cytokine is decreased because of binding of the chimeric protein to the soluble ligand.

In embodiments, the chimeric protein used in method of stimulating CD4+ cells to suppress the amount and/or activity of CD8+ T cells comprises: (a) a first domain comprising a portion of VSIG4 that is capable of binding a VSIG4 ligand/receptor, (b) a second domain comprising a portion of IL2 that is capable of binding an IL2 receptor, and (c) a linker linking the first domain and the second domain and comprising a hinge-CH2-CH3 Fc domain.

In embodiments, the chimeric protein used in method of stimulating CD4+ cells to suppress the amount and/or activity of CD8+ T cells comprises: (a) a first domain comprising a portion of CTLA4 that is capable of binding a CTLA4 ligand/receptor, (b) a second domain comprising a portion of IL2 that is capable of binding an IL2 receptor, and (c) a linker linking the first domain and the second domain and comprising a hinge-CH2-CH3 Fc domain. In embodiments, the IL2 receptor is a high-affinity IL2 receptor that is expressed by regulatory T cells, e.g., the portion of IL2 comprises one or more mutations relative to a corresponding portion of wild-type IL2 which provides preferential binding to the high-affinity IL2 receptor that is expressed by regulatory T cells.

In embodiments, the chimeric protein used in method of stimulating CD4+ cells to suppress the amount and/or activity of CD8+ T cells comprises: (a) a first domain comprising a portion of CTLA4 that is capable of binding a CTLA4 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of stimulating CD4+ cells to suppress the amount and/or activity of CD8+ T cells comprises: (a) a first domain comprising a portion of B7H3 that is capable of binding a B7H3 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of stimulating CD4+ cells to suppress the amount and/or activity of CD8+ T cells comprises: (a) a first domain comprising a portion of B7H4 that is capable of binding a B7H4 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of stimulating CD4+ cells to suppress the amount and/or activity of CD8+ T cells comprises: (a) a first domain comprising a portion of ICOSL that is capable of binding an ICOSL ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of stimulating CD4+ cells to suppress the amount and/or activity of CD8+ T cells comprises: (a) a first domain comprising a portion of ILDR2 that is capable of binding an ILDR2 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of stimulating CD4+ cells to suppress the amount and/or activity of CD8+ T cells comprises: (a) a first domain comprising a portion of BTNL2 that is capable of binding a BTNL2 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of stimulating CD4+ cells to suppress the amount and/or activity of CD8+ T cells comprises: (a) a first domain comprising a portion of PD-L1 that is capable of binding PD-1, (b) a second domain comprising a portion of BTNL2 that is capable of binding a BTNL2 ligand/receptor, and (c) a linker linking the first domain and the second domain and comprising a hinge-CH2-CH3 Fc domain.

In embodiments, the chimeric protein used in method of stimulating CD4+ cells to suppress the amount and/or activity of CD8+ T cells comprises: (a) a first domain comprising a portion of CSF3 that is capable of binding a CSF3 ligand/receptor, (b) a second domain comprising a portion of TL1A that is capable of binding a TL1A ligand/receptor, and (c) a linker linking the first domain and the second domain and comprising a hinge-CH2-CH3 Fc domain.

In embodiments, the chimeric protein used in method of stimulating CD4+ cells to suppress the amount and/or activity of CD8+ T cells comprises: (a) a first domain comprising a portion of CTLA4 that is capable of binding a CTLA4 ligand/receptor, (b) a second domain comprising a portion of TL1A that is capable of binding a TL1A ligand/receptor, and (c) a linker linking the first domain and the second domain and comprising a hinge-CH2-CH3 Fc domain.

In embodiments, the chimeric protein used in method of stimulating CD4+ cells to suppress the amount and/or activity of CD8+ T cells comprises: (a) a first domain comprising a portion of CTLA4 that is capable of binding a CTLA4 ligand/receptor, (b) a second domain comprising a portion of SEMA3E that is capable of binding a SEMA3E ligand/receptor, and (c) a linker linking the first domain and the second domain and comprising a hinge-CH2-CH3 Fc domain.

In embodiments, the chimeric protein used in method of stimulating CD4+ cells to suppress the amount and/or activity of CD8+ T cells comprises: (a) a first domain comprising a portion of MadCAM that is capable of binding a MadCAM ligand/receptor, (b) a second domain comprising a portion of CCL25 that is capable of binding a CCL25 ligand/receptor, and (c) a linker linking the first domain and the second domain and comprising a hinge-CH2-CH3 Fc domain.

In embodiments, the chimeric protein used in method of stimulating CD4+ cells to suppress the amount and/or activity of CD8+ T cells comprises: (a) a first domain comprising a portion of TNFR2 that is capable of binding a TNFR2 ligand/receptor, (b) a second domain comprising a portion of TGFβ that is capable of binding a TGFβ ligand/receptor, and (c) a linker linking the first domain and the second domain and comprising a hinge-CH2-CH3 Fc domain.

In embodiments, the chimeric protein used in method of stimulating CD4+ cells to suppress the amount and/or activity of CD8+ T cells comprises: (a) a first domain comprising an extracellular domain of IL-6R that is capable of binding a IL-6R ligand/receptor, (b) a second domain comprising a portion of IL-35 that is capable of binding a IL-35 ligand/receptor, and (c) a linker linking the first domain and the second domain and comprising a hinge-CH2-CH3 Fc domain. In embodiments, the first domain comprises an extracellular domain of IL-6ST and/or IL-6R. In embodiments, the second domain comprises a portion of EBI3 and/or IL-12A. In embodiments, the chimeric protein is heterodimeric.

In embodiments, the chimeric protein used in method of stimulating CD4+ cells to suppress the amount and/or activity of CD8+ T cells comprises: (a) a first domain comprising an extracellular domain of integrin α4β7 that is capable of binding an integrin α4β7 ligand/receptor, (b) a second domain comprising a portion of IL-35 that is capable of binding a IL-35 ligand/receptor, and (c) a linker linking the first domain and the second domain and comprising a hinge-CH2-CH3 Fc domain. In embodiments, the first domain comprises an extracellular domain of integrin α4 and/or integrin β7. In embodiments, the second domain comprises a portion of EBI3 and/or IL-12A. In embodiments, the chimeric protein is heterodimeric.

An aspect of the present invention is a method of increasing the amount and/or immunosuppressive activity of CD4+ cells comprising administering to a subject in need thereof an effective amount of a pharmaceutical composition comprising a therapeutically effective amount of the chimeric protein of any of the herein disclosed aspects or embodiments, wherein the amount and/or immunosuppressive activity of CD4+ T cells is increased compared to the amount and/or immunosuppressive activity of CD4+ T cells prior to the administration of the pharmaceutical composition. An aspect of the present invention is a method of increasing the amount and/or immunosuppressive activity of CD4+ cells comprising administering to a subject in need thereof an effective amount of a pharmaceutical composition comprising a therapeutically effective amount of the chimeric protein of any of the herein disclosed aspects or embodiments, wherein the amount and/or immunosuppressive activity of CD4+ T cells is increased compared to the amount and/or immunosuppressive activity of CD4+ T cells without the administration of the pharmaceutical composition. In embodiments, the amount and/or activity of CD4+ T cells is increased by at least 5%, or at least 10%, or at least 20%, or at least 30%, or at least 40%, or at least 50%, or at least 60%, or at least 70%, or at least 80%, or at least 90%%, or at least 100%, or at least 150%, or at least 200%, or at least 250%, or at least 300%, or at least 350%%, or at least 400%, or at least 550%, or at least 500%. In embodiments, the method pushes the T helper axis towards a less inflammatory state and/or decreases inflammation.

An aspect of the present invention is a method of increasing the amount and/or immunosuppressive activity of CD4+ cells in a subject in need thereof, the method comprising: contacting lymphocytes from the subject with an effective amount of a pharmaceutical composition comprising a therapeutically effective amount of the chimeric protein of any of the herein disclosed aspects or embodiments, wherein the amount and/or immunosuppressive activity of CD4+ T cells is increased compared to the amount and/or immunosuppressive activity of CD4+ T cells prior to the administration of the pharmaceutical composition. An aspect of the present invention is a method of increasing the amount and/or immunosuppressive activity of CD4+ cells in a subject in need thereof, the method comprising: contacting lymphocytes from the subject with an effective amount of a pharmaceutical composition comprising a therapeutically effective amount of the chimeric protein of any of the herein disclosed aspects or embodiments, wherein the amount and/or immunosuppressive activity of CD4+ T cells is increased compared to the amount and/or immunosuppressive activity of CD4+ T cells without the administration of the pharmaceutical composition. In embodiments, the lymphocytes from the subject are contacted with the pharmaceutical composition in vivo. In embodiments, the lymphocytes from the subject are contacted with the pharmaceutical composition ex vivo. In embodiments, the method further comprises extracting peripheral blood mononuclear cells comprising lymphocytes from the subject. In embodiments, the method further comprises culturing and/or expanding the lymphocytes ex vivo. In embodiments, the method further comprises administering the cultured and/or expanded lymphocytes to the subject. In embodiments, the method pushes the T helper axis towards a less inflammatory state and/or decreases inflammation.

In embodiments, the amount and/or immunosuppressive activity of CD4+ cells is increased because of reduction in the amount and/or activity of an immunostimulatory cytokine. In embodiments, the immunostimulatory cytokine is selected from IL-2, IL-4, IL-6, IL-7, IL-10, IL-12, IL-17, IL-21, IL-22, IFNα, IFNγ, GM-CSF and INFα. In embodiments, the amount of the immunostimulatory cytokine is decreased by at least 5%, or at least 10%, or at least 20%, or at least 30%, or at least 40%, or at least 50%, or at least 60%, or at least 70%, or at least 80%, or at least 90%. In embodiments, the amount and/or immunosuppressive activity of CD4+ cells is increased because of increase in the amount and/or activity of an immunosuppressive cytokine. In embodiments, immunosuppressive cytokine is selected from the IL-1Ra, IL-4, IL-10, IL-11, IL-13, TGFβ, IL-33, IL-35, and IL-37. In embodiments, the amount and/or activity of immunosuppressive cytokine is increased by at least 5%, or at least 10%, or at least 20%, or at least 30%, or at least 40%, or at least 50%, or at least 60%, or at least 70%, or at least 80%, or at least 90%%, or at least 100%, or at least 150%, or at least 200%, or at least 250%, or at least 300%, or at least 350%%, or at least 400%, or at least 550%, or at least 500%.

In embodiments, the chimeric protein used in the method of increasing the amount and/or immunosuppressive activity of CD4+ cells has a general structure of: N terminus-(a)-(b)-(c)-C terminus in which (a) is a first domain comprising a portion of the extracellular domain of a transmembrane protein, a secreted protein, or a membrane-anchored extracellular protein, (c) is a second domain comprising a portion of the extracellular domain of a transmembrane protein, a secreted protein, or a membrane-anchored extracellular protein, and (b) is a linker adjoining the first domain and the second domain. In this aspect, either or both of the first domain and the second domain decreases self-directed immune system activity when bound to its ligand/receptor. In embodiments, the portion of the first domain comprises a transmembrane protein, a secreted protein, or a membrane-anchored extracellular protein selected from B7H3, B7H4, BTNL2, CTLA4, CSF3, ICOSL, ILDR2, PD-L1, TNFR2, IL-6R, MadCAM, integrin α4β7, and VSIG4. In embodiments, the portion of the second domain comprises a transmembrane protein, a secreted protein, or a membrane-anchored extracellular protein selected from BTNL2, IL2, PD-L1, SEMA3E, IL-35, CCL25, TGFβ, and TL1A. In embodiments, the cytokine is decreased because of binding of the chimeric protein to the soluble ligand. In embodiments, the immunostimulatory cytokine is decreased because of binding of the chimeric protein to the soluble ligand.

In embodiments, the chimeric protein used in method of increasing the amount and/or immunosuppressive activity of CD4+ cells comprises: (a) a first domain comprising a portion of VSIG4 that is capable of binding a VSIG4 ligand/receptor, (b) a second domain comprising a portion of IL2 that is capable of binding an IL2 receptor, and (c) a linker linking the first domain and the second domain and comprising a hinge-CH2-CH3 Fc domain.

In embodiments, the chimeric protein used in method of increasing the amount and/or immunosuppressive activity of CD4+ cells comprises: (a) a first domain comprising a portion of CTLA4 that is capable of binding a CTLA4 ligand/receptor, (b) a second domain comprising a portion of IL2 that is capable of binding an IL2 receptor, and (c) a linker linking the first domain and the second domain and comprising a hinge-CH2-CH3 Fc domain. In embodiments, the IL2 receptor is a high-affinity IL2 receptor that is expressed by regulatory T cells, e.g., the portion of IL2 comprises one or more mutations relative to a corresponding portion of wild-type IL2 which provides preferential binding to the high-affinity IL2 receptor that is expressed by regulatory T cells.

In embodiments, the chimeric protein used in method of increasing the amount and/or immunosuppressive activity of CD4+ cells comprises: (a) a first domain comprising a portion of CTLA4 that is capable of binding a CTLA4 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of increasing the amount and/or immunosuppressive activity of CD4+ cells comprises: (a) a first domain comprising a portion of B7H3 that is capable of binding a B7H3 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of increasing the amount and/or immunosuppressive activity of CD4+ cells comprises: (a) a first domain comprising a portion of B7H4 that is capable of binding a B7H4 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of increasing the amount and/or immunosuppressive activity of CD4+ cells comprises: (a) a first domain comprising a portion of ICOSL that is capable of binding an ICOSL ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of increasing the amount and/or immunosuppressive activity of CD4+ cells comprises: (a) a first domain comprising a portion of ILDR2 that is capable of binding an ILDR2 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of increasing the amount and/or immunosuppressive activity of CD4+ cells comprises: (a) a first domain comprising a portion of BTNL2 that is capable of binding a BTNL2 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of increasing the amount and/or immunosuppressive activity of CD4+ cells comprises: (a) a first domain comprising a portion of PD-L1 that is capable of binding PD-1, (b) a second domain comprising a portion of BTNL2 that is capable of binding a BTNL2 ligand/receptor, and (c) a linker linking the first domain and the second domain and comprising a hinge-CH2-CH3 Fc domain.

In embodiments, the chimeric protein used in method of increasing the amount and/or immunosuppressive activity of CD4+ cells comprises: (a) a first domain comprising a portion of CSF3 that is capable of binding a CSF3 ligand/receptor, (b) a second domain comprising a portion of TL1A that is capable of binding a TL1A ligand/receptor, and (c) a linker linking the first domain and the second domain and comprising a hinge-CH2-CH3 Fc domain.

In embodiments, the chimeric protein used in method of increasing the amount and/or immunosuppressive activity of CD4+ cells comprises: (a) a first domain comprising a portion of CTLA4 that is capable of binding a CTLA4 ligand/receptor, (b) a second domain comprising a portion of TL1A that is capable of binding a TL1A ligand/receptor, and (c) a linker linking the first domain and the second domain and comprising a hinge-CH2-CH3 Fc domain.

In embodiments, the chimeric protein used in method of increasing the amount and/or immunosuppressive activity of CD4+ cells comprises: (a) a first domain comprising a portion of CTLA4 that is capable of binding a CTLA4 ligand/receptor, (b) a second domain comprising a portion of SEMA3E that is capable of binding a SEMA3E ligand/receptor, and (c) a linker linking the first domain and the second domain and comprising a hinge-CH2-CH3 Fc domain.

In embodiments, the chimeric protein used in method of increasing the amount and/or immunosuppressive activity of CD4+ cells comprises: (a) a first domain comprising a portion of MadCAM that is capable of binding a MadCAM ligand/receptor, (b) a second domain comprising a portion of CCL25 that is capable of binding a CCL25 ligand/receptor, and (c) a linker linking the first domain and the second domain and comprising a hinge-CH2-CH3 Fc domain.

In embodiments, the chimeric protein used in method of increasing the amount and/or immunosuppressive activity of CD4+ cells comprises: (a) a first domain comprising a portion of TNFR2 that is capable of binding a TNFR2 ligand/receptor, (b) a second domain comprising a portion of TGFβ that is capable of binding a TGFβ ligand/receptor, and (c) a linker linking the first domain and the second domain and comprising a hinge-CH2-CH3 Fc domain.

In embodiments, the chimeric protein used in method of increasing the amount and/or immunosuppressive activity of CD4+ cells comprises: (a) a first domain comprising an extracellular domain of IL-6R that is capable of binding a IL-6R ligand/receptor, (b) a second domain comprising a portion of IL-35 that is capable of binding a IL-35 ligand/receptor, and (c) a linker linking the first domain and the second domain and comprising a hinge-CH2-CH3 Fc domain. In embodiments, the first domain comprises an extracellular domain of IL-6ST and/or IL-6R. In embodiments, the second domain comprises a portion of EBI3 and/or IL-12A. In embodiments, the chimeric protein is heterodimeric.

In embodiments, the chimeric protein used in method of increasing the amount and/or immunosuppressive activity of CD4+ cells comprises: (a) a first domain comprising an extracellular domain of integrin α4β7 that is capable of binding an integrin α4β7 ligand/receptor, (b) a second domain comprising a portion of IL-35 that is capable of binding a IL-35 ligand/receptor, and (c) a linker linking the first domain and the second domain and comprising a hinge-CH2-CH3 Fc domain. In embodiments, the first domain comprises an extracellular domain of integrin α4 and/or integrin β7. In embodiments, the second domain comprises a portion of EBI3 and/or IL-12A. In embodiments, the chimeric protein is heterodimeric.

An aspect of the present invention is a method of treating inflammatory bowel disease (IBD) comprising administering to a subject in need thereof an effective amount of a pharmaceutical composition comprising a therapeutically effective amount of the chimeric protein of any of the herein disclosed aspects or embodiments. In embodiments, the inflammatory bowel disease (IBD), is selected from Crohn's disease (CD), and ulcerative colitis (UC), collagenous colitis, lymphocytic colitis, ischemic colitis, diversion colitis, Behcet's disease, and indeterminate colitis. In embodiments, the method pushes the T helper axis towards a less inflammatory state and/or decreases inflammation.

An aspect of the present invention is a method of treating inflammatory bowel disease in a subject in need thereof, the method comprising: contacting lymphocytes from the subject with an effective amount of a pharmaceutical composition comprising a therapeutically effective amount of the chimeric protein of any of the herein disclosed aspects or embodiments. In embodiments, the lymphocytes from the subject are contacted with the pharmaceutical composition in vivo. In embodiments, the lymphocytes from the subject are contacted with the pharmaceutical composition ex vivo. In embodiments, the method further comprises extracting peripheral blood mononuclear cells comprising lymphocytes from the subject. In embodiments, the method further comprises culturing and/or expanding the lymphocytes ex vivo. In embodiments, the method further comprises administering the cultured and/or expanded lymphocytes to the subject. In embodiments, the inflammatory bowel disease (IBD), is selected from Crohn's disease (CD), and ulcerative colitis (UC), collagenous colitis, lymphocytic colitis, ischemic colitis, diversion colitis, Behcet's disease, and indeterminate colitis. In embodiments, the method pushes the T helper axis towards a less inflammatory state and/or decreases inflammation.

In embodiments, the method increases amount and/or immunosuppressive activity of CD4+ cells. In embodiments, the CD4+ cells are regulatory T cells (Tregs) selected from natural Tregs (nTregs), IL-10-producing type 1 Tregs (Tr1 cells), TGF-β-producing Th3 cells, CD8+ Tregs, NKT regulatory cells and pTreg. In embodiments, the amount and/or activity of CD4+ cells is increased by at least 5%, or at least 10%, or at least 20%, or at least 30%, or at least 40%, or at least 50%, or at least 60%, or at least 70%, or at least 80%, or at least 90%%, or at least 100%, or at least 150%, or at least 200%, or at least 250%, or at least 300%, or at least 350%%, or at least 400%, or at least 550%, or at least 500%. In embodiments, the method increases the amount and/or activity of an immunosuppressive cytokine. In embodiments, immunosuppressive cytokine is selected from the IL-1Ra, IL-4, IL-10, IL-11, IL-13, TGFβ, IL-33, IL-35, and IL-37. In embodiments, the amount and/or activity of CD4+ cells is increased by at least 5%, or at least 10%, or at least 20%, or at least 30%, or at least 40%, or at least 50%, or at least 60%, or at least 70%, or at least 80%, or at least 90%%, or at least 100%, or at least 150%, or at least 200%, or at least 250%, or at least 300%, or at least 350%%, or at least 400%, or at least 550%, or at least 500%.

In embodiments, the method decreases the amount and/or activity of CD8+ cells. In embodiments, the amount and/or activity of CD8+ cells is decreased by at least 5%, or at least 10%, or at least 20%, or at least 30%, or at least 40%, or at least 50%, or at least 60%, or at least 70%, or at least 80%, or at least 90%. In embodiments, the method reduces the amount and/or activity of an immunostimulatory cytokine. In embodiments, the immunostimulatory cytokine is selected from IL-2, IL-4, IL-6, IL-7, IL-10, IL-12, IL-17, IL-21, IL-22, IFNa, IFNγ, GM-CSF and INFα. In embodiments, the amount of the immunostimulatory cytokine is decreased by at least 5%, or at least 10%, or at least 20%, or at least 30%, or at least 40%, or at least 50%, or at least 60%, or at least 70%, or at least 80%, or at least 90%.

In embodiments, the chimeric protein used in the method of treating inflammatory bowel disease has a general structure of: N terminus-(a)-(b)-(c)-C terminus in which (a) is a first domain comprising a portion of the extracellular domain of a transmembrane protein, a secreted protein, or a membrane-anchored extracellular protein, (c) is a second domain comprising a portion of the extracellular domain of a transmembrane protein, a secreted protein, or a membrane-anchored extracellular protein, and (b) is a linker adjoining the first domain and the second domain. In this aspect, either or both of the first domain and the second domain decreases self-directed immune system activity when bound to its ligand/receptor. In embodiments, the portion of the first domain comprises a transmembrane protein, a secreted protein, or a membrane-anchored extracellular protein selected from B7H3, B7H4, BTNL2, CTLA4, CSF3, ICOSL, ILDR2, PD-L1, TNFR2, IL-6R, MadCAM, integrin α4β7, and VSIG4. In embodiments, the portion of the second domain comprises a transmembrane protein, a secreted protein, or a membrane-anchored extracellular protein selected from BTNL2, IL2, PD-L1, SEMA3E, IL-35, CCL25, TGFβ, and TL1A. In embodiments, the cytokine is decreased because of binding of the chimeric protein to the soluble ligand. In embodiments, the immunostimulatory cytokine is decreased because of binding of the chimeric protein to the soluble ligand.

In embodiments, the chimeric protein used in method of treating inflammatory bowel disease comprises: (a) a first domain comprising a portion of VSIG4 that is capable of binding a VSIG4 ligand/receptor, (b) a second domain comprising a portion of IL2 that is capable of binding an IL2 receptor, and (c) a linker linking the first domain and the second domain and comprising a hinge-CH2-CH3 Fc domain.

In embodiments, the chimeric protein used in method of treating inflammatory bowel disease comprises: (a) a first domain comprising a portion of CTLA4 that is capable of binding a CTLA4 ligand/receptor, (b) a second domain comprising a portion of IL2 that is capable of binding an IL2 receptor, and (c) a linker linking the first domain and the second domain and comprising a hinge-CH2-CH3 Fc domain. In embodiments, the IL2 receptor is a high-affinity IL2 receptor that is expressed by regulatory T cells, e.g., the portion of IL2 comprises one or more mutations relative to a corresponding portion of wild-type IL2 which provides preferential binding to the high-affinity IL2 receptor that is expressed by regulatory T cells.

In embodiments, the chimeric protein used in method of treating inflammatory bowel disease comprises: (a) a first domain comprising a portion of CTLA4 that is capable of binding a CTLA4 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of treating inflammatory bowel disease comprises: (a) a first domain comprising a portion of B7H3 that is capable of binding a B7H3 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of treating inflammatory bowel disease comprises: (a) a first domain comprising a portion of B7H4 that is capable of binding a B7H4 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of treating inflammatory bowel disease comprises: (a) a first domain comprising a portion of ICOSL that is capable of binding an ICOSL ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of treating inflammatory bowel disease comprises: (a) a first domain comprising a portion of ILDR2 that is capable of binding an ILDR2 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of treating inflammatory bowel disease comprises: (a) a first domain comprising a portion of BTNL2 that is capable of binding a BTNL2 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of treating inflammatory bowel disease comprises: (a) a first domain comprising a portion of PD-L1 that is capable of binding PD-1, (b) a second domain comprising a portion of BTNL2 that is capable of binding a BTNL2 ligand/receptor, and (c) a linker linking the first domain and the second domain and comprising a hinge-CH2-CH3 Fc domain.

In embodiments, the chimeric protein used in method of treating inflammatory bowel disease comprises: (a) a first domain comprising a portion of CSF3 that is capable of binding a CSF3 ligand/receptor, (b) a second domain comprising a portion of TL1A that is capable of binding a TL1A ligand/receptor, and (c) a linker linking the first domain and the second domain and comprising a hinge-CH2-CH3 Fc domain.

In embodiments, the chimeric protein used in method of treating inflammatory bowel disease comprises: (a) a first domain comprising a portion of CTLA4 that is capable of binding a CTLA4 ligand/receptor, (b) a second domain comprising a portion of TL1A that is capable of binding a TL1A ligand/receptor, and (c) a linker linking the first domain and the second domain and comprising a hinge-CH2-CH3 Fc domain.

In embodiments, the chimeric protein used in method of treating inflammatory bowel disease comprises: (a) a first domain comprising a portion of CTLA4 that is capable of binding a CTLA4 ligand/receptor, (b) a second domain comprising a portion of SEMA3E that is capable of binding a SEMA3E ligand/receptor, and (c) a linker linking the first domain and the second domain and comprising a hinge-CH2-CH3 Fc domain.

In embodiments, the chimeric protein used in method of treating inflammatory bowel disease comprises: (a) a first domain comprising a portion of MadCAM that is capable of binding a MadCAM ligand/receptor, (b) a second domain comprising a portion of CCL25 that is capable of binding a CCL25 ligand/receptor, and (c) a linker linking the first domain and the second domain and comprising a hinge-CH2-CH3 Fc domain.

In embodiments, the chimeric protein used in method of treating inflammatory bowel disease comprises: (a) a first domain comprising a portion of TNFR2 that is capable of binding a TNFR2 ligand/receptor, (b) a second domain comprising a portion of TGFβ that is capable of binding a TGFβ ligand/receptor, and (c) a linker linking the first domain and the second domain and comprising a hinge-CH2-CH3 Fc domain.

In embodiments, the chimeric protein used in method of treating inflammatory bowel disease comprises: (a) a first domain comprising an extracellular domain of IL-6R that is capable of binding a IL-6R ligand/receptor, (b) a second domain comprising a portion of IL-35 that is capable of binding a IL-35 ligand/receptor, and (c) a linker linking the first domain and the second domain and comprising a hinge-CH2-CH3 Fc domain. In embodiments, the first domain comprises an extracellular domain of IL-6ST and/or IL-6R. In embodiments, the second domain comprises a portion of EBI3 and/or IL-12A. In embodiments, the chimeric protein is heterodimeric.

In embodiments, the chimeric protein used in method of treating inflammatory bowel disease comprises: (a) a first domain comprising an extracellular domain of integrin α4β7 that is capable of binding an integrin α4β7 ligand/receptor, (b) a second domain comprising a portion of IL-35 that is capable of binding a IL-35 ligand/receptor, and (c) a linker linking the first domain and the second domain and comprising a hinge-CH2-CH3 Fc domain. In embodiments, the first domain comprises an extracellular domain of integrin α4 and/or integrin β7. In embodiments, the second domain comprises a portion of EBI3 and/or IL-12A. In embodiments, the chimeric protein is heterodimeric.

An aspect of the present invention is a method of treating a condition caused by or associated with TNFα-mediated apoptosis comprising administering to a subject in need thereof an effective amount of a pharmaceutical composition comprising a therapeutically effective amount of the chimeric protein of any of the herein disclosed aspects or embodiments. In embodiments, the condition caused by or associated with TNFα-mediated apoptosis is selected from an inflammatory bowel disease (IBD), inflammation, autoimmune disease, and allergy. In embodiments, the inflammatory bowel disease (IBD) is selected from Crohn's disease (CD), and ulcerative colitis (UC), collagenous colitis, lymphocytic colitis, ischemic colitis, diversion colitis, Behcet's disease, and indeterminate colitis. In embodiments, the autoimmune disease is selected from ankylosing spondylitis, diabetes mellitus, Grave's disease, Hashimoto's thyroiditis, hypersensitivity reactions (e.g., allergies, hay fever, asthma, and acute edema cause type I hypersensitivity reactions), inflammatory bowel diseases (e.g., colitis ulcerosa and Crohn's disease), multiple sclerosis, psoriasis, psoriasis, rheumatoid arthritis, sarcoidosis, Sjögren's syndrome, systemic lupus erythematosus, and vasculitis. In embodiments, the method pushes the T helper axis towards a less inflammatory state and/or decreases inflammation.

An aspect of the present invention is a method of treating a condition caused by or associated with TNFα-mediated apoptosis in a subject in need thereof, the method comprising: contacting lymphocytes from the subject with an effective amount of a pharmaceutical composition comprising a therapeutically effective amount of the chimeric protein of any of the herein disclosed aspects or embodiments. In embodiments, the lymphocytes from the subject are contacted with the pharmaceutical composition in vivo. In embodiments, the lymphocytes from the subject are contacted with the pharmaceutical composition ex vivo. In embodiments, the method further comprises extracting peripheral blood mononuclear cells comprising lymphocytes from the subject. In embodiments, the method further comprises culturing and/or expanding the lymphocytes ex vivo. In embodiments, the method further comprises administering the cultured and/or expanded lymphocytes to the subject. In embodiments, the inflammatory bowel disease (IBD), is selected from Crohn's disease (CD), and ulcerative colitis (UC), collagenous colitis, lymphocytic colitis, ischemic colitis, diversion colitis, Behcet's disease, and indeterminate colitis. In embodiments, the method pushes the T helper axis towards a less inflammatory state and/or decreases inflammation.

In embodiments, the method reduces the binding of binding of TNFα to a TNFα ligand/receptor. In embodiments, the TNFα ligand/receptor is type I receptor (TNFRI). In embodiments, the method reduces the activation of caspase-dependent cell death. In embodiments, the method reduces the recruitment of TRADD (TNFR-associated death domain), TRAFs (TNFR-associated factors) and/or RIP. In embodiments, the method reduces the formation of a cytoplasmic TRADD complex comprising FADD (FAS-associated death domain) and pro-caspase 8. In embodiments, the method reduces the activation of caspase 8 and/or the initiation of an apoptotic signaling cascade.

In embodiments, the method increases amount and/or immunosuppressive activity of CD4+ cells. In embodiments, the CD4+ cells are regulatory T cells (Tregs) selected from natural Tregs (nTregs), IL-10-producing type 1 Tregs (Tr1 cells), TGF-β-producing Th3 cells, CD8+ Tregs, NKT regulatory cells and pTreg. In embodiments, the amount and/or activity of CD4+ cells is increased by at least 5%, or at least 10%, or at least 20%, or at least 30%, or at least 40%, or at least 50%, or at least 60%, or at least 70%, or at least 80%, or at least 90%%, or at least 100%, or at least 150%, or at least 200%, or at least 250%, or at least 300%, or at least 350%%, or at least 400%, or at least 550%, or at least 500%. In embodiments, the method increases the amount and/or activity of an immunosuppressive cytokine. In embodiments, immunosuppressive cytokine is selected from the IL-1Ra, IL-4, IL-10, IL-11, IL-13, TGFβ, IL-33, IL-35, and IL-37. In embodiments, the amount and/or activity of CD4+ cells is increased by at least 5%, or at least 10%, or at least 20%, or at least 30%, or at least 40%, or at least 50%, or at least 60%, or at least 70%, or at least 80%, or at least 90%%, or at least 100%, or at least 150%, or at least 200%, or at least 250%, or at least 300%, or at least 350%%, or at least 400%, or at least 550%, or at least 500%.

In embodiments, the method decreases the amount and/or activity of CD8+ cells. In embodiments, the amount and/or activity of CD8+ cells is decreased by at least 5%, or at least 10%, or at least 20%, or at least 30%, or at least 40%, or at least 50%, or at least 60%, or at least 70%, or at least 80%, or at least 90%. In embodiments, the method reduces the amount and/or activity of an immunostimulatory cytokine. In embodiments, the immunostimulatory cytokine is selected from IL-2, IL-4, IL-6, IL-7, IL-10, IL-12, IL-17, IL-21, IL-22, IFNα, IFNγ, GM-CSF and TNFα. In embodiments, the amount of the immunostimulatory cytokine is decreased by at least 5%, or at least 10%, or at least 20%, or at least 30%, or at least 40%, or at least 50%, or at least 60%, or at least 70%, or at least 80%, or at least 90%.

In embodiments, the chimeric protein used in the method of treating a condition caused by or associated with TNFα-mediated apoptosis has a general structure of: N terminus-(a)-(b)-(c)-C terminus in which (a) is a first domain comprising a portion of the extracellular domain of a transmembrane protein, a secreted protein, or a membrane-anchored extracellular protein, (c) is a second domain comprising a portion of the extracellular domain of a transmembrane protein, a secreted protein, or a membrane-anchored extracellular protein, and (b) is a linker adjoining the first domain and the second domain. In this aspect, either or both of the first domain and the second domain decreases self-directed immune system activity when bound to its ligand/receptor. In embodiments, the portion of the first domain comprises a transmembrane protein, a secreted protein, or a membrane-anchored extracellular protein selected from B7H3, B7H4, BTNL2, CTLA4, CSF3, ICOSL, ILDR2, PD-L1, TNFR2, IL-6R, MadCAM, integrin α4β7, and VSIG4. In embodiments, the portion of the second domain comprises a transmembrane protein, a secreted protein, or a membrane-anchored extracellular protein selected from BTNL2, IL2, PD-L1, SEMA3E, IL-35, CCL25, TGFβ, and TL1A. In embodiments, the cytokine is decreased because of binding of the chimeric protein to the soluble ligand. In embodiments, the immunostimulatory cytokine is decreased because of binding of the chimeric protein to the soluble ligand.

In embodiments, the chimeric protein used in method of treating a condition caused by or associated with TNFα-mediated apoptosis comprises: (a) a first domain comprising a portion of VSIG4 that is capable of binding a VSIG4 ligand/receptor, (b) a second domain comprising a portion of IL2 that is capable of binding an IL2 receptor, and (c) a linker linking the first domain and the second domain and comprising a hinge-CH2-CH3 Fc domain.

In embodiments, the chimeric protein used in method of treating a condition caused by or associated with TNFα-mediated apoptosis comprises: (a) a first domain comprising a portion of CTLA4 that is capable of binding a CTLA4 ligand/receptor, (b) a second domain comprising a portion of IL2 that is capable of binding an IL2 receptor, and (c) a linker linking the first domain and the second domain and comprising a hinge-CH2-CH3 Fc domain. In embodiments, the IL2 receptor is a high-affinity IL2 receptor that is expressed by regulatory T cells, e.g., the portion of IL2 comprises one or more mutations relative to a corresponding portion of wild-type IL2 which provides preferential binding to the high-affinity IL2 receptor that is expressed by regulatory T cells.

In embodiments, the chimeric protein used in method of treating a condition caused by or associated with TNFα-mediated apoptosis comprises: (a) a first domain comprising a portion of CTLA4 that is capable of binding a CTLA4 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of treating a condition caused by or associated with TNFα-mediated apoptosis comprises: (a) a first domain comprising a portion of B7H3 that is capable of binding a B7H3 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of treating a condition caused by or associated with TNFα-mediated apoptosis comprises: (a) a first domain comprising a portion of B7H4 that is capable of binding a B7H4 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of treating a condition caused by or associated with TNFα-mediated apoptosis comprises: (a) a first domain comprising a portion of ICOSL that is capable of binding an ICOSL ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of treating a condition caused by or associated with TNFα-mediated apoptosis comprises: (a) a first domain comprising a portion of ILDR2 that is capable of binding an ILDR2 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of treating a condition caused by or associated with TNFα-mediated apoptosis comprises: (a) a first domain comprising a portion of BTNL2 that is capable of binding a BTNL2 ligand/receptor, (b) a second domain comprising a portion of PD-L1 that is capable of binding PD-1, and (c) a linker linking the first domain and the second domain and comprising a hinge-CH2-CH3 Fc domain.

In embodiments, the chimeric protein used in method of treating a condition caused by or associated with TNFα-mediated apoptosis comprises: (a) a first domain comprising a portion of PD-L1 that is capable of binding PD-1, (b) a second domain comprising a portion of BTNL2 that is capable of binding a BTNL2 ligand/receptor, and (c) a linker linking the first domain and the second domain and comprising a hinge-CH2-CH3 Fc domain.

In embodiments, the chimeric protein used in method of treating a condition caused by or associated with TNFα-mediated apoptosis comprises: (a) a first domain comprising a portion of CSF3 that is capable of binding a CSF3 ligand/receptor, (b) a second domain comprising a portion of TL1A that is capable of binding a TL1A ligand/receptor, and (c) a linker linking the first domain and the second domain and comprising a hinge-CH2-CH3 Fc domain.

In embodiments, the chimeric protein used in method of treating a condition caused by or associated with TNFα-mediated apoptosis comprises: (a) a first domain comprising a portion of CTLA4 that is capable of binding a CTLA4 ligand/receptor, (b) a second domain comprising a portion of TL1A that is capable of binding a TL1A ligand/receptor, and (c) a linker linking the first domain and the second domain and comprising a hinge-CH2-CH3 Fc domain.

In embodiments, the chimeric protein used in method of treating a condition caused by or associated with TNFα-mediated apoptosis comprises: (a) a first domain comprising a portion of CTLA4 that is capable of binding a CTLA4 ligand/receptor, (b) a second domain comprising a portion of SEMA3E that is capable of binding a SEMA3E ligand/receptor, and (c) a linker linking the first domain and the second domain and comprising a hinge-CH2-CH3 Fc domain.

In embodiments, the chimeric protein used in method of treating a condition caused by or associated with TNFα-mediated apoptosis comprises: (a) a first domain comprising a portion of MadCAM that is capable of binding a MadCAM ligand/receptor, (b) a second domain comprising a portion of CCL25 that is capable of binding a CCL25 ligand/receptor, and (c) a linker linking the first domain and the second domain and comprising a hinge-CH2-CH3 Fc domain.

In embodiments, the chimeric protein used in method of treating a condition caused by or associated with TNFα-mediated apoptosis comprises: (a) a first domain comprising a portion of TNFR2 that is capable of binding a TNFR2 ligand/receptor, (b) a second domain comprising a portion of TGFβ that is capable of binding a TGFβ ligand/receptor, and (c) a linker linking the first domain and the second domain and comprising a hinge-CH2-CH3 Fc domain.

In embodiments, the chimeric protein used in method of treating a condition caused by or associated with TNFα-mediated apoptosis comprises: (a) a first domain comprising an extracellular domain of IL-6R that is capable of binding a IL-6R ligand/receptor, (b) a second domain comprising a portion of IL-35 that is capable of binding a IL-35 ligand/receptor, and (c) a linker linking the first domain and the second domain and comprising a hinge-CH2-CH3 Fc domain. In embodiments, the first domain comprises an extracellular domain of IL-6ST and/or IL-6R. In embodiments, the second domain comprises a portion of EBI3 and/or IL-12A. In embodiments, the chimeric protein is heterodimeric.

In embodiments, the chimeric protein used in method of treating a condition caused by or associated with TNFα-mediated apoptosis comprises: (a) a first domain comprising an extracellular domain of integrin α4β7 that is capable of binding an integrin α4β7 ligand/receptor, (b) a second domain comprising a portion of IL-35 that is capable of binding a IL-35 ligand/receptor, and (c) a linker linking the first domain and the second domain and comprising a hinge-CH2-CH3 Fc domain. In embodiments, the first domain comprises an extracellular domain of integrin α4 and/or integrin β7. In embodiments, the second domain comprises a portion of EBI3 and/or IL-12A. In embodiments, the chimeric protein is heterodimeric.

In embodiments, the hinge-CH2-CH3 Fc domain comprises at least one cysteine residue capable of forming a disulfide bond. In embodiments, the hinge-CH2-CH3 Fc domain is derived from IgG (e.g., IgG1, IgG2, IgG3, and IgG4), IgA (e.g., IgA1 and IgA2), IgD, or IgE. In embodiments, the IgG is IgG4, e.g., a human IgG4. In embodiments, the IgG is IgG1, e.g., a human IgG1. In embodiments, the linker comprises an amino acid sequence that is at least 95% identical to the amino acid sequence of SEQ ID NO: 1, SEQ ID NO: 2, or SEQ ID NO: 3.

In embodiments, any of the methods disclosed herein further comprise administering to the subject an anti-inflammatory drug, e.g., a non-steroidal anti-inflammatory or a corticosteroid. In embodiments, the pharmaceutical composition and the anti-inflammatory drug are provided simultaneously (e.g., as two distinct pharmaceutical compositions or as a single pharmaceutical composition), the pharmaceutical composition is administered after the anti-inflammatory drug is administered, or the pharmaceutical composition is administered before the anti-inflammatory drug is administered. In embodiments, the non-steroidal anti-inflammatory is selected from the group consisting of acetyl salicylic acid (aspirin), benzyl-2,5-diacetoxybenzoic acid, celecoxib, diclofenac, etodolac, etofenamate, fulindac, glycol salicylate, ibuprofen, indomethacin, ketoprofen, methyl salicylate, nabumetone, naproxen, oxaprozin, phenylbutazone, piroxicam, salicylic acid, salicylmides, and Vimovo® (a combination of naproxen and esomeprazole magnesium). In embodiments, the corticosteroid is selected from the group consisting of alpha-methyl dexamethasone, amcinafel, amcinafide, beclomethasone dipropionate, beclomethasone dipropionate, betamethasone and the balance of its esters, betamethasone benzoate, betamethasone dipropionate, betamethasone valerate, beta-methyl betamethasone, bethamethasone, chloroprednisone, clescinolone, clobetasol valerate, clocortelone, cortisone, cortodoxone, desonide, desoxymethasone, dexamethasone, dichlorisone, diflorasone diacetate, diflucortolone valerate, difluorosone diacetate, difluprednate, fluadrenolone, flucetonide, fluclorolone acetonide, flucloronide, flucortine butylester, fludrocortisone, flumethasone pivalate, flunisolide, fluocinonide, fluocortolone, fluoromethalone, fluosinolone acetonide, fluperolone, fluprednidene (fluprednylidene) acetate, fluprednisolone, fluradrenolone acetonide, flurandrenolone, halcinonide, hydrocortisone, hydrocortisone acetate, hydrocortisone butyrate, hydroxyltriamcinolone, medrysone, meprednisone, methylprednisolone, paramethasone, prednisolone, prednisone, triamcinolone, and triamcinolone acetonide.

In embodiments, any of the methods disclosed herein further comprise administering to the subject an immunosuppressive agent. In embodiments, the pharmaceutical composition and the immunosuppressive agent are provided simultaneously (e.g., as two distinct pharmaceutical compositions or as a single pharmaceutical composition), the pharmaceutical composition is administered after the immunosuppressive agent is administered, or the pharmaceutical composition is administered before the immunosuppressive agent is administered. In embodiments, the immunosuppressive agent is selected from the group consisting of an antibody (e.g., basiliximab, daclizumab, and muromonab), an anti-immunophilin (e.g., cyclosporine, tacrolimus, and sirolimus), an antimetabolite (e.g., azathioprine and methotrexate), a cytostatic (such as alkylating agents), a cytotoxic antibiotic, an interferon, a mycophenolate, an opioid, a small biological agent (e.g., fingolimod and myriocin), and a TNF binding protein.

In embodiments, any of the methods disclosed herein further comprise administering to the subject an anti-inflammatory drug (as disclosed herein) and an immunosuppressive agent (as disclosed herein). The order of administration of the pharmaceutical composition comprising a therapeutically effective amount of the chimeric protein, the anti-inflammatory drug, and the immunosuppressive agent is not limited. As examples, the pharmaceutical composition may be administered before the anti-inflammatory drug and the immunosuppressive agent (e.g., which are formulated into a single pharmaceutical composition or as two pharmaceutical compositions); the pharmaceutical composition may be administered before the anti-inflammatory drug and after the immunosuppressive agent; the pharmaceutical composition may be administered with the anti-inflammatory drug (e.g., in a single pharmaceutical composition or in two pharmaceutical compositions) and before the immunosuppressive agent; the anti-inflammatory drug and the immunosuppressive agent may be administered in a single pharmaceutical composition or in two pharmaceutical compositions before the pharmaceutical composition is administered; and the pharmaceutical composition, the anti-inflammatory drug, and the immunosuppressive agent may be administered together, e.g., in a single composition.

In embodiments, the method treats an autoimmune disease selected from ankylosing spondylitis, diabetes mellitus, Grave's disease, Hashimoto's thyroiditis, hypersensitivity reactions (e.g., allergies, hay fever, asthma, and acute edema cause type I hypersensitivity reactions), inflammatory bowel diseases (e.g., colitis ulcerosa and Crohn's disease), multiple sclerosis, psoriasis, psoriasis, rheumatoid arthritis, sarcoidosis, Sjögren's syndrome, systemic lupus erythematosus, and vasculitis.

### Administration, Dosing, and Treatment Regimens

Routes of administration include, for example: intradermal, intratumoral, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin.

As examples, administration results in the release of chimeric protein (and/or an anti-inflammatory drug and/or an immunosuppressive agent) disclosed herein into the bloodstream (via enteral or parenteral administration), or alternatively, the chimeric protein (and/or an anti-inflammatory drug and/or an immunosuppressive agent) is administered directly to the site of active disease.

Any chimeric protein (and/or an anti-inflammatory drug and/or an immunosuppressive agent) disclosed herein can be administered orally. Any chimeric protein (and/or an anti-inflammatory drug and/or an immunosuppressive agent) can also be administered by any other convenient route, for example, by intravenous infusion or bolus injection, by absorption through epithelial or mucocutaneous linings (e.g., oral mucosa, rectal and intestinal mucosa, etc.). Administration can be systemic or local. Various delivery systems are known, e.g., encapsulation in liposomes, microparticles, microcapsules, or capsules, and can be used to facilitate administration.

Dosage forms suitable for parenteral administration (e.g., intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art.

The dosage of any chimeric protein (and/or an anti-inflammatory drug and/or an immunosuppressive agent) disclosed herein as well as the dosing schedule can depend on various parameters, including, but not limited to, the disease being treated, the subject's general health, and the administering physician's discretion.

Any chimeric protein disclosed herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of an anti-inflammatory drug and/or an immunosuppressive agent, to a subject in need thereof.

In embodiments, a chimeric protein and an anti-inflammatory drug and/or an immunosuppressive agent are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, 1 day apart, 2 days apart, 3 days apart, 4 days apart, 5 days apart, 6 days apart, 1 week apart, 2 weeks apart, 3 weeks apart, or 4 weeks apart.

The dosage of any chimeric protein (and/or an anti-inflammatory drug and/or an immunosuppressive agent) disclosed herein can depend on several factors including the severity of the condition, whether the condition is to be treated or prevented, and the age, weight, and health of the subject to be treated. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

For administration of any chimeric protein disclosed herein by parenteral injection, the dosage may be about 0.1 mg to about 250 mg per day, about 1 mg to about 20 mg per day, or about 3 mg to about 5 mg per day. Generally, when orally or parenterally administered, the dosage of any chimeric protein disclosed herein may be about 0.1 mg to about 1500 mg per day, or about 0.5 mg to about 10 mg per day, or about 0.5 mg to about 5 mg per day, or about 200 to about 1,200 mg per day (e.g., about 200 mg, about 300 mg, about 400 mg, about 500 mg, about 600 mg, about 700 mg, about 800 mg, about 900 mg, about 1,000 mg, about 1,100 mg, about 1,200 mg per day).

In embodiments, administration of the chimeric protein disclosed herein is by parenteral injection at a dosage of about 0.1 mg to about 1500 mg per treatment, or about 0.5 mg to about 10 mg per treatment, or about 0.5 mg to about 5 mg per treatment, or about 200 to about 1,200 mg per treatment (e.g., about 200 mg, about 300 mg, about 400 mg, about 500 mg, about 600 mg, about 700 mg, about 800 mg, about 900 mg, about 1,000 mg, about 1,100 mg, about 1,200 mg per treatment).

In embodiments, a suitable dosage of the chimeric protein is in a range of about 0.01 mg/kg to about 100 mg/kg of body weight, or about 0.01 mg/kg to about 10 mg/kg of body weight of the subject, for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg body weight, inclusive of all values and ranges therebetween.

In embodiments, delivery of a chimeric protein disclosed herein (and/or an anti-inflammatory drug and/or an immunosuppressive agent) can be in a vesicle, in particular a liposome (see Langer, 1990, Science 249:1527-1533; Treat et al, in Liposomes in Therapy of Infectious Disease and Cancer, Lopez-Berestein and Fidler (eds.), Liss, New York, pp. 353-365 (1989).

A chimeric protein (and/or an anti-inflammatory drug and/or an immunosuppressive agent) disclosed herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In embodiments, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105).

In embodiments, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Administration of any chimeric protein (and/or an anti-inflammatory drug and/or an immunosuppressive agent) disclosed herein can, independently, be one to four times daily or one to four times per month or one to six times per year or once every two, three, four or five years. Administration can be for the duration of one day or one month, two months, three months, six months, one year, two years, three years, and may even be for the life of the subject.

The dosage regimen utilizing any chimeric protein (and/or an anti-inflammatory drug and/or an immunosuppressive agent) disclosed herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific compound of the invention employed. Any chimeric protein (and/or an anti-inflammatory drug and/or an immunosuppressive agent) disclosed herein can be administered in a single daily dose, or the total daily dosage can be administered in divided doses of two, three or four times daily. Furthermore, any chimeric protein (and/or an anti-inflammatory drug and/or an immunosuppressive agent) disclosed herein can be administered continuously rather than intermittently throughout the dosage regimen.

### Cells and Nucleic Acids

An aspect of the present invention is an expression vector comprising a nucleic acid encoding the chimeric protein of any of the herein disclosed aspects or embodiments. The expression vector comprises a nucleic acid encoding the chimeric protein disclosed herein. In embodiments, the expression vector comprises DNA or RNA. In embodiments, the expression vector is a mammalian expression vector.

An expression vector may be produced by cloning the nucleic acids encoding the three fragments (the first domain, followed by a linker sequence, followed by the second) into a vector (plasmid, viral or other). Accordingly, in embodiments, the present chimeric proteins are engineered as such.

Both prokaryotic and eukaryotic vectors can be used for expression of the chimeric protein. Prokaryotic vectors include constructs based on E. coli sequences (see, e.g., Makrides, Microbiol Rev 1996, 60:512-538). Non-limiting examples of regulatory regions that can be used for expression in E. coli include lac, trp, Ipp, phoA, recA, tac, T3, T7 and λP_{L}. Non-limiting examples of prokaryotic expression vectors may include the λgt vector series such as λgt11 (Huynh et al., in “DNA Cloning Techniques, Vol. I: A Practical Approach,” 1984, (D. Glover, ed.), pp. 49-78, IRL Press, Oxford), and the pET vector series (Studier et al., Methods Enzymol 1990, 185:60-89). Prokaryotic host-vector systems cannot perform much of the post-translational processing of mammalian cells, however. Thus, eukaryotic host-vector systems may be particularly useful. A variety of regulatory regions can be used for expression of the chimeric proteins in mammalian host cells. For example, the SV40 early and late promoters, the cytomegalovirus (CMV) immediate early promoter, and the Rous sarcoma virus long terminal repeat (RSV-LTR) promoter can be used. Inducible promoters that may be useful in mammalian cells include, without limitation, promoters associated with the metallothionein II gene, mouse mammary tumor virus glucocorticoid responsive long terminal repeats (MMTV-LTR), the β-interferon gene, and the hsp70 gene (see, Williams et al., Cancer Res 1989, 49:2735-42; and Taylor et al., Mol Cell Biol 1990, 10:165-75). Heat shock promoters or stress promoters also may be advantageous for driving expression of the chimeric proteins in recombinant host cells.

In embodiments, expression vectors of the invention comprise a nucleic acid encoding the chimeric proteins, or a complement thereof, operably linked to an expression control region, or complement thereof, that is functional in a mammalian cell. The expression control region is capable of driving expression of the operably linked blocking and/or stimulating agent encoding nucleic acid such that the blocking and/or stimulating agent is produced in a human cell transformed with the expression vector.

Expression control regions are regulatory polynucleotides (sometimes referred to herein as elements), such as promoters and enhancers, that influence expression of an operably linked nucleic acid. An expression control region of an expression vector of the invention is capable of expressing operably linked encoding nucleic acid in a human cell. In embodiments, the cell is a tumor cell. In another embodiment, the cell is a non-tumor cell. In embodiments, the expression control region confers regulatable expression to an operably linked nucleic acid. A signal (sometimes referred to as a stimulus) can increase or decrease expression of a nucleic acid operably linked to such an expression control region. Such expression control regions that increase expression in response to a signal are often referred to as inducible. Such expression control regions that decrease expression in response to a signal are often referred to as repressible. Typically, the amount of increase or decrease conferred by such elements is proportional to the amount of signal present; the greater the amount of signal, the greater the increase or decrease in expression.

In embodiments, the present invention contemplates the use of inducible promoters capable of effecting high level of expression transiently in response to a cue. For example, when in the proximity of a tumor cell, a cell transformed with an expression vector for the chimeric protein (and/or an anti-inflammatory drug and/or an immunosuppressive agent) comprising such an expression control sequence is induced to transiently produce a high level of the agent by exposing the transformed cell to an appropriate cue. Illustrative inducible expression control regions include those comprising an inducible promoter that is stimulated with a cue such as a small molecule chemical compound. Particular examples can be found, for example, in U.S. Pat. Nos. 5,989,910, 5,935,934, 6,015,709, and 6,004,941, each of which is incorporated herein by reference in its entirety.

Expression control regions and locus control regions include full-length promoter sequences, such as native promoter and enhancer elements, as well as subsequences or polynucleotide variants which retain all or part of full-length or non-variant function. As used herein, the term “functional” and grammatical variants thereof, when used in reference to a nucleic acid sequence, subsequence or fragment, means that the sequence has one or more functions of native nucleic acid sequence (e.g., non-variant or unmodified sequence).

As used herein, “operable linkage” refers to a physical juxtaposition of the components so described as to permit them to function in their intended manner. In the example of an expression control element in operable linkage with a nucleic acid, the relationship is such that the control element modulates expression of the nucleic acid. Typically, an expression control region that modulates transcription is juxtaposed near the 5′ end of the transcribed nucleic acid (i.e., “upstream”). Expression control regions can also be located at the 3′ end of the transcribed sequence (i.e., “downstream”) or within the transcript (e.g., in an intron). Expression control elements can be located at a distance away from the transcribed sequence (e.g., 100 to 500, 500 to 1000, 2000 to 5000, or more nucleotides from the nucleic acid). A specific example of an expression control element is a promoter, which is usually located 5′ of the transcribed sequence. Another example of an expression control element is an enhancer, which can be located 5′ or 3′ of the transcribed sequence, or within the transcribed sequence.

Expression systems functional in human cells are well known in the art, and include viral systems. Generally, a promoter functional in a human cell is any DNA sequence capable of binding mammalian RNA polymerase and initiating the downstream (3′) transcription of a coding sequence into mRNA. A promoter will have a transcription initiating region, which is usually placed proximal to the 5′ end of the coding sequence, and typically a TATA box located 25-30 base pairs upstream of the transcription initiation site. The TATA box is thought to direct RNA polymerase II to begin RNA synthesis at the correct site. A promoter will also typically contain an upstream promoter element (enhancer element), typically located within 100 to 200 base pairs upstream of the TATA box. An upstream promoter element determines the rate at which transcription is initiated and can act in either orientation. Of particular use as promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived from SV40. Introns may also be included in expression constructs.

There is a variety of techniques available for introducing nucleic acids into viable cells. Techniques suitable for the transfer of nucleic acid into mammalian cells in vitro include the use of liposomes, electroporation, microinjection, cell fusion, polymer-based systems, DEAE-dextran, viral transduction, the calcium phosphate precipitation method, etc. For in vivo gene transfer, a number of techniques and reagents may also be used, including liposomes; natural polymer-based delivery vehicles, such as chitosan and gelatin; viral vectors are also suitable for in vivo transduction. In some situations, it is desirable to provide a targeting agent, such as an antibody or ligand specific for a tumor cell surface membrane protein. Where liposomes are employed, proteins which bind to a cell surface membrane protein associated with endocytosis may be used for targeting and/or to facilitate uptake, e.g., capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al, J. Biol. Chem. 262, 4429-4432 (1987); and Wagner et al, Proc. Natl. Acad. Sci. USA 87, 3410-3414 (1990).

Where appropriate, gene delivery agents such as, e.g., integration sequences can also be employed. Numerous integration sequences are known in the art (see, e.g., Nunes-Duby et al., Nucleic Acids Res. 26:391-406, 1998; Sadwoski, J. Bacteriol., 165:341-357, 1986; Bestor, Cell, 122(3):322-325, 2005; Plasterk et al, TIG 15:326-332, 1999; Kootstra et al., Ann. Rev. Pharm. Toxicol., 43:413-439, 2003). These include recombinases and transposases. Examples include Cre (Sternberg and Hamilton, J. Mol. Biol., 150:467-486, 1981), lambda (Nash, Nature, 247, 543-545, 1974), Flp (Broach, et al., Cell, 29:227-234, 1982), R (Matsuzaki, et al., J. Bacteriology, 172:610-618, 1990), cpC31 (see, e.g., Groth et al., J. Mol. Biol. 335:667-678, 2004), sleeping beauty, transposases of the mariner family (Plasterk et al., supra), and components for integrating viruses such as AAV, retroviruses, and antiviruses having components that provide for virus integration such as the LTR sequences of retroviruses or lentivirus and the ITR sequences of AAV (Kootstra et al., Ann. Rev. Pharm. Toxicol., 43:413-439, 2003). In addition, direct and targeted genetic integration strategies may be used to insert nucleic acid sequences encoding the chimeric fusion proteins including CRISPR/CAS9, zinc finger, TALEN, and meganuclease gene-editing technologies.

In embodiments, the expression vectors for the expression of the chimeric proteins (and/or an anti-inflammatory drug and/or an immunosuppressive agent) are viral vectors. Many viral vectors useful for gene therapy are known (see, e.g., Lundstrom, Trends Biotechnol., 21: 1 17, 122, 2003. Illustrative viral vectors include those selected from Antiviruses (LV), retroviruses (RV), adenoviruses (AV), adeno-associated viruses (AAV), and a viruses, though other viral vectors may also be used. For in vivo uses, viral vectors that do not integrate into the host genome are suitable for use, such as a viruses and adenoviruses. Illustrative types of a viruses include Sindbis virus, Venezuelan equine encephalitis (VEE) virus, and Semliki Forest virus (SFV). For in vitro uses, viral vectors that integrate into the host genome are suitable, such as retroviruses, AAV, and Antiviruses. In embodiments, the invention provides methods of transducing a human cell in vivo, comprising contacting a solid tumor in vivo with a viral vector of the invention.

Another aspect of the present invention is a host cell comprising the expression vector of the preceding aspect and embodiments.

Expression vectors can be introduced into host cells for producing the present chimeric proteins. Cells may be cultured in vitro or genetically engineered, for example. Useful mammalian host cells include, without limitation, cells derived from humans, monkeys, and rodents (see, for example, Kriegler in “Gene Transfer and Expression: A Laboratory Manual,” 1990, New York, Freeman & Co.). These include monkey kidney cell lines transformed by SV40 (e.g., COS-7, ATCC CRL 1651); human embryonic kidney lines (e.g., 293, 293-EBNA, or 293 cells subcloned for growth in suspension culture, Graham et al, J Gen Virol 1977, 36:59); baby hamster kidney cells (e.g., BHK, ATCC CCL 10); Chinese hamster ovary-cells-DHFR (e.g., CHO, Urlaub and Chasin, Proc Natl Acad Sci USA 1980, 77:4216); DG44 CHO cells, CHO-K1 cells, mouse sertoli cells (Mather, Biol Reprod 1980, 23:243-251); mouse fibroblast cells (e.g., NIH-3T3), monkey kidney cells (e.g., CV1 ATCC CCL 70); African green monkey kidney cells. (e.g., VERO-76, ATCC CRL-1587); human cervical carcinoma cells (e.g., HELA, ATCC CCL 2); canine kidney cells (e.g., MDCK, ATCC CCL 34); buffalo rat liver cells (e.g., BRL 3A, ATCC CRL 1442); human lung cells (e.g., W138, ATCC CCL 75); human liver cells (e.g., Hep G2, HB 8065); and mouse mammary tumor cells (e.g., MMT 060562, ATCC CCL51). Illustrative cancer cell types for expressing the chimeric proteins disclosed herein include mouse fibroblast cell line, NI H3T3, mouse Lewis lung carcinoma cell line, LLC, mouse mastocytoma cell line, P815, mouse lymphoma cell line, EL4 and its ovalbumin transfectant, E.G7, mouse melanoma cell line, B16F10, mouse fibrosarcoma cell line, MC57, and human small cell lung carcinoma cell lines, SCLC #2 and SCLC #7.

Host cells can be obtained from normal or affected subjects, including healthy humans, cancer patients, and patients with an infectious disease, private laboratory deposits, public culture collections such as the American Type Culture Collection (ATCC), or from commercial suppliers.

Cells that can be used for production of the present chimeric proteins in vitro, ex vivo, and/or in vivo include, without limitation, epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells (e.g., as obtained from bone marrow), umbilical cord blood, peripheral blood, and fetal liver. The choice of cell type depends on the type of tumor or infectious disease being treated or prevented, and can be determined by one of skill in the art.

Production and purification of Fc-containing macromolecules (such as monoclonal antibodies) has become a standardized process, with minor modifications between products. For example, many Fc containing macromolecules are produced by human embryonic kidney (HEK) cells (or variants thereof) or Chinese Hamster Ovary (CHO) cells (or variants thereof) or in some cases by bacterial or synthetic methods. Following production, the Fc containing macromolecules that are secreted by HEK or CHO cells are purified through binding to Protein A columns and subsequently ‘polished’ using various methods. Generally speaking, purified Fc containing macromolecules are stored in liquid form for some period of time, frozen for extended periods of time or in some cases lyophilized. In embodiments, production of the chimeric proteins contemplated herein may have unique characteristics as compared to traditional Fc containing macromolecules. In certain examples, the chimeric proteins may be purified using specific chromatography resins, or using chromatography methods that do not depend upon Protein A capture. In embodiments, the chimeric proteins may be purified in an oligomeric state, or in multiple oligomeric states, and enriched for a specific oligomeric state using specific methods. Without being bound by theory, these methods could include treatment with specific buffers including specified salt concentrations, pH and additive compositions. In other examples, such methods could include treatments that favor one oligomeric state over another. The chimeric proteins obtained herein may be additionally ‘polished’ using methods that are specified in the art. In embodiments, the chimeric proteins are highly stable and able to tolerate a wide range of pH exposure (between pH 3-12), are able to tolerate a large number of freeze/thaw stresses (greater than 3 freeze/thaw cycles) and are able to tolerate extended incubation at high temperatures (longer than 2 weeks at 40 degrees C.). In embodiments, the chimeric proteins are shown to remain intact, without evidence of degradation, deamidation, etc. under such stress conditions.

Subjects and/or Animals

In embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In embodiments, the subject and/or animal is a non-mammal, such, for example, a zebrafish. In embodiments, the subject and/or animal may comprise fluorescently-tagged cells (with e.g., GFP). In embodiments, the subject and/or animal is a transgenic animal comprising a fluorescent cell.

In embodiments, the subject and/or animal is a human. In embodiments, the human is a pediatric human. In embodiments, the human is an adult human. In embodiments, the human is a geriatric human. In embodiments, the human may be referred to as a patient.

In certain embodiments, the human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In embodiments, the subject is a non-human animal, and therefore the invention pertains to veterinary use. In a specific embodiment, the non-human animal is a household pet. In another specific embodiment, the non-human animal is a livestock animal.

### Kits and Medicaments

Aspects of the present invention provide kits that can simplify the administration of any chimeric protein or pharmaceutical composition as disclosed herein.

An illustrative kit of the invention comprises any chimeric protein and/or pharmaceutical composition disclosed herein in unit dosage form. In embodiments, the unit dosage form is a container, such as a pre-filled syringe, which can be sterile, containing any agent disclosed herein and a pharmaceutically acceptable carrier, diluent, excipient, or vehicle. The kit can further comprise a label or printed instructions instructing the use of any agent disclosed herein. The kit may also include a lid speculum, topical anesthetic, and a cleaning agent for the administration location. The kit can also further comprise one or more additional agent disclosed herein. In embodiments, the kit comprises a container containing an effective amount of a composition of the invention and an effective amount of another composition, such those disclosed herein.

The chimeric protein of any of the herein disclosed aspects or embodiments may be used as a medicament in the treatment of an autoimmune disease, e.g., selected from ankylosing spondylitis, diabetes mellitus, Grave's disease, Hashimoto's thyroiditis, hypersensitivity reactions (e.g., allergies, hay fever, asthma, and acute edema cause type I hypersensitivity reactions), inflammatory bowel diseases (e.g., colitis ulcerosa and Crohn's disease), multiple sclerosis, psoriasis, psoriasis, rheumatoid arthritis, sarcoidosis, Sjögren's syndrome, systemic lupus erythematosus, and vasculitis.

The present invention includes the use of the chimeric protein of any of the herein-disclosed aspects or embodiments in the manufacture of a medicament.

Another aspect of the present invention is a chimeric protein of any one the embodiments disclosed herein for use as a medicament.

Another aspect of the present invention is a chimeric protein of any one the embodiments disclosed herein for use in the treatment of an autoimmune disease.

Another aspect of the present invention is a chimeric protein of any one the embodiments disclosed herein for use in the treatment of an inflammatory disease.

Another aspect of the present invention is a chimeric protein of any one the embodiments disclosed herein in the manufacture of a medicament.

Another aspect of the present invention is a pharmaceutical composition comprising a chimeric protein of any one the embodiments disclosed herein for use as a medicament.

Another aspect of the present invention is a pharmaceutical composition comprising a chimeric protein of any one the embodiments disclosed herein in the manufacture of a medicament.

Another aspect of the present invention is a pharmaceutical composition comprising a chimeric protein of any one the embodiments disclosed herein for use in the treatment of an autoimmune disease.

Another aspect of the present invention is a pharmaceutical composition comprising a chimeric protein of any one the embodiments disclosed herein for use in the treatment of an autoimmune disease, or an inflammatory disease.

Any aspect or embodiment disclosed herein can be combined with any other aspect or embodiment as disclosed herein.

The invention will be further described in the following examples, which do not limit the scope of the invention described in the claims.

### EXAMPLES

The examples herein are provided to illustrate advantages and benefits of the present technology and to further assist a person of ordinary skill in the art with preparing or using the chimeric proteins of the present technology. The examples herein are also presented in order to more fully illustrate the preferred aspects of the present technology. The examples should in no way be construed as limiting the scope of the present technology, as defined by the appended claims. The examples can include or incorporate any of the variations, aspects or embodiments of the present technology described above. The variations, aspects or embodiments described above may also further each include or incorporate the variations of any or all other variations, aspects or embodiments of the present technology.

### Example 1: Construction and Characterization of an Illustrative CSF3- and TL1A-Based Chimeric Protein

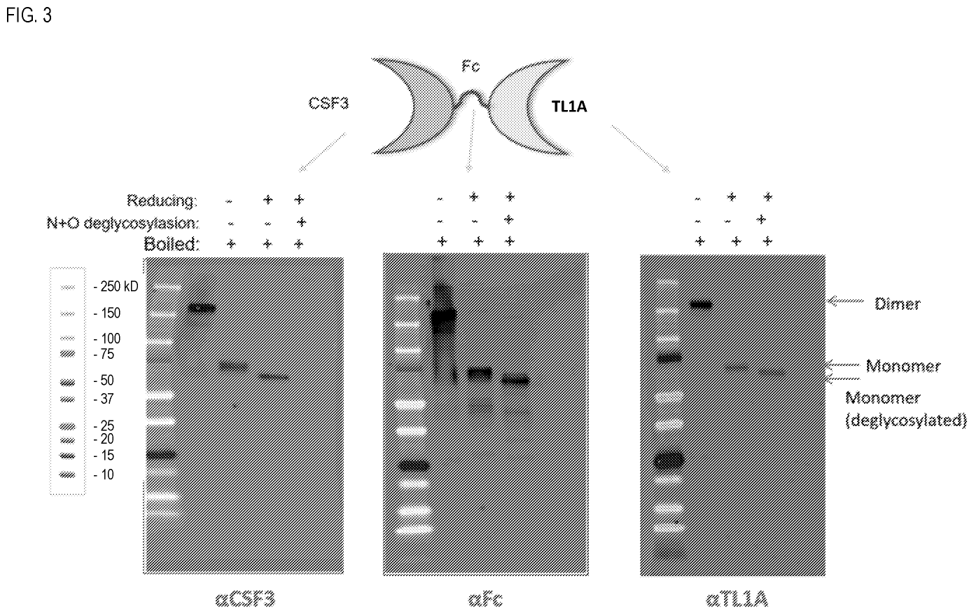

A construct encoding a murine CSF3- and TL1A-based chimeric protein was generated. The “mCSF3-Fc-TL1A” construct included a murine sequence of CSF3 fused to a murine extracellular domain (ECD) of TL1A via a hinge-CH2-CH3 Fc domain derived from IgG1. See, FIG. 3 (top).

The construct was codon optimized for expression inhuman embryonic kidney 293 (293) cells, transfected into 293 cells and individual clones were selected for high expression. High expressing clones were then used for small-scale manufacturing in stirred bioreactors in serum-free media and the relevant chimeric fusion proteins were purified with Protein A binding resin columns.

The mCSF3-Fc-TL1A construct was transiently expressed in 293 cells and purified using protein-A affinity chromatography. Western blot analyses were performed to validate the detection and binding of all three components of mCSF3-Fc-TL1A with their respective binding partners (FIG. 3, bottom). The Western blots indicated the presence of a dominant dimer band in the non-reduced lanes (FIG. 3, lane 2 in each blot), which was reduced to a glycosylated monomeric band in the presence of the reducing agent, β-mercaptoethanol (FIG. 3, lane 3 in each blot). As shown in FIG. 3, lane 4 in each blot, the chimeric protein ran as a monomer at the predicted molecular weight of about 67.3 kDa in the presence of both a reducing agent (β-mercaptoethanol) and a deglycosylation agent.

### Example 2: Further Characterization of the Binding Affinity of the Different Domains of the mCSF3-Fc-TL1A Chimeric Protein Using ELISA

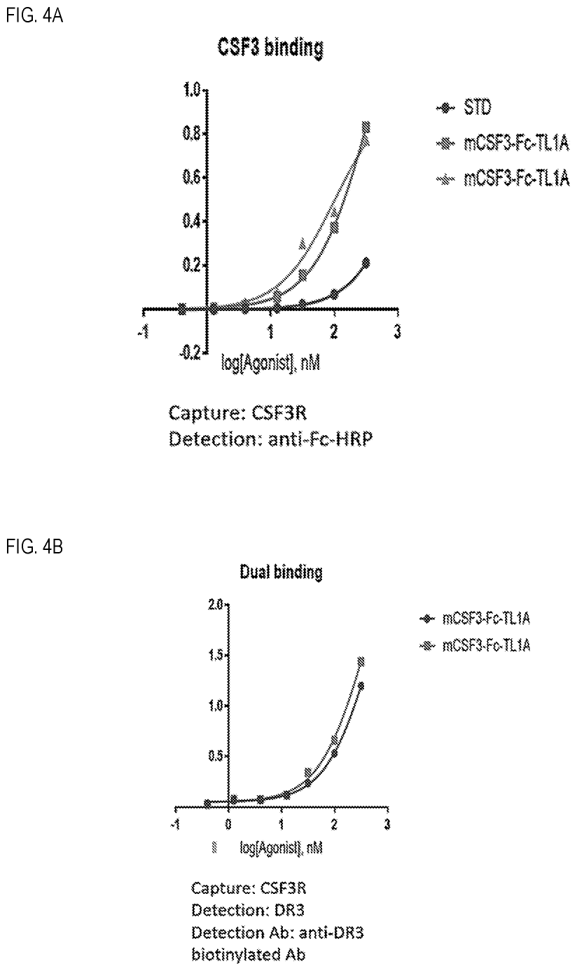

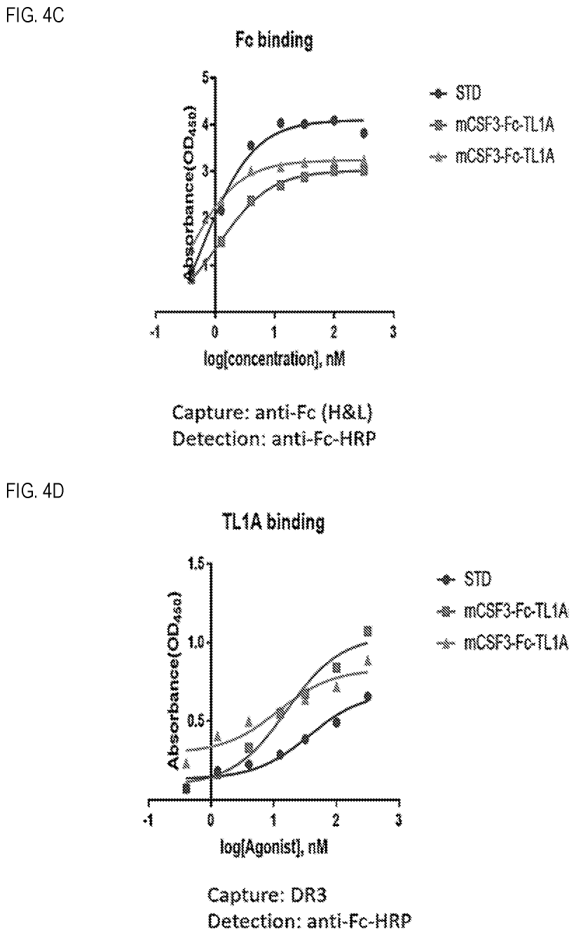

Functional ELISA (enzyme-linked immunosorbent assay) were performed to demonstrate the binding affinity of the different domains of the mCSF3-Fc-TL1A chimeric protein to their respective binding partners. As shown in FIG. 4A and FIG. 4B, binding of the CSF3 domain of the mCSF3-Fc-TL1A chimeric protein (obtained from two distinct syntheses) was characterized by capturing to a plate-bound recombinant mouse CSF3R-Fc protein and detecting via an anti-Fc-HRP antibody and HRP staining (FIG. 4A) or detecting via antibody labeled DR3 (FIG. 4B).

As shown in FIG. 4C, binding of the Fc portion of the mCSF3-Fc-TL1A chimeric protein was characterized by capturing the chimeric protein to a plate-bound mouse IgG Fc gamma antibody and detecting via an HRP conjugated anti-mouse Fc (H+L) antibody. A mouse whole IgG was used to generate a standard curve.

As shown in FIG. 4D, binding of the TL1A domain of the mCSF3-Fc-TL1A chimeric protein (obtained from two distinct syntheses) was characterized by capturing to a plate-bound recombinant mouse DR3-Fc protein and detecting via an anti-Fc-HRP antibody and HRP staining.

The data shown in FIG. 4A to FIG. 4D demonstrates that the ligand/receptor binding domains of mCSF3-Fc-TL1A effectively interacted with their binding partners in concentration-dependent manners and with high affinity.

### Example 3: The mCSF3-Fc-TL1A Chimeric Protein Increases the Frequency of Regulatory T Cells In Vivo

Regulatory T cells (Tregs), formerly known as suppressor T cells, are a subpopulation of T cells that modulate the immune system, maintain tolerance to self-antigens, and prevent autoimmune disease. Tregs are immunosuppressive and generally suppress or downregulate induction and proliferation of effector T cells.

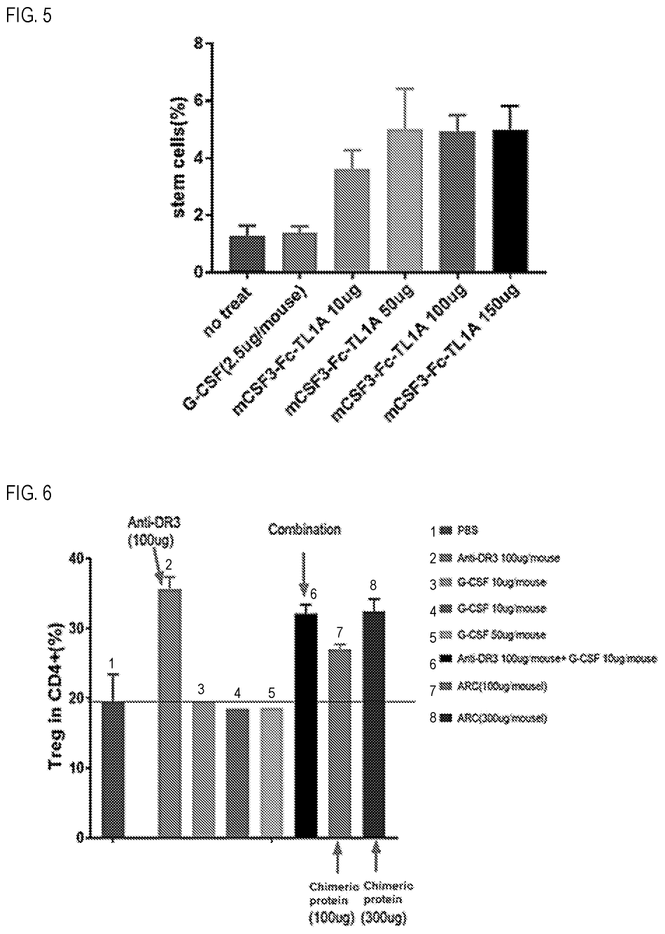

In these experiments, mice were administered mCSF3-Fc-TL1A (10 μg, 50 μg, 100 μg, or 150 μg), G-CSF (at 2.5 μg), or a sham treatment (PBS). Numbers of CD34+ lineage negative stem cells and regulatory T cells (Tregs) in a blood sample were measured before treatment and after treatment; relative frequencies of Tregs to stem cells were calculated. In the control mice, after the sham or G-CSF treatment, Tregs constituted about 1.5% of the stem cells in blood samples; see, FIG. 5. Surprisingly, the mice that were administered the mCSF3-Fc-TL1A chimeric protein had significant increases in frequency of Tregs. Indeed, the 50 μg, 100 μg, or 150 μg treatments provided roughly equivalent increases in Tregs, to about 4.5%; this is an about a three-fold increase in the frequency of Tregs relative to controls. Four mice were used for each experimental group.

In additional experiments, mice were administered a sham treatment (PBS), anti-DR3 antibody (at 100 μg), G-CSF (at 10 μg or 50 μg), a combination of the anti-DR3 antibody (at 100 μg) and G-CSF (at 10 μg), or the mCSF3-Fc-TL1A chimeric protein (at 100 μg or 300 μg). Numbers of CD4+ T cells, including the numbers regulatory T cells (Tregs), in a blood sample were measured before treatment and after treatment; relative frequencies of Tregs to overall CD4+ cells were calculated. In the control mice, after the sham or any of the G-CSF-alone treatments, Tregs constituted about 20% of the CD4+ cells in blood samples; see, FIG. 6. The anti-DR3 antibody-alone treatment increased the frequency of Tregs the greatest, to about 35% of the CD4+ cells. The 300 μg mCSF3-Fc-TL1A chimeric protein and the combination treatment of the anti-DR3 antibody and G-CSF equally increased the frequency of Tregs, to over 30%.

In conclusion, the mCSF3-Fc-TL1A chimeric protein is effective, in vivo, in increasing the frequency of Tregs relative to other CD4+ T cells and was as effective in increasing the frequency relative to combination treatments. As discussed elsewhere herein, an advantage of the present invention is ease of use and ease of production. This is because, in the present invention, two distinct immunotherapy agents are combined into a single product which may allow for a single manufacturing process instead of two independent manufacturing processes. In addition, administration of a single agent instead of two separate agents allows for easier administration and greater patient compliance. Further, in contrast to, for example, monoclonal antibodies (e.g., the anti-DR3 antibody), which are large multimeric proteins containing numerous disulfide bonds and post-translational modifications such as glycosylation, the present chimeric proteins are easier and more cost effective to manufacture.

In conclusion, the mCSF3-Fc-TL1A chimeric protein is effective in vivo in increasing the frequency of Tregs, which is a cell type that suppresses or downregulates induction and proliferation of effector T cells and contributes to preventing autoimmune diseases.

### Example 4. Construction and Characterization of an Illustrative VSIG4- and IL2-Based Chimeric Protein

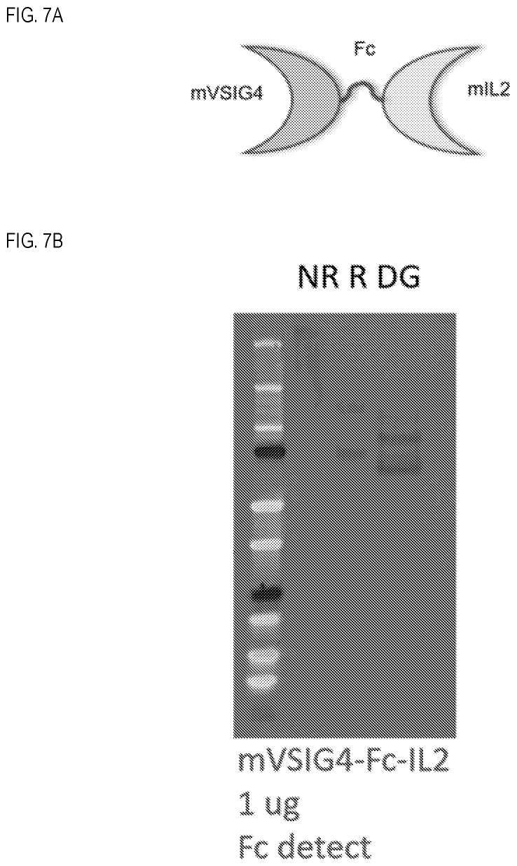

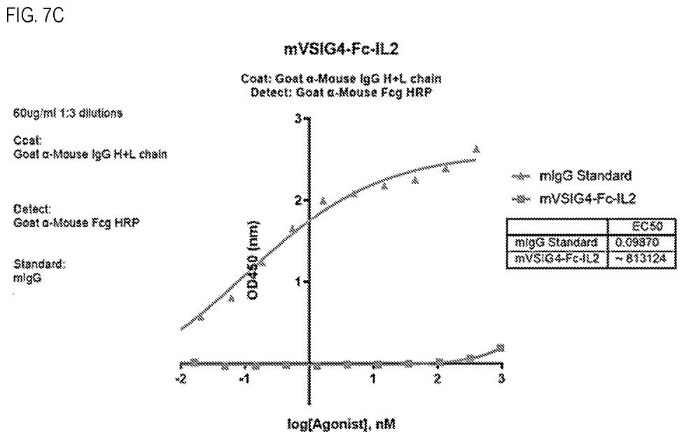

A construct encoding a murine VSIG4- and IL2-based chimeric protein was generated. The “mVSIG4-Fc-IL2” construct included a murine extracellular domain (ECD) of VSIG4 fused to a portion of murine IL2 via a hinge-CH2-CH3 Fc domain derived from IgG1. See, FIG. 7A.

The construct was prepared as described in Example 1 above. A western blot analysis was performed to validate the detection and binding of the Fc domain of mVSIG4-Fc-IL2 to an anti-Fc antibody (FIG. 7B).

Functional ELISA was performed to demonstrate the binding affinity of the Fc binding domain of mVSIG4-Fc-IL2 to an anti-Fc antibody. As shown in FIG. 7C, binding of the Fc portion of the mVSIG4-Fc-IL2 chimeric protein was characterized by capturing the chimeric protein to a plate-bound mouse IgG Fc gamma antibody and detecting via an HRP conjugated anti-mouse Fc (H+L) antibody. A mouse whole IgG was used to generate a standard curve. The starting concentration for the chimeric protein was 60 μg/ml.

### Example 5. Construction and Characterization of an Illustrative PD-L1- and BTNL2-Based Chimeric Protein

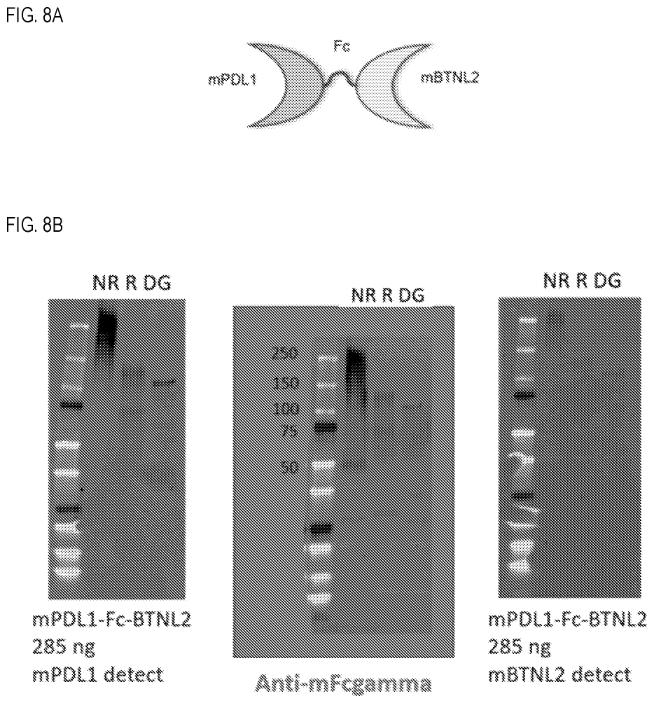

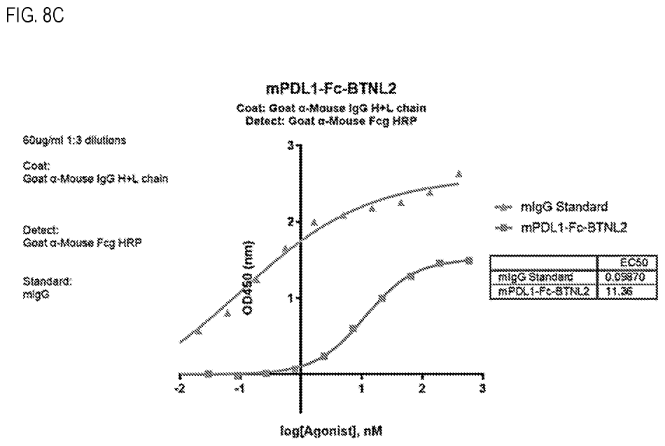

A construct encoding a murine PD-L1- and BTNL2-based chimeric protein was generated. The “mPD-L1-Fc-BTNL2” construct included a murine extracellular domain (ECD) of PD-L1 fused to the ECD of BTNL2 via a hinge-CH2-CH3 Fc domain derived from IgG1. See, FIG. 8A.

The construct was prepared as described in Example 1 above. Western blot analyses were performed to validate the detection and binding of all three components of mPD-L1-Fc-BTNL2 with their respective binding partners: PD-L1 (FIG. 8B, left), Fc (FIG. 8B, middle), and BTNL2 (FIG. 8B, right). Each western blot has untreated samples (i.e., without reducing agent or deglycosylation agent “NR”) of the mPD-L1-Fc-BTNL2 chimeric protein, samples treated with the reducing agent, β-mercaptoethanol (“R”), and samples treated with a deglycosylation agent and the reducing agent (“DG”) are shown.

Functional ELISA was performed to demonstrate the binding affinity of the Fc binding domain of mPD-L1-Fc-BTNL2 to an anti-Fc antibody. As shown in FIG. 8C, binding of the Fc portion of the mPD-L1-Fc-BTNL2 chimeric protein was characterized by capturing the chimeric protein to a plate-bound mouse IgG Fc gamma antibody and detecting via an HRP conjugated anti-mouse Fc (H+L) antibody. A mouse whole IgG was used to generate a standard curve. The starting concentration for the chimeric protein was 60 μg/ml.

### Example 6. Construction and Characterization of an Illustrative CTLA4- and SEMA3E-Based Chimeric Protein

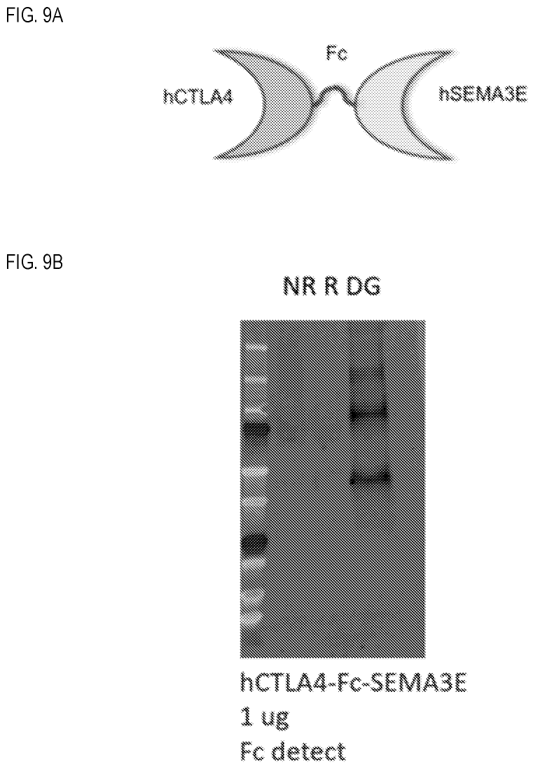

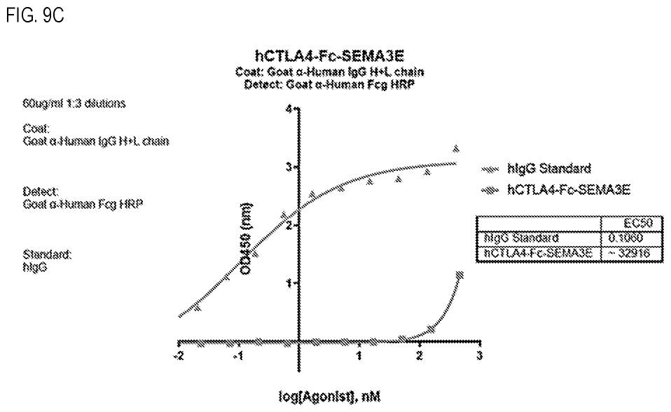

A construct encoding a human CTLA4- and SEMA3E-based chimeric protein was generated. The “hCTLA4-Fc-SEMA3E” construct included an extracellular domain (ECD) of human CTLA4 fused to a portion of human SEMA3E via a hinge-CH2-CH3 Fc domain derived from IgG1. See, FIG. 9A.

The construct was prepared as described in Example 1 above. A western blot analysis was performed to validate the detection and binding of the Fc domain of hCTLA4-Fc-SEMA3E to an anti-Fc antibody (FIG. 9B).

Functional ELISA was performed to demonstrate the binding affinity of the Fc binding domain of hCTLA4-Fc-SEMA3E to an anti-Fc antibody. As shown in FIG. 9C, binding of the Fc portion of the hCTLA4-Fc-SEMA3E chimeric protein was characterized by capturing the chimeric protein to a plate-bound mouse IgG Fc gamma antibody and detecting via an HRP conjugated anti-mouse Fc (H+L) antibody. A mouse whole IgG was used to generate a standard curve. The starting concentration for the chimeric protein was 60 μg/ml.

### Example 7. Construction and Characterization of an Illustrative ILDR2- and PD-L1-Based Chimeric Protein

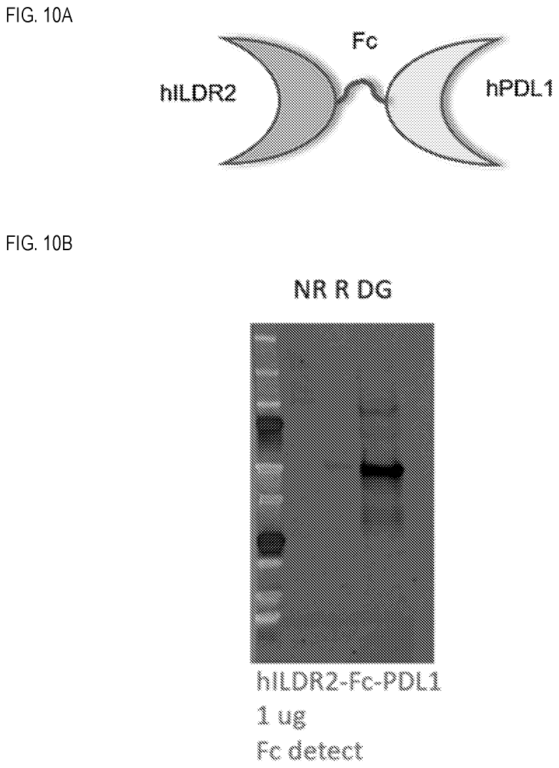

A construct encoding a human ILDR2- and PD-L1-based chimeric protein was generated. The “hILDR2-Fc-PD-L1” construct included an extracellular domain (ECD) of human ILDR2 fused to an ECD of human PD-L1 via a hinge-CH2-CH3 Fc domain derived from IgG1. See, FIG. 10A.

The construct was prepared as described in Example 1 above. A western blot analysis was performed to validate the detection and binding of the Fc domain of hILDR2-Fc-PD-L1 to an anti-Fc antibody (FIG. 10B).

Functional ELISA was performed to demonstrate the binding affinity of the Fc binding domain of hILDR2-Fc-PD-L1 to an anti-Fc antibody. As shown in FIG. 10C, binding of the Fc portion of the hILDR2-Fc-PD-L1 chimeric protein was characterized by capturing the chimeric protein to a plate-bound mouse IgG Fc gamma antibody and detecting via an HRP conjugated anti-mouse Fc (H+L) antibody. A mouse whole IgG was used to generate a standard curve. The starting concentration for the chimeric protein was 60 μg/ml.

### Example 8: Construction and Characterization of an Illustrative Human IL-6R- and Human IL-35-Based Chimeric Protein

A construct encoding a human IL-6R- and IL-35-based chimeric protein was generated. The “human IL-6R-Fc-IL-35” construct included an extracellular domain (ECD) of human IL-6R fused to an ECD of human IL-35 via a hinge-CH2-CH3 Fc domain derived from IgG1.

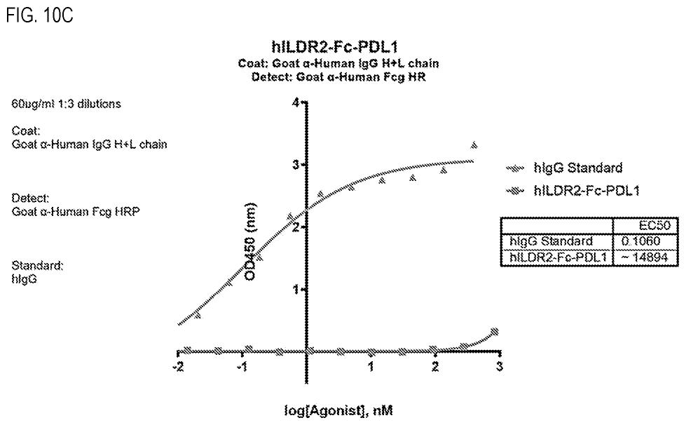

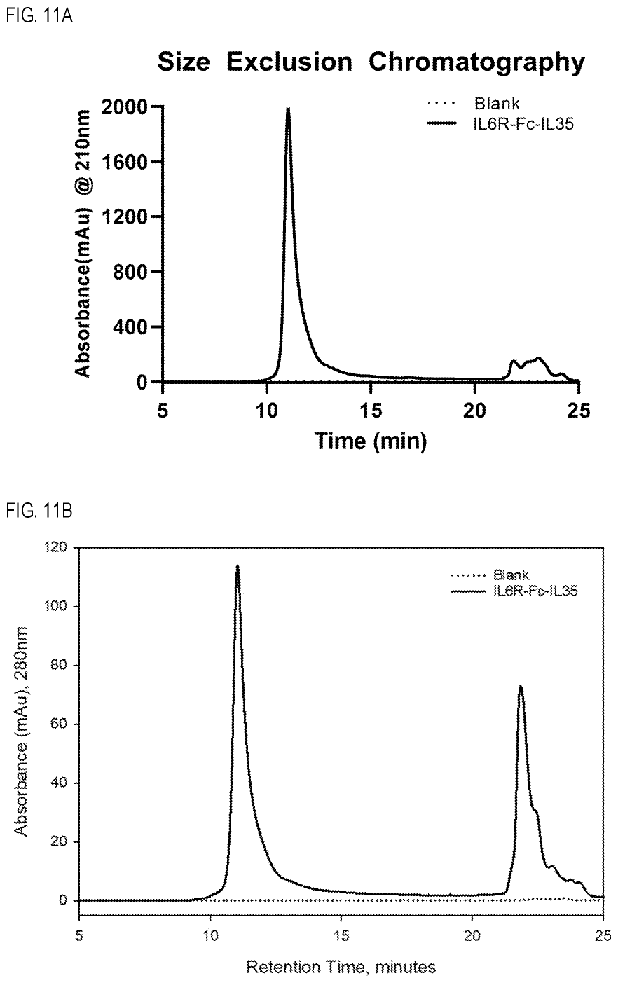

The construct was prepared as described in Example 1 above. The purified human IL-6R-Fc-IL-35 was analyzed by Size Exclusion Chromatography (SEC). As shown in FIG. 11A and FIG. 11B, the human IL-6R-Fc-IL-35 protein was pure. It ran with expected molecular weight. The large peak in FIG. 11B (Absorbance at 220 nm) between 20-25 minutes is like to be because of buffer components. Absence of that peak in FIG. 11A (Absorbance at 280 nm), along with The protein peak between 10-15 minutes was consistent and sharp between the 2 wavelengths, suggesting that the human IL-6R-Fc-IL-35 chimeric protein was very homogeneous.

### Example 9: The Human IL-6R-Fc-IL-35 Chimeric Protein Acts as an IL-6-Sink

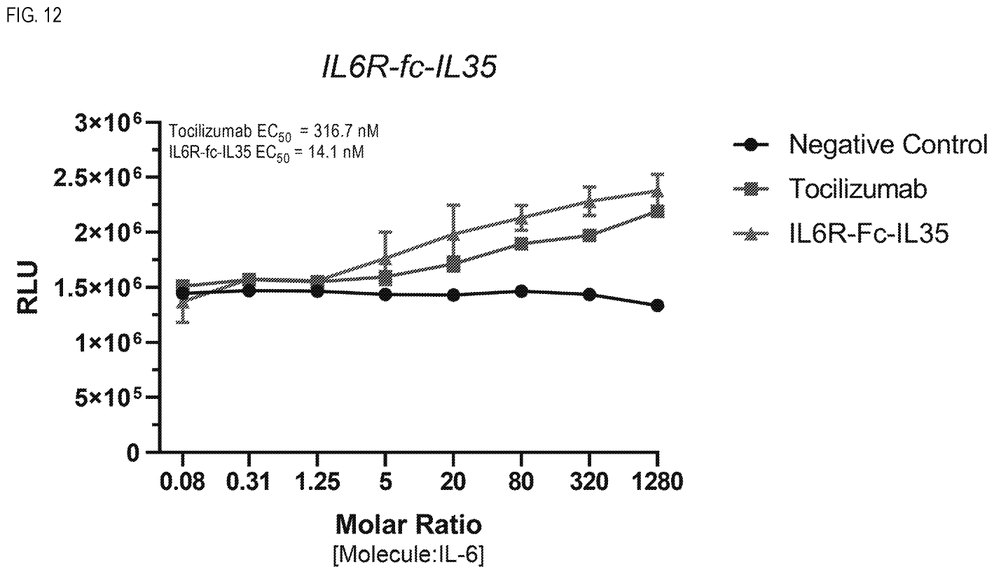

One of the objectives of this experiment was to understand whether the human IL-6R-Fc-IL-35 chimeric protein can effectively bind and neutralize IL-6. Towards those objectives, the DS-1 cell line, which is an IL-6 dependent B cell line, was used. The DS-1 cells, which depend on IL-6 for survival, do not synthesize IL-6. Thus, IL-6 must be added exogenously to cultivate these cells in vitro. DS-1 cells were cultured for 24 hours in the presence of IL-6 and increasing molar ratios to IL-6 of tocilizumab, the IL-6R-Fc-IL-35 chimeric protein or a control chimeric protein. Tocilizumab, which is an IL-6 receptor-blocker recombinant humanised monoclonal antibody directed against interleukin-6 (IL-6) receptor, was used as a positive control. The control chimeric protein, which does not bind IL-6 or IL-6 receptor, was used as a negative control for IL-6 binding. To quantitate apoptosis, the induction of caspase 3/7 was measured by a luciferase assay. The data are shown in FIG. 12. Increasing RLUs indicate increasing cell death caused by caspase 3/7 activation. As shown in FIG. 12, the IL-6R-Fc-IL-35 chimeric protein induced a dose-dependent apoptosis in DS-1 cells with an EC₅₀ of 14.1 nM. In comparison, the control chimeric protein, which acted as a negative control showed no increase in apoptosis even at very high molar ratios (FIG. 12). Tocilizumab also exhibited induced dose-dependent apoptosis in DS cells with an EC₅₀ of 316.7 nM. These data show that the IL-6 side of the molecule is approximately 22 times more potent at sequestering IL-6 compared to tocilizumab.

These data demonstrate that the human IL-6R-Fc-IL-35 chimeric protein acts as an IL-6 sink with a low nM EC₅₀. Therefore, the human IL-6R-Fc-IL-35 chimeric protein may be used in therapeutic methods where neutralizing IL-6 is desired.

### Example 10: The Human IL-6R-Fc-IL-35 Chimeric Protein Regulates Cell Proliferation and Cytokine Production by CD4+ Cells

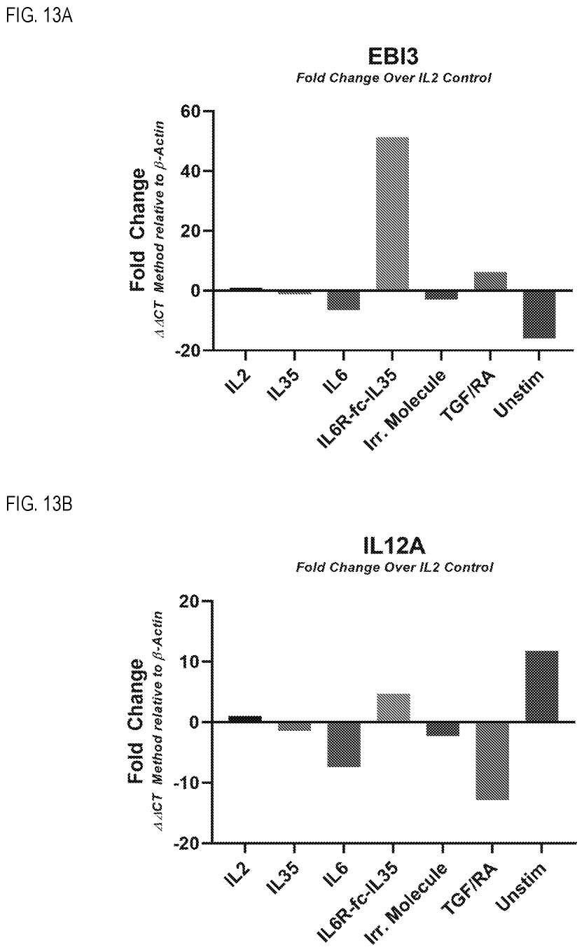

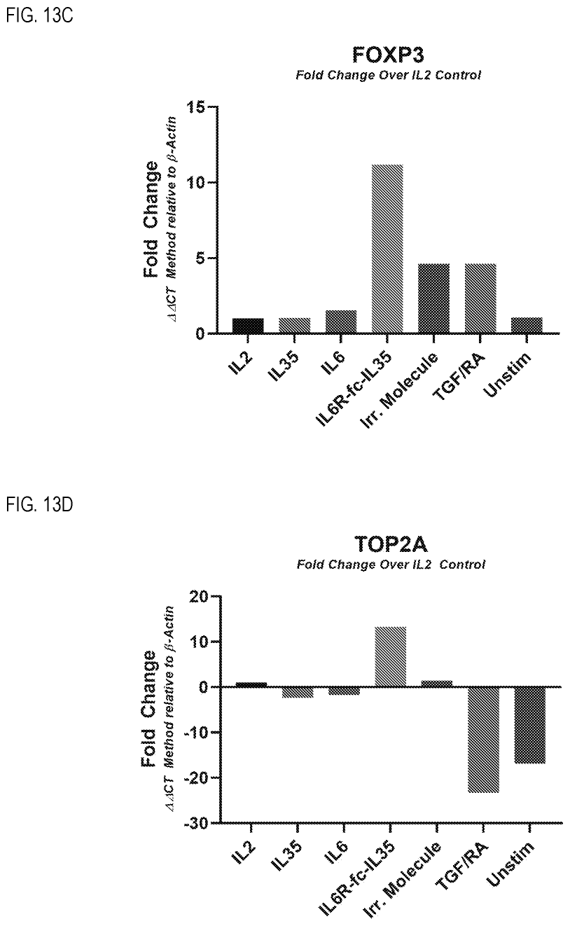

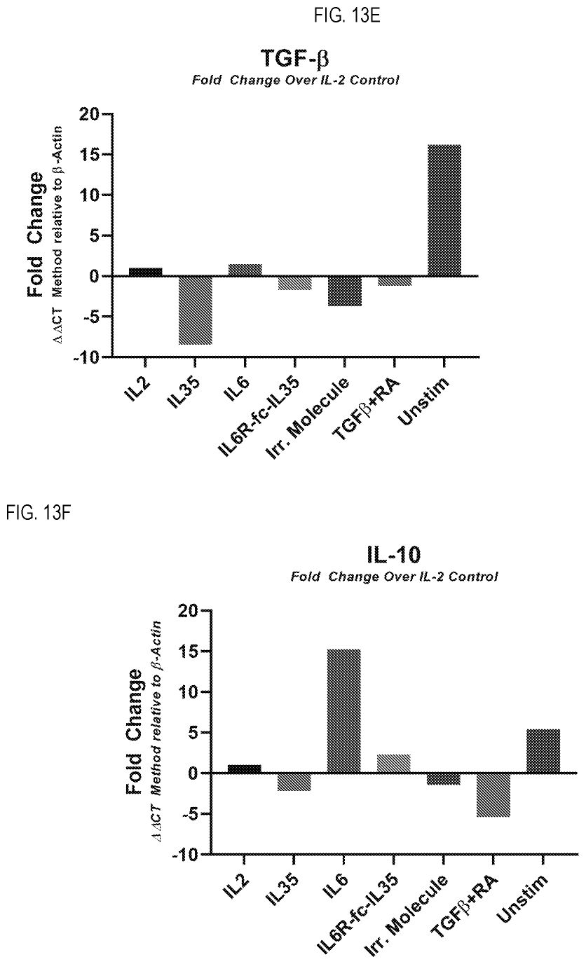

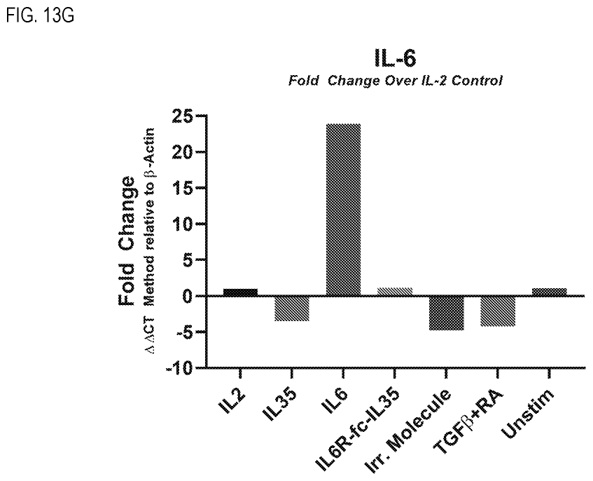

One of the objectives of this experiment was to understand the effect of the human IL-6R-Fc-IL-35 chimeric protein on CD4 T cells. Towards those objectives, purified, naïve splenic CD4 T cells were cultured in the presence of anti-CD3/anti-CD28 beads and one of IL-35, the IL-6R-Fc-IL-35 chimeric protein, IL-2, and TGF-β/Retinoic Acid (RA) for 9 days. since retinoic acid; with TGFβ (TGF-6/RA) is a Treg inducer, this molecule served as a control. An unrelated chimeric protein was used as a negative control. On day 9, the cells were harvested, and mRNA isolated. Reverse transcriptase-qPCR was performed to quantify the relative levels of mRNA of various genes. FIG. 13A to FIG. 13G show the data as fold change over control using the delta delta CT method of relative quantitation. IL-35 is known to induce itself, in a positive feedback loop. As shown in FIG. 13A and FIG. 13B, EBI3 and IL-12A, the components of IL-35, were induced by the human IL-6R-Fc-IL-35 chimeric protein. In contrast, IL-2, or TGF-β/RA or the unrelated chimeric protein did not consistently induce EBI3 and IL-12A.

These data indicate that the human IL-6R-Fc-IL-35 chimeric protein can induce IL-35 production. Therefore, the human IL-6R-Fc-IL-35 chimeric protein may be used in therapeutic methods where induction of IL-35 is desired.

As shown in FIG. 13C, the human IL-6R-Fc-IL-35 chimeric protein induced FOXP3, the master regulator of iTreg and nTreg, compared to IL-2. TGF/RA also induced FOXP3 compared to IL-2, albeit to a lesser extent compared to that of the human IL-6R-Fc-IL-35 chimeric protein (FIG. 13C). FOXP3 is essential for the production and normal function of regulatory T cells, which play an important role in preventing autoimmunity.

These data demonstrate that the human IL-6R-Fc-IL-35 chimeric protein induces the production of a master regulator of iTreg and nTreg. Accordingly, the human IL-6R-Fc-IL-35 chimeric protein may be used in therapeutic methods where suppressing an immune response or decreasing the severity of autoimmunity is desired.

As shown in FIG. 13D, the human IL-6R-Fc-IL-35 chimeric protein, as compared to IL-2, induced TOP2A, which is a marker of cellular proliferation. In contrast, TGF-β/RA, or any other molecules tested herein did not induce TOP2A (FIG. 13D). These data demonstrate that the human IL-6R-Fc-IL-35 chimeric protein promotes the development of Tregs while also being more permissive for the lymphocyte proliferation. Accordingly, the human IL-6R-Fc-IL-35 chimeric protein may be used in therapeutic methods where suppressing an immune response or decreasing the severity of autoimmunity is desired, wherein the method has lesser side effects compared to conventionally used immunosuppressants.

IL-35 is known to repress the production of TGF-β and IL-10. Therefore, the effect of the human IL-6R-Fc-IL-35 chimeric protein, as compared to IL-2, on the expression of TGF-β and IL-10 was studied. As shown in FIG. 13E, the human IL-6R-Fc-IL-35 chimeric protein suppressed the TGF-β expression. This was similar to IL-35, although to a lesser extent FIG. 13F). Interestingly, as shown in FIG. 13F, the human IL-6R-Fc-IL-35 chimeric protein did not inhibit the expression of IL-10, unlike IL-35. Indeed, the IL-10 production was induced 2× compared to the control (FIG. 13F). IL-10 is an anti-inflammatory cytokine, which inhibits the activity of Th1 cells, NK cells, and macrophages, all of which are required for optimal immune responses against pathogens but also contribute to tissue damage during an overactive immune response such as autoimmunity.

As shown in FIG. 13G, the human IL-6R-Fc-IL-35 chimeric protein, as compared to IL-2, did not induce or inhibit the expression of IL-6, while neutralizing any preexisting IL-6, as shown above.

These data demonstrate that the human IL-6R-Fc-IL-35 chimeric protein suppresses TGF-β expression but allows normal or slightly induced IL-10 production. Accordingly, the human IL-6R-Fc-IL-35 chimeric protein may be used in therapeutic methods for suppressing an immune response or decreasing the severity of autoimmunity and preventing tissue damage.

### Example 11: The CD4+ Cells Stimulated by the Human IL-6R-Fc-IL-35 Chimeric Protein Suppress CD8 Cell Proliferation

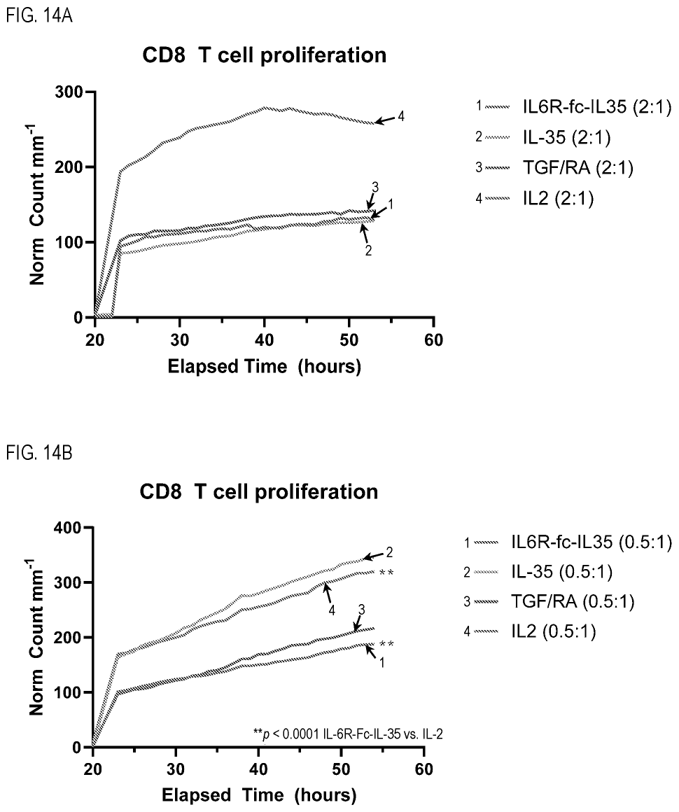

One of the objectives of this experiment was to understand whether CD4 T cells stimulated by the human IL-6R-Fc-IL-35 chimeric protein have any effects on CD8 cells. Towards those objectives, naïve splenic CD4 T cells were cultured in the presence of anti-CD3/anti-CD28 beads and one of IL-35, the human IL-6R-Fc-IL-35 chimeric protein, IL-2, and TGFβ/RA for 9 days. Syngeneic CD8 T cells were purified on day 9 and stained with carboxyfluorescein succinimidyl ester (CFSE). Varying numbers of CD4 cells to a fixed number of CD8 T cells were co-cultured in the presence of fresh anti-CD3/anti-CD28 beads. Proliferation was assessed by live imaging using the Incucyte live-cell imaging platform for 3 days. As shown in FIG. 14A, when CD4 cells:CD8 T cell ratio was 2:1, each of IL-35, the human IL-6R-Fc-IL-35 chimeric protein, and TGFβ/RA inhibited the proliferation of CD8 cells compared to IL-2, which promoted T-cell proliferation. As shown in FIG. 14B, when CD4 cells:CD8 T cell ratio was 0.5:1, the human IL-6R-Fc-IL-35 chimeric protein (p<0.0001), and TGFβ/RA inhibited the proliferation of CD8 cells compared to IL-2, which promoted T-cell proliferation. Interestingly, IL-35 did not inhibit CD8 cell proliferation. Therefore, the CD4 T cells cultured in the presence of the human IL-6R-Fc-IL-35 chimeric protein or TGF-6/RA were more potent suppressers CD8 cell proliferation than CD4 T cells cultured in the presence of IL-35.

These data demonstrate that the human IL-6R-Fc-IL-35 chimeric protein promotes the inhibition of proliferation of CD8 cells by CD4 cells. Accordingly, the human IL-6R-Fc-IL-35 chimeric protein may be used in therapeutic methods where suppressing an immune response or decreasing the severity of autoimmunity.

### Example 12. Construction and Characterization of an Illustrative Murine IL-6R- and IL-35-Based Chimeric Protein

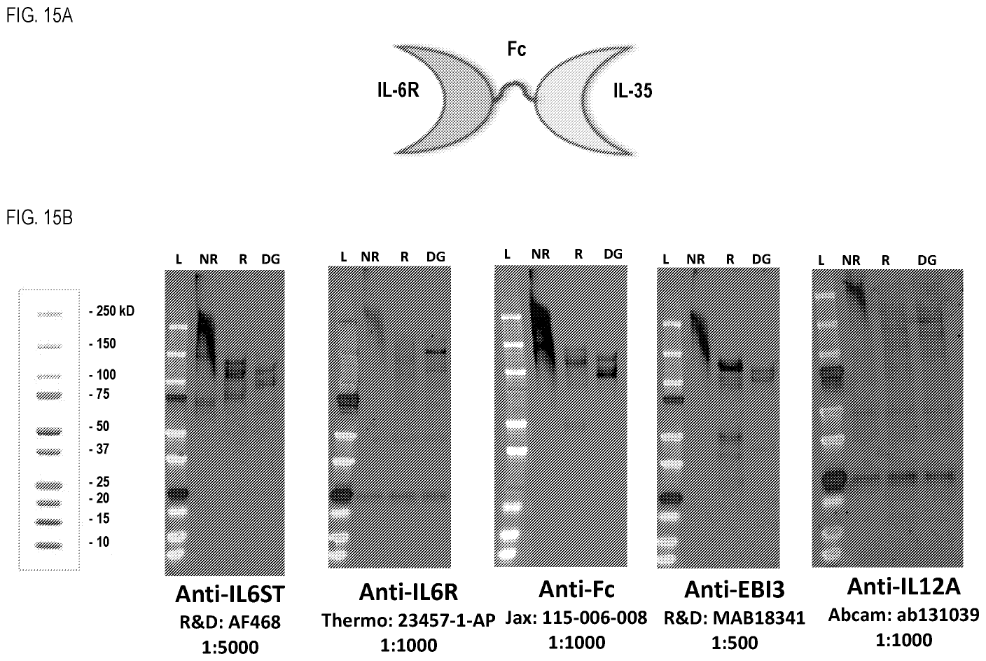

A construct encoding a murine IL-6R- and IL-35-based chimeric protein was generated. The “mIL-6R-Fc-IL-35” construct included an extracellular domain (ECD) of murine IL-6R fused to an ECD of murine IL-35 via a hinge-CH2-CH3 Fc domain derived from IgG1. See, FIG. 15A.

The construct was prepared as described in Example 1 above. A western blot analysis was performed to validate the detection and binding of the Fc domain of the mIL-6R-Fc-IL-35 chimeric protein to anti-IL-6ST, anti-IL-6R, anti-Fc, anti-EBI3 and anti-IL-12A antibodies (FIG. 15B). As shown, both subunits of IL-6R and IL-35 were detected, as would be expected of the heterodimeric protein.

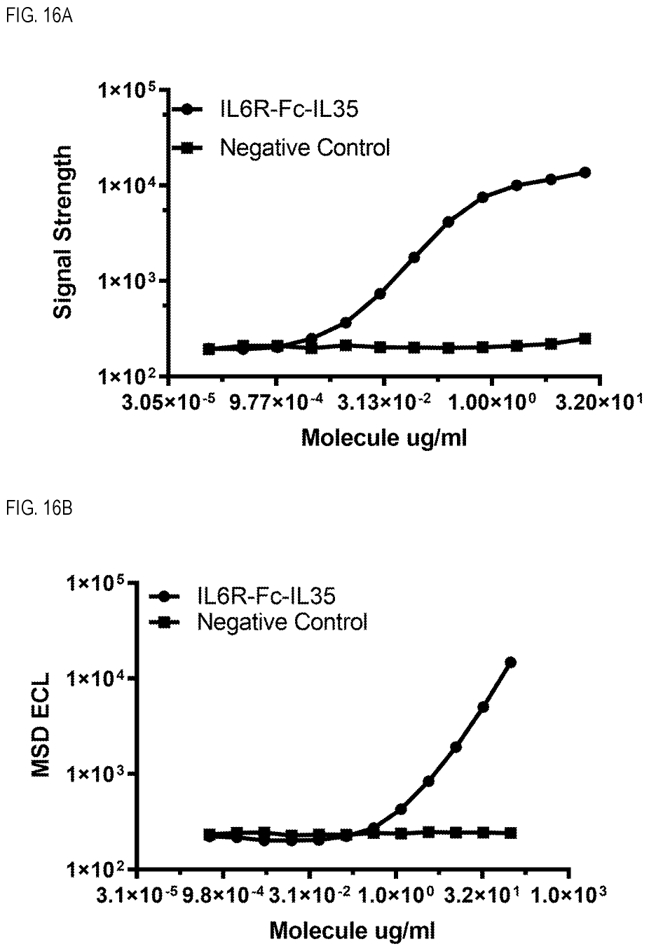

To understand whether the mIL-6R-Fc-IL-35 chimeric protein can simultaneous bind to ligands of both mIL-6R and mIL-35, a sandwich ELISA was performed. An anti-IL-6ST antibody was coated on plates. Increasing amounts of the IL-6R-Fc-IL-35 chimeric protein or the TNFR2-Fc-TGFβ chimeric protein were added to the plate for capture by the plate-bound anti-IL-6ST antibody. The binding was detected using an anti-IL-12A antibody. As shown in FIG. 16A, the IL-6R-Fc-IL-35 chimeric protein but not the TNFR2-Fc-TGFβ chimeric protein showed a dose-dependent signal. In another experiment, an anti-IL-6R antibody was coated on plates. Increasing amounts of the IL-6R-Fc-IL-35 chimeric protein or the TNFR2-Fc-TGFβ chimeric protein were added to the plate for capture by the plate-bound anti-IL-6R antibody. The binding was detected using an anti-IL-27B antibody. As shown in FIG. 16B, the IL-6R-Fc-IL-35 chimeric protein but not the TNFR2-Fc-TGFβ chimeric protein showed a dose-dependent signal.

These data demonstrate that the IL-6R-Fc-IL-35 chimeric protein can simultaneously bind to ligands of both mIL-6R and mIL-35. These data also illustrate that the IL-6R-Fc-IL-35 chimeric protein contains both subunits of both IL-6R and IL-35.

### Example 13. Construction and Characterization of an Illustrative Murine MadCAM- and CCL25-Based Chimeric Protein

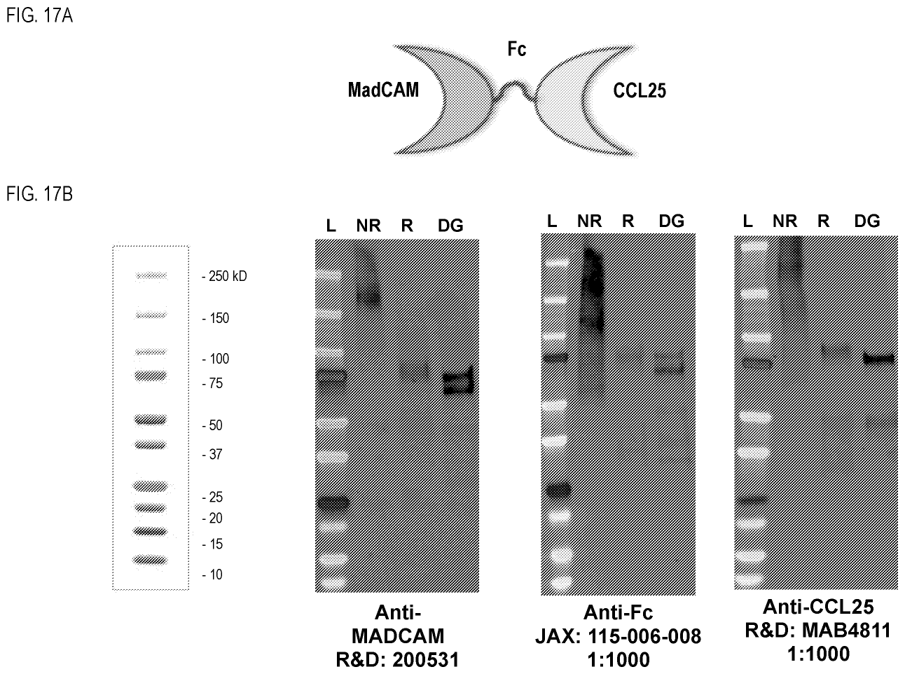

A construct encoding a murine MadCAM- and CCL25-based chimeric protein was generated. The “mMadCAM-Fc-CCL25” construct included an extracellular domain (ECD) of murine MadCAM fused to an ECD of murine CCL25 via a hinge-CH2-CH3 Fc domain derived from IgG1. See, FIG. 17A.

The construct was prepared as described in Example 1 above. A western blot analysis was performed to validate the detection and binding of the MadCAM, Fc and CCL25 domains of the mMadCAM-Fc-CCL25 chimeric protein to anti-MadCAM, anti-Fc, and anti-CCL25 antibodies (FIG. 17B).

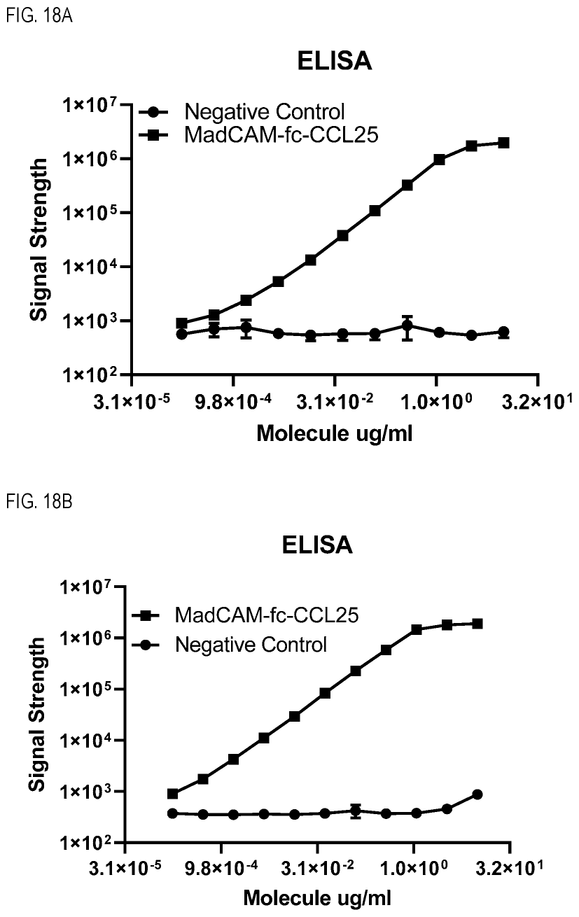

To understand whether the mMadCAM-Fc-CCL25 chimeric protein can simultaneous bind to ligands of both MadCAM and CCL25, a sandwich ELISA was performed. An anti-CCL25 antibody was coated on plates. Increasing amounts of the MadCAM-Fc-CCL25 chimeric protein or the TNFR2-Fc-TGFβ chimeric protein were added to the plate for capture by the plate-bound anti-CCL25 antibody. The binding was detected using an anti-MadCAM antibody. As shown in FIG. 18A, the mMadCAM-Fc-CCL25 chimeric protein but not the TNFR2-Fc-TGFβ chimeric protein showed a dose-dependent signal. In another experiment, an anti-MadCAM antibody was coated on plates. Increasing amounts of the MadCAM-Fc-CCL25 chimeric protein or the TNFR2-Fc-TGFβ chimeric protein were added to the plate for capture by the plate-bound anti-MadCAM antibody. The binding was detected using an anti-CCL25 antibody. As shown in FIG. 18B, the mMadCAM-Fc-CCL25 chimeric protein but not the TNFR2-Fc-TGFβ chimeric protein showed a dose-dependent signal.

These data demonstrate that the mMadCAM-Fc-CCL25 chimeric protein can simultaneously bind to ligands of both mIL-6R and mIL-35.

### Example 14. Construction and Characterization of an Illustrative Murine α4β7- and IL-35-Based Chimeric Protein

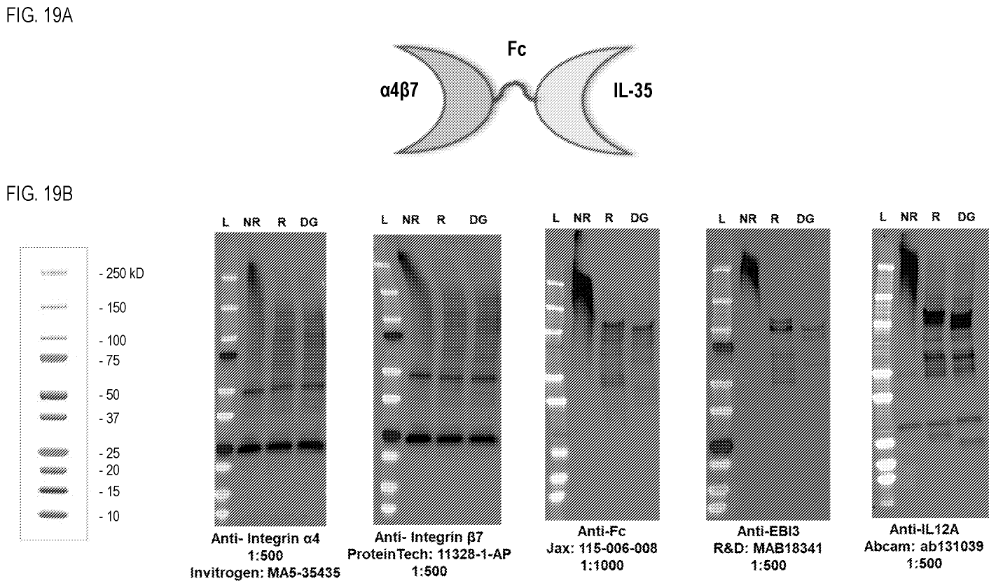

A construct encoding a murine α4β7- and IL-35-based chimeric protein was generated. The “α4β7-Fc-IL-35” construct included an extracellular domain (ECD) of murine α4β7 fused to an ECD of murine IL-35 via a hinge-CH2-CH3 Fc domain derived from IgG1. See, FIG. 19A.

The construct was prepared as described in Example 1 above. A western blot analysis was performed to validate the detection and binding of the α4, β7, Fc, and IL-35 domains of the α4β7-Fc-IL-35 chimeric protein to an anti-α4, anti-37, anti-Fc, anti-EBI3 or anti-IL-12A antibodies (FIG. 19B).

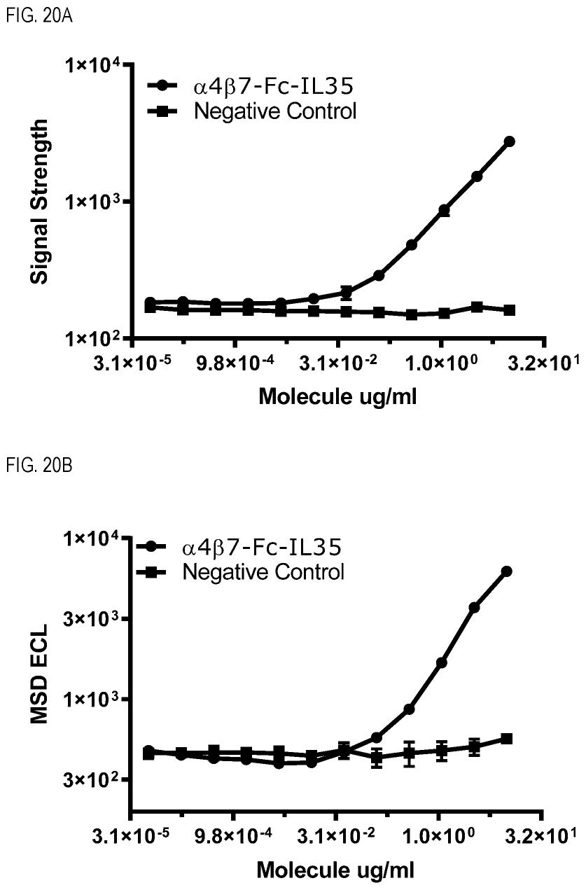

To understand whether the α4β7-Fc-IL-35 chimeric protein can simultaneous bind to ligands of α4β7 and IL-35, a sandwich ELISA was performed. An anti-α4 antibody was coated on plates. Increasing amounts of the α4β7-Fc-IL-35 chimeric protein or the TNFR2-Fc-TGFβ chimeric protein were added to the plate for capture by the plate-bound anti-α4 antibody. The binding was detected using an anti-IL27B antibody. As shown in FIG. 20A, the α4β7-Fc-IL-35 chimeric protein but not the TNFR2-Fc-TGFβ chimeric protein showed a dose-dependent signal. In another experiment, an anti-IL-12A antibody was coated on plates. Increasing amounts of the α4β7-Fc-IL-35 chimeric protein or the TNFR2-Fc-TGFβ chimeric protein were added to the plate for capture by the plate-bound anti-IL-12A antibody. The binding was detected using an anti-37 antibody. As shown in FIG. 20B, the α4β7-Fc-IL-35 chimeric protein but not the TNFR2-Fc-TGFβ chimeric protein showed a dose-dependent signal.

These data demonstrate that the α4β7-Fc-IL-35 chimeric protein can simultaneously bind to ligands of both mIL-6R and mIL-35. These data also illustrate that the IL-6R-Fc-IL-35 chimeric protein contains both subunits of both α4β7 and IL-35.

### Example 15. Construction and Characterization of an Illustrative Murine TNFR2- and TGFβ-Based Chimeric Protein

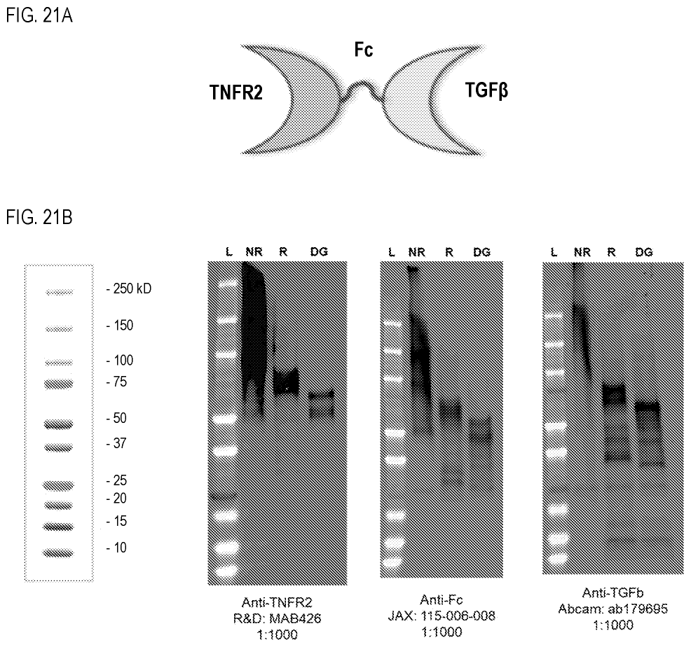

A construct encoding a murine TNFR2- and TGFβ-based chimeric protein was generated. The “TNFR2-Fc-TGF3” construct included an extracellular domain (ECD) of murine TNFR2 fused to an ECD of murine TGFβ via a hinge-CH2-CH3 Fc domain derived from IgG1. See, FIG. 21A.

The construct was prepared as described in Example 1 above. A western blot analysis was performed to validate the detection and binding of the TNFR2, Fc, and TGFβ domains of the TNFR2-Fc-TGFβ chimeric protein to an anti-TNFR2, anti-Fc, or anti-TGFβ antibodies (FIG. 21B).

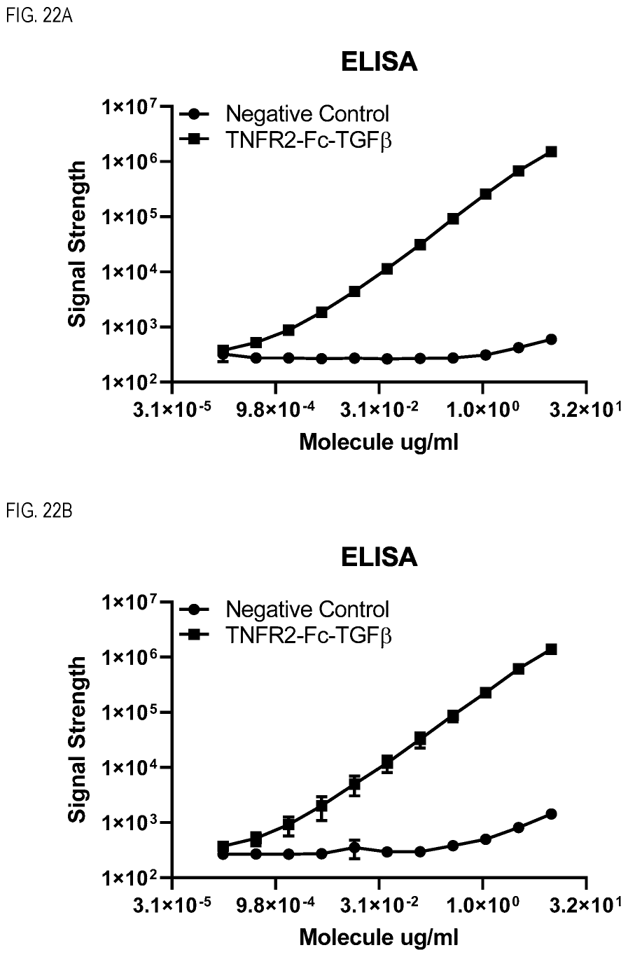

To understand whether the TNFR2-Fc-TGF3 chimeric protein can simultaneous bind to ligands of TNFR2 and-TGFβ, a sandwich ELISA was performed. An anti-TGFβ antibody was coated on plates. Increasing amounts of the TNFR2-Fc-TGFβ chimeric protein or the MadCAM-Fc-CCL chimeric protein were added to the plate for capture by the plate-bound anti-TGFβ antibody. The binding was detected using an anti-TNFR2 antibody. As shown in FIG. 22A, the TNFR2-Fc-TGFβ chimeric protein but not the MadCAM-Fc-CCL chimeric protein showed a dose-dependent signal. In another experiment, an anti-TNFR2 antibody was coated on plates. Increasing amounts of the TNFR2-Fc-TGFβ chimeric protein or the MadCAM-Fc-CCL chimeric protein were added to the plate for capture by the plate-bound anti-TNFR2 antibody. The binding was detected using an anti-TGFβ antibody. As shown in FIG. 22B, the TNFR2-Fc-TGFβ chimeric protein but not the MadCAM-Fc-CCL chimeric protein showed a dose-dependent signal.

These data demonstrate that the TNFR2-Fc-TGFβ chimeric protein can simultaneously bind to ligands of both TNFR2 and TGFβ.

### Example 16. In Vivo Efficacy of the Chimeric Proteins of the Present Disclosure in Mouse Model of Colitis

One of the objectives of this experiment was to understand the differences in the mesenteric lymph node (MLN) cell populations between normal and subjects suffering from colitis. Towards those objectives, C57BL/6 mice were weighed and sorted into treatment groups. On day 0, experimental treatment group animals were administered 3% dextran sodium sulfate (DSS) in their drinking water, ad libitum for 8 days. Control animals were administered unmodified drinking water. On days 0, 1, and 2, experimental treatment group animals were intraperitoneally administered 100 μg of one of the α4β7-Fc-IL-35, MadCAM-Fc-CCL25, IL-6R-Fc-IL-35, and TNFR2-Fc-TGFβ chimeric proteins, once daily. Control animals were administered 100 μg of murine IgG. The animals were monitored daily for signs of distress and weighed. On Day 9, DSS containing drinking water was replaced with unmodified drinking water. On day 11, all animals were sacrificed, and mesenteric lymph nodes (MLN) were harvested for analysis. Cells were extracted from MLN. A 15-parameter FACS panel was designed to phenotypically characterize the cellular composition of the MLN. The 15-parameter FACS panel is shown in the Table below:

15-parameter FACS Panel

| ID | Antigen | Fluorophore |
|---|---|---|
| 1 | B220 | BV421 |
| 2 | F4/80 | BV510 |
| 3 | CD11b | BV570 |
| 4 | Ly6G | BV605 |
| 5 | CD4 | BV650 |
| 6 | SiglecF | PE |
| 7 | CCR7 | PE/Dazzle594 |
| 8 | CD11c | PE/Cy5 |
| 9 | CD3 | PE/Cy7 |
| 10 | CD19 | AF488 |
| 11 | CCR5 | PerCP |
| 12 | MHC Class II | AF647 |
| 13 | CD73 | AF700 |
| 14 | CD8 | APC/Cy7 |
| 15 | SSC-A | 405 nm |

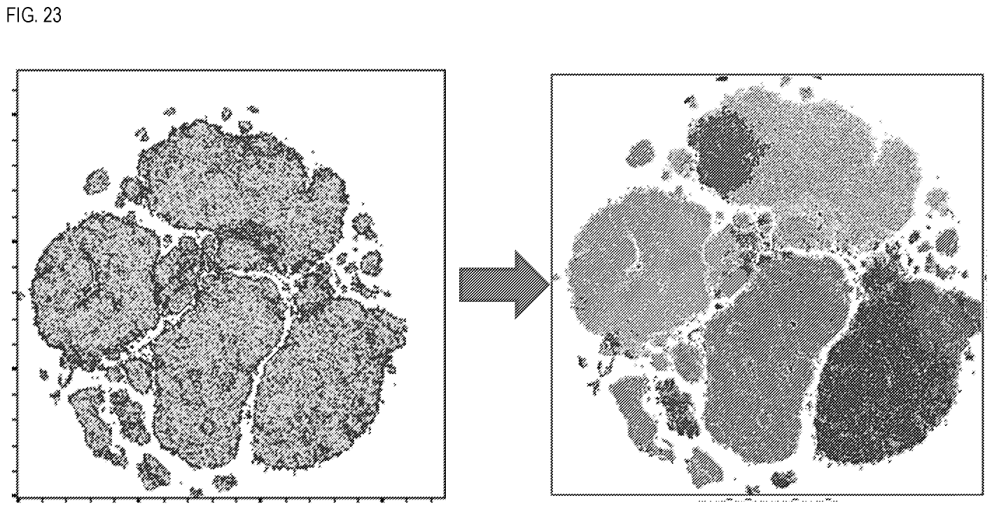

MLN cells from all animals were stained with the 15-parameter FACS panel and flow cytometry was carried out using a cytometer. The data were analyzed using FlowJo. The data was subjected to dimensionality reduction with t-distributed stochastic neighbor embedding (t-SNE) and phenotypic populations mapped with X-Shift. Population identity was established by visual inspection of antigen histogram staining patterns. As shown in FIG. 23, the MLN cell populations could be separated in distinct phenotypic populations of distinct X-shifts when untreated and DSS-treated mice were compared.

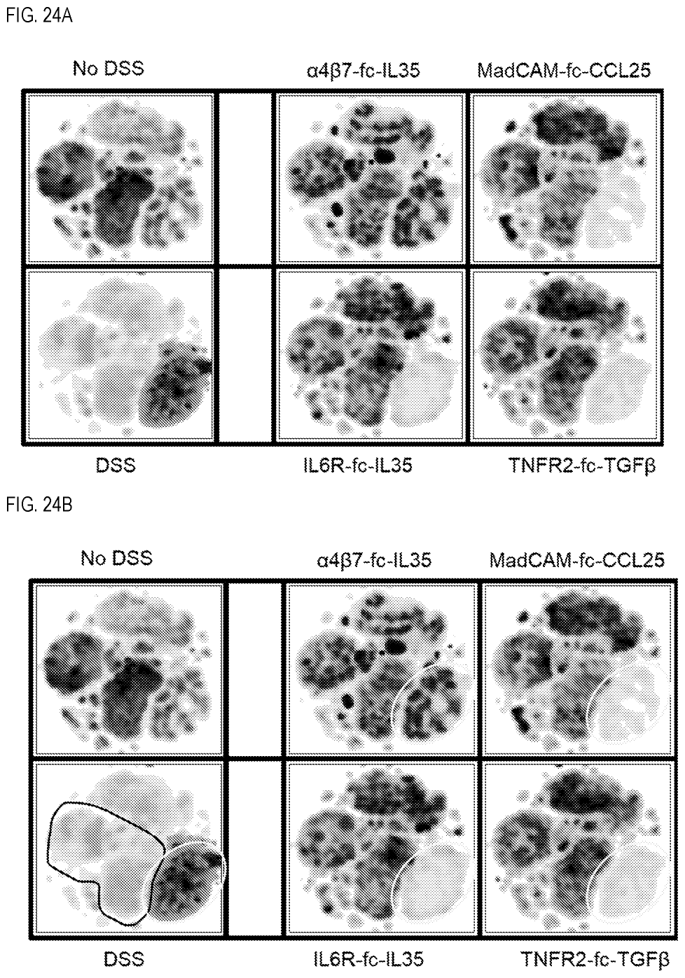

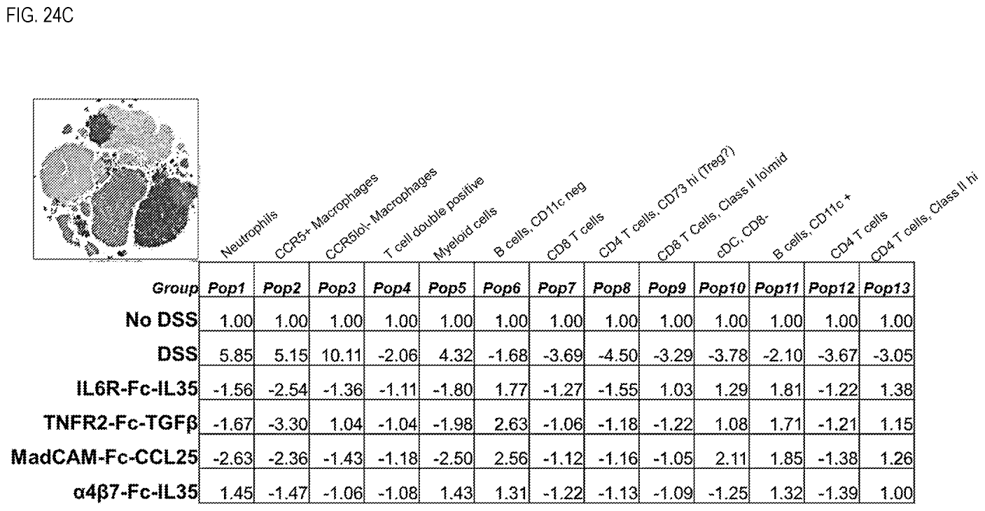

To understand whether the chimeric proteins of the present disclosure restored the cell populations to a normal-like state, a comparison of the t-SNE density plots from the mesenteric lymph node (MLN) cell populations of normal mice, the mice treated with DSS and murine IgG, and the mice treated with DSS and a chimeric protein disclosed herein was made. As shown in FIG. 24A, the DSS treatment led to intensification of some areas and reduction of intensity on other areas compared to control mice (no DSS). The treatment with the MadCAM-Fc-CCL25, IL-6R-Fc-IL-35, and TNFR2-Fc-TGFβ chimeric proteins altered the population towards normal state. FIG. 24B marks an area of the lower, right part of t-SNE density plots with light colored ovals. As shown in FIG. 24B, the DSS treatment led to intensification of the signal within the region bracketed by the oval compared to control mice (no DSS). Interestingly, the treatment with the MadCAM-Fc-CCL25, IL-6R-Fc-IL-35, and TNFR2-Fc-TGFβ chimeric proteins decreased the cells within the area bracketed by the ovals (FIG. 24B). Further, as shown in FIG. 24B, the DSS treatment led to a reduction of intensity of the signal within another region to the left of oval, which is bracketed a black shape compared to control mice (no DSS). The treatment with the MadCAM-Fc-CCL25, IL-6R-Fc-IL-35, and TNFR2-Fc-TGFβ chimeric proteins increased the number of cells within the area corresponding to the area bracketed by the black shape (FIG. 24B). FIG. 24C illustrate the phenotypic differences in cells from MLN of mice induced to have colitis using DSS Population differences among treatment groups is shown. FIG. 24C tabulates the differences in relative abundance of various cell types. As shown in FIG. 24C, the DSS treatment led to significant alterations in the cellular composition of MLN cells compared to control mice (no DSS). For example, neutrophils, CCR5lo/− macrophages, and other myeloid lineage cells, which are known to be involved in inflammation, were markedly increased by DSS treatment. In addition, CD4 Tregulatory cells, which are known to inhibit proinflammatory cells, are notably decreased by DSS treatment. (FIG. 24C). Interestingly, the treatment with the MadCAM-Fc-CCL25, IL-6R-Fc-IL-35, and TNFR2-Fc-TGFβ chimeric proteins restored the cell populations to a normal-like state (FIG. 24C). Moreover, the treatment with the MadCAM-Fc-CCL25, IL-6R-Fc-IL-35, TNFR2-Fc-TGFβ and α4β7-Fc-IL-35 chimeric proteins decreased neutrophils and CCR5lo/− macrophages compared to the DSS-treated mice (FIG. 24C).

These data demonstrate that the chimeric proteins disclosed herein decrease inflammatory cells and reverse the cellular changes in lymph nodes brought about by colitis. Accordingly, the chimeric proteins disclosed herein may be used in therapeutic methods where suppressing inflammation and treating conditions such as colitis and inflammatory bowel disease (IBD).

### Example 17. The TNFR2-Fc-TGFβ Chimeric Protein Protects Cells from TNF-α Mediated Apoptosis

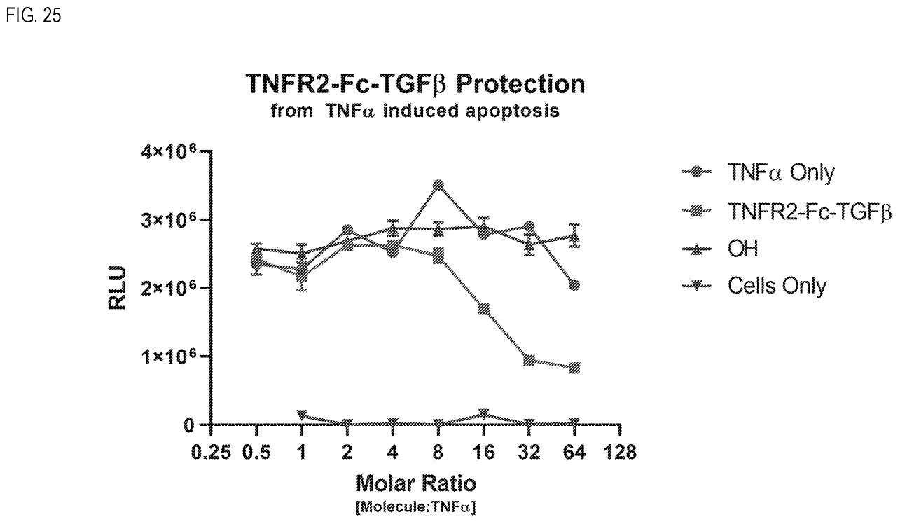

TNFα-induced apoptosis is believed to play a role in inflammation, autoimmunity and disorders such as IBD. One of the objectives of this experiment was to understand the effect of the TNFR2-Fc-TGFβ chimeric protein on INFα-induced apoptosis. Towards those objectives, L929 cells, which are known to be highly sensitive to TNF-α induced apoptosis, were used as an experimental model system. Fixed numbers of L929 cells were incubated in microtiter plates with 10 ng/ml of TNF-α for 24 hours. Control cells were grown without TNFα. Increasing molar ratios of the TNFR2-Fc-TGFβ chimeric protein or an irrelevant chimeric protein (OH) that is not known to protect cells from apoptosis were titrated into the plates. After 24 hours, the cells were assessed for cell death using the Caspase 3/7 CytoGlo system on the Promega GloMax Luminometer. As expected, L929 cells only (inverted triangles) showed a background level of apoptosis (FIG. 25). The addition of OH line (triangles) showed no evidence of a protective effect. The TNF-α only line (filled circles) caused the maximal amount of cell death induced in 24 hours (FIG. 25). On the other hand, as shown in FIG. 25, addition of the TNFR2-fc-TGFβ chimeric protein (squares) showed protection from apoptosis.

These data demonstrate that the TNFR2-Fc-TGFβ chimeric protein decreases TNFα-induced apoptosis. Accordingly, the TNFR2-Fc-TGFβ chimeric protein may be used in therapeutic methods where suppressing TNFα-induced apoptosis is desired. Accordingly, the TNFR2-Fc-TGFβ chimeric protein is useful for treating inflammation, autoimmunity and disorders such as IBD.

### Example 18. In Vivo Efficacy of the TNFR2-Fc-TGFβ Chimeric Protein in Mouse Model of Colitis

One of the objectives of this experiment was to understand whether the TNFR2-Fc-TGFβ chimeric protein is effective against ulcerative colitis and human Crohn's disease. Towards those objectives, the 4,6-trinitrobenzenesulfonic acid (TNBS)-induced colitis model was used. The TNBS-induced colitis model is a commonly utilized animal model that mimics the clinical pathology of Crohn's Disease. For example, this model is IL-12 mediated and involves components of both innate and adaptive immunity.

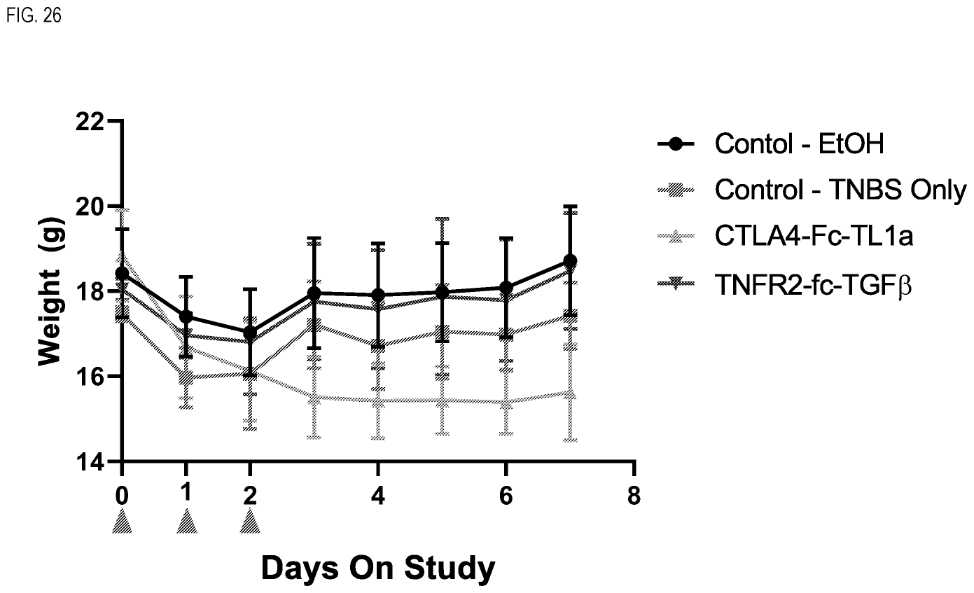

In brief, SJL mice were weighed and sorted into the following treatment groups: (1) control mice lacking colitis (ethanol only), (2) control mice having colitis (TNBS in ethanol), (3) the TNFR2-Fc-TGFβ chimeric protein treatment group, and (4) the CLTA4-Fc-TL1A chimeric protein treatment group. The control mice having colitis (TNBS in ethanol) and treatment group animals were administered a colonic instillation of 2.5% TNBS in ethanol on day 0. Control mice lacking colitis (ethanol only) were administered colonic instillation of ethanol alone. The TNFR2-Fc-TGFβ or CLTA4-Fc-TL1A chimeric proteins were intraperitoneally administered on days 0, 1, and 2, once daily. The control mice having colitis (TNBS in ethanol) group and the control mice lacking colitis (ethanol only) were intraperitoneally administered 100 μg of murine IgG on days 0, 1, and 2, once daily. The animals were monitored daily for signs of distress and weighed. The data are shown in FIG. 26. The body weights of the control mice lacking colitis (ethanol only; circles) show background changes (FIG. 26). In comparison, the control mice having colitis (TNBS in ethanol, squares) group showed an acute weight loss (FIG. 26) because of TNBS treatment-induced colonic damage. The treatment with the CLTA4-Fc-TL1A chimeric protein (triangles in FIG. 26) exacerbated disease when given concurrent with disease induction. In contrast, as shown in FIG. 26, the treatment with the TNFR2-Fc-TGFβ chimeric protein (inverted triangles) prevented severe weight loss. These results demonstrate that the TNFR2-Fc-TGFβ chimeric protein ameliorated severity of acute disease induced by TNBS.

On Day 8, all animals were sacrificed, and MLN were harvested for analysis. An 11-parameter FACS panel was designed to phenotypically characterize the cellular composition of the MLN. The 11-parameter FACS panel is shown in the Table below:

11-parameter FACS Panel

| ID | Antigen | Fluorophore |
|---|---|---|
| 1 | B220 | BV421 |
| 2 | F4/80 | BV510 |
| 3 | CD11b | BV570 |
| 4 | Ly6G | BV605 |
| 5 | CD4 | BV650 |
| 6 | SiglecF | PE |
| 7 | CD11c | PE/Cy5 |
| 8 | CD3 | PE/Cy7 |
| 9 | CD19 | AF488 |
| 10 | MHC Class II | AF647 |
| 11 | CD8 | APC/Cy7 |

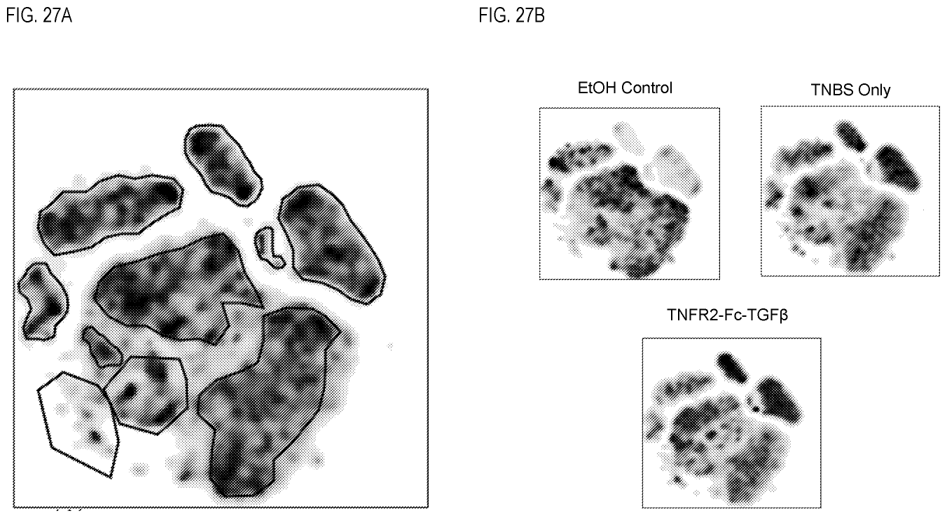

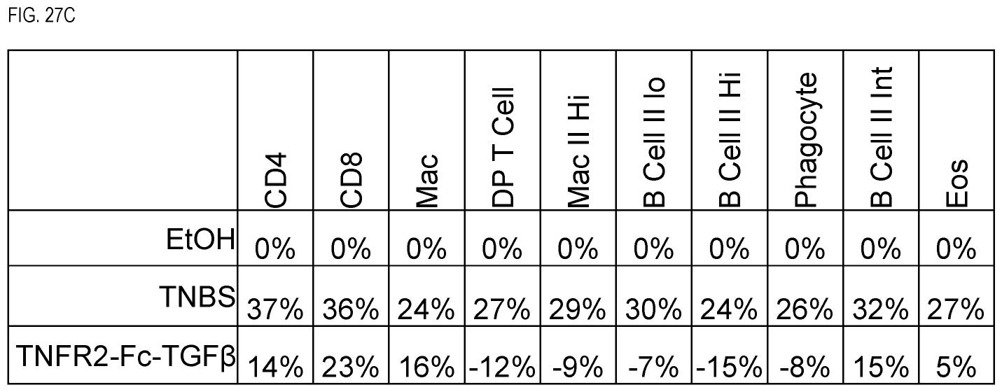

MLN cells from all animals were stained with the 11-parameter FACS panel and flow cytometry was carried out using a cytometer. The data were analyzed using FlowJo. The data was subjected to dimensionality reduction with t-distributed stochastic neighbor embedding (t-SNE) and phenotypic populations mapped with FIowSOM. Population identity was established by visual inspection of antigen histogram staining patterns. Population differences among treatment groups is shown in FIG. 27A.

To understand whether the TNFR2-Fc-TGFβ chimeric protein restored the cell populations to a normal-like state, a comparison of the t-SNE density plots from the mesenteric lymph node (MLN) cell populations of (1) control mice lacking colitis (treated with ethanol only), (2) control mice having colitis (treated with TNBS in ethanol), and (3) the TNFR2-Fc-TGFβ chimeric protein treatment group (the mice having colitis treated with the TNFR2-Fc-TGFβ chimeric protein) was made. As shown in FIG. 27B, the TNBS treatment led to intensification of some areas and reduction of intensity on other areas compared to control mice. The treatment with the TNFR2-Fc-TGFβ chimeric protein altered the population towards normal state. FIG. 27C illustrate the phenotypic differences in cells from MLN of mice induced to have colitis using DSS Population differences among treatment groups is shown. FIG. 27C tabulates the differences in relative abundance of various cell types. As shown in FIG. 27C, the TNBS treatment led to elevation in various cell types. Interestingly, the treatment with the TNFR2-Fc-TGFβ chimeric protein restored the cell populations to a normal-like state (FIG. 27C).

These data demonstrate that the TNFR2-Fc-TGFβ chimeric protein decreases inflammatory cells and reverse the cellular changes in lymph nodes brought about by ulcerative colitis and human Crohn's disease. Accordingly, the chimeric proteins disclosed herein may be used in therapeutic methods where suppressing inflammation and treating conditions such as ulcerative colitis and human Crohn's disease.

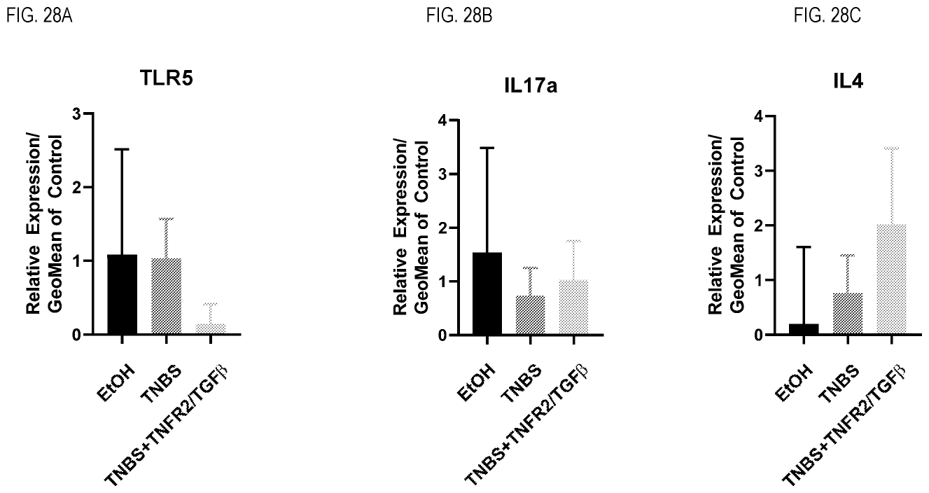

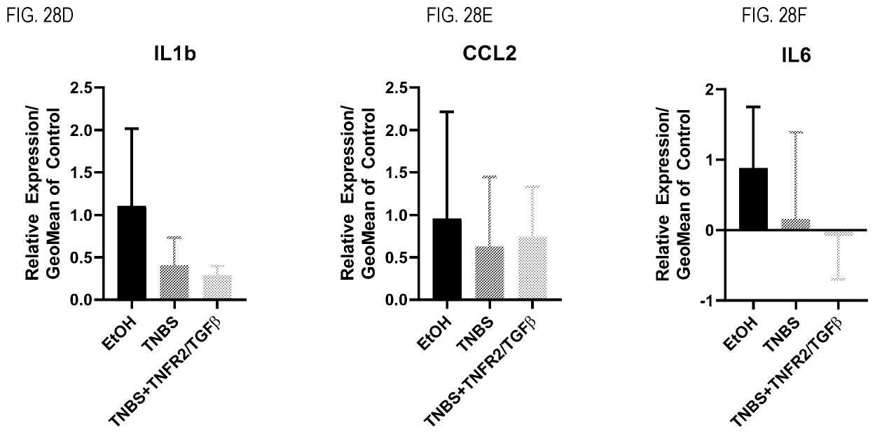

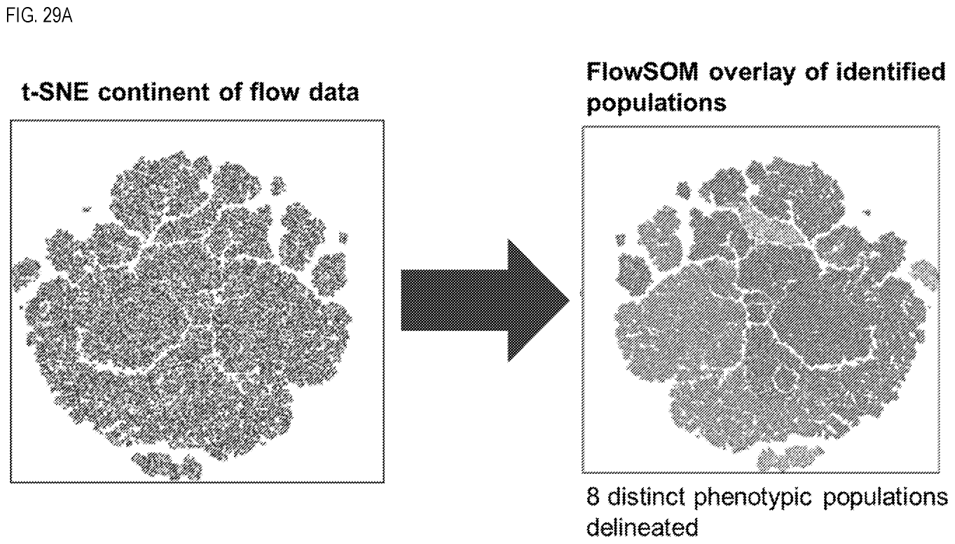

To understand the changes in gene expression, quantiplex gene analysis was performed. Briefly, total mRNA was isolated from the MLN of study animals. 39 transcripts were examined for expression differences among treatment groups by the quantiplex gene assay. Those transcripts were TGF-β, IL23R, IL-1β, IL-1RAP, IL-5, TLR4, IL-6RA, MyD88, CCL2, IL-17a, TLR5, IL-2, TLR7, EOMES, IL-1r1, IL-22RA, TLR9, IL-6, TLR8, IL-21, IL-18RAP, IL-1RA, IL-12a, IL-3, NFAT, IL-15, TLR2, IL4, TLR3, BLIMP1, FoxP3, TLR6, Jun, IL-7, IL-18, TLR1, IL-12b, TNF-α, and IL-12a. As shown in FIG. 28A, the expression of TLR5 decreased when colitic mice were also administered the TNFR2-Fc-TGFβ chimeric protein. TLR5 is a receptor that detects bacterial molecular patterns and initiates immune response. It's reduction suggests that innate cells would be rendered less responsive to bacteria, which would be protective in colitis. As shown in FIG. 28B, the expression of IL-17A remained substantially unchanged, indicating that the function of TH17 (gut specific adaptive immunity) was unaffected by treatment. Thus, the ability to respond to parasitic and viral pathogens would be unaffected. As shown in IL-4 (FIG. 28C), the expression of IL4, which is a TH2 polarizing cytokine, was elevated with treatment with the TNFR2-Fc-TGFβ chimeric protein. This observation suggests that suggesting that the treatment with the TNFR2-Fc-TGFβ chimeric protein skewed the T helper axis towards a less inflammatory state. As shown in FIG. 28D and in FIG. 28F, the expression of the proinflammatory cytokines IL-1B and IL-6 was reduced with the treatment with the TNFR2-Fc-TGFβ chimeric protein, suggesting that the TNFR2-Fc-TGFβ chimeric protein reduces inflammation. As shown in CCL-2 FIG. 28E, the expression of CCL2, which is a chemokine that recruits adaptive memory cells, remained unchanged. This observation suggests that the adaptive responses would be unaffected by treatment.

Collectively, these data demonstrate that the TNFR2-Fc-TGFβ chimeric protein skews the T helper axis towards a less inflammatory state, decreases inflammation, and reduced reactivity to normal flora, while keeping the adaptive responses unaffected. Accordingly, the chimeric proteins disclosed herein may be used in therapeutic methods where suppressing inflammation and treating conditions such as ulcerative colitis and human Crohn's disease.

### Example 19. The Chimeric Protein Disclosed Herein Induce Different Subpopulations of CD4 Cells

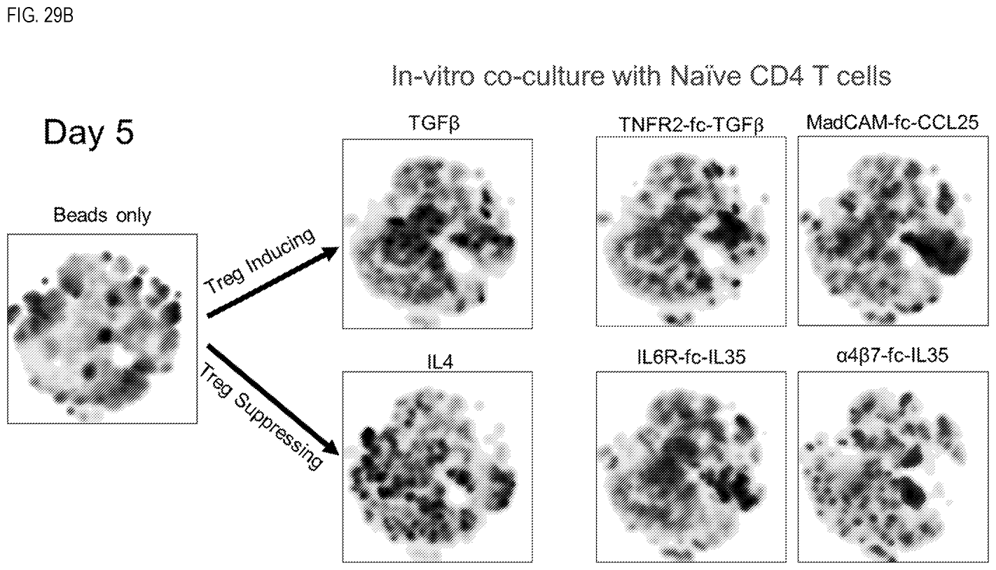

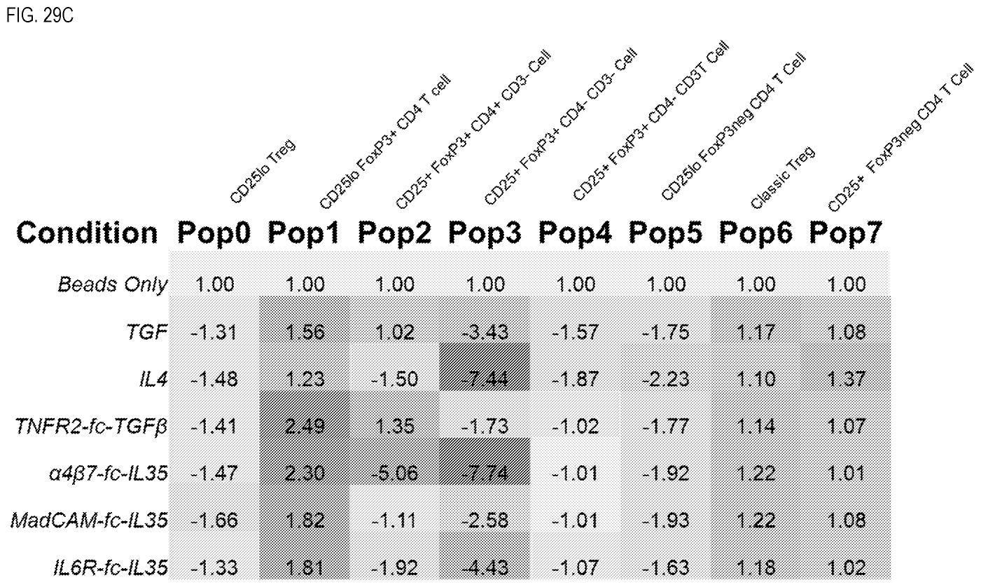

Naïve CD4 T cells were isolated from the spleens of FoxP3 RFP knock-in mice (FIR mice) by magnetic bead separation. These cells were antigen inexperienced and were FoxP3 negative. 10⁵ naïve CD4 T cells were cultured for 5 days with activating anti-CD3/anti-CD28 beads in the presence of one of IL-4, TGFβ, MadCAM-Fc-CCL25, TNFR2-Fc-TGFβ, α4β7-Fc-IL35, and IL6R-Fc-IL35. On Day 5, the cells where harvested and stained with a four parameter FACS panel was designed to interrogate basic CD4 Treg differentiation. The markers used in the four parameter FACS panel were CD3, CD4, and CD25. FoxP3 was visualized by the expression of RFP. Samples were then immediately analyzed by flow cytometry. Analysis was performed using FlowJo software. A t-SNE algorithm was applied to the data to generate a continent. FIowSOM was then applied to the continent to identify phenotypic populations. Populations were named by visual inspection and comparisons performed. FIowSOM was used to generate boundaries for 8 potential phenotypes. As shown in FIG. 29A, the t-SNE density plot of showed eight distinct phenotypic populations of CD4 cells. The differences resulting from treatment conditions (the treatment with of IL-4, TGFβ, MadCAM-Fc-CCL25, TNFR2-Fc-TGFβ, α4β7-Fc-IL35, IL6R-Fc-IL35) were then visualized. Since IL-4 and TGFβ promote and suppress Treg differentiation, respectively, they served as positive and negative control, respectively. As shown in FIG. 29B, like TGFβ, and unlike IL-4, the MadCAM-Fc-CCL25, TNFR2-Fc-TGFβ, α4β7-Fc-IL35, and IL6R-Fc-IL35 chimeric proteins showed a t-SNE pattern that was most consistent with Treg formation. Next, different cell types induced during this experiment were quantitated. As shown in FIG. 29C again shows that the MadCAM-Fc-CCL25, TNFR2-Fc-TGFβ, α4β7-Fc-IL35, and IL6R-Fc-IL35 chimeric proteins promoted the formation of various subtypes of Treg cells.

These data demonstrate that the chimeric protein disclosed herein promote the formation of Treg cells. Accordingly, the chimeric proteins disclosed herein may be used in therapeutic methods where suppressing inflammation and treating conditions such as autoimmune diseases.

### INCORPORATION BY REFERENCE

All patents and publications referenced herein are hereby incorporated by reference in their entireties.

Specifically, additional teachings related to the present invention are found, in one or more of WO2018/157162; WO2018/157165; WO2018/157164; WO2018/157163; and WO2017/059168, the contents of each of which is incorporated herein by reference in its entirety.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

### EQUIVALENTS

While the invention has been disclosed in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments disclosed specifically herein. Such equivalents are intended to be encompassed in the scope of the following claims.

What is claimed is:

1. A chimeric protein of a general structure of:
N terminus-(a)-(b)-(c)-C terminus,
wherein:
(a) is a first domain comprising a portion of the extracellular domain of a transmembrane protein, a secreted protein, or a membrane-anchored extracellular protein,
(c) is a second domain comprising a portion of the extracellular domain of a transmembrane protein, a secreted protein, or a membrane-anchored extracellular protein, and
(b) is a linker adjoining the first domain and the second domain,
wherein either or both of the first domain and the second domain decreases self-directed immune system activity when bound to its ligand/receptor.

2. The chimeric protein of claim 1, wherein the portion of the first domain is capable of binding the native ligand/receptor for the transmembrane protein, the secreted protein, or the membrane-anchored extracellular protein.

3. The chimeric protein of claim 1 or claim 2, wherein the portion of the second domain is capable of binding the native ligand/receptor for the transmembrane protein, the secreted protein, or the membrane-anchored extracellular protein.

4. The chimeric protein of any one of claims 1 to 3, wherein the first domain comprises substantially the entire extracellular domain of the transmembrane protein, substantially the entire secreted protein, or substantially the entire membrane-anchored extracellular protein.

5. The chimeric protein of any one of claims 1 to 4, wherein the second domain comprises substantially the entire extracellular domain of the transmembrane protein, substantially the entire secreted protein, or substantially the entire membrane-anchored extracellular protein.

6. The chimeric protein of any one of claims 1 to 5, wherein binding the portion of the first domain to its ligand/receptor decreases immune system activity by activating an immune inhibitory signal or inhibiting an immune activating signal.

7. The chimeric protein of any one of claims 1 to 6, wherein binding the portion of the second domain to its ligand/receptor decreases immune system activity by activating an immune inhibitory signal or by inhibiting an immune activating signal.

8. The chimeric protein of any one of claims 1 to 7, wherein the portion of the first domain comprises a transmembrane protein, a secreted protein, or a membrane-anchored extracellular protein selected from B7H3, B7H4, BTNL2, CTLA4, CSF3, ICOSL, ILDR2, PD-L1, TNFR2, IL-6R, MadCAM, integrin α4β7, and VSIG4.

9. The chimeric protein of any one of claims 1 to 8, wherein the portion of the second domain comprises a transmembrane protein, a secreted protein, or a membrane-anchored extracellular protein selected from BTNL2, IL2, PD-L1, SEMA3E, IL-35, CCL25, TGFβ, and TL1A.

10. The chimeric protein of any one of claims 1 to 9, wherein the first domain comprises a portion of VSIG4 and the second domain comprises a portion of IL2.

11. The chimeric protein of any one of claims 1 to 9, wherein the first domain comprises a portion of CTLA4 and the second domain comprises a portion of IL2.

12. The chimeric protein of any one of claims 9 to 11, wherein the portion of IL2 comprises one or more mutations relative to a corresponding portion of wild-type IL2 wherein the one or more mutations provide preferential binding to a high-affinity IL2 receptor that is expressed by regulatory T cells.

13. The chimeric protein of any one of claims 1 to 9, wherein the first domain comprises a portion of CTLA4 and the second domain comprises a portion of PD-L1.

14. The chimeric protein of any one of claims 1 to 9, wherein the first domain comprises a portion of B7H3 and the second domain comprises a portion of PD-L1.

15. The chimeric protein of any one of claims 1 to 9, wherein the first domain comprises a portion of B7H4 and the second domain comprises a portion of PD-L1.

16. The chimeric protein of any one of claims 1 to 9, wherein the first domain comprises a portion of ICOSL and the second domain comprises a portion of PD-L1.

17. The chimeric protein of any one of claims 1 to 9, wherein the first domain comprises a portion of ILDR2 and the second domain comprises a portion of PD-L1.

18. The chimeric protein of any one of claims 1 to 9, wherein the first domain comprises a portion BTNL2 of and the second domain comprises a portion of PD-L1 or the first domain comprises a portion of PD-L1 and the second domain comprises a portion of BTNL2.

19. The chimeric protein of any one of claims 1 to 9, wherein the first domain comprises a portion of CSF3 and the second domain comprises a portion of TL1A.

20. The chimeric protein of any one of claims 1 to 9, wherein the first domain comprises a portion of CTLA4 and the second domain comprises a portion of TL1A.

21. The chimeric protein of any one of claims 1 to 9, wherein the first domain comprises a portion of CTLA4 and the second domain comprises a portion of SEMA3E.

22. The chimeric protein of any one of claims 1 to 8, wherein the first domain comprises a portion of TNFR2 and the second domain comprises an extracellular domain of a transmembrane protein selected from GITRL and TL1A.

23. The chimeric protein of any one of claims 1 to 8, wherein the first domain comprises a portion of CTLA4 and the second domain comprises an extracellular domain of a transmembrane protein selected from GITRL and TL1A.

24. The chimeric protein of any one of claims 1 to 9, wherein the first domain comprises an extracellular domain of IL-6R and the second domain comprises a portion of IL-35.

25. The chimeric protein of claim 24, wherein the first domain comprises an extracellular domain of IL-6ST and/or IL-6R and/or the second domain comprises a portion of EBI3 and/or IL-12A.

26. The chimeric protein of claim 24 or claim 25, wherein the chimeric protein is a heterodimer.

27. The chimeric protein of any one of claims 1 to 9, wherein the first domain comprises an extracellular domain of MadCAM and the second domain comprises a portion of CCL25.

28. The chimeric protein of any one of claims 1 to 9, wherein the first domain comprises an extracellular domain of TNFR2 and the second domain comprises a portion of TGFβ.

29. The chimeric protein of any one of claims 1 to 9, wherein the first domain comprises an extracellular domain of integrin α4β7 and the second domain comprises a portion of IL-35.

30. The chimeric protein of claim 29, wherein the first domain comprises an extracellular domain of integrin α4 and/or integrin β7, and/or the second domain comprises a portion of EBI3 and/or IL-12A.

31. The chimeric protein of claim 29 or claim 30, wherein the chimeric protein is a heterodimer.

32. The chimeric protein of any one of claims 1 to 9, wherein the chimeric protein is capable of contemporaneously binding a TNFR2 ligand and a ligand/receptor of a Type II transmembrane protein selected from BTNL2C-type lectin domain (CLEC) family members, GITRL TL1A, IL-10, or TGF-beta.

33. The chimeric protein of claim 32, wherein the CLEC family member is selected from AlCL/CLEC-2B, ASGR1/ASGPR1, ASGR2, C1q R1/CD93, CD161, CD161/NK1.1, CD23/Fc epsilon RII, CD302/CLEC13A, CD72, CD94, Chondrolectin, CLEC-1, CLEC10A/CD301, CLEC12B, CLEC14A, CLEC16A, CLEC17A, CLEC18A, CLEC18B, CLEC18C, CLEC-2/CLEC1B, CLEC-2A, CLEC3A, CLEC3B/Tetranectin, CLEC4B2/mDCAR1, CLEC4D/CLECSF8, CLEC4E, CLEC4F/CLECSF13, CLEC9a, CLECL1/DCAL-1, CL-K1/COLEC11, CL-L1/COLEC10, CL-P1/COLEC12, DCAR/CLEC4B, DCIR/CLEC4A, DCIR4/CLEC4A1, DC-SIGN/CD209, DC-SIGN+FDC-SIGNR, DC-SIGNR/CD299, DC-SIGNR/CD299, DEC-205/CD205, Dectin-1/CLEC7A, Dectin-2/CLEC6A, DLEC/CLEC4C/BDCA-2, Ficolin-1, Ficolin-2, Ficolin-3, Klre-1, KLRG2, Langerin/CD207, Layilin, LOX-1/OLR1, LSECtin/CLEC4G, MBL, MBL-1, MBL-2, MDL-1/CLEC5A, MGL1/2 (CD301a/b), MGL1/CD301a, MGL2/CD301b, MGL2/CD301b, MICL/CLEC12A, MMR/CD206, Mrc2, NKG2A/CD159a, NKG2A/NKG2B Isoform 2, NKG2C/CD159c, NKG2D/CD314, NKG2E, NKG2H, NKp80/KLRF1, OCIL/CLEC2d, OCILRP2/CLEC2i, PLA2R1, QBRICK/FREM1, Reg1, Reg1A, Reg1B, Reg2, Reg3A, Reg3B, Reg3D, Reg3G, Reg4, SCGF/CLEC11a, SFTPA1, SIGNR1/CD209b, SIGNR3/CD209d, SIGNR4/CD209e, SIGNR7/CD209g, and SP-D.

34. The chimeric protein of any one of claims 1 to 33, wherein binding of either or both of the first domain and the second domains to its ligand/receptor occurs with slow off rates (Koff), which provides a long interaction of a receptor and its ligand.

35. The chimeric protein of claim 34, wherein the long interaction provides a prolonged decrease in immune system activity which comprises sustained activation of an immune inhibitory signal and/or a sustained inhibition of an immune activating signal.

36. The chimeric protein of claim 35, wherein the sustained activation of the immune inhibitory signal and/or the sustained inhibition of the immune activating signal reduces the activity or proliferation of an immune cell.

37. The chimeric protein of claim 36, wherein the immune cell is a B cell or a T cell.

38. The chimeric protein of any one of claims 36 to 37, wherein the sustained activation of the immune inhibitory signal and/or the sustained inhibition of the immune activating signal decreases synthesis and/or decreases release of a pro-inflammatory cytokine.

39. The chimeric protein of any one of claims 36 to 38, wherein the sustained activation of the immune inhibitory signal and/or the sustained inhibition of the immune activating signal increases synthesis and/or increases release of an anti-inflammatory cytokine.

40. The chimeric protein of any one of claims 36 to 39, wherein the sustained activation of the immune inhibitory signal and/or the sustained inhibition of the immune activating signal decreases tissue damage caused by an immune response.

41. The chimeric protein of any one of claims 36 to 40, wherein the sustained activation of the immune inhibitory signal and/or the sustained inhibition of the immune activating signal decreases antibody production and/or decreases secretion of antibodies by a B cell.

42. The chimeric protein of claim 41, wherein the antibody recognizes a self-antigen.

43. The chimeric protein of any one of claims 36 to 42, wherein the sustained activation of the immune inhibitory signal and/or the sustained inhibition of the immune activating signal decreases the activity of and/or decreases the number of T cytotoxic cells.

44. The chimeric protein of claim 43, wherein the T cytotoxic cells recognize a self-antigen and kill cells presenting or expressing the self-antigen.

45. The chimeric protein of any one of claims 36 to 44, wherein the sustained activation of the immune inhibitory signal and/or the sustained inhibition of the immune activating signal increases the activity and/or increases the number of T regulatory cells.

46. The chimeric protein of any one of claims 1 to 45, wherein the linker is a polypeptide selected from a flexible amino acid sequence, an IgG hinge region, and an antibody sequence.

47. The chimeric protein of any one of claims 1 to 46, wherein the linker comprises at least one cysteine residue capable of forming a disulfide bond and/or comprises a hinge-CH2-CH3 Fc domain.

48. The chimeric protein of claim 47, wherein the hinge-CH2-CH3 Fc domain is derived from IgG, IgA, IgD, or IgE.

49. The chimeric protein of claim 48, wherein the IgG is selected from IgG1, IgG2, IgG3, and IgG4 and the IgA is selected from IgA1 and IgA2.

50. The chimeric protein of claim 49, wherein the IgG is IgG4.

51. The chimeric protein of claim 50, wherein the IgG4 is a human IgG4.

52. The chimeric protein of any one of claims 47 to 51, wherein the linker comprises an amino acid sequence that is at least 95% identical to the amino acid sequence of SEQ ID NO: 1, SEQ ID NO: 2, or SEQ ID NO: 3.

53. The chimeric protein of claim 49, wherein the IgG is IgG1.

54. The chimeric protein of claim 53, wherein the IgG1 is a human IgG1.

55. A chimeric protein comprising:
(a) a first domain comprising a portion of VSIG4 that is capable of binding a VSIG4 ligand/receptor,
(b) a second domain comprising a portion of IL2 that is capable of binding an IL2 receptor, and
(c) a linker linking the first domain and the second domain and comprising a hinge-CH2-CH3 Fc domain.

56. A chimeric protein comprising:
(a) a first domain comprising a portion of CTLA4 that is capable of binding a CTLA4 ligand/receptor,
(b) a second domain comprising a portion of IL2 that is capable of binding an IL2 receptor, and
(c) a linker linking the first domain and the second domain and comprising a hinge-CH2-CH3 Fc domain.

57. The chimeric protein of claim 55 or claim 56, wherein the IL2 receptor is a high-affinity IL2 receptor that is expressed by regulatory T cells.

58. The chimeric protein of claim 57, wherein the portion of IL2 comprises one or more mutations relative to a corresponding portion of wild-type IL2 which provides preferential binding to the high-affinity IL2 receptor that is expressed by regulatory T cells.

59. A chimeric protein comprising:
(a) a first domain comprising a portion of CTLA4 that is capable of binding a CTLA4 ligand/receptor,
(b) a second domain comprising a portion of PD-L1 that is capable of binding PD-1, and
(c) a linker linking the first domain and the second domain and comprising a hinge-CH2-CH3 Fc domain.

60. A chimeric protein comprising:
(a) a first domain comprising a portion of B7H3 that is capable of binding a B7H3 ligand/receptor,
(b) a second domain comprising a portion of PD-L1 that is capable of binding PD-1, and
(c) a linker linking the first domain and the second domain and comprising a hinge-CH2-CH3 Fc domain.

61. A chimeric protein comprising:
(a) a first domain comprising a portion of B7H4 that is capable of binding a B7H4 ligand/receptor,
(b) a second domain comprising a portion of PD-L1 that is capable of binding PD-1, and
(c) a linker linking the first domain and the second domain and comprising a hinge-CH2-CH3 Fc domain.

62. A chimeric protein comprising:
(a) a first domain comprising a portion of ICOSL that is capable of binding an ICOSL ligand/receptor,
(b) a second domain comprising a portion of PD-L1 that is capable of binding PD-1, and
(c) a linker linking the first domain and the second domain and comprising a hinge-CH2-CH3 Fc domain.

63. A chimeric protein comprising:
(a) a first domain comprising a portion of ILDR2 that is capable of binding an ILDR2 ligand/receptor,
(b) a second domain comprising a portion of PD-L1 that is capable of binding PD-1, and
(c) a linker linking the first domain and the second domain and comprising a hinge-CH2-CH3 Fc domain.

64. A chimeric protein comprising:
(a) a first domain comprising a portion of BTNL2 that is capable of binding a BTNL2 ligand/receptor,
(b) a second domain comprising a portion of PD-L1 that is capable of binding PD-1, and
(c) a linker linking the first domain and the second domain and comprising a hinge-CH2-CH3 Fc domain.

65. A chimeric protein comprising:
(a) a first domain comprising a portion of PD-L1 that is capable of binding PD-1,
(b) a second domain comprising a portion of BTNL2 that is capable of binding a BTNL2 ligand/receptor, and
(c) a linker linking the first domain and the second domain and comprising a hinge-CH2-CH3 Fc domain.

66. A chimeric protein comprising:
(a) a first domain comprising a portion of CSF3 that is capable of binding a CSF3 ligand/receptor,
(b) a second domain comprising a portion of TL1A that is capable of binding a TL1A ligand/receptor, and
(c) a linker linking the first domain and the second domain and comprising a hinge-CH2-CH3 Fc domain.

67. A chimeric protein comprising:
(a) a first domain comprising a portion of CTLA4 that is capable of binding a CTLA4 ligand/receptor,
(b) a second domain comprising a portion of TL1A that is capable of binding a TL1A ligand/receptor, and
(c) a linker linking the first domain and the second domain and comprising a hinge-CH2-CH3 Fc domain.

68. A chimeric protein comprising:
(a) a first domain comprising a portion of CTLA4 that is capable of binding a CTLA4 ligand/receptor,
(b) a second domain comprising a portion of SEMA3E that is capable of binding a SEMA3E ligand/receptor, and
(c) a linker linking the first domain and the second domain and comprising a hinge-CH2-CH3 Fc domain.

69. A chimeric protein comprising:
(a) a first domain comprising a portion of MadCAM that is capable of binding a MadCAM ligand/receptor,
(b) a second domain comprising a portion of CCL25 that is capable of binding a CCL25 ligand/receptor, and
(c) a linker linking the first domain and the second domain and comprising a hinge-CH2-CH3 Fc domain.

70. A chimeric protein comprising:
(a) a first domain comprising a portion of TNFR2 that is capable of binding a TNFR2 ligand/receptor,
(b) a second domain comprising a portion of TGFβ that is capable of binding a TGFβ ligand/receptor, and
(c) a linker linking the first domain and the second domain and comprising a hinge-CH2-CH3 Fc domain.

71. A chimeric protein comprising:
(a) a first domain comprising an extracellular domain of IL-6R that is capable of binding a IL-6R ligand/receptor,
(b) a second domain comprising a portion of IL-35 that is capable of binding a IL-35 ligand/receptor, and
(c) a linker linking the first domain and the second domain and comprising a hinge-CH2-CH3 Fc domain.

72. The chimeric protein of claim 71, wherein:
the first domain comprises an extracellular domain of IL-6ST and/or IL-6R; and/or
the second domain comprises a portion of EBI3 and/or IL-12A.

73. A chimeric protein comprising:
(a) a first domain comprising an extracellular domain of integrin α4β7 that is capable of binding an integrin α4β7 ligand/receptor,
(b) a second domain comprising a portion of IL-35 that is capable of binding a IL-35 ligand/receptor, and
(c) a linker linking the first domain and the second domain and comprising a hinge-CH2-CH3 Fc domain.

74. The chimeric protein of claim 73, wherein:
the first domain comprises an extracellular domain of integrin α4 and/or integrin β7; and/or
the second domain comprises a portion of EBI3 and/or IL-12A.

75. The chimeric protein of any one of claims 71-74, wherein the chimeric protein is heterodimeric.

76. The chimeric protein of any one of claims 55 to 75, wherein the hinge-CH2-CH3 Fc domain comprises at least one cysteine residue capable of forming a disulfide bond.

77. The chimeric protein of claim 76, wherein the hinge-CH2-CH3 Fc domain is derived from IgG, IgA, IgD, or IgE.

78. The chimeric protein of claim 77, wherein the IgG is selected from IgG1, IgG2, IgG3, and IgG4 and the IgA is selected from IgA1 and IgA2.

79. The chimeric protein of claim 78, wherein the IgG is IgG4.

80. The chimeric protein of claim 79, wherein the IgG4 is a human IgG4.

81. The chimeric protein of any one of claims 55 to 80, wherein the linker comprises an amino acid sequence that is at least 95% identical to the amino acid sequence of SEQ ID NO: 1, SEQ ID NO: 2, or SEQ ID NO: 3.

82. The chimeric protein of claim 78, wherein the IgG is IgG1.

83. The chimeric protein of claim 82, wherein the IgG1 is a human IgG1.

84. The chimeric protein of any one of claims 1 to 83, wherein the chimeric protein is a recombinant fusion protein.

85. The chimeric protein of any one of claims 1 to 84 for use as a medicament in the treatment of an autoimmune disease.

86. An expression vector comprising a nucleic acid encoding the chimeric protein of any one of claims 1 to 85.

87. A host cell comprising the expression vector of claim 86.

88. A pharmaceutical composition, comprising a therapeutically effective amount of the chimeric protein of any one of claims 1-85.

89. The chimeric protein of any one of claims 1-85, for use as a medicament.

90. The chimeric protein of any one of claims 1-85, for use in the treatment of an autoimmune disease.

91. The chimeric protein of any one of claims 1-51, for use in the treatment of an inflammatory disease.

92. Use of the chimeric protein of any one of claims 1 to 85, in the manufacture of a medicament.

93. The pharmaceutical composition of claim 88, for use as a medicament.

94. Use of the pharmaceutical of composition claim 88, in the manufacture of a medicament.

95. The pharmaceutical composition of claim 88, for use in the treatment of an autoimmune disease.

96. The pharmaceutical composition of claim 88, for use in the treatment of an autoimmune disease, or an inflammatory disease.

97. A method of treating an autoimmune disease comprising administering to a subject in need thereof an effective amount of the pharmaceutical composition of claim 88.

98. The method of claim 97, further comprising administering to the subject an anti-inflammatory drug.

99. The method of claim 98, wherein the anti-inflammatory drug is a non-steroidal anti-inflammatory or a corticosteroid.

100. The method of claim 98 or claim 99, wherein the pharmaceutical composition and the anti-inflammatory drug are administered simultaneously, e.g., as two distinct pharmaceutical compositions or as a single pharmaceutical composition.

101. The method of claim 98 or claim 99, wherein the pharmaceutical composition is administered after the anti-inflammatory drug is administered.

102. The method of claim 98 or claim 99, wherein the pharmaceutical composition is administered before the anti-inflammatory drug is administered.

103. The method of any one of claims 99 to 102, wherein the non-steroidal anti-inflammatory is selected from the group consisting of acetyl salicylic acid (aspirin), benzyl-2,5-diacetoxybenzoic acid, celecoxib, diclofenac, etodolac, etofenamate, fulindac, glycol salicylate, ibuprofen, indomethacin, ketoprofen, methyl salicylate, nabumetone, naproxen, oxaprozin, phenylbutazone, piroxicam, salicylic acid, salicylmides, and Vimovo® (a combination of naproxen and esomeprazole magnesium).

104. The method of any one of claims 99 to 102, wherein the corticosteroid is selected from the group consisting of alpha-methyl dexamethasone, amcinafel, amcinafide, beclomethasone dipropionate, beclomethasone dipropionate, betamethasone and the balance of its esters, betamethasone benzoate, betamethasone dipropionate, betamethasone valerate, beta-methyl betamethasone, bethamethasone, chloroprednisone, clescinolone, clobetasol valerate, clocortelone, cortisone, cortodoxone, desonide, desoxymethasone, dexamethasone, dichlorisone, diflorasone diacetate, diflucortolone valerate, difluorosone diacetate, difluprednate, fluadrenolone, flucetonide, fluclorolone acetonide, flucloronide, flucortine butylester, fludrocortisone, flumethasone pivalate, flunisolide, fluocinonide, fluocortolone, fluoromethalone, fluosinolone acetonide, fluperolone, fluprednidene (fluprednylidene) acetate, fluprednisolone, fluradrenolone acetonide, flurandrenolone, halcinonide, hydrocortisone, hydrocortisone acetate, hydrocortisone butyrate, hydroxyltriamcinolone, medrysone, meprednisone, methylprednisolone, paramethasone, prednisolone, prednisone, triamcinolone, and triamcinolone acetonide.

105. The method of claim 97, further comprising administering to the subject an immunosuppressive agent.

106. The method of claim 105, wherein the pharmaceutical composition and the immunosuppressive agent are administered simultaneously, e.g., as two distinct pharmaceutical compositions or as a single pharmaceutical composition.

107. The method of claim 105, wherein the pharmaceutical composition is administered after the immunosuppressive agent is administered.

108. The method of claim 105, wherein the pharmaceutical composition is administered before the immunosuppressive agent is administered.

109. The method of any one of claims 105 to 108, wherein the immunosuppressive agent is selected from the group consisting of an antibody (e.g., basiliximab, daclizumab, and muromonab), an anti-immunophilin (e.g., cyclosporine, tacrolimus, and sirolimus), an antimetabolite (e.g., azathioprine and methotrexate), a cytostatic (such as alkylating agents), a cytotoxic antibiotic, an interferon, a mycophenolate, an opioid, a small biological agent (e.g., fingolimod and myriocin), and a TNF binding protein.

110. The method of any one of claims 97 to 109, further comprising administering to the subject an anti-inflammatory drug and an immunosuppressive agent.

111. The method of any one of claims 97 to 110 or the chimeric protein of claim 90 or the pharmaceutical composition of claim 95 or claim 96, wherein the autoimmune disease is selected from ankylosing spondylitis, diabetes mellitus, Grave's disease, Hashimoto's thyroiditis, hypersensitivity reactions (e.g., allergies, hay fever, asthma, and acute edema cause type I hypersensitivity reactions), inflammatory bowel diseases (e.g., colitis ulcerosa and Crohn's disease), multiple sclerosis, psoriasis, psoriasis, rheumatoid arthritis, sarcoidosis, Sjögren's syndrome, systemic lupus erythematosus, and vasculitis.


SEQUENCE LISTING (106 sequences)

SEQ ID NO: 1 - protein, 217 aa, Artificial Sequence
  Synthetic sequence_Fc Domain linker
APEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGK

SEQ ID NO: 2 - protein, 217 aa, Artificial Sequence
  Synthetic sequence_Fc Domain linker
APEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGK

SEQ ID NO: 3 - protein, 217 aa, Artificial Sequence
  Synthetic sequence_Fc Domain linker
APEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 4 - protein, 11 aa, Artificial Sequence
  Synthetic sequence_Joining Linker 1
SKYGPPCPSCP

SEQ ID NO: 5 - protein, 11 aa, Artificial Sequence
  Synthetic sequence_Joining Linker 1
SKYGPPCPPCP

SEQ ID NO: 6 - protein, 6 aa, Artificial Sequence
  Synthetic sequence_linker
SKYGPP

SEQ ID NO: 7 - protein, 6 aa, Artificial Sequence
  Synthetic sequence_Joining linker2
IEGRMD

SEQ ID NO: 8 - protein, 9 aa, Artificial Sequence
  Synthetic sequence
GGGVPRDCG

SEQ ID NO: 9 - protein, 15 aa, Artificial Sequence
  Synthetic sequence_linker
IEGRMDGGGGAGGGG

SEQ ID NO: 10 - protein, 8 aa, Artificial Sequence
  Synthetic sequence_Joining Linker
GGGSGGGS

SEQ ID NO: 11 - protein, 12 aa, Artificial Sequence
  Synthetic sequence_Joining Linker
GGGSGGGGSGGG

SEQ ID NO: 12 - protein, 14 aa, Artificial Sequence
  Synthetic sequence_Joining Linker
EGKSSGSGSESKST

SEQ ID NO: 13 - protein, 4 aa, Artificial Sequence
  Synthetic sequence_Joining Linker
GGSG

SEQ ID NO: 14 - protein, 12 aa, Artificial Sequence
  Synthetic sequence_Joining Linker
GGSGGGSGGGSG

SEQ ID NO: 15 - protein, 15 aa, Artificial Sequence
  Synthetic sequence_Joining Linker
EAAAKEAAAKEAAAK

SEQ ID NO: 16 - protein, 20 aa, Artificial Sequence
  Synthetic sequence_Joining Linker
EAAAREAAAREAAAREAAAR

SEQ ID NO: 17 - protein, 17 aa, Artificial Sequence
  Synthetic sequence_Joining Linker
GGGGSGGGGSGGGGSAS

SEQ ID NO: 18 - protein, 9 aa, Artificial Sequence
  Synthetic sequence_Joining Linker
GGGGAGGGG

SEQ ID NO: 19 - protein, 2 aa, Artificial Sequence
  Synthetic sequence_Joining Linker
GS

SEQ ID NO: 20 - protein, 6 aa, Artificial Sequence
  Synthetic sequence_Joining Linker
GSGSGS

SEQ ID NO: 21 - protein, 10 aa, Artificial Sequence
  Synthetic sequence_Joining Linker
GSGSGSGSGS

SEQ ID NO: 22 - protein, 7 aa, Artificial Sequence
  Synthetic sequence_Joining Linker
GGGGSAS

SEQ ID NO: 23 - protein, 20 aa, Artificial Sequence
  Synthetic sequence_Joining Linker
APAPAPAPAPAPAPAPAPAP

SEQ ID NO: 24 - protein, 4 aa, Artificial Sequence
  Synthetic sequence_linker
CPPC

SEQ ID NO: 25 - protein, 5 aa, Artificial Sequence
  Synthetic sequence_linker
GGGGS

SEQ ID NO: 26 - protein, 10 aa, Artificial Sequence
  Synthetic sequence_linker
GGGGSGGGGS

SEQ ID NO: 27 - protein, 15 aa, Artificial Sequence
  Synthetic sequence_linker
GGGGSGGGGSGGGGS

SEQ ID NO: 28 - protein, 20 aa, Artificial Sequence
  Synthetic sequence_linker
GGGGSGGGGSGGGGSGGGGS

SEQ ID NO: 29 - protein, 25 aa, Artificial Sequence
  Synthetic sequence_linker
GGGGSGGGGSGGGGSGGGGSGGGGS

SEQ ID NO: 30 - protein, 30 aa, Artificial Sequence
  Synthetic sequence_linker
GGGGSGGGGSGGGGSGGGGSGGGGSGGGGS

SEQ ID NO: 31 - protein, 35 aa, Artificial Sequence
  Synthetic sequence_linker
GGGGSGGGGSGGGGSGGGGSGGGGSGGGGSGGGGS

SEQ ID NO: 32 - protein, 40 aa, Artificial Sequence
  Synthetic sequence_linker
GGGGSGGGGSGGGGSGGGGSGGGGSGGGGSGGGGSGGGGS

SEQ ID NO: 33 - protein, 16 aa, Artificial Sequence
  Synthetic sequence_linker
GGSGGSGGGGSGGGGS

SEQ ID NO: 34 - protein, 8 aa, Artificial Sequence
  Synthetic sequence_linker
GGGGGGGG

SEQ ID NO: 35 - protein, 6 aa, Artificial Sequence
  Synthetic sequence_linker
GGGGGG

SEQ ID NO: 36 - protein, 5 aa, Artificial Sequence
  Synthetic sequence_linker
EAAAK

SEQ ID NO: 37 - protein, 10 aa, Artificial Sequence
  Synthetic sequence_linker
EAAAKEAAAK

SEQ ID NO: 38 - protein, 15 aa, Artificial Sequence
  Synthetic sequence_linker
EAAAKEAAAKEAAAK

SEQ ID NO: 39 - protein, 12 aa, Artificial Sequence
  Synthetic sequence_linker
AEAAAKEAAAKA

SEQ ID NO: 40 - protein, 17 aa, Artificial Sequence
  Synthetic sequence_linker
AEAAAKEAAAKEAAAKA

SEQ ID NO: 41 - protein, 22 aa, Artificial Sequence
  Synthetic sequence_linker
AEAAAKEAAAKEAAAKEAAAKA

SEQ ID NO: 42 - protein, 27 aa, Artificial Sequence
  Synthetic sequence_linker
AEAAAKEAAAKEAAAKEAAAKEAAAKA

SEQ ID NO: 43 - protein, 46 aa, Artificial Sequence
  Synthetic sequence_linker
AEAAAKEAAAKEAAAKEAAAKALEAEAAAKEAAAKEAAAKEAAAKA

SEQ ID NO: 44 - protein, 5 aa, Artificial Sequence
  Synthetic sequence_linker
PAPAP

SEQ ID NO: 45 - protein, 18 aa, Artificial Sequence
  Synthetic sequence_linker
KESGSVSSEQLAQFRSLD

SEQ ID NO: 46 - protein, 12 aa, Artificial Sequence
  Synthetic sequence_linker
GSAGSAAGSGEF

SEQ ID NO: 47 - protein, 5 aa, Artificial Sequence
  Synthetic sequence_linker
GGGSE

SEQ ID NO: 48 - protein, 5 aa, Artificial Sequence
  Synthetic sequence_linker
GSESG

SEQ ID NO: 49 - protein, 5 aa, Artificial Sequence
  Synthetic sequence_linker
GSEGS

SEQ ID NO: 50 - protein, 35 aa, Artificial Sequence
  Synthetic sequence_linker
GEGGSGEGSSGEGSSSEGGGSEGGGSEGGGSEGGS

SEQ ID NO: 51 - protein, 234 aa, Artificial Sequence
  Synthetic sequence_Modular linker
SKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMD

SEQ ID NO: 52 - protein, 234 aa, Artificial Sequence
  Synthetic sequence_Modular linker
SKYGPPCPSCPAPEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGKIEGRMD

SEQ ID NO: 53 - protein, 234 aa, Artificial Sequence
  Synthetic sequence_Modular linker
SKYGPPCPSCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMD

SEQ ID NO: 54 - protein, 234 aa, Artificial Sequence
  Synthetic sequence_Modular linker
SKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMD

SEQ ID NO: 55 - protein, 234 aa, Artificial Sequence
  Synthetic sequence_Modular linker
SKYGPPCPPCPAPEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGKIEGRMD

SEQ ID NO: 56 - protein, 234 aa, Artificial Sequence
  Synthetic sequence_Modular linker
SKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMD

SEQ ID NO: 57 - protein, 178 aa, Artificial Sequence
  Synthetic sequence_portion of CSF3
ATPLGPASSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 58 - protein, 195 aa, Artificial Sequence
  Synthetic sequence_extracellular domain of TL1A
SQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 59 - protein, 607 aa, Artificial Sequence
  Synthetic sequence_CSF3-Fc-TL1A chimeric protein
ATPLGPASSLPQSFLLKCLEQVRKIQGDGAALQEKLVSECATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDSQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 60 - protein, 126 aa, Artificial Sequence [sequence omitted]

SEQ ID NO: 61 - protein, 555 aa, Artificial Sequence
  Synthetic sequence_CTLA4-Fc-TL1A chimeric protein
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDSQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 62 - protein, 220 aa, Artificial Sequence
  Synthetic sequence_extracellular domain of PD-L1
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 63 - protein, 580 aa, Artificial Sequence
  Synthetic sequence_CTLA4-Fc-PD-L1 chimeric protein
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 64 - protein, 438 aa, Artificial Sequence
  Synthetic sequence_extracellular domain of B7H3
LEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEA

SEQ ID NO: 65 - protein, 892 aa, Artificial Sequence
  Synthetic sequence_B7H3-Fc-PD-L1 chimeric protein
LEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEASKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 66 - protein, 226 aa, Artificial Sequence
  Synthetic sequence_extracellular domain of B7H4
LIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSH

SEQ ID NO: 67 - protein, 680 aa, Artificial Sequence
  Synthetic sequence_B7H4-Fc-PD-L1 chimeric protein
LIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 68-69 - skipped (2 entries)

SEQ ID NO: 70 - protein, 166 aa, Artificial Sequence
  Synthetic sequence_extracellular domain of ILDR2
LQVTVPDKKKVAMLFQPTVLRCHFSTSSHQPAVVQWKFKSYCQDRMGESLGMSSTRAQSLSKRNLEWDPYLDCLDSRRTVRVVASKQGSTVTLGDFYRGREITIVHDADLQIGKLMWGDSGLYYCIITTPDDLEGKNEDSVELLVLGRTGLLADLLPSFAVEIMPE

SEQ ID NO: 71 - protein, 620 aa, Artificial Sequence
  Synthetic sequence_ILDR2-Fc-PD-L1 chimeric protein
LQVTVPDKKKVAMLFQPTVLRCHFSTSSHQPAVVQWKFKSYCQDRMGESLGMSSTRAQSLSKRNLEWDPYLDCLDSRRTVRVVASKQGSTVTLGDFYRGREITIVHDADLQIGKLMWGDSGLYYCIITTPDDLEGKNEDSVELLVLGRTGLLADLLPSFAVEIMPEVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGIIEGRMDFTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTH

SEQ ID NO: 72 - protein, 432 aa, Artificial Sequence
  Synthetic sequence_extracellular domain of BTNL2
KQSEDFRVIGPAHPILAGVGEDALLTCQLLPKRTTMHVEVRWYRSEPSTPVFVHRDGVEVTEMQMEEYRGWVEWIENGIAKGNVALKIHNIQPSDNGQYWCHFQDGNYCGETSLLLKVAGLGSAPSIHMEGPGESGVQLVCTARGWFPEPQVYWEDIRGEKLLAVSEHRIQDKDGLFYAEATLVVRNASAESVSCLVHNPVLTEEKGSVISLPEKLQTELASLKVNGPSQPILVRVGEDIQLTCYLSPKANAQSMEVRWDRSHRYPAVHVYMDGDHVAGEQMAEYRGRTVLVSDAIDEGRLTLQILSARPSDDGQYRCLFEKDDVYQEASLDLKVVSLGSSPLITVEGQEDGEMQPMCSSDGWFPQPHVPWRDMEGKTIPSSSQALTQGSHGLFHVQTLLRVTNISAVDVTCSISIPFLGEEKIATFSLSGW

SEQ ID NO: 73 - protein, 886 aa, Artificial Sequence
  Synthetic sequence_BTNL2-Fc-PD-L1 chimeric protein
KQSEDFRVIGPAHPILAGVGEDALLTCQLLPKRTTMHVEVRWYRSEPSTPVFVHRDGVEVTEMQMEEYRGWVEWIENGIAKGNVALKIHNIQPSDNGQYWCHFQDGNYCGETSLLLKVAGLGSAPSIHMEGPGESGVQLVCTARGWFPEPQVYWEDIRGEKLLAVSEHRIQDKDGLFYAEATLVVRNASAESVSCLVHNPVLTEEKGSVISLPEKLQTELASLKVNGPSQPILVRVGEDIQLTCYLSPKANAQSMEVRWDRSHRYPAVHVYMDGDHVAGEQMAEYRGRTVLVSDAIDEGRLTLQILSARPSDDGQYRCLFEKDDVYQEASLDLKVVSLGSSPLITVEGQEDGEMQPMCSSDGWFPQPHVPWRDMEGKTIPSSSQALTQGSHGLFHVQTLLRVTNISAVDVTCSISIPFLGEEKIATFSLSGWSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 74 - protein, 886 aa, Artificial Sequence
  Synthetic sequence_PD-L1-Fc-BTNL2 chimeric protein
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDKQSEDFRVIGPAHPILAGVGEDALLTCQLLPKRTTMHVEVRWYRSEPSTPVFVHRDGVEVTEMQMEEYRGWVEWIENGIAKGNVALKIHNIQPSDNGQYWCHFQDGNYCGETSLLLKVAGLGSAPSIHMEGPGESGVQLVCTARGWFPEPQVYWEDIRGEKLLAVSEHRIQDKDGLFYAEATLVVRNASAESVSCLVHNPVLTEEKGSVISLPEKLQTELASLKVNGPSQPILVRVGEDIQLTCYLSPKANAQSMEVRWDRSHRYPAVHVYMDGDHVAGEQMAEYRGRTVLVSDAIDEGRLTLQILSARPSDDGQYRCLFEKDDVYQEASLDLKVVSLGSSPLITVEGQEDGEMQPMCSSDGWFPQPHVPWRDMEGKTIPSSSQALTQGSHGLFHVQTLLRVTNISAVDVTCSISIPFLGEEKIATFSLSGW

SEQ ID NO: 75 - protein, 133 aa, Artificial Sequence
  Synthetic sequence_portion of IL2
APTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYACETATIVEFLNRWITFSQSIISTLT

SEQ ID NO: 76 - protein, 133 aa, Artificial Sequence
  Synthetic sequence_portion of IL2
APTSSSTKKTQLQLEHLLLHLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFSLSIISTLT

SEQ ID NO: 77 - protein, 133 aa, Artificial Sequence [sequence omitted]

SEQ ID NO: 78 - protein, 133 aa, Artificial Sequence
  Synthetic sequence_portion of IL2
APTSSSTKKTQLQLEHLLLDLQMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFCQSIISTLT

SEQ ID NO: 79 - protein, 493 aa, Artificial Sequence
  Synthetic sequence_CTLA4-Fc-IL2 chimeric protein
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDAPTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYACETATIVEFLNRWITFSQSIISTLT

SEQ ID NO: 80 - protein, 493 aa, Artificial Sequence
  Synthetic sequence_CTLA4-Fc-IL2 chimeric protein
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDAPTSSSTKKTQLQLEHLLLHLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFSLSIISTLT

SEQ ID NO: 81 - protein, 493 aa, Artificial Sequence
  Synthetic sequence_CTLA4-Fc-IL2 chimeric protein
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 82 - protein, 493 aa, Artificial Sequence
  Synthetic sequence_CTLA4-Fc-IL2 chimeric protein
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDAPTSSSTKKTQLQLEHLLLDLQMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFCQSIISTLT

SEQ ID NO: 83 - protein, 750 aa, Artificial Sequence
  Synthetic sequence_portion of SEMA3E
ADTTHPRLRLSHKELLNLNRTSIFHSPFGFLDLHTMLLDEYQERLFVGGRDLVYSLSLERISDGYKEIHWPSTALKMEECIMKGKDAGECANYVRVLHHYNRTHLLTCGTGAFDPVCAFIRVGYHLEDPLFHLESPRSERGRGRCPFDPSSSFISTLIGSELFAGLYSDYWSRDAAIFRSMGRLAHIRTEHDDERLLKEPKFVGSYMIPDNEDRDDNKVYFFFTEKALEAENNAHAIYTRVGRLCVNDVGGQRILVNKWSTFLKARLVCSVPGMNGIDTYFDELEDVFLLPTRDHKNPVIFGLFNTTSNIFRGHAICVYHMSSIRAAFNGPYAHKEGPEYHWSVYEGKVPYPRPGSCASKVNGGRYGTTKDYPDDAIRFARSHPLMYQAIKPAHKKPILVKTDGKYNLKQIAVDRVEAEDGQYDVLFIGTDNGIVLKVITIYNQEMESMEEVILEELQIFKDPVPIISMEISSKRQQLYIGSASAVAQVRFHHCDMYGSACADCCLARDPYCAWDGISCSRYYPTGTHAKRRFRRQDVRHGNAAQQCFGQQFVGDALDKTEEHLAYGIENNSTLLECTPRSLQAKVIWFVQKGRETRKEEVKTDDRVVKMDLGLLFLRLHKSDAGTYFCQTVEHSFVHTVRKITLEVVEEEKVEDMFNKDDEEDRHHRMPCPAQSSISQGAKPWYKEFLQLIGYSNFQRVEEYCEKVWCTDRKRKKLKMSPSKWKYANPQEKKLRSKPEHYRLPRHTLDS

SEQ ID NO: 84 - protein, 1110 aa, Artificial Sequence
  Synthetic sequence_CTLA4-Fc-SEMA3E chimeric protein
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDADTTHPRLRLSHKELLNLNRTSIFHSPFGFLDLHTMLLDEYQERLFVGGRDLVYSLSLERISDGYKEIHWPSTALKMEECIMKGKDAGECANYVRVLHHYNRTHLLTCGTGAFDPVCAFIRVGYHLEDPLFHLESPRSERGRGRCPFDPSSSFISTLIGSELFAGLYSDYWSRDAAIFRSMGRLAHIRTEHDDERLLKEPKFVGSYMIPDNEDRDDNKVYFFFTEKALEAENNAHAIYTRVGRLCVNDVGGQRILVNKWSTFLKARLVCSVPGMNGIDTYFDELEDVFLLPTRDHKNPVIFGLFNTTSNIFRGHAICVYHMSSIRAAFNGPYAHKEGPEYHWSVYEGKVPYPRPGSCASKVNGGRYGTTKDYPDDAIRFARSHPLMYQAIKPAHKKPILVKTDGKYNLKQIAVDRVEAEDGQYDVLFIGTDNGIVLKVITIYNQEMESMEEVILEELQIFKDPVPIISMEISSKRQQLYIGSASAVAQVRFHHCDMYGSACADCCLARDPYCAWDGISCSRYYPTGTHAKRRFRRQDVRHGNAAQQCFGQQFVGDALDKTEEHLAYGIENNSTLLECTPRSLQAKVIWFVQKGRETRKEEVKTDDRVVKMDLGLLFLRLHKSDAGTYFCQTVEHSFVHTVRKITLEVVEEEKVEDMFNKDDEEDRHHRMPCPAQSSISQGAKPWYKEFLQLIGYSNFQRVEEYCEKVWCTDRKRKKLKMSPSKWKYANPQEKKLRSKPEHYRLPRHTLDS

SEQ ID NO: 85 - protein, 264 aa, Artificial Sequence
  Synthetic sequence_extracellular domain of VSIG4
RPILEVPESVTGPWKGDVNLPCTYDPLQGYTQVLVKWLVQRGSDPVTIFLRDSSGDHIQQAKYQGRLHVSHKVPGDVSLQLSTLEMDDRSHYTCEVTWQTPDGNQVVRDKITELRVQKLSVSKPTVTTGSGYGFTVPQGMRISLQCQARGSPPISYIWYKQQTNNQEPIKVATLSTLLFKPAVIADSGSYFCTAKGQVGSEQHSDIVKFVVKDSSKLLKTKTEAPTTMTYPLKATSTVKQSWDWTTDMDGYLGETSAGPGKSLP

SEQ ID NO: 86 - protein, 631 aa, Artificial Sequence
  Synthetic sequence_VSIG4-Fc-IL2 chimeric protein
RPILEVPESVTGPWKGDVNLPCTYDPLQGYTQVLVKWLVQRGSDPVTIFLRDSSGDHIQQAKYQGRLHVSHKVPGDVSLQLSTLEMDDRSHYTCEVTWQTPDGNQVVRDKITELRVQKLSVSKPTVTTGSGYGFTVPQGMRISLQCQARGSPPISYIWYKQQTNNQEPIKVATLSTLLFKPAVIADSGSYFCTAKGQVGSEQHSDIVKFVVKDSSKLLKTKTEAPTTMTYPLKATSTVKQSWDWTTDMDGYLGETSAGPGKSLPSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDAPTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYACETATIVEFLNRWITFSQSIISTLT

SEQ ID NO: 87 - protein, 631 aa, Artificial Sequence
  Synthetic sequence_VSIG4-Fc-IL2 chimeric protein
RPILEVPESVTGPWKGDVNLPCTYDPLQGYTQVLVKWLVQRGSDPVTIFLRDSSGDHIQQAKYQGRLHVSHKVPGDVSLQLSTLEMDDRSHYTCEVTWQTPDGNQVVRDKITELRVQKLSVSKPTVTTGSGYGFTVPQGMRISLQCQARGSPPISYIWYKQQTNNQEPIKVATLSTLLFKPAVIADSGSYFCTAKGQVGSEQHSDIVKFVVKDSSKLLKTKTEAPTTMTYPLKATSTVKQSWDWTTDMDGYLGETSAGPGKSLPSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDAPTSSSTKKTQLQLEHLLLHLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFSLSIISTLT

SEQ ID NO: 88 - protein, 631 aa, Artificial Sequence
  Synthetic sequence_VSIG4-Fc-IL2 chimeric protein
RPILEVPESVTGPWKGDVNLPCTYDPLQGYTQVLVKWLVQRGSDPVTIFLRDSSGDHIQQAKYQGRLHVSHKVPGDVSLQLSTLEMDDRSHYTCEVTWQTPDGNQVVRDKITELRVQKLSVSKPTVTTGSGYGFTVPQGMRISLQCQARGSPPISYIWYKQQTNNQEPIKVATLSTLLFKPAVIADSGSYFCTAKGQVGSEQHSDIVKFVVKDSSKLLKTKTEAPTTMTYPLKATSTVKQSWDWTTDMDGYLGETSAGPGKSLPSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 89 - protein, 631 aa, Artificial Sequence
  Synthetic sequence_VSIG4-Fc-IL2 chimeric protein
RPILEVPESVTGPWKGDVNLPCTYDPLQGYTQVLVKWLVQRGSDPVTIFLRDSSGDHIQQAKYQGRLHVSHKVPGDVSLQLSTLEMDDRSHYTCEVTWQTPDGNQVVRDKITELRVQKLSVSKPTVTTGSGYGFTVPQGMRISLQCQARGSPPISYIWYKQQTNNQEPIKVATLSTLLFKPAVIADSGSYFCTAKGQVGSEQHSDIVKFVVKDSSKLLKTKTEAPTTMTYPLKATSTVKQSWDWTTDMDGYLGETSAGPGKSLPSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDAPTSSSTKKTQLQLEHLLLDLQMILNGINNYKNPKLTRMLTFKFYMPKKATELKHLQCLEEELKPLEEVLNLAQSKNFHFDPRDVVSNINVFVLELKGSETTFMCEYADETATIVEFLNRWITFCQSIISTLT

SEQ ID NO: 90 - protein, 235 aa, Artificial Sequence
  Synthetic sequence_extracellular domain of TNFR2
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGD

SEQ ID NO: 91 - protein, 299 aa, Artificial Sequence
  Synthetic sequence_extracellular domain of MadCAM
QSLQVKPLQVEPPEPVVAVALGASRQLTCRLACADRGASVQWRGLDTSLGAVQSDTGRSVLTVRNASLSAAGTRVCVGSCGGRTFQHTVQLLVYAFPDQLTVSPAALVPGDPEVACTAHKVTPVDPNALSFSLLVGGQELEGAQALGPEVQEEEEEPQGDEDVLFRVTERWRLPPLGTPVPPALYCQATMRLPGLELSHRQAIPVLHSPTSPEPPDTTSPESPDTTSPESPDTTSQEPPDTTSPEPPDKTSPEPAPQQGSTHTPRSPGSTRTRRPEISQAGPTQGEVIPTGSSKPAGDQ

SEQ ID NO: 92 - protein, 150 aa, Artificial Sequence
  Synthetic sequence_extracellular domain of CCL25
MNLWLLACLVAGFLGAWAPAVHTQGVFEDCCLAYHYPIGWAVLRRAWTYRIQEVSGSCNLPAAIFYLPKRHRKVCGNPKSREVQRAMKLLDARNKVFAKLHHNTQTFQAGPHAVKKLSSGNSKLSSSKFSNPISSSKRNVSLLISANSGL

SEQ ID NO: 93 - protein, 679 aa, Artificial Sequence
  Synthetic sequence_MadCAM-Fc-CCL25 chimeric protein
MEWSWVFLFFLSVTTGVHSQSLQVKPLQVEPPEPVVAVALGASRQLTCRLACADRGASVQWRGLDTSLGAVQSDTGRSVLTVRNASLSAAGTRVCVGSCGGRTFQHTVQLLVYAFPDQLTVSPAALVPGDPEVACTAHKVTPVDPNALSFSLLVGGQELEGAQALGPEVQEEEEEPQGDEDVLFRVTERWRLPPLGTPVPPALYCQATMRLPGLELSHRQAIPVLHSPTSPEPPDTTSPESPDTTSPESPDTTSQEPPDTTSPEPPDKTSPEPAPQQGSTHTPRSPGSTRTRRPEISQAGPTQGEVIPTGSSKPAGDQSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDQGVFEDCCLAYHYPIGWAVLRRAWTYRIQEVSGSCNLPAAIFYLPKRHRKVCGNPKSREVQRAMKLLDARNKVFAKLHHNTQTFQAGPHAVKKLSSGNSKLSSSKFSNPISSSKRNVSLLISANSGL

SEQ ID NO: 94 - protein, 345 aa, Artificial Sequence
  Synthetic sequence_extracellular domain of IL-6RA
APRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLP

SEQ ID NO: 95 - protein, 597 aa, Artificial Sequence
  Synthetic sequence_extracellular domain of IL-6ST
ELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIE

SEQ ID NO: 96 - protein, 229 aa, Artificial Sequence
  Synthetic sequence_portion of EBI3
MTPQLLLALVLWASCPPCSGRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 97 - protein, 219 aa, Artificial Sequence
  Synthetic sequence_portion of IL-12A
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 98 - protein, 1062 aa, Artificial Sequence
  Synthetic sequence_Alpha Chain (gp130-Fc-IL-12A or IL-6ST-Fc- IL-12A) of the IL-6R-Fc-IL-35 chimeric protein
MEWSWVFLFFLSVTTGVHSELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEGSGSRKGGKRGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKDEGGEDGSGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 99 - protein, 823 aa, Artificial Sequence
  Synthetic sequence_Beta Chain (IL-6RA-Fc-IL27B or IL-6RA-Fc- EBI3) of the IL-6R-Fc-IL-35 chimeric protein
MEWSWVFLFFLSVTTGVHSLAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLPGSGSDEGGEDGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKRKGGKRGSGSRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 100 - protein, 235 aa, Artificial Sequence; identical to SEQ ID NO: 90
  Synthetic sequence_extracellular domain of TNFR2

SEQ ID NO: 101 - protein, 390 aa, Artificial Sequence
  Synthetic sequence_extracellular domain of TNFR2
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 102 - protein, 600 aa, Artificial Sequence
  Synthetic sequence_TNFR2-Fc-TGFbeta chimeric protein
MEFGLSWVFLVAIIKGVQCLPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 103 - protein, 943 aa, Artificial Sequence
  Synthetic sequence_portion of integrin alpha4
NVDTESALLYQGPHNTLFGYSVVLHSHGANRWLLVGAPTANWLANASVINPGAIYRCRIGKNPGQTCEQLQLGSPNGEPCGKTCLEERDNQWLGVTLSRQPGENGSIVTCGHRWKNIFYIKNENKLPTGGCYGVPPDLRTELSKRIAPCYQDYVKKFGENFASCQAGISSFYTKDLIVMGAPGSSYWTGSLFVYNITTNKYKAFLDKQNQVKFGSYLGYSVGAGHFRSQHTTEVVGGAPQHEQIGKAYIFSIDEKELNILHEMKGKKLGSYFGASVCAVDLNADGFSDLLVGAPMQSTIREEGRVFVYINSGSGAVMNAMETNLVGSDKYAARFGESIVNLGDIDNDGFEDVAIGAPQEDDLQGAIYIYNGRADGISSTFSQRIEGLQISKSLSMFGQSISGQIDADNNGYVDVAVGAFRSDSAVLLRTRPVVIVDASLSHPESVNRTKFDCVENGWPSVCIDLTLCFSYKGKEVPGYIVLFYNMSLDVNRKAESPPRFYFSSNGTSDVITGSIQVSSREANCRTHQAFMRKDVRDILTPIQIEAAYHLGPHVISKRSTEEFPPLQPILQQKKEKDIMKKTINFARFCAHENCSADLQVSAKIGFLKPHENKTYLAVGSMKTLMLNVSLFNAGDDAYETTLHVKLPVGLYFIKILELEEKQINCEVTDNSGVVQLDCSIGYIYVDHLSRIDISFLLDVSSLSRAEEDLSITVHATCENEEEMDNLKHSRVTVAIPLKYEVKLTVHGFVNPTSFVYGSNDENEPETCMVEKMNLTFHVINTGNSMAPNVSVEIMVPNSFSPQTDKLFNILDVQTTTGECHFENYQRVCALEQQKSAMQTLKGIVRFLSKTDKRLLYCIKADPHCLNFLCNFGKMESGKEASVHIQLEGRPSILEMDETSALKFEIRATGFPEPNPRVIELNKDENVAHVLLEGLHHQRPKRYFT

SEQ ID NO: 104 - protein, 704 aa, Artificial Sequence
  Synthetic sequence_portion of integrin beta7
ELDAKIPSTGDATEWRNPHLSMLGSCQPAPSCQKCILSHPSCAWCKQLNFTASGEAEARRCARREELLARGCPLEELEEPRGQQEVLQDQPLSQGARGEGATQLAPQRVRVTLRPGEPQQLQVRFLRAEGYPVDLYYLMDLSYSMKDDLERVRQLGHALLVRLQEVTHSVRIGFGSFVDKTVLPFVSTVPSKLRHPCPTRLERCQSPFSFHHVLSLTGDAQAFEREVGRQSVSGNLDSPEGGFDAILQAALCQEQIGWRNVSRLLVFTSDDTFHTAGDGKLGGIFMPSDGHCHLDSNGLYSRSTEFDYPSVGQVAQALSAANIQPIFAVTSAALPVYQELSKLIPKSAVGELSEDSSNVVQLIMDAYNSLSSTVTLEHSSLPPGVHISYESQCEGPEKREGKAEDRGQCNHVRINQTVTFWVSLQATHCLPEPHLLRLRALGFSEELIVELHTLCDCNCSDTQPQAPHCSDGQGHLQCGVCSCAPGRLGRLCECSVAELSSPDLESGCRAPNGTGPLCSGKGHCQCGRCSCSGQSSGHLCECDDASCERHEGILCGGFGRCQCGVCHCHANRTGRACECSGDMDSCISPEGGLCSGHGRCKCNRCQCLDGYYGALCDQCPGCKTPCERHRDCAECGAFRTGPLATNCSTACAHTNVTLALAPILDDGWCKERTLDNQLFFFLVEDDARGTVVLRVRPQEKGADH

SEQ ID NO: 105 - protein, 896 aa, Artificial Sequence
  Synthetic sequence_Alpha Chain (ITG4A-Fc-IL-12A or integrin alpha4-Fc- IL-12A) of the alpha4beta7-Fc-IL-35 chimeric protein
MEFGLSWVFLVAIIKGVQCYNVDTESALLYQGPHNTLFGYSVVLHSHGANRWLLVGAPTANWLANASVINPGAIYRCRIGKNPGQTCEQLQLGSPNGEPCGKTCLEERDNQWLGVTLSRQPGENGSIVTCGHRWKNIFYIKNENKLPTGGCYGVPPDLRTELSKRIAPCYQDYVKKFGENFASCQAGISSFYTKDLIVMGAPGSSYWTGSLFVYNITTNKYKAFLDKQNQVKFGSYLGYSVGAGHFRSQHTTEVVGGAPQHEQIGKAYIFSIDEKELNILHEMKGKKLGSYFGASVCAVDLNADGFSDLLVGAPMQSTIREEGRVFVYINSGSGAVMNAMETNLVGSDKYAARFGESIVNLGDIDNDGFEDVAIGAPQEDDLQGAIYIYNGRADGISSTFSQRIEGLQISKSLSMFGQSISGQIDADNNGYVDVAVGAFRSDSAVLLRTRGSGSRKGGKRGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKDEGGEDGSGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 106 - protein, 951 aa, Artificial Sequence
  Synthetic sequence_Beta Chain (ITGB7-Fc- IL27B or integrin beta7-Fc- EBI3) of the alpha4beta7-Fc-IL-35 chimeric protein
MEFGLSWVFLVAIIKGVQCMVALPMVLVLLLVLSRGESELDAKIPSTGDATEWRNPHLSMLGSCQPAPSCQKCILSHPSCAWCKQLNFTASGEAEARRCARREELLARGCPLEELEEPRGQQEVLQDQPLSQGARGEGATQLAPQRVRVTLRPGEPQQLQVRFLRAEGYPVDLYYLMDLSYSMKDALERVRQLGHALLVRLQEVTHSVRIGFGSFVDKTVLPFVSTVPSKLRHPCPTRLERCQSPFSFHHVLSLTGDAQAFEREVGRQSVSGNLDSPEGGFDAILQAALCQEQIGWRNVSRLLVFTSDDTFHTAGDGKLGGIFMPSDGHCHLDSNGLYSRSTEFDYPSVGQVAQALSAANIQPIFAVTSAALPVYQELSKLIPKSAVGELSEDSSNVVQLIMDAYNSLSSTVTLEHSSLPPGVHISYESQCEGPEKREGKAEDRGQCNHVRINQTVTFWVSLQATHCLPEPHLLRLRALGFSEELIVELHTLCGSGSDEGGEDGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKRKGGKRGSGSRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK